SEQUENCE LISTING (32 sequences)

SEQ ID NO: 1 - protein, 208 aa, Homo sapiens
MWKWILTHCASAFPHLPGCCCCCFLLLFLVSSVPVTCQALGQDMVSPEATNSSSSSFSSPSSAGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 2 - protein, 145 aa, Homo sapiens
GRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 3 - protein, 376 aa, Artificial Sequence
  Description of Artificial Sequence Synthetic polypeptide
MASYPCHQHASAFDQAARSRGHNNRRTALRPRRQQKATEVRLEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTIFLDRHPIAFMLCYPAARYLMGSMTPQAVLAFVALIPPTLPGTNIVLGALPEDRHIDRLAKRQRPGERLDLAMLAAIRRVYGLLANTVRYLQGGGSWREDWGQLSGAAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 4 - protein, 538 aa, Artificial Sequence
  Description of Artificial Sequence Synthetic polypeptide
MHHHHHHMGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHSGGGGSGGGGSASYPCHQHASAFDQAARSRGHNNRRTALRPRRQQKATEVRLEQKMPTLLRVYIDGPHGMGKTTTTQLLVALGSRDDIVYVPEPMTYWRVLGASETIANIYTTQHRLDQGEISAGDAAVVMTSAQITMGMPYAVTDAVLAPHIGGEAGSSHAPPPALTIFLDRHPIAFMLCYPAARYLMGSMTPQAVLAFVALIPPTLPGTNIVLGALPEDRHIDRLAKRQRPGERLDLAMLAAIRRVYGLLANTVRYLQGGGSWREDWGQLSGAAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 5 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 6 - protein, 335 aa, Artificial Sequence
  Description of Artificial Sequence Synthetic polypeptide
MEQKLISEEDLGSGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHSGHGGGGSGGGGSMEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPDGHGHHHHHH

SEQ ID NO: 7 - protein, 335 aa, Artificial Sequence
  Description of Artificial Sequence Synthetic polypeptide
MHHHHHHGSMEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPDGHGGGGSGGGGSGRHVRSYNHLQGDVRWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHSGHGEQKLISEEDL

SEQ ID NO: 8 - protein, 145 aa, Artificial Sequence
  Description of Artificial Sequence Synthetic polypeptide
GRHVRSYNHLQGDVRWRKLFSFTKYFLKIEKNGKVSGTKKENCPYSILEITSVEIGVVAVKAINSNYYLAMNKKGKLYGSKEFNNDCKLKERIEKNGYNTYASFNWQHNGRQMYVALNGKGAPRRGQKTRRANTSAHFLPMVVHS

SEQ ID NO: 9 - protein, 145 aa, Artificial Sequence
  Description of Artificial Sequence Synthetic polypeptide
GRHVRSYNHLQGDVAWRKLFSFTKYFLKIEKNGKVSGTKKENCPYSILEITSVEIGVVAVKAINSNYYLAMNKKGKLYGSKEFNNDCKLKERIEENGYNTYASFNWQHNGRQMYVALNGKGAPRRGQKTRRKNTSAHFLPMVVHS

SEQ ID NO: 10 - protein, 145 aa, Artificial Sequence
  Description of Artificial Sequence Synthetic polypeptide
GRHVRSYNHLQGDVAWRKLFSFTKYFLKIEKNGKVSGTKKENCPYSILEIRSVEIGVVAVKAINSNYYLAMNKKGKLYGSKEFNNDCKLKERIEANGYNTYASFNWQHNGRQMYVALNGKGAPRRGQKTRRANTSAHFLPMVVHS

SEQ ID NO: 11 - protein, 416 aa, Artificial Sequence
  Description of Artificial Sequence Synthetic polypeptide
MEQKLISEEDLGSGRHVRSYNHLQGDVAWRKLFSFTKYFLKIEKNGKVSGTKKENCPYSILEIRSVEIGVVAVKAINSNYYLAMNKKGKLYGSKEFNNDCKLKERIEANGYNTYASFNWQHNGRQMYVALNGKGAPRRGQKTRRANTSAHFLPMVVHSGHGGGGSGGGGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKLEGHGHHHHHH

SEQ ID NO: 12 - protein, 11 aa, Human immunodeficiency virus
YGRKKRRQRRR

SEQ ID NO: 13 - protein, 16 aa, Drosophila sp.
RQIKIWFQNRRMKWKK

SEQ ID NO: 14 - protein, 19 aa, Unknown
  Description of Unknown SynB1 peptide
RGGRLSYSRRRFSTSTGRA

SEQ ID NO: 15 - protein, 27 aa, Unknown
  Description of Unknown Transportan peptide
GWTLNSAGYLLGKINLKALAALAKKIL

SEQ ID NO: 16 - protein, 34 aa, Herpes simplex virus
DAATATRGRSAASRPTERPRAPARSASRPRRPVE

SEQ ID NO: 17 - protein, 9 aa, Artificial Sequence
  Description of Artificial Sequence Synthetic peptide
RRRRRRRRR

SEQ ID NO: 18 - protein, 9 aa, Artificial Sequence
  Description of Artificial Sequence Synthetic peptide
KKKKKKKKK

SEQ ID NO: 19 - protein, 11 aa, Mus sp.
YARVRRRGPRR

SEQ ID NO: 20 - protein, 9 aa, Unknown
  Description of Unknown Sim-2 peptide
AKAARQAAR

SEQ ID NO: 21 - protein, 26 aa, Unknown
  Description of Unknown MTS peptide
AAVALLPAVLLALLAPAAADQNQLMP

SEQ ID NO: 22 - protein, 21 aa, Unknown
  Description of Unknown Pep-1 peptide
KETWWETWWTEWSQPKKKRKV

SEQ ID NO: 23 - protein, 21 aa, Unknown
  Description of Unknown Pep-2 peptide
KETWFETWFTEWSQPKKKRKV

SEQ ID NO: 24 - protein, 4 aa, Artificial Sequence
  Description of Artificial Sequence Synthetic peptide
AGVF

SEQ ID NO: 25 - protein, 4 aa, Artificial Sequence
  Description of Artificial Sequence Synthetic peptide
GFLG

SEQ ID NO: 26 - protein, 4 aa, Artificial Sequence
  Description of Artificial Sequence Synthetic peptide
ALAL

SEQ ID NO: 27 - protein, 5 aa, Artificial Sequence
  Description of Artificial Sequence Synthetic peptide
ALALA

SEQ ID NO: 28 - protein, 6 aa, Artificial Sequence
  Description of Artificial Sequence Synthetic 6xHis tag
HHHHHH

SEQ ID NO: 29 - protein, 10 aa, Artificial Sequence
  Description of Artificial Sequence Synthetic peptide
GGGGSGGGGS

SEQ ID NO: 30 - protein, 13 aa, Artificial Sequence
  Description of Artificial Sequence Synthetic peptide
GGSGGGGSGGGGS

SEQ ID NO: 31 - protein, 5 aa, Artificial Sequence
  Description of Artificial Sequence Synthetic peptide
GGGGS

SEQ ID NO: 32 - protein, 10 aa, Artificial Sequence
  Description of Artificial Sequence Synthetic peptide
EQKLISEEDL

(19) United States
(12) Patent Application Publication
(10) Pub. No.: US 2015/0307576 A1
(43) Pub. Date: Oct. 29, 2015

(54) FGF-10 COMPLEXES

(71) Applicant: PERMEON BIOLOGICS, INC., Cambridge, MA (US)

(72) Inventors: Katherine S. Bowdish, Boston, MA (US); Erik M. Vogan, Medford, MA (US); John F. Ross, Arlington, MA (US); Ann DeWitt, Cambridge, MA (US)

(73) Assignee: PERMEON BIOLOGICS, INC., Cambridge, MA (US)

(21) Appl. No.: 14/650,207

(22) PCT Filed: Dec. 6, 2013
(86) PCT No.: PCT/US13/73709

§ 371 Date: Jun. 5, 2015

Related U.S. Application Data
(60) Provisional application No. 61/734,876, filed on Dec. 7, 2012.
(60) Provisional application No. 61/879,359, filed on Sep. 18, 2013.

(51) Int. Cl. C07K 14/50; C12N 9/12; C12N 9/10; C07K 14/47

(52) U.S. Cl. CPC C07K 14/50; C07K 14/4703; C12N 9/1211; C12N 9/10; C07K 2319/10; C07K 2319/21; C07K 2319/41; A61K 38/00

(57) ABSTRACT

The present disclosure provides complexes comprising an FGF-10 portion and a heterologous protein or peptide, as well as methods of using such complexes.

### RELATED APPLICATIONS

This application claims the benefit of U.S. provisional application 61/734,876, filed Dec. 7, 2012, and U.S. provisional application 61/879,359, filed Sep. 18, 2013. The disclosures of each of the foregoing applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Dec. 6, 2013, is named 109354-0013-WO1_SL.txt and is 31,752 bytes in size.

### BACKGROUND OF THE DISCLOSURE

Fibroblast growth factor 10 (FGF-10) is a member of the FGF family. FGF family members possess broad mitogenic and cell survival activities, and are involved in a variety of biological processes, including embryonic development, cell growth, morphogenesis, tissue repair, tumor growth and invasion. The primary receptor mediating these activities of FGF-10 is FGFR-2b (also known as FGFR2-IIIb). FGF-10 can also bind FGFR-1b; albeit with only about 10% of the binding affinity of FGF-10 to FGFR-2b.

In humans, the FGF-10 polypeptide has an initial precursor, unprocessed, naturally occurring form. This unprocessed (or precursor) human polypeptide is 208 amino acids in length (See SEQ ID NO: 1). The precursor polypeptide is processed at the N-terminus to yield a mature form of 171 amino acids in length.

The present disclosure, however, is based on a different property of FGF-10 polypeptide: the ability to interact with proteoglycans on cells and to penetrate cells, such as via endocytosis.

### SUMMARY OF THE DISCLOSURE

The present disclosure is based on the appreciation that domains of FGF-10 polypeptides penetrate cells. Interestingly, this cell penetration activity is not dependent on binding of FGF-10 to the FGFR-2b, and there is little correlation between FGFR-2b expression and FGF-10 mediated cell penetration. Moreover, cell penetrating domains of FGF-10 can be conjugated to cargo proteins or peptides to facilitate penetration of that cargo into cells. These features of FGF-10 polypeptides provide the opportunity to make and use complexes comprising domains of FGF-10 polypeptides, as well as variants thereof.

The present disclosure provides compositions and methods suitable for use in delivering therapeutics into cells, particularly into cells of the liver and other organs of the abdominal cavity. Specifically, the present disclosure provides complexes comprising a cell penetrating portion of FGF-10 for delivery of therapeutics into cells and tissues in humans and in non-human animals.

In a first aspect the disclosure provides a complex comprising (i) an FGF-10 portion comprising a domain of a full length, unprocessed, naturally occurring fibroblast growth factor receptor 10 (FGF-10) polypeptide having a net positive charge and a charge per molecular weight ratio greater than that of a corresponding full length, unprocessed, naturally occurring FGF-10 polypeptide and (ii) a cargo portion comprising a heterologous protein or peptide or a small organic molecule. In certain embodiments, the complex does not include a full length, unprocessed, naturally occurring FGF-10 polypeptide.

In a second aspect, the disclosure provides a complex comprising (i) an FGF-10 portion consisting of a domain of a full length, unprocessed, naturally occurring fibroblast growth factor receptor 10 (FGF-10) polypeptide having a net positive charge and a charge per molecular weight ratio greater than that of a corresponding full length, unprocessed, naturally occurring FGF-10 polypeptide and (ii) a cargo portion comprising a heterologous protein or peptide or a small organic molecule. In certain embodiments, the complex does not include a full length, unprocessed, naturally occurring FGF-10 polypeptide.

In a third aspect, the disclosure provides a complex comprising (i) an FGF-10 portion comprising a domain of a full length, unprocessed, naturally occurring fibroblast growth factor receptor 10 (FGF-10) polypeptide having a net positive charge and a charge per molecular weight ratio greater than that of a corresponding full length, unprocessed, naturally occurring FGF-10 polypeptide, which domain is a variant that retains cell penetrating activity and (ii) a cargo portion comprising a heterologous protein or peptide or a small organic molecule. In certain embodiments, the complex does not include a full length, unprocessed, naturally occurring FGF-10 polypeptide.

In a fourth aspect, the disclosure provides a complex comprising (i) an FGF-10 portion consisting of a domain of a full length, unprocessed, naturally occurring fibroblast growth factor receptor 10 (FGF-10) polypeptide having a net positive charge and a charge per molecular weight ratio greater than that of a corresponding full length, unprocessed, naturally occurring FGF-10 polypeptide, which domain is a variant that retains cell penetrating activity and (ii) a cargo portion comprising a heterologous protein or peptide or a small organic molecule. In certain embodiments, the complex does not include a full length, unprocessed, naturally occurring FGF-10 polypeptide.

In a fifth aspect, the disclosure provides a complex comprising (i) an FGF-10 portion comprising a cell penetrating variant of a full length, unprocessed, naturally occurring fibroblast growth factor receptor 10 (FGF-10) polypeptide and (ii) a cargo portion comprising a heterologous protein or peptide or a small organic molecule.

In a sixth aspect, the disclosure provides a complex suitable for cell penetration comprising an FGF-10 portion comprising (A) (i) a full length, unprocessed, naturally occurring fibroblast growth factor receptor 10 (FGF-10) polypeptide or (ii) a mature, naturally occurring fibroblast growth factor 10 (FGF-10) polypeptide and (B) a cargo portion comprising a heterologous protein or peptide or a small organic molecule for delivery into a cell.

The disclosure contemplates that any of the embodiments set forth below may further describe any of the foregoing or following aspects of the invention. Moreover, such embodiments may be combined with one another.

In certain embodiments, the complex further comprises a linker that interconnects the FGF-10 portion and the cargo portion.

In certain embodiments, the FGF-10 polypeptide is a human FGF-10 polypeptide.

In certain embodiments, the domain of a full length, unprocessed, naturally occurring FGF-10 polypeptide is a domain of a full length, unprocessed, naturally occurring human FGF-10 polypeptide.

In certain embodiments, the FGF10 portion comprises a variant, and the variant comprises one, two, three, four, or five amino acid substitutions, deletions, and/or additions relative to the corresponding domain of the naturally occurring FGF-10 polypeptide. In certain embodiments, the variant has decreased binding affinity for FGFR2b relative to a naturally occurring, mature FGF-10 polypeptide. In certain embodiments, the variant has decreased mitogenic activity relative to a naturally occurring, mature FGF-10 polypeptide. In certain embodiments, the FGF-10 portion, and/or the domain and/or the complex has decreased binding affinity for FGFR2b relative to a naturally occurring, mature FGF-10 polypeptide. In certain embodiments, the FGF-10 portion, and/or the domain and/or the complex has decreased mitogenic activity relative to a naturally occurring, mature FGF-10 polypeptide. In certain embodiments, the variant has the same or substantially the same net theoretical charge and/or charge per molecular weight ratio as the corresponding domain of the naturally occurring FGF-10 polypeptide.

In certain embodiments, the domain of an FGF10 polypeptide has a charge/molecular weight ratio of at least 0.75, but the full length, unprocessed, naturally occurring human FGF-10 polypeptide has a charge/molecular weight ratio of less than 0.75. In certain embodiments, the domain has a charge per molecular weight ratio greater than that of the naturally occurring, mature form of the corresponding FGF-10 polypeptide. In certain embodiments, other than the domain, the complex does not include sufficient additional amino acid sequence from said FGF-10 polypeptide contiguous with said domain such that the charge/molecular weight ratio of the FGF-10 portion would be less than 0.75.

In certain embodiments, the domain of FGF10 polypeptide used in a complex of the disclosure is less than 171 amino acid residues. In certain embodiments, the domain of FGF10 used in the complex is less than 150 amino acid residues. In certain embodiments, the domain is less than or equal to 145 amino acid residues.

In certain embodiments, the domain is greater than or equal to 141 amino acid residues. In certain embodiments, the domain comprises an amino acid sequence set forth in SEQ ID NO: 2.

In certain embodiments, the domain is a variant having one, two, three, four, or five amino acid substitutions, deletions, or additions relative to the amino acid sequence set forth in SEQ ID NO: 2. In certain embodiments, the domain is a variant having an amino acid sequence at least 80%, 85%, 90%, 91%, 92%, 93%, 94%, 95%, 96%, 97%, 98%, 99%, or greater than 99% identical to SEQ ID NO: 2. In certain embodiments, the variant has decreased binding affinity for FGFR2b relative to a naturally occurring, mature FGF-10 polypeptide. In certain embodiments, the variant has decreased mitogenic activity relative to a naturally occurring, mature FGF-10 polypeptide. In certain embodiments, the domain is a variant having one, two, three, or four amino acid substitutions, relative to SEQ ID NO: 2. For example, the FGF-10 portion comprises SEQ ID NO: 2, in the presence of one, two, three, or four amino acid substitutions, such as substitutions that result in a variant having decreased binding affinity for FGFR2b relative to that of a naturally occurring, mature FGF-10 polypeptide and/or decreased mitogenic activity relative to a naturally occurring, mature FGF-10 polypeptide.

In certain embodiments, the domain of FGF10 used in a complex of the disclosure has a charge/molecular weight ratio of at least 1.0. In certain embodiments, the domain has a charge/molecular weight ratio of at least 0.9. In certain embodiments, the domain of FGF10 used in the complex has a molecular weight of at least about 14 kDa (or of about 14 kDa), at least about 15 kDa (or of about 15 kDa), or at least about 16 kDa (or of about 16 kDa). However, smaller domains, such as domains having a molecular weight of about 7 kDa, 8 kDa, 9 kDa, 10 kDa, 11 kDa, 12 kDa, or about 13 kDa are also contemplated. Note that molecular weight may refer to predicted or theoretical molecular weight.

In certain embodiments, the domain of FGF10 used has a theoretical net charge of about +12, about +13, about +14, about +15, or about +16. However, domains having a less positive net charge, such as about +7, +8, +9, +10, or +11 are also contemplated.

In certain embodiments, the domain does not consist of residues 69-208 of SEQ ID NO: 1. In certain embodiments, the domain does not consist of a mature, naturally occurring FGF-10 polypeptide. In certain embodiments, the domain corresponds to the mature FGF10 polypeptide.

In certain embodiments, the complex can penetrate a cell. In certain embodiments, the complex can penetrate a liver cell. In certain embodiments, the complex is penetrates cells non-ubiquitously, such that preferential penetration of certain cell and tissues types occurs (e.g., liver, kidney, pancreas.

In certain embodiments, the cargo portion comprises a heterologous polypeptide or peptide. In other embodiments, the cargo portion comprises a small organic molecule, such as an organic molecule of less than 1000, less than 750, less than 650, or less than about 500 amu.

In certain embodiments, the cargo portion does not include a ligand binding domain of an FGF receptor. In certain embodiments, the cargo portion does not include a native binding partner of FGF-10. In certain embodiments, neither the cargo portion nor the complex includes heparin. In certain embodiments, neither the cargo portion nor the complex includes a ligand binding domain of an FGF receptor and/or a native binding partner of FGF-10.

In certain embodiments, the heterologous polypeptide or peptide is an enzyme. In certain embodiments, the enzyme is selected from a kinase, a phosphatase, a ligase, a protease, an oxidoreductase, a transferase, a hydrolase, a hydroxylase, a lyase, an isomerase, a dehydrogenase, an aminotransferase, a hexosamidase, a glucosidase, or a glucosyltransferase. In certain embodiments, the enzyme is selected from an enzyme that degrades glycosaminoglycans, glycolipids, or sphingolipids; an enzyme that degrades glycoproteins; an enzyme that degrades amino acids; or an enzyme that degrades fatty acids; or an enzyme involved in energy metabolism. In certain embodiments, the enzyme that is endogenously expressed in healthy subjects. In certain embodiments, the enzyme is not a recombinase. In certain embodiments, an endogenous activity of the enzyme in healthy subjects is in liver. In certain embodiments, the enzyme is a thymidine kinase.

In certain embodiments, the heterologous polypeptide or peptide is a transcription factor.

In certain embodiments, the heterologous polypeptide or peptide is a tumor suppressor protein. In certain embodiments, the tumor suppressor protein is p16, or a functional fragment thereof.

In certain embodiments, the heterologous polypeptide or peptide is a co-factor or member of a protein complex.

In certain embodiments, the heterologous polypeptide or peptide is a target binding moiety that binds to and inhibits a target. In certain embodiments, the heterologous polypeptide or peptide comprises an antibody or antibody mimic. In certain embodiments, the target binding moiety comprises a ligand binding domain of a receptor or a receptor binding domain of a ligand. In certain embodiments, the target binding moiety comprises a full length antibody molecule. In certain embodiments, the target binding moiety comprises an antibody fragment. In certain embodiments, the antibody fragment is a single chain antibody (scFv), a F(ab′)2 fragment, a Fab fragment, or an Fd fragment. In certain embodiments, the target binding moiety comprises a bispecific antibody. In certain embodiments, the target binding moiety comprises an antibody-mimic comprising a protein scaffold. In certain embodiments, the antibody mimic comprises a DARPin polypeptide or an Anticalin® polypeptide.

In certain embodiments, the target binding moiety binds to and inhibits a target expressed or present in liver. In other embodiments, the target binding moiety binds to and inhibits a target expressed or present in one or more tissues in which the FGF10 portion preferentially localizes (e.g., liver, kidney, pancreas, etc.). In certain embodiments, the endogenous activity of the heterologous polypeptide or peptide is as a member of a complex with a polypeptide expressed or present in liver. In certain embodiments, the endogenous activity of the heterologous polypeptide or peptide is as a member of a complex with a polypeptide expressed or present in one or more tissues in which the FGF10 portion preferentially localizes. In other words, in certain embodiments, the target binding moiety binds to a target expressed in particular tissues, and the complexes of the disclosure facilitate delivery of the target binding moiety to such tissues.

In certain embodiments, the FGF-10 portion and the cargo portion are associated non-covalently. In certain embodiments, the FGF-10 portion and the cargo portion are associated via a covalent interconnection. In certain embodiments, the FGF-10 portion and the cargo portion are interconnected by a linker. In certain embodiments, the FGF-10 portion and the cargo portion are directly interconnected without a linker. In certain embodiments, the association between the FGF-10 portion and the cargo portion includes at least one peptide bond. In certain embodiments, the FGF-10 portion and the cargo portion are associated via at least one covalent bond and at least one non-covalent interaction.

In certain embodiments, the FGF10 portion and the cargo portion form a fusion protein.

In certain embodiments, the complex further comprises one or more tags to facilitate production, purification, or detection of the complex.

In certain embodiments, the FGF-10 portion is N-terminal to the cargo portion. In other embodiments, the FGF-10 portion is C-terminal to the cargo portion.

In another aspect, the disclosure provides a nucleic acid comprising a nucleotide sequence encoding a complex of the disclosure.

In another aspect, the disclosure provides a vector comprising a nucleic acid encoding a complex of the disclosure.

In another aspect, the disclosure provides a host cell comprising a vector of the disclosure.

In another aspect, the disclosure provides a method of making a fusion protein. The method entails (i) providing a host cell containing a vector comprising a nucleic acid that encodes a complex of the disclosure in culture media and culturing the host cell under suitable condition for expression of protein therefrom and (ii) expressing the fusion protein.

In certain embodiments, a complex of the disclosure has greater than 50% (e.g., 50%, 60%, 65%, 70%, 75%, 80%, 85% 90%, 95%, 97%, 98%, 99%, 100%, or even greater than 100%) of the native activity of the cargo portion. In other words, the cargo portion, presented as part of a complex of the disclosure, has at least 50% of the activity of the native cargo portion alone. In certain embodiments, the cargo portion is an enzyme, and the complex has greater than 50% of the native activity of the enzyme.

In certain embodiments, the complex comprises a modification selected from glycosylation, phosphorylation or pegylation. In other embodiments, the complex is not glycosylated.

In another aspect, the disclosure provides a composition comprising a complex of the disclosure and a pharmaceutically acceptable carrier.

In another aspect, the disclosure provides, a method of delivering a cargo portion into a cell. The method entails providing a complex of the disclosure and contacting cells with the complex.

In another aspect, the disclosure provides method of delivering a cargo portion into a cell of the liver. The method entails providing a complex of the disclosure and contacting cells with the complex.

In another aspect, the disclosure provides a method of delivering a therapeutic protein into cells or tissues of the abdominal cavity. The method entails providing a complex of the disclosure and administering said complex to a subject in need thereof via intraperitoneal administration.

In certain embodiments, the subject in need thereof is a subject with primary or metastatic cancer in the abdominal cavity. In certain embodiments, the primary or metastatic cancer is associated with liver, kidney, pancreas, or ovary. In certain embodiments, the primary or metastatic cancer comprises a mutation that decreases the expression and/or activity of p16.

In another aspect, the disclosure provides method of delivering a target binding moiety into cells, such as cells of the liver, kidney, ovary, or pancreas. The method entails providing a complex of the disclosure and administering said complex to a subject in need thereof.

In certain embodiments, the target binding moiety binds to and inhibits a target expressed or present inside the cells. In certain embodiments, the cells are cells of the liver, kidney, pancreas, or ovary. In certain embodiments, the cells are cells in which a cell penetrating FGF10 portion localizes preferentially (e.g., localization is not ubiquitous across all cells).

In certain embodiments, the FGF-10 portion comprises an E158K/K195A FGF-10 variant. In other embodiments, the FGF-10 portion comprises an R78A FGF-10 variant. In certain embodiments, the FGF-10 portion comprises one, two, three, or four substitutions selected from: R78A, T114A, E158K, E158A, or K195A. In certain embodiments, the FGF-portion comprises or consists of the amino acid sequence set forth in SEQ ID NO: 2 in the presence of one, two, three, four, or five amino acid substitutions, wherein the amino acid substitutions decrease the mitogenic activity and/or FGFR binding activity of the FGF-10 portion, relative to the naturally occurring FGF-10 portion. In certain embodiments, the FGF-10 portion comprises an amino acid sequence set forth in SEQ ID NO: 8. In other embodiments, the FGF-10 portion comprises an amino acid sequence set forth in SEQ ID NO: 9. In other embodiments, the FGF-10 portion comprises an amino acid sequence set forth in SEQ ID NO: 10. In any of the foregoing, the disclosure contemplates complexes in which the FGF-10 portion consists of or consists essentially of any of these or other FGF-10 sequences provided herein. When the FGF10 portion consists essentially of a particular sequence, the complex does not include substantial additional contiguous sequence from the native FGF-10 polypeptide contiguous with the FGF-10 portion.

In certain embodiments, the disclosure provides a complex, such as a fusion protein, comprising an FGF10 portion and a cargo portion, wherein the FGF10 portion comprises or consists of the amino acid sequence set forth in SEQ ID NO: 2, in the presence of one, two, three, four, or five amino acid substitutions, additions, or deletions (e.g., a variant FGF10).

In certain embodiments, the complex or fusion protein does not include the full length, naturally occurring FGF10 sequence set forth in SEQ ID NO: 1. In certain embodiments, the FGF10 portion comprises one, two, three, four, or five amino acid substitutions, and such a variant FGF10 portion has decreased mitogenic activity and/or FGFR binding relative to native FGF10 in the absence of the amino acid substitution(s). In certain embodiments, the FGF10 portion comprises one, two, three, or four amino acid substitutions. In certain embodiments, the amino acid substitutions are selected from: R78A, T114A, E158K, E158A, or K195A. In certain embodiments, the one, two, three, four, or five amino acid substitutions do not substantially alter surface positive charge or net charge of the FGF10 portion (e.g., the change in charge on the FGF10 portion is +1, 0, or −1).

In certain embodiments of complexes of the disclosure, the (i) FGF-10 portion comprises or consists of a variant having 1, 2, 3, 4, or 5 amino acid substitutions and having decreased mitogenic activity and/or FGFR binding relative to native FGF10, such as the substitutions described above, and (ii) the cargo portion comprises a polypeptide, peptide, or small molecule; with the proviso that the cargo portion and/or the complex does not include one or more of: full length, unprocessed, naturally occurring FGF-10, a ligand binding domain of an FGF receptor, a native binding partner of FGF-10, and/or heparin. The cargo portion may be any of the cargo portions described herein.

In another aspect, the disclosure provides a complex comprising the amino acid sequence set forth in SEQ ID NO: 4, in the presence or absence of the N-terminal tag, and in the presence or absence of the N-terminal methionine.

The disclosure contemplates all combinations of any of the foregoing aspects and embodiments, as well as combinations with any of the embodiments set forth in the detailed description and examples.

### BRIEF DESCRIPTION OF THE DRAWINGS

FIG. 1 depicts immobilized metal affinity chromatography (IMAC) purification of Hisx6-FGF10-Myc. The FGF10 portion is a domain of human FGF10 having a charge per molecular weight ratio greater than that of full length, unprocessed, naturally occurring human FGF10.

FIG. 2 depicts cation exchange chromatography of Hisx6-FGF10-Myc. The FGF10 portion is a domain of human FGF10 having a charge per molecular weight ratio greater than that of full length, unprocessed, naturally occurring human FGF10.

FIG. 3 summarizes the purification of Hisx6-FGF10-Myc.

FIG. 4 depicts IMAC purification of the following conjugate: Hisx6-FGF10-GS10-TK. The FGF10 portion is a domain of human FGF10 having a charge/molecular weight ratio greater than that of full length, unprocessed, naturally occurring human FGF10.

FIG. 5 depicts cation exchange chromatography of Hisx6-FGF10-GS10-TK. The FGF10 portion is a domain of human FGF10 having a charge/molecular weight ratio greater than that of full length, unprocessed, naturally occurring human FGF10.

FIG. 6 depicts SEC purification of Hisx6-FGF10-GS10-TK. The FGF10 portion is a domain of human FGF10 having a charge/molecular weight ratio greater than that of full length, unprocessed, naturally occurring human FGF10.

FIG. 7 depicts overlayed SEC column chromatograms indicating 75% stability of the His6-FGF10-myc protein following multiple freeze thaw cycles.

FIGS. 8A-8D depicts the results of experiments demonstrating FGF10-mediated cell penetration.

FIG. 9 provides a schematic representation of an HSV-TK cell-based assay for evaluating cell penetration and cargo protein function following cell penetration.

FIG. 10 depicts the results of FGF10-TK-induced cell death in the HSV-TK MTT assay. OD values measure the amount of MTT dye metabolized by live cells, and thus is a measure of the degree of cell death in the well. For each protein dose, the four bars correspond to (from left to right): no protein+0 uM chloroquine; no protein+100 uM chloroquine; FGF10-TK+0 uM chloroquine; FGF10-TK+100 uM chloroquine. The three sets of bars to the left depict experiments performed at differing doses of protein in the presence of 3 uM gangiclovir

FIG. 11 depicts the results of experiments in which ug of 125I-protein per ml of blood plasma were injected via tail vein, and where blood samples were collected from mice at 5 minutes, 30 minutes, 1 hour, and 6 hours after injection for +36GFP and FGF10 and then at 5 minutes, 1 hour, 6 hours and 24 hours after injection for Tat-TK, +36GFP-TK, and FGF10-TK. Concentration was determined by a measurement of TCA precipitable radioactivity. Error bars represent standard deviation of data from 2 mice where data was available.

FIG. 12 depicts the percent of initial dose present in the blood plasma where blood samples were collected from mice at 5 minutes, 30 minutes, 1 hour, and 6 hours for +36GFP and FGF10 and then at 5 minutes, 1 hour, 6 hours and 24 hours for Tat-TK, +36GFP-TK, and FGF10-TK. This protein concentration data was adjusted by TCA precipitable counts. The initial dose concentration was determined by taking the initial dose given to the animal as determined by counting radioactivity in an aliquot of the formulated dose and then assuming this dose is distributed uniformly in the blood compartment of a mouse, estimated at 1.7 ml. Error bars represent the standard deviation of data from 2 mice where data was available.

FIG. 13 depicts the percent of initial dose present in blood versus plasma after 5 minutes. This protein concentration data is adjusted by TCA precipitable counts.

FIG. 14 provides images from liver MARG.

FIG. 15 provides images from kidney MARG.

FIG. 16 depicts immobilized metal affinity chromatography (IMAC) purification of Myc-FGF10-(G₄S)₂-p16-Hisx6. The FGF10 portion is a domain of human FGF10 having a charge per molecular weight ratio greater than that of full length, unprocessed, naturally occurring human FGF10.

FIG. 17 depicts cation exchange chromatography of Myc-FGF10-(G₄S)₂-p16-Hisx6.

FIG. 18 depicts SEC chromatogram of Myc-FGF10-(G₄S)₂-p16-Hisx6.

FIG. 19 summarizes the purification of Myc-FGF10-(G₄S)₂-p16-Hisx6.

FIGS. 20A-20C summarizes experiments in which cell penetration of p16, +36GFP-p16, and FGF10-p16 was evaluated in three different cell lines: HepG2, HeLa, and SW626.

FIG. 21 summarizes experiments evaluating cell viability of SKOV-3 cells following treatment with p16 or p16 fusion proteins, in the presence of varying concentrations of an endosome escape agent. For each concentration of endosome escape agent indicated on the graph, results are depicted from left to right, as follows: p16 alone; +36GFP-p16 fusion protein; FGF10-p16 fusion protein; no test article.

FIG. 22 summarizes experiments evaluating cell viability of SKOV-3 cells following treatment with the CDK4/6 inhibitor PD033299.

### DETAILED DESCRIPTION OF THE DISCLOSURE

(i) Overview

This disclosure provides an exemplary application of Intraphilin™ technology in which a member of a class of Surf+ Penetrating Polypeptides is delivered into a cell or is used to deliver a cargo molecule into a cell. In the present application, certain Surf+ Penetrating Polypeptides are complexed with cargo polypeptides, peptides, or small organic molecules, and these conjugates are useful for delivering the cargo into cells. The particular Surf+ Penetrating Polypeptides for use herein are domains of a fibroblast growth factor 10 (FGF10) polypeptide; particularly domains that are at least 4 kDa and have a charge per molecular weight ratio greater than that of full length, unprocessed, naturally occurring FGF10 (herein referred to as “FGF10-related Surf+ Penetrating Polypeptides” or “FGF-10-related Surf+ Penetrating Polypeptides”). Additional suitable Surf+ Penetrating Polypeptides are variants of full length FGF10 or domains thereof, which variants are at least 4 kDa and have a charge per molecular weight ratio greater than that of full length, unprocessed, naturally occurring FGF10 (for the avoidance of doubt, these are also examples of “FGF10-related Surf+ Penetrating Polypeptides” or “FGF-10-related Surf+ Penetrating Polypeptides”). The complexes of the disclosure have a variety of uses, including facilitating delivery of cargo to cells of the liver, kidney, ovaries, and other tissues of the abdominal cavity. Because cell penetration is not ubiquitous; but rather, these FGF10 domains preferentially localize to certain tissues, despite the fact that these tissues are not sites of high expression of the cognate receptor for FGF10, complexes that include an FGF-10 portion, or variants thereof, as a Surf+ Penetrating Polypeptide are particularly useful for preferentially delivering therapeutics into particular cells and tissues.

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “complex of the disclosure” is used to refer to a complex comprising an FGF-10 portion associated with a cargo polypeptide, peptide, or small molecule. In certain embodiments, the FGF-10 portion comprises an FGF10-related Surf+ Penetrating Polypeptide, such as a domain of an FGF-10 polypeptide of at least 4 kDa and having net positive charge, surface positive charge, and a charge per molecular weight ratio greater than that of full length, unprocessed, naturally occurring FGF10.

The term “FGF-10 related Surf+ Penetrating Polypeptide” refers to a Surf+ Penetrating Polypeptide in which cell penetration activity is mediated by all or a portion of FGF-10 or an FGF-10 variant having structural and functional features of a Surf+ Penetrating Polypeptide.

The terms “FGF10” and “FGF-10” are used interchangeably herein.

(ii) Surf+ Penetrating Polypeptides

A “Surf+ Penetrating Polypeptide”, as used herein, is a polypeptide capable of promoting entry into a cell and having, at least, the following characteristics: mass of at least 4 kDa, net positive charge, and presence of surface positive charge such that the polypeptide is capable of promoting entry into a cell. Often, the Surf+ Penetrating Polypeptide also has a charge/molecular weight ratio of at least 0.75. The Surf+ Penetrating Polypeptide can itself enter into a cell and/or can be associated with an agent, such as a polypeptide, peptide, or small organic molecule, such that it also promotes entry into the cell of the agent (also referred to as “cargo portion”). The cargo portion is heterologous to the Surf+ Penetrating Polypeptide portion. In other words, the cargo portion is not the same protein, whether from the same or differing species, as the Surf+ Penetrating Polypeptide. In the context of the present disclosure, the heterologous cargo portion does not, for example, comprise an FGF-10 polypeptide, including unprocessed or mature forms of FGF-10.

In certain embodiments, Surf+ Penetrating Polypeptides have a mass of at least 4 kDa and a charge/molecular weight ratio of at least 0.75 or of greater than 0.75. A Surf+ Penetrating Polypeptide may be a human polypeptide, including a full length, naturally occurring human polypeptide or a variant of a full length, naturally occurring human polypeptide having one or more amino acid additions, deletions, or substitutions (e.g., 1, 2, 3, 4, 5, 6, 7, 8, 9, 10, etc.). Moreover, such human polypeptides include domains of full length naturally occurring human polypeptides or a variant of such a domain having one or more amino acid additions, deletions, or substitutions (e.g., 1, 2, 3, 4, 5, 6, 7, 8, 9, 10, etc.). Variants for use in the present disclosure retain cell penetration activity. For the avoidance of doubt, the term “human polypeptide” includes domains (e.g., structural and functional fragments) unless otherwise specified. Further, Surf+ Penetrating Polypeptides include human or non-human proteins engineered to have one or more regions of surface positive charge and a charge/molecular weight ratio of at least 0.75, including supercharged polypeptides.

In the context of the present disclosure, Surf+ Penetrating Polypeptides for use in the complexes and methods of the disclosure are domains or variants of an FGF-10 polypeptide. In other words, the FGF-10 portion of the disclosed complexes includes a Surf+ Penetrating Polypeptide, referred to herein as an FGF10-related Surf+ Penetrating Polypeptide. In other words, FGF-10 polypeptides, or domains thereof, or variants of either of the foregoing having suitable size (at least 4 kDa), net positive charge, surface positive charge, and cell penetration characteristics are Surf+ Penetrating Polypeptides. The present disclosure provides complexes that include an FGF-10 portion with cell penetration activity and a cargo portion (heterologous protein, peptide, or small organic molecule). Any such complexes may be used to deliver the cargo into a cell, such as into cells of the liver, kidney, or ovaries. These tissues are exemplary of tissues into which a cell penetrating domain of FGF-10 preferentially localizes.

In the present context, a “variant of a human polypeptide” is a polypeptide that differs from a naturally occurring (full length or domain) human polypeptide by one or more amino acid substitutions, additions or deletions. In certain embodiments, these changes in amino acid sequence may be to increase the overall net charge of the polypeptide and/or to increase the surface charge of the polypeptide (e.g., to supercharge a polypeptide). Alternatively, changes in amino acid sequence may be for other purposes, such as to provide a suitable site for pegylation or to facilitate production. In still other embodiments, changes in the amino acid sequence are made to decrease or inhibit a native function of FGF-10 without interfering with cell penetrating activity. For example, changes in the amino acid sequence may be to decrease the mitogenic activity of native FGF-10 and/or to decrease binding affinity for its native reception: FGFR-2b.

Regardless of the specific changes made, the variant of the human polypeptide will be sufficiently similar based on sequence and/or structure to its naturally occurring human polypeptide such that the variant is more closely related to the naturally occurring human protein than it is to a protein from a non-human organism. In certain embodiments, the amino acid sequence of the variant is at least 80%, 85%, 90%, 95%, 97%, 98%, or 99% identical to that of its naturally occurring human protein. In certain embodiments, the variant of the naturally occurring human polypeptide is an FGF10-related Surf+ Penetrating Polypeptide having cell penetrating activity and a charge/molecular weight ratio of at least 0.75 or of greater than 0.75, but the naturally occurring, full length, unprocessed human FGF-10 polypeptide from which the variant is derived does not have cell penetrating activity and/or has a charge/molecular weight ratio of less than 0.75. In certain embodiments, the variant does not result in further supercharging of the polypeptide. For example, the variant results in a change in amino acid sequence but not a change in the net charge, surface charge and/or charge/molecular weight ratio of the polypeptide. In certain embodiments, the variant comprises 1, 2, 3, 4, 5, 6, 7, 8, 9, or 10 amino acid substitutions, additions, and/or deletions (where each change is independently selected from any substitution, addition and/or deletion). In certain embodiments, the variant comprises at least 1, 2, 3, 4, 5, 6, 7, 8, 9, or 10 amino acid substitutions, additions, and/or deletions (where each change is independently selected from any substitution, addition and/or deletion). In certain embodiments, the variant comprises greater than 10 amino acid substitutions, additions, and/or deletions (where each change is independently selected from any substitution, addition and/or deletion).

In the context of the present disclosure, in certain embodiments, the Surf+ Penetrating Polypeptide is a domain or variant of an FGF-10 polypeptide. In other words, the FGF-10 portion of the complex contains the FGF-10 related Surf+ Penetrating Polypeptide. The FGF-10 portion generally comprises (or consists of) an FGF-10 polypeptide, or a domain thereof, or a variant of either of the foregoing, wherein the FGF-10 polypeptide, domain, or variant has a surface positive charge, a net positive charge, and cell penetrating activity. The FGF-10 polypeptide, or domain thereof, or variant of either of the foregoing for use in the complexes of the present disclosure is a Surf+ Penetrating Polypeptide (specifically, FGF-10 related Surf+ Penetrating Polypeptide). As noted above, the variant may have an altered charge relative to the corresponding portion of FGF10 or the variant may have about the same charge.

In certain embodiments, any one or more of the features of a Surf+ Penetrating Polypeptide described herein is a feature of the FGF-10 polypeptide, or domain thereof, or variant of either of the foregoing suitable for use in the complexes and methods of the disclosure.

In certain embodiments, the FGF-10 portion comprises an FGF10-related Surf+ Penetrating Polypeptide. In certain embodiments, the FGF-10 portion comprises a domain of full length, naturally occurring, unprocessed FGF-10 (such as human FGF-10) having a charge/molecular weight ratio greater than that of the corresponding full length, unprocessed FGF-10 polypeptide. In the context of the native human polypeptide, full length, unprocessed FGF-10 has 208 amino acids (See SEQ ID NO: 1) and a charge per molecular weight ratio of 0.68. Thus, an exemplary domain suitable for use as an FGF10-related Surf+ Penetrating Polypeptide includes, for example, the 171 amino acid mature form of human FGF-10 (which is an N-terminal truncation of the unprocessed polypeptide) having a charge per molecular weight ratio of 0.78. Another exemplary domain suitable for use as an FGF10-related Surf+ Penetrating Polypeptide is a 145 amino acid domain set forth in SEQ ID NO: 2 and corresponding to residues 64-208 of the full length, unprocessed, naturally occurring human FGF-10 polypeptide. This 145 amino acid domain has a charge per molecular weight ratio of 1.01, surface positive charge, a net positive charge, and a predicted molecular weight of 16.8 kDa.

In certain embodiments, a native or variant domain of FGF-10 (e.g., a Surf+ Penetrating Polypeptide) has a mass of at least 4, 5, 6, 7, 8, 9, 10, 11, 12, 13, 14, 15, 16, 17, 18, 19, or 20 kDa. For example, the native or variant domain of FGF-10 (e.g., a Surf+ Penetrating Polypeptide) may have a mass of about 4, 5, 6, 7, 8, 9, 10, 11, 12, 13, 14, 15, 16, 17, 18, 19, or about 20 kDa. By way of another example, a native or variant domain of FGF-10 (e.g., a Surf+ Penetrating Polypeptide) may have a mass of about 4-24 kDa, about 5-24 kDa, about 4-20 kDa, about 5-18 kDa, about 5-17 kDa, about 7-17 kDa, about 10-19 kDa, and the like. In certain embodiments, the predicted molecular weight of the native or variant domain of FGF-10 (e.g., a Surf+ Penetrating Polypeptide) is about 5 kDa, about 7.5 kDa, about 10 kDa, about 12.5 kDa, about 15 kDa, about 16.8 kDa, about 17.5 kDa, about 20 kDa, about 21.5 kDa, or about 24 kDa. It should be understood that the mass of the Surf+ Penetrating Polypeptide, including the minimal mass of 4 kDa, refers to monomer mass. However, in certain embodiments, a Surf+ Penetrating Polypeptide for use as part of a complex is a dimer, trimer, tetramer, or a higher order multimer. Moreover, it should be understood that the foregoing examples of the mass of the FGF-10 polypeptide, domain, or variant do not include any additional mass due to the inclusion of linkers, epitope tags, and the like that may be included in a complex that includes an FGF-10 portion. Certainly, however, such features are contemplated and would increase the mass of the overall complex or fusion protein. Additionally, predicted molecular weight may vary due to, for example, glycosylation. Thus, in certain embodiments, reference to molecular weight refers to predicted or theoretical molecular weight.

In certain embodiments, an FGF-10-related Surf+ Penetrating Polypeptide for use in the present disclosure is selected to minimize the number of disulfide bonds. In other words, the FGF-10-related Surf+ Penetrating Polypeptide may have not more than 2 or 3 or 4 disulfide bonds (e.g., the polypeptide has 0, 1, 2, 3 or 4 disulfide bonds). An FGF-10-related Surf+ Penetrating Polypeptide for use in the present disclosure may also be selected to minimize the number of cysteines. In other words, the FGF-10-related Surf+ Penetrating Polypeptide may have not more than 2 cysteines, or not more than 4 cysteines, not more than 6 cysteines or not more than 8 cysteines (e.g., 0, 1, 2, 3, 4, 5, 6, 7, 8 cysteines). An FGF-10-related Surf+ Penetrating Polypeptide for use in the present disclosure may also be selected to minimize glycosylation sites. In other words, the polypeptide may have not more than 1 or 2 or 3 glycosylation sites (e.g., N-linked or O-linked glycosylation; 0, 1, 2 or 3 sites). For example, the domain of native FGF-10 set forth in SEQ ID NO: 2 (an example of a Surf+ Penetrating Polypeptide) has two cysteines but does not have any disulfide bonds.

As defined above, an FGF-10-related Surf+ Penetrating Polypeptide (in this case an FGF-10 polypeptide or a domain or variant thereof) has surface positive charge. The Surf+ Penetrating Polypeptide also has an overall net positive charge under physiological conditions. Note that when the FGF-10-related Surf+ Penetrating Polypeptide is a domain of a naturally occurring polypeptide, the overall net positive charge is that of the domain. For example, in certain embodiments, the FGF-10-related Surf+ Penetrating Polypeptide has an overall net positive charge of at least +5, +8, +10, +12, +14, +15, +16, +17, +18, +19, or +20. By way of further example, a FGF-10-related Surf+ Penetrating Polypeptide may have an overall net positive charge of about +5, +8, +10, +12, +14, +15, +16, +17, +18, +19, +20, or greater than +20. In certain embodiments, under physiological conditions, the FGF-10-related Surf+ Penetrating Polypeptide has a pI greater than or equal to 9, such as a pI of about 9 to about 13 or a pI of between 9 and 13 (inclusive or exclusive). In other embodiments, under physiological conditions, the FGF-10-related Surf+ Penetrating Polypeptide has a pI greater than 9 or greater than 9.5, but less than 10. In other embodiments, under physiological conditions, the FGF-10-related Surf+ Penetrating Polypeptide has a pI of about 9-9.5, or about 9-10, or about 9.5-10, or about 10-10.5, or about 10-10.3. In other embodiments, under physiological conditions, the FGF-10-related Surf+ Penetrating Polypeptide has a pI of about 10-11, about 10.5-11, about 11-12, about 11.5-12, about 12-13, or about 12.5-13. Note that a FGF10-related Surf+ Penetrating Polypeptide may be a polypeptide that has been modified, such as to increase surface charge and/or overall net positive charge as compared to the unmodified protein, and the modified polypeptide may have increased stability and/or increased cell penetrating ability in comparison to the unmodified polypeptide. In some cases, the modified polypeptide may have cell penetrating ability where the unmodified polypeptide did not. Moreover, the modified polypeptide may have been modified to decrease or eliminate a native activity of FGF-10 (other than cell penetration), such as mitogenic activity and/or affinity for its primary cognate receptor.

Theoretical net charge serves as a convenient short hand. In certain embodiments, the theoretical net charge on the FGF-10-related Surf+ Penetrating Polypeptide (e.g., in this case an FGF-10 polypeptide or a domain or variant thereof) is at least +5, +6, +7, +8, +9, +10, +11, +12, +13, +14, +15, +16, +17, +18, +19, +20, +21, +22, +23, +24, or +25. In other embodiments, the theoretical net charge on the FGF-10-related Surf+ Penetrating Polypeptide (e.g., in this case an FGF-10 polypeptide or a domain or variant thereof) is about +5, +6, +7, +8, +9, +10, +11, +12, +13, +14, +15, +16, +17, +18, +19, +20, +21, +22, +23, +24, or +25. For example, the theoretical net charge on the naturally occurring FGF-10-related Surf+ Penetrating Polypeptide can be, e.g., at least +5, at least +10, at least +15, at least +20, or about +5 to +10, +5 to +15, +10 to +20, +15 to +20, +20 to +25, and the like. Note that a FGF-10-related Surf+ Penetrating Polypeptide may be a polypeptide that has been modified, such as to increase surface charge and/or overall net positive charge as compared to the unmodified protein, and the modified polypeptide may have increased stability and/or increased cell penetrating ability in comparison to the unmodified polypeptide. In some cases, the modified polypeptide may have cell penetrating ability where the unmodified polypeptide did not. Moreover, the modified polypeptide may have been modified to decrease or eliminate a native activity of FGF-10 (other than cell penetration), such as mitogenic activity and/or affinity for its primary cognate receptor.

In certain embodiments, the FGF-10-related Surf+ Penetrating Polypeptide (in this case an FGF-10 polypeptide or a domain or variant thereof) has a charge:molecular weight ratio (e.g., also referred to as charge/MW or charge/molecular weight) of at least approximately 0.75, 0.78, 0.8, 1.0, 1.1, 1.2, 1.3, 1.4, 1.5, 1.6, 1.7, 1.8, 1.9, 2.0, 2.5, or 3.0. This ratio is the ratio of the theoretical net charge of the FGF-10-related Surf+ Penetrating Polypeptide to its molecular weight in kilodaltons. In certain embodiments, the charge/molecular weight ratio is about 0.75-2.0. In certain embodiments, the charge/molecular weight ratio of the FGF-10-related Surf+ Penetrating Polypeptide is greater than 0.75. In certain embodiments, the FGF-10-related Surf+ Penetrating Polypeptide is a domain of a naturally occurring, unprocessed human polypeptide where the domain has a charge/molecular weight ratio of at least 0.75 or of greater than 0.75, but the corresponding full length, naturally occurring human polypeptide has a charge/molecular weight ratio of less than 0.75. In certain embodiments, the FGF-10-related Surf+ Penetrating Polypeptide (in this case an FGF-10 polypeptide or a domain or variant thereof) is a domain of a full length, naturally occurring, unprocessed FGF-10 polypeptide having a charge/molecular weight ratio greater than that of the corresponding full length, unprocessed polypeptide. For example, in the case of human FGF-10, the full length, unprocessed, naturally occurring polypeptide has a charge/molecular weight ratio of 0.68. Thus, in certain embodiments, domains suitable as a Surf+ Penetrating Polypeptide are domains of human FGF-10 having a charge/molecular weight ratio of greater than 0.68. In other embodiments, the charge/molecular weight ratio is greater than that of the mature FGF-10 polypeptide (e.g., in the case of native human FGF-10, the domain would have a charge/molecular weight ratio of greater than 0.78). Generally, the molecular weight used in these calculations is the predicted molecular weight based on the amino acid content of the protein.

In certain embodiments, the FGF-10-related Surf+ Penetrating Polypeptide (in this case an FGF-10 polypeptide or a domain or variant thereof) has a charge:molecular weight ratio of at least approximately 0.75 or of greater than 0.75. In certain embodiments, the FGF-10-related Surf+ Penetrating Polypeptide has a charge:molecular weight ratio of at least approximately 0.8. In certain embodiments, the FGF-10-related Surf+ Penetrating Polypeptide has a charge:molecular weight ratio of at least approximately 1.0. In certain embodiments, the FGF-10-related Surf+ Penetrating Polypeptide has a charge:molecular weight ratio of at least approximately 1.2. In certain embodiments, the FGF-10-related Surf+ Penetrating Polypeptide has a charge:molecular weight ratio of at least approximately 1.4. In certain embodiments, the FGF-10-related Surf+ Penetrating Polypeptide has a charge:molecular weight ratio of at least approximately 1.5. In certain embodiments, the FGF-10-related Surf+ Penetrating Polypeptide has a charge:molecular weight ratio of at least approximately 1.6. In certain embodiments, the FGF-10-related Surf+ Penetrating Polypeptide has a charge:molecular weight ratio of at least approximately 1.7. In certain embodiments, the FGF-10-related Surf+ Penetrating Polypeptide has a charge:molecular weight ratio of at least approximately 1.8. In certain embodiments, the FGF-10-related Surf+ Penetrating Polypeptide has a charge:molecular weight ratio of at least approximately 1.9. In certain embodiments, the FGF-10-related Surf+ Penetrating Polypeptide has a charge:molecular weight ratio of at least approximately 2.0. In certain embodiments, the FGF-10-related Surf+ Penetrating Polypeptide has a charge:molecular weight ratio of at least approximately 2.5. In certain embodiments, the FGF-10-related Surf+ Penetrating Polypeptide has a charge:molecular weight ratio of at least approximately 3.0.

In certain embodiments, the FGF10-related Surf+ Penetrating Polypeptide penetrates cells via endocytosis. In certain embodiments, the FGF10-related Surf+ Penetrating Polypeptide binds to cell surface proteoglycans.

In certain embodiments, the FGF10-related Surf+ Penetrating Polypeptide has tertiary structure. The presence of such tertiary structure distinguishes Surf+ Penetrating Polypeptides from unstructured, short cell penetrating peptides (CPPs) such as poly-arginine and poly-lysine and also distinguishes Surf+ Penetrating Polypeptides from cell penetrating peptides that have some secondary structure but no tertiary structure, such as penetratin and antenapedia.

As noted above, FGF10-related Surf+ Penetrating Polypeptides, such as domains and variants of FGF10, are distinguishable based on numerous characteristics from various short cell penetrating peptides known in the art. For example, Surf+ Penetrating Polypeptides, such as FGF10-related Surf+ Penetrating Polypeptides, are distinguishable based on size, shape and structure, charge distribution and the like. Moreover, in certain embodiments, FGF10-related Surf+ Penetrating Polypeptides and complexes comprising an FGF10-related Surf+ Penetrating Polypeptide have improved cell penetration characteristics compared to short CPPs or complexes comprising short CPPs. Nevertheless, to provide further clarity, in certain embodiments, complexes of the disclosure do not further include a short CPP. Additional exemplary support is provided herein.

In certain embodiments, a complex of the disclosure and/or the FGF10-related Surf+ Penetrating Polypeptide portion of a complex of the disclosure do not include a full length sequence for HIV-Tat, or the portion thereof known in the art as imparting cell penetration activity. In certain embodiments, a complex of the disclosure and/or the FGF10-related Surf+ Penetrating Polypeptide portion of a complex of the disclosure does not contain the protein transduction domain of HIV-Tat, for example, does not contain the contiguous amino acid sequence YGRKKRRQRRR (SEQ ID NO: 12). In certain embodiments, a complex of the disclosure comprising a FGF10-related Surf+ Penetrating Polypeptide penetrates cells more efficiently than a complex comprising all or a portion of HIV-Tat fused to the same cargo and/or preferentially penetrates certain cell types and/or has longer half-life.

In certain embodiments, a complex of the disclosure and/or the FGF10-related Surf+ Penetrating Polypeptide portion of a complex of the disclosure do not include the protein transduction domain of an antennapedia protein, such as the Drosophilia antennapedia protein or a mammalian ortholog thereof. In certain embodiments, a complex of the disclosure and/or the FGF10-related Surf+ Penetrating Polypeptide portion of a complex of the disclosure does not include the protein transduction domain of the h-region of fibroblast growth factor 4 (FGF-4). In certain embodiments, a complex of the disclosure and/or the cargo portion of the complex of the disclosure do not include an FGF receptor or the ligand binding domain of an FGF receptor. In certain embodiments, a complex of the disclosure does not include an FGF polypeptide, other than the FGF10 polypeptide, or portion or variant thereof, that comprises the FGF10 portion.

In certain embodiments, a complex of the disclosure and/or the FGF10-related Surf+ Penetrating Polypeptide portion of a complex of the disclosure do not include the 16 amino acid residue sequence referred to as penetratin: RQIKIWFQNRRMKWKK (SEQ ID NO: 13). In certain embodiments, a complex of the disclosure and/or the FGF10-related Surf+ Penetrating Polypeptide portion of a complex of the disclosure do not include the 19 amino acid residue sequence referred to as SynB1: RGGRLSYSRRRFSTSTGRA (SEQ ID NO: 14). In certain embodiments, a complex of the disclosure and/or the FGF10-related Surf+ Penetrating Polypeptide portion of a complex of the disclosure do not include the following amino acid sequence referred to as transportan: GWTLNSAGYLLGKINLKALAALAKKIL (SEQ ID NO: 15).

In certain embodiments, a complex of the disclosure and/or the FGF10-related Surf+ Penetrating Polypeptide portion of a complex of the disclosure do not include HSV-1 structural protein Vp22 (DAATATRGRSAASRPTERPRAPARSASRPRRPVE (SEQ ID NO: 16)). In certain embodiments, a complex of the disclosure and/or the FGF10-related Surf+ Penetrating Polypeptide portion of a complex of the disclosure does not include 9 (or, optionally, does not include 7 or 8) consecutive arginine residues (e.g., poly-Arg9 (SEQ ID NO: 17)). In other embodiments, a complex of the disclosure and/or the FGF10-related Surf+ Penetrating Polypeptide portion of a complex of the disclosure does not include 9 (or, optionally, does not include 7 or 8) consecutive lysine residues (e.g., poly-Lys9 (SEQ ID NO: 18)). In certain embodiments, a complex of the disclosure and/or the FGF10-related Surf+ Penetrating Polypeptide portion of a complex of the disclosure does not include the PTD of mouse transcription factor Mph-1 (YARVRRRGPRR) (SEQ ID NO: 19), Sim-2 (AKAARQAAR (SEQ ID NO: 20)). HIV-1 viral protein Tat (YGRKKRRQRRR (SEQ ID NO: 12)), Antennapedia protein (Antp) of Drosophila (RQIKIWFQNRRMKWKK) (SEQ ID NO: 13), MTS (AAVALLPAVLLALLAPAAADQNQLMP (SEQ ID NO: 21)), and short amphipathic peptide carriers Pep-1 (KETWWETWWTEWSQPKKKRKV (SEQ ID NO: 22)) and Pep-2 (KETWFETWFTEWSQPKKKRKV (SEQ ID NO: 23)).

In certain embodiments, regardless of whether a complex of the disclosure also includes a short cell penetration peptide, such as 9 consecutive arginine (SEQ ID NO: 17) or lysine (SEQ ID NO: 18) residues or TAT, the complex of the disclosure still has one or more preferential cell penetration or other preferential characteristics in comparison to a complex that lacks the FGF10-related Surf+ Penetrating Polypeptide portion. For example, in certain embodiments, a complex of the disclosure comprising an FGF10-related Surf+ Penetrating Polypeptide penetrates cells more efficiently than a complex comprising all or a portion of HIV-Tat fused to the same cargo and/or preferentially penetrates certain cell types and/or has longer half-life.

The foregoing provides description for characteristics of FGF10-related Surf+ Penetrating Polypeptides and sub-categories of FGF10-related Surf+ Penetrating Polypeptides. The disclosure contemplates that any such FGF10-related Surf+ Penetrating Polypeptide (in this case an FGF-10 polypeptide or a domain or variant thereof) for use in the present disclosure may be described based on presence or absence of any one or any combination of any of the foregoing features. Additional features and specific examples of polypeptides having such features are described in greater detail below. Such features and combinations of features (including combinations with features set forth above) may also be used to describe the Surf+ Penetrating Polypeptide for use in accordance with the claimed disclosure. Any such polypeptides or categories or sub-categories may be used as part of a complex of the disclosure (e.g., the disclosure provides complexes comprising any such polypeptides) and may be combined with any of the exemplary classes of cargo described below.

Full length, naturally occurring, unprocessed human FGF-10 is a 208 amino acid polypeptide, and the amino acid sequence is set forth in SEQ ID NO: 1. This full length, naturally occurring human protein has a molecular weight of about 23.44 and a theoretical net charge of +16. The charge per molecular weight ratio of this unprocessed, naturally occurring polypeptide is about 0.68.

An exemplary FGF10-related Surf+ Penetrating Polypeptide is a domain of this full-length, naturally occurring, unprocessed polypeptide. One such exemplary domain for which cell penetration activity has been confirmed is set forth in SEQ ID NO: 2. This domain is a 145 amino acid domain from residue 64-208 of the full length, unprocessed, human polypeptide. This particular domain has a theoretical molecular weight of about 16.78 and a theoretical net charge of +17. The charge per molecular weight ratio is about 1.01. This domain is an example of an FGF10-related Surf+ Penetrating Polypeptide with a charge per molecular weight ratio greater than that of the full length, naturally occurring, unprocessed polypeptide. Additionally, this domain also has a charge per molecular weight ratio greater than that of the mature, naturally occurring FGF-10 polypeptide. Moreover, this domain is an example of an FGF10-related Surf+ Penetrating Polypeptide with a net charge greater than that of the full length, naturally occurring, unprocessed polypeptide. Additionally, this is an example of a sub-category of Surf+ Penetrating Polypeptides where the domain has a charge per molecular weight ratio of greater than 0.75, but the full length, unprocessed, naturally occurring polypeptide has a charge per molecular weight ratio less than 0.75.

This particular domain is merely exemplary of FGF10-related Surf+ Penetrating Polypeptides for use in the complexes of the disclosure. Other suitable domains and variants can be readily identified and tested using, for example, the assays provided herein to confirm that the domain or variant retains cell penetrating activity.

In certain embodiments, the disclosure provides complexes in which the FGF10-related Surf+ Penetrating Polypeptide has at least the following characteristics: surface positive charge, mass of at least 4 kDa, charge/molecular weight ratio of at least 0.75 or of greater than 0.75, and is a domain of a naturally occurring, unprocessed, human FGF-10 polypeptide. In certain embodiments, the selected domain has a charge per molecular weight ratio greater than that of the corresponding naturally occurring, mature human polypeptide. In other embodiments, the selected domain has a charge per molecular weight ratio of at least 0.75 or greater than 0.75, but the full length, naturally occurring human polypeptide has a charge per molecular weight ratio of less than 0.75. In other embodiments, the selected domain has a net theoretical charge greater than that of the corresponding full length, naturally occurring, unprocessed human polypeptide. In other embodiments, the selected domain has a net theoretical charge that is the same or approximately the same as that of the corresponding full length, naturally occurring, unprocessed polypeptide.

The disclosure contemplates the use of any of the specified domains of full length, naturally occurring, unprocessed human FGF-10, as well as other domains and variants having the charge and molecular weight characteristics of a Surf+ Penetrating Polypeptide. Moreover, the disclosure contemplates the use of variants of full length, naturally occurring, unprocessed or FGF10 polypeptides having the charge and molecular weight characteristics of a Surf+ Penetrating Polypeptide. Further, the disclosure contemplates that complexes may comprise a full length naturally occurring human polypeptide, even though only a domain of said human polypeptide functions as a Surf+ Penetrating Polypeptide. In such cases, the additional polypeptide sequence can optionally be used to interconnect the FGF10-related Surf+ Penetrating Polypeptide to the cargo portion. Thus, in certain embodiments, the disclosure provides complexes comprising a FGF-10 portion that comprises an FGF10-related Surf+ Penetrating Polypeptide. Such a Surf+ Penetrating Polypeptide may optionally be provided with additional sequence endogenously present in, for example, the naturally occurring polypeptide from which the Surf+ Penetrating Polypeptide is a domain or may be present without additional sequence endogenously present in the naturally occurring polypeptide from which the Surf+ Penetrating Polypeptide is a domain. In certain embodiments, the presence of additional sequence from the same naturally occurring polypeptide does not result in the FGF10 portion comprising the FGF10-related Surf+ Penetrating Polypeptide having a charge/molecular weight ratio of less than 0.75. However, in certain embodiments, the presence of additional sequence from the same naturally occurring polypeptide results in the FGF-10 portion comprising the FGF10-related Surf+ Penetrating Polypeptide having a charge/molecular weight ratio of less than 0.75. For the avoidance of doubt, the “FGF-10 portion” may include both the FGF10-related Surf+ Penetrating Polypeptide (the FGF10 moiety that functions as a Surf+ Penetrating Polypeptide) and additional sequence from the same or similar naturally or non-naturally occurring polypeptide. This FGF10 portion does not include heterologous linker sequence, nuclear localization signals, or additional portions intended to have an independent and distinct biological function (e.g., a moiety to increase the half life of the complex).

The foregoing are exemplary of FGF10-related Surf+ Penetrating Polypeptides that can be used as part of the complexes of the disclosure. For the avoidance of doubt, it should be understood that domains of the naturally occurring FGF10 polypeptides may be modified, such as by introducing one or more amino acid substitutions, deletions or additions. The resulting domain will still be considered a domain of a naturally occurring polypeptide as long as the domain is readily identifiable based on sequence and/or structure as a domain of that naturally occurring protein.

Although specific examples of suitable domains, including variants, of FGF-10 that are Surf+ Penetrating Polypeptides have been provided, it should be appreciated that other fragments of the corresponding naturally occurring human proteins may also be suitable, such as an overlapping fragment that retains the surface positive charge of the recited fragment but is shorter or longer (e.g., the starting or ending residue is different but the functional core of surface positive charge is retained; the fragment retains the essential structure of the recited fragment). Fragments that retain the essential structure but differ in length may differ in mass, length, and/or charge/molecular weight ratio. However, essential structure and presence of surface charge and net positive charge (although not necessarily the identical net charge) are maintained. In certain embodiments, charge/molecular weight ratio of at least 0.75 is also maintained.

In certain embodiments, the FGF10-related Surf+ Penetrating Polypeptide portion of a complex of the disclosure is or comprises a domain of a human polypeptide, such as a domain of a naturally occurring human polypeptide. A complex may comprise the domain outside of its context in its full length, naturally occurring protein (e.g., the complex does not include the full length human polypeptide from which the domain is a portion). Alternatively, the domain may be provided in the context of its full length polypeptide or in the context of additional polypeptide sequence (but less than all) from the naturally occurring protein FGF10 polypeptide from which the FGF10-related Surf+ Penetrating Polypeptide is a domain (e.g., the complex does include the full length human polypeptide from which the domain is an identified portion).

In some embodiments, the FGF-10 portion of a complex of the disclosure comprises a domain of the FGF-10 polypeptide set forth in SEQ ID NO: 1. For example, in some embodiments, a complex comprises an FGF-10 portion comprising a domain of the FGF-10 polypeptide set forth in SEQ ID NO: 1 (e.g., the domain is, in certain embodiments, an FGF10-related Surf+ Penetrating Polypeptide). In certain embodiments, the complex includes at least about 10%, 15%, 20%, 25%, 30%, 35%, 40%, 45%, 50%, 55%, 60%, 65%, 70%, 75%, 80%, 85%, 90%, 95%, 96%, 97%, 98%, or 100% of the full length, unprocessed, naturally occurring FGF-10 polypeptide, such as the polypeptide having the amino acid sequence set forth in SEQ ID NO: 1, provided as contiguous amino acid residues.

Regardless of the specific FGF-10 portion or FGF10-related Surf+ Penetrating Polypeptide or category of FGF10-related Surf+ Penetrating Polypeptide used in a complex, the disclosure contemplates embodiments in which the complex comprises a domain of a full length, naturally occurring human protein, but does not include the full length, naturally occurring human protein as a contiguous amino acid sequence. However, even when a domain of a full length, naturally occurring human protein is used as the FGF-10 portion and provides the Surf+ Penetrating Polypeptide function for a complex, the disclosure contemplates embodiments in which that domain is provided in the context of the full length (or substantially full length), naturally occurring protein—such that the complex comprises the full length, naturally occurring human protein, or when the Surf+ Polypeptide portion includes additional polypeptide sequence (more sequence than is necessary or sufficient to achieve cell penetration).

For illustrative purposes, the disclosure has provided exemplary FGF-10 portions, including FGF10-related Surf+ Penetrating Polypeptides (in this cases cell penetrating FGF-10 polypeptides and domains thereof and variants of either of the foregoing), including human polypeptides. However, Surf+ Penetrating Polypeptides suitable for use also include polypeptides from other species, such as mouse, rat, monkey, etc. Accordingly, the disclosure contemplates use of naturally occurring polypeptides (and domains thereof having characteristics of Surf+ Penetrating Polypeptides) from these other organisms. Accordingly, in one embodiment, the disclosure provides a complex in which the FGF-10 portion is a naturally occurring mammalian polypeptide (such as mouse, rat, monkey, etc.) or domain thereof.

Supercharging

In addition, in certain embodiments, FGF10-related Surf+ Penetrating Polypeptides include naturally occurring or non-human proteins that may be or have been further modified to increase positive charge (e.g., supercharged). These include polypeptides that, prior to supercharging, have a charge/molecular weight ratio of at least 0.75 or of greater than 0.75, as well as polypeptides that do not have a charge/molecular weight ratio of at least 0.75 prior to supercharging. Thus, the disclosure contemplates FGF-10 variants (full length or domains) that have been modified to increase positive charge and/or charge/molecular weight ratio.

FGF10-related Surf+ Penetrating Polypeptides can be naturally-occurring, or can be produced by changing one or more conserved or non-conserved amino acids on or near the surface of a protein to more polar or charged amino acid residues. The amino acid residues to be modified may be hydrophobic, hydrophilic, charged, or a combination thereof. FGF10-related Surf+ Penetrating Polypeptides can also be produced by the attachment of charged moieties to the protein in order to supercharge the protein.

A naturally occurring FGF10-related Surf+ Penetrating Polypeptides, or a protein to be modified for supercharging, may be derived from any species of plant, animal, and/or microorganism. In certain embodiments, the protein is a mammalian protein. In certain embodiments, the protein is a human protein. In certain embodiments, the naturally occurring FGF10-related Surf+ Penetrating Polypeptide, or the protein to be modified, is derived from an organism typically used in research. For example, the naturally occurring Surf+ Penetrating Polypeptide, or the protein to be modified, may be from a primate (e.g., ape, monkey), rodent (e.g., rabbit, hamster, gerbil), pig, dog, cat, fish (e.g., Danio rerio), nematode (e.g., C. elegans), yeast (e.g., Saccharomyces cerevisiae), or bacteria (e.g., E. coli). In certain embodiments, the protein is non-immunogenic. In other certain embodiments, the protein is non-antigenic. In certain embodiments, the protein does not have inherent biological activity or has been modified to have no or reduced biological activity. In certain embodiments, the protein is chosen based on its targeting ability.

In certain embodiments of the disclosure, the term supercharging is used to refer to changes made to the FGF10-related Surf+ Penetrating Polypeptide or changes made to a polypeptide such that it functions as and meets the definition of a FGF10-related Surf+ Penetrating Polypeptide, but do not include changes in charge or charge density that result from association with the cargo portion.

In some embodiments, the naturally occurring FGF10-related Surf+ Penetrating Polypeptides, or the protein to be modified is one whose structure has been characterized, for example, by NMR or X-ray crystallography. In some embodiments, the naturally occurring FGF10-related Surf+ Penetrating Polypeptides, or the protein to be modified, is one whose structure has been predicted, for example, by threading homology modeling or de novo structure prediction. In some embodiments, the naturally occurring FGF10-related Surf+ Penetrating Polypeptides, or the protein to be modified, is one whose structure has been correlated and/or related to biochemical activity (e.g., enzymatic activity, protein-protein interactions, etc.). In certain embodiments, the inherent biological activity of a modified protein is reduced or eliminated to reduce the risk of deleterious and/or undesired effects. Alternatively, the biological activity of the modified protein can be increased or potentiated, or a non-naturally occurring biological activity of the protein may be generated as a result of the charge modification concomitant with the creation of the charged-modified FGF10-related Surf+ Penetrating Polypeptides.

For embodiments in which a protein is modified to generate an FGF10-related Surf+ Penetrating Polypeptides, the surface residues of a protein to be modified may be identified using any method known in the art. In certain embodiments, surface residues are identified by computer modeling of the protein. In certain embodiments, the three-dimensional structure of the protein is known and/or determined, and surface residues are identified by visualizing the structure of the protein. Homology modeling and de novo structure prediction are two methods for modeling the 3-D structure of a protein; such methods are particularly useful in the absence of an NMR or crystal structure. In some embodiments, surface residues are predicted using computer software. In certain particular embodiments, an Accessible Surface Area (ASA) is used to predict surface exposure. A high ASA value indicates a surface exposed residue, whereas a low ASA value indicates the exclusion of solvent interactions with the residue. In certain particular embodiments, an Average Neighbor Atoms per Sidechain Atom (AvNAPSA) value is used to predict surface exposure. AvNAPSA is an automated measure of surface exposure which has been implemented as a computer program. A low AvNAPSA value indicates a surface exposed residue, whereas a high value indicates a residue in the interior of the protein. In certain embodiments, the software is used to predict the secondary structure and/or tertiary structure of a protein, and surface residues or near-surface residues are identified based on this prediction. In some embodiments, the prediction of surface residues is based on hydrophobicity and hydrophilicity of the residues and their clustering in the primary sequence of the protein. Besides in silico methods, surface residues of the protein may also be identified using various biochemical techniques, for example, protease cleavage, surface modification, derivatization, labeling, hydrogen-deuterium exchange experiments, etc. We note that such modeling is also useful for identifying domains of a full length protein that possess characteristics of an FGF10-related Surf+ Penetrating Polypeptide.

Optionally, of the surface residues, it is then determined which are conserved or important to the functioning of the protein. However, conserved amino acids may be modified even if the underlying biological activity of the protein is to be retained, reduced, enhanced or augmented by one or more non-naturally occurring biological activities. Identification of conserved residues can be determined using any method known in the art. In certain embodiments, conserved residues are identified by aligning the primary sequence of the protein of interest with related proteins. These related proteins may be from the same family of proteins. Related proteins may also be the same protein from a different species. For example, conserved residues may be identified by aligning the sequences of the same protein from different species. For example, proteins of similar function or biological activity may be aligned. Preferably, 2, 3, 4, 5, 6, 7, 8, 9, 10 or more than 10 different sequences are used to determine the conserved amino acids in the protein. In certain embodiments, a residue is considered conserved if over 50%, over 60%, over 70%, over 75%, over 80%, over 90%, or over 95% of the sequences have the same amino acid in a particular position. In other embodiments, the residue is considered conserved if over 50%, over 60%, over 70%, over 75%, over 80%, over 90%, or over 95% of the sequences have the same or a similar (e.g., valine, leucine, and isoleucine; glycine and alanine; glutamine and asparagine; or aspartate and glutamate) amino acid in a particular position. Many software packages are available for aligning and comparing protein sequences as described herein. As would be appreciated by one of skill in the art, either the conserved residues may be determined first or the surface residues may be determined first. The order does not matter. In certain embodiments, a computer software package may determine surface residues and/or conserved residues, and may optionally do so simultaneously. Important residues in the protein may also be identified by mutagenesis of the protein. For example, alanine scanning of the protein can be used to determine the important amino acid residues in the protein. In some embodiments, site-directed mutagenesis may be used. In certain embodiments, conserving the original biological activity of the protein is not important, and therefore, the steps of identifying the conserved residues and preserving them are not performed.

Each of the surface residues is identified as hydrophobic or hydrophilic. In certain embodiments, residues are assigned a hydrophobicity score. For example, each surface residue may be assigned an octanol/water log P value. Other hydrophobicity parameters may also be used. Such scales for amino acids have been discussed in: Janin, 1979, Nature, 277:491; Wolfenden et al., 1981, Biochemistry, 20:849; Kyte et al., 1982, J. Mol. Biol., 157:105; Rose et al., 1985, Science, 229:834; Cornette et al., 1987, J. Mol. Biol., 195:659; Charton and Charton, 1982, J. Theor. Biol., 99:629; each of which is incorporated by reference. Any of these hydrophobicity parameters may be used in the inventive method to determine which residues to modify. In certain embodiments, hydrophilic or charged residues are identified for modification. Near-surface residues are residues that are either a) not surface residues but immediately adjacent in primary amino acid sequence or within a three-dimensional structure or b) not surface residues that can become surface residues upon the alteration of a polypeptide's tertiary structure. The contribution of near-surface residues in a Surf+ Penetrating Polypeptide is determined using the methods described herein.

In certain embodiments, for generation of FGF10-related Surf+ Penetrating Polypeptides, at least one identified surface residue or near-surface residue is chosen for modification. In certain embodiments, hydrophobic residue(s) are chosen for modification. In other embodiments, hydrophilic and/or charged residue(s) are chosen for modification. In certain embodiments, more than one residue is chosen for modification. In certain embodiments, 1, 2, 3, 4, 5, 6, 7, 8, 9, 10 or more than 10 of the identified residues are chosen for modification. In certain embodiments, over 10, over 15, over 20, or over 25 residues are chosen for modification.

In certain embodiments, multiple variants of a protein, each with different modifications, are produced and tested to determine the best variant in terms of delivery of a biological moiety to a cell, pharmacokinetics, stability, biocompatibility, and/or biological activity, or a biophysical property such as expression level. In some embodiments, a library of protein variants is generated in an in vivo system containing an expression host such as phage, bacteria, yeast or mammalian cells, or in an in vitro system such as mRNA display, ribosome display, or polysome display. Such a library may contain 10, 10², 10³, 10⁴, 10⁵, 10⁶, 10⁷, 10⁸, 10⁹, or over 10⁹, possible variants (including substitutions, deletions of one or more residues, and insertion of one or more residues). By testing the variants resulting from this library, additional FGF10-related Surf+ Penetrating Polypeptides may be created.

In certain embodiments, residues chosen for modification are mutated into more hydrophilic residues (including positively charged residues). Typically, residues are mutated into more hydrophilic natural amino acids. In certain embodiments, residues are mutated into amino acids that are positively charged at physiological pH. For example, a residue may be changed to an arginine, or lysine, or histidine. In certain embodiments, all the residues to be modified are changed into the same alternate residue. For example, all the chosen residues are changed to an arginine residue, a lysine residue or a histidine residue. In other embodiments, the chosen residues are changed into different residues (e.g., the change at each position is independent selected); however, all the final residues are positively charged at physiological pH. In certain embodiments, to create a positively charged protein, all the residues to be mutated are converted to arginine or lysine or histidine residues, or a combination thereof. To give but another example, all the chosen residues for modification are aspartate, glutamate, asparagine, and/or glutamine, and these residues are mutated into arginine, lysine or histidine.

In some embodiments, a protein may be modified to increase the overall net charge on the protein. In certain embodiments, the theoretical net charge is increased, relative to its unmodified protein, by at least +1, at least +2, at least +3, at least +4, at least +5, at least +6, at least +7, at least +8, at least +9, or at least +10. In certain embodiments, the chosen amino acids are changed (each change being independently selected) into non-ionic, polar residues (e.g., cysteine, serine, threonine, tyrosine, glutamine, and asparagine). In some embodiments, increasing the overall net charge comprises increasing the total number of positively charged residues on or near the surface.

In certain embodiments, the amino acid residues mutated to charged amino acids residues are separated from each other by at least 1, at least 2, at least 3, at least 4, at least 5, at least 6, at least 7, at least 8, at least 9, at least 10, at least 15, at least 20, or at least 25 amino acid residues in the primary amino acid sequence. In certain embodiments, the amino acid residues mutated to positively charged amino acids residues (e.g., arginine, lysine or histidine) are separated from each other by at least 1, at least 2, at least 3, at least 4, at least 5, at least 6, at least 7, at least 8, at least 9, at least 10, at least 15, at least 20, or at least 25 amino acid residues in the primary amino acid sequence. In certain embodiments, fewer than two or only two, three, four or five consecutive amino acids are modified to generate a charge-modified Surf+ Penetrating Polypeptide. Alternatively, wherein a surface projection is present in the polypeptide, more than two, three, four, five, six, seven, eight, nine, or ten consecutive amino acids are modified to generate a charged-modified Surf+ Penetrating Polypeptide.

In certain embodiments, a surface exposed loop, helix, turn, or other secondary structure may contain only 1, 2, 3, 4, 5, 6, 7, 8, 9, 10, 15, 20, 25, 30 or more than 30 charged residues. Distributing the charged residues over the surface of the protein may allow for more stable proteins. In certain embodiments, only 1, 2, 3, 4, or 5 residues per 15-20 amino acids of the primary sequence are mutated to charged amino acids (e.g., arginine, lysine or histidine). In certain embodiments, on average only 1, 2, 3, 4, or 5 residues per 10 amino acids of the primary sequence are mutated to charged amino acids (e.g., arginine, lysine or histidine). In certain embodiments, on average only 1, 2, 3, 4, or 5 residues per 15 amino acids of the primary sequence are mutated to charged amino acids (e.g., arginine, lysine or histidine). In certain embodiments, on average only 1, 2, 3, 4, or 5 residues per 20 amino acids of the primary sequence are mutated to charged amino acids (e.g., arginine, lysine or histidine). In certain embodiments, on average only 1, 2, 3, 4, or 5 residues per 25 amino acids of the primary sequence are mutated to charged amino acids (e.g., arginine, lysine or histidine). In certain embodiments, on average only 1, 2, 3, 4, or 5 residues per 30 amino acids of the primary sequence are mutated to charged amino acids (e.g., arginine, lysine or histidine).

In certain embodiments, at least 50%, at least 60%, at least 70%, at least 80%, or at least 90% of the mutated charged amino acid residues of a charge-modified Surf+ Penetrating Polypeptide are solvent exposed. In certain embodiments, at least 50%, at least 60%, at least 70%, at least 80%, or at least 90% of the mutated charged amino acids residues of the charge-modified Surf+ Penetrating Polypeptide are on the surface of the protein. In certain embodiments, less than 5%, less than 10%, less than 20%, less than 30%, less than 40%, less than 50% of the mutated charged amino acid residues are not solvent exposed. In certain embodiments, less than 5%, less than 10%, less than 20%, less than 30%, less than 40%, less than 50% of the mutated charged amino acid residues are internal amino acid residues.

In some embodiments, amino acids are selected for modification using one or more predetermined criteria. For example, to generate a superpositively charged protein, ASA or AvNAPSA values may be used to identify aspartic acid, glutamic acid, asparagine, and/or glutamine residues with ASA values above a certain threshold value or AvNAPSA values below a certain threshold value, and one or more (e.g., all) of these residues may be changed to arginine, lysine or histidine. In some embodiments, to generate a superpositively charged protein, ASA calculations are used to identify aspartic acid, glutamic acid, asparagine, and/or glutamine residues with ASA above a certain threshold value, and one or more (e.g., all) of these are changed to arginine, lysine or histidine. In some embodiments, to generate a superpositively charged protein, AvNAPSA is used to identify aspartic acid, glutamic acid, asparagine, and/or glutamine residues with AvNAPSA below a certain threshold value, and one or more (e.g., all) of these are changed to arginines. In some embodiments, to generate a superpositively charged protein, AvNAPSA is used to identify aspartic acid, glutamic acid, asparagine, and/or glutamine residues with AvNAPSA below a certain threshold value, and one or more (e.g., all) of these are changed to lysines. In other embodiments, to generate a superpositively charged protein, AvNAPSA is used to identify aspartic acid, glutamic acid, asparagine, and/or glutamine residues with AvNAPSA below a certain threshold value, and one or more (e.g., all) of these are changed to histidines.

In some embodiments, solvent-exposed residues are identified by the number of neighbors. In general, residues that have more neighbors are less solvent-exposed than residues that have fewer neighbors. In some embodiments, solvent-exposed residues are identified by half sphere exposure, which accounts for the direction of the amino acid side chain (Hamelryck, 2005, Proteins, 59:8-48; incorporated herein by reference). In some embodiments, solvent-exposed residues are identified by computing the solvent exposed surface area, accessible surface area, and/or solvent excluded surface of each residue. See, e.g., Lee et al., J. Mol. Biol. 55(3):379-400, 1971; Richmond, J. Mol. Biol. 178:63-89, 1984; each of which is incorporated herein by reference.

The desired modifications or mutations in the protein may be accomplished using any techniques known in the art. Recombinant DNA techniques for introducing such changes in a protein sequence are well known in the art. In certain embodiments, the modifications are made by site-directed mutagenesis of the polynucleotide encoding the protein. Other techniques for introducing mutations are discussed in Molecular Cloning: A Laboratory Manual, 2nd Ed., ed. by Sambrook, Fritsch, and Maniatis (Cold Spring Harbor Laboratory Press: 1989); the treatise, Methods in Enzymnology (Academic Press, Inc., N.Y.); Ausubel et al. Current Protocols in Molecular Biology (John Wiley & Sons, Inc., New York, 1999); each of which is incorporated herein by reference. The modified protein is expressed and tested. In certain embodiments, a series of variants is prepared, and each variant is tested to determine its biological activity and its stability. The variant chosen for subsequent use may be the most stable one, the most active one, or the one with the greatest overall combination of activity and stability. After a first set of variants is prepared an additional set of variants may be prepared based on what is learned from the first set. Variants are typically created and over-expressed using recombinant techniques known in the art.

As noted throughout, variants for use in the claimed complexes are not only supercharged variants. Rather, they also include variants that change a native function of the FGF10 portion. Thus, in certain embodiments, a variant FGF10 portion may be modified to alter charge characteristics, such as net charge, charge distribution, or charge per molecular weight ratio. Alternatively, in certain embodiments, a variant FGF10 portion may be modified to alter a native function of the FGF10 portion, such as to decrease an endogenous activity of native FGF10 (e.g., reduce mitogenicity, reduce affinity for FGF10 receptor). In certain embodiments, a variant is modified to influence both charge characteristics and to reduce a native function of the FGF10 portion. Note that when generating variants with decreased endogenous FGF10 activity, suitable residues for substitution can be selected by reviewing models of FGF10. Unlike when supercharging a domain, substitutions to decrease endogenous FGF10 activity will not necessarily be on the surface of the molecule.

As would be appreciated by one of skill in the art, protein fragments, functional protein domains, and homologous proteins are also considered to be within the scope of this disclosure. For example, provided herein is any protein fragment of a reference protein (meaning a polypeptide sequence at least one amino acid residue shorter than a reference polypeptide sequence but otherwise identical) 10, 20, 30, 40, 50, 60, 70, 80, 90, 100 or greater than 100 amino acids in length In another example, any protein that includes a stretch of about 20, about 30, about 40, about 50, or about 100 amino acids which are about 80%, about 90%, about 95%, or about 100% identical to any of the sequences described herein can be utilized in accordance with the disclosure. In certain embodiments, a protein sequence to be utilized in accordance with the disclosure includes 1, 2, 3, 4, 5, 6, 7, 8, 9, 10, or more mutations as shown in any of the sequences provided or referenced herein.

In certain embodiments, the variant includes 1, 2, 3, 4, 5, 6, 7, 8, 9, or 10 amino acid substitutions, deletions, and/or additions (each of which is independently selected) relative to all or a corresponding portion of SEQ ID NO: 1 or 2. In certain embodiments, the variant comprises an amino acid sequence at least 80%, 85%, 90%, 91%, 92%, 93%, 94%, 95%, 96%, 97%, 98%, or 99% identical to all or a corresponding portion of SEQ ID NO: 1 or 2. In certain embodiments, the variant includes 1, 2, 3, 4, 5, 6, 7, 8, 9, or 10 amino acid substitutions, deletions, and/or additions (each of which is independently selected) relative to all or a corresponding portion of naturally occurring, mature FGF10, such as human FGF10. In certain embodiments, the variant comprises an amino acid sequence at least 80%, 85%, 90%, 91%, 92%, 93%, 94%, 95%, 96%, 97%, 98%, or 99% identical to all or a corresponding portion of naturally occurring, mature FGF10, such as human FGF10. Suitable variants for use in the context of the present disclosure retain cell penetrating activity and are FGF10-related Surf+ Penetrating Polypeptides.

In certain the FGF10 portion comprises a variant (an FGF10-related Surf+ Penetrating Polypeptide) which includes 1, 2, 3, 4, 5, 6, 7, 8, 9, or 10 amino acid substitutions, deletions, and/or additions (each of which is independently selected) relative to all or a corresponding portion 2, 8, or 9. In certain embodiments, the variant comprises an amino acid sequence at least 80%, 85%, 90%, 91%, 92%, 93%, 94%, 95%, 96%, 97%, 98%, or 99% identical to all or a corresponding portion of SEQ ID NO: 2, 8, or 9. Suitable variants for use in the context of the present disclosure retain cell penetrating activity and are FGF10-related Surf+ Penetrating Polypeptides.

In certain embodiments, in addition to being a FGF10-related Surf+ Penetrating Polypeptide, an endogenous activity of FGF-10 is decreased or substantially eliminated in the variant polypeptide. For example, the variant may have decreased mitogenic activity and/or decreased affinity for its cognate receptor FGFR2b. Exemplary variants in which an endogenous activity of FGF-10 is decreased are set forth in SEQ ID NO: 8 and SEQ ID NO: 9. For example, an FGF-10 variant or fragment having two substitutions (E158K/K195A; where the numbering of the amino acid residues is relative to the full length, unprocessed, naturally occurring protein) displays decreased binding to the FGFR2b receptor by approximately a factor of 4 without affecting the binding of FGF10 to heparin. By way of further example, an FGF-10 variant or fragment having one substitution (R78A; where the numbering of the amino acid residues is relative to the full length, unprocessed, naturally occurring protein) displays an approximately 4-fold decrease in binding to the FGFR2b receptor along with a significant decrease in mitogenic activity. By way of further example, an FGF-10 variant or fragment having one substitution (T114 modified to either arginine or alanine; where the numbering of the amino acid residues is relative to the full length, unprocessed, naturally occurring protein) displays reduced binding to FGFR2b relative to the wild-type protein as well as reduced mitogenic activity.

In certain embodiments, the FGF-10 portion comprises an E158K/K195A FGF-10 variant. In other embodiments, the FGF-10 portion comprises an R78A FGF-10 variant. In certain embodiments, the FGF-10 portion comprises one, two, three, or four substitutions selected from: R78A, T114A, E158K, E158A, or K195A. In certain embodiments, the FGF-portion comprises or consists of the amino acid sequence set forth in SEQ ID NO: 2 in the presence of one, two, three, four, or five amino acid substitutions, wherein the amino acid substitutions decrease the mitogenic activity and/or FGFR binding activity of the FGF-10 portion, relative to the naturally occurring FGF-10 portion. In certain embodiments, the FGF-10 portion comprises an amino acid sequence set forth in SEQ ID NO: 8. In other embodiments, the FGF-10 portion comprises an amino acid sequence set forth in SEQ ID NO: 9. In other embodiments, the FGF-10 portion comprises an amino acid sequence set forth in SEQ ID NO: 10.

In certain embodiments, the disclosure provides a complex, such as a fusion protein, comprising an FGF10 portion and a cargo portion, wherein the FGF10 portion comprises or consists of the amino acid sequence set forth in SEQ ID NO: 2, in the presence of one, two, three, four, or five amino acid substitutions, additions, or deletions (e.g., a variant FGF10). In certain embodiments, the complex or fusion protein does not include the full length, naturally occurring FGF10 sequence set forth in SEQ ID NO: 1. In certain embodiments, the FGF10 portion comprises one, two, three, four, or five amino acid substitutions, and such a variant FGF10 portion has decreased mitogenic activity and/or FGFR binding relative to native FGF10 in the absence of the amino acid substitution(s). In certain embodiments, the FGF10 portion comprises one, two, three, or four amino acid substitutions. In certain embodiments, the amino acid substitutions are selected from: R78A, T114A, E158K, E158A, or K195A. In certain embodiments, the one, two, three, four, or five amino acid substitutions do not substantially alter surface positive charge or net charge of the FGF10 portion (e.g., the change in charge on the FGF10 portion is +1, 0, or −1).

Complexes of the disclosure comprise an FGF-10 portion, and the FGF-10 portion comprises an FGF10-related Surf+ Penetrating Polypeptide. The foregoing indicates exemplary FGF10-related Surf+ Penetrating Polypeptides, including variants. Suitable variants include variants that increase the net positive charge, the surface positive charge, and/or the charge per molecular weight ratio, as well as variants that decrease or eliminate an endogenous function of native FGF10. Given that cell penetrating FGF10 portions exist (e.g., SEQ ID NO: 2), variants with increased net positive charge, surface positive charge, and/or the charge per molecular weight ratio are not required. In certain embodiments, suitable variants decrease or eliminate an endogenous function of native FGF10. In certain embodiments, the FGF-10 portion comprises or consists of a variant having one, two, three, four, or five amino acid substitutions, relative to the corresponding domain of naturally occurring FGF-10, such as human FGF-10, but that the amino acid substitutions do not substantially alter net theoretical charge relative to the corresponding domain of naturally occurring FGF-10 (e.g., the change in charge on the FGF10 portion is +1, 0, or −1).

In certain embodiments of complexes of the disclosure, the (i) FGF-10 portion comprises or consists of a variant having 1, 2, 3, 4, or 5 amino acid substitutions and having decreased mitogenic activity and/or FGFR binding relative to native FGF10, such as the substitutions described above, and (ii) the cargo portion comprises a polypeptide, peptide, or small molecule; with the proviso that the cargo portion and/or the complex does not include one or more of: full length, unprocessed, naturally occurring FGF-10, a ligand binding domain of an FGF receptor, a native binding partner of FGF-10, and/or heparin. The cargo portion may be any of the cargo portions described herein.

(iii) Cargo

The disclosure provides complexes for use for delivery into cells and tissues, particularly into cells and tissues that are preferentially penetrated by an FGF10 portion and complexes comprising an FGF10 portion, such as liver and kidney. These complexes comprise an FGF10 portion (where, as detailed above, the FGF10 portion comprises or consists of all or a portion of an FGF10 polypeptide, or a variant thereof—for example, an FGF10-related Surf+ Penetrating Polypeptide) and a cargo portion. The cargo portion may be a protein, peptide, or small organic molecule. Generally, the cargo is one with therapeutic or cell modulating activity that requires transport into cells to achieve. Below various categories of cargo, as well as specific examples of cargo are described. These specific examples of cargo are merely illustrative. Complexes comprising an FGF10 domain and a cargo have substantial utility, for example, for delivering materials into cells of the liver, kidney, ovaries, and other cells and tissues of the abdominal cavity and GI tract. It should be understood that the cargo portion is heterologous to the FGF10 portion. In other words, the cargo portion does not include an FGF10 polypeptide from the same or different species. Moreover, in certain embodiments, the cargo portion (or the complex) does not include an FGF receptor or a ligand binding domain of an FGF receptor. In certain embodiments, the cargo portion (or the complex) does not include a polypeptide or peptide that endogenously binds FGF-10 in vivo under physiological conditions.

Proteins and Peptides

In certain embodiments, the cargo portion of the complex is a protein or peptide. In the context of a complex with an FGF10 portion the protein or peptide maintains its functional activity, such as enzymatic activity, target binding and inhibitory activity, transcription factor activity, tumor suppressor activity, and the like. Exemplary categories of proteins and peptides that may serve as cargo are described in more detail below. However, the disclosure contemplates that virtually any protein or peptide can be used as the cargo portion of a complex of the disclosure. In certain embodiments, the protein or peptide is one whose activity is needed in cells of the liver or the kidney. For example, the protein or peptide may be one that, under naturally occurring circumstances would be functional in the liver and/or kidney, and delivery is useful for augmenting or replacing activity that is supposed to be endogenously active in one or both of those tissues. By way of further example, the protein or peptide may be one designed to inhibit activity of a target that is expressed or misexpressed in the liver or kidney, and delivery is useful for inhibiting that activity. By way of further example, the protein or peptide may be one that inhibits activity of a target expressed or present in a tissue in which a cell penetrating FGF10 portion preferentially localizes, such as liver, kidney.

In certain embodiments, the cargo portion is a polypeptide or peptide but does not include an antibody or antibody mimic. In certain embodiments, the cargo portion does not include an antibody that binds specifically to FGF-10. In certain embodiments, the cargo portion does not include an enzyme. In certain embodiments, the cargo portion does not include a transcription factor.

Enzymes

In certain embodiments, the cargo portion comprises an enzyme. Without being bound by theory, complexes in which the cargo portion is an enzyme are suitable for enzyme replacement strategies in which subjects are unable to produce an enzyme having proper activity (at all or, at least, in sufficient quantities) necessary for normal function and, in some case, essential for life.

When provided as a complex with the FGF-10 portion, the enzyme portion (cargo portion comprising an enzyme) is delivered into cells where it can provide needed enzymatic activity. In certain embodiments, the enzyme being delivered is needed in the liver, kidney, or pancreas. In certain embodiments, the enzyme being delivered is one that is endogenously expressed in the liver, kidney, and/or pancreas of healthy subjects. Of course, it will be understood that the enzyme may but need not be endogenously expressed only in those tissues. Moreover, throughout the application, it is understood that although the FGF-10 portion does not localize ubiquitously into all cells (e.g., has preferential localization to particularly tissues), that does not mean that delivery is or is intended to be solely into a particular cell type where the cargo is needed.

An enzyme is a protein that can catalyze the rate of a chemical reaction within a cell. Enzymes are long, linear chains of amino acids that fold to produce a three-dimensional product having an active site containing catalytic amino acid residues. Substrate specificity is determined by the properties and spatial arrangement of the catalytic amino acid residues forming the active site.

As used herein an “enzyme” refers to a biologically active enzyme. The term “enzyme” further refers to “simple enzymes” which are composed wholly of protein, or “complex enzymes”, also referred to as “holoenzymes” which are composed of a protein component (the “apozyme”) and a relatively small organic molecule (the “co-enzyme”, when the organic molecule is non-covalently bound to the protein or “prosthetic group”, when the organic molecule is covalently bound to the protein).

As used herein the term an “enzyme” also refers to a gene for an enzyme and includes the full-length DNA sequence, a fragment thereof or a sequence capable of hybridizing thereto.

Classification of enzymes is conventionally based on the type of reaction catalyzed.

In certain embodiments of the disclosure the enzyme is selected from the group consisting of: a kinase, a phosphatase, a ligase, an oxidoreductase, a transferase, a hydrolase, a hydroxylase, a lyase, an isomerase, a dehydrogenase, an aminotransferase, a hexosamidase, a glucosidase, or a glucosyltransferase. The categories of enzymes are well known in the art and one of skill in the art can readily envision one or more examples of each category of enzyme. As noted above, in certain embodiments, the enzyme is a member of one of these categories and is endogenously expressed in the liver, kidney, and/or pancreas of healthy subjects (e.g., healthy humans).

To illustrate, a brief description of these categories of enzymes is provided. “Oxidoreductases” catalyze oxidation-reduction reactions. “Transferases” catalyze the transfer of a group (e.g a methyl group or a glycosyl group) from a donor compound to an acceptor compound. “Hydrolases” catalyze the hydrolytic cleavage of C—O, C—N, C—C and some other bonds, including phosphoric anhydride bonds. “Hydroxylases” catalyze the formation of a hydroxyl group on a substrate by incorporation of one atom (monooxygenases) or two atoms (dioxygenases) of oxygen. “Lyases” are enzymes cleaving C—C, C—O, C—N, and other bonds by elimination, leaving double bonds or rings, or conversely adding groups to double bonds. “Isomerases” catalyse intra-molecular rearrangements and, according to the type of isomerism, they may be called racemases, epimerases, cis-trans-isomerases, isomerases, tautomerases, mutases or cycloisomerases. “Ligases” catalyze bond formation between two compounds using the energy derived from the hydrolysis of a diphosphate bond in ATP or a similar triphosphate in ATP.

Other categories of enzymes, characterized by their substrate rather than the type of reaction catalyzed include the following: an enzyme that degrades glycosaminoglycans, glycolipids, or sphingolipids; an enzyme that degrades glycoproteins; an enzyme that degrades amino acids; an enzyme that degrades fatty acids; or an enzyme involved in energy metabolism. These categories of enzymes may, in some cases, overlap with the categories of enzymes described based on reaction catalyzed. Regardless of whether described based on substrate, reaction catalyzed, or both, one of skill in the art can readily envision examples of these classes of enzymes. Any of these are suitable for use in the present disclosure as a cargo portion. In certain embodiments, the enzyme is a member of one or more of these categories (based on substrate and/or reaction) and is endogenously expressed in the liver, kidney, and/or pancreas of healthy subjects (e.g., healthy humans). In certain embodiments, of any of the foregoing, the enzyme is a human enzyme (e.g., an enzyme that is typically expressed endogenously in humans). In certain embodiments, the enzyme is a mammalian enzyme.

In certain embodiments, an enzyme for use as a cargo portion in the present disclosure is not a ligase. In certain embodiments, an enzyme for use as a cargo portion in the present disclosure is not a kinase. In certain embodiments, an enzyme for use as a cargo portion in the present disclosure is not a recombinase.

Enzymes can function intracellularly or extracellularly. Intracellular enzymes are those whose endogenous function is inside a cell, such as in the cytoplasm or in a specific subcellular organelle. Such enzymes are responsible for catalyzing the reactions in the cellular metabolic pathways, for example, glycolysis. In the context of the present disclosure, delivery of intracellular enzymes is particularly preferred. In certain embodiments of the disclosure, the enzyme moiety is specifically targeted to an intracellular organelle in which the wild-type enzyme is constitutively or inducibly expressed.

In certain embodiments of the disclosure, the enzyme is a “kinase”, which catalyzes phosphoryltransfer reactions in all cells. Kinases are particularly prominent in signal transduction and co-ordination of complex functions such as the cell cycle. Non-limiting examples include tyrosine kinases, deoxyribonucleoside kinases, monophosphate kinases and diphosphate kinases.

In certain embodiments, the enzyme is a “dehydrogenase”. Dehydrogenases catalyze the removal of hydrogen from a substrate and the transfer of the hydrogen to an acceptor in an oxidation-reduction reaction. Widely implemented in the citric acid cycle, also referred to as the tricarboxylic acid cycle (TCA cycle) or the Krebs cycle, in which energy is generated in the matrix of the mitochondria through the oxidation of acetate derived from carbohydrates, fats and protein into carbon dioxide and water. Non-limiting examples of dehydrogenases include, medium-chain-acyl-CoA-dehydrogenase, very long-chain-acyl-CoA-dehydrogenase and isobutyryl-CoA-dehydrogenase.

In certain embodiments, the enzyme is an “aminotransferase” or “transaminase”. Such enzymes catalyze the transfer of an amino group from a donor molecule to a recipient molecule. The donor molecule is usually an amino acid while the recipient (acceptor) molecule is usually an alpha-2 keto acid.

In certain embodiments, the cargo portion is an enzyme. For example, the enzyme may be a human protein endogenously expressed in humans. Alternatively, the enzyme may be a non-human protein and/or a protein that is not endogenously expressed in humans.

Exemplary categories of enzymes suitable for use as cargo are: kinases, phosphatases, ligases, proteases, oxidoreductases, transferases, hydrolases, hydroxylases, lyases, isomerases, dehydrogenases, aminotransferases, hexosamidases, glucosidases, or glucosyltransferases. Thus, in certain embodiments, the cargo is an enzyme selected from the group consisting of a kinase, a phosphatase, a ligase, a protease, an oxidoreductase, a transferase, a hydrolase, a hydroxylase, a lyase, an isomerase, a dehydrogenase, an aminotransferase, a hexosamidase, a glucosidase, or a glucosyltransferase. In certain embodiments, the enzyme is a human enzyme endogenously expressed in human subjects.

Further exemplary categories of enzymes are: an enzyme that degrades glycosaminoglycans, glycolipids, or sphingolipids; an enzyme that degrades glycoproteins; an enzyme that degrades amino acids; an enzyme that degrades fatty acids; or an enzyme involved in energy metabolism. In certain embodiments, the enzyme is a human enzyme endogenously expressed in human subjects.

In certain embodiments, the enzyme is not a recombinase and/or is not a non-human protein.

In certain embodiments, the enzyme is a thymidine kinase, such as HSV-TK or a variant thereof.

The understanding in the art of enzymes is high, and examples of various human enzymes abound in the scientific and lay literature. One of skill in the art can select the appropriate enzyme and can readily obtain its amino acid sequence.

The disclosure contemplates that sometimes a particular protein is not itself an enzyme, but is necessary for enzymatic or other catalytic or functional activity. Accordingly, in certain embodiments, the cargo portion comprises a co-factor, accessory protein, or member of a multi-protein complex. Preferably, such a co-factor, accessory protein, or member of a multi-protein complex is a human protein or peptide. The protein or peptide should maintain its ability to bind to its endogenous cognate binding partners when provided as part of a complex (provided that for embodiments in which the complex is disrupted after cell penetration, the protein or peptide should maintain its ability to bind to its endogenous cognate binding partner(s) before and/or after complex disruption).

Tumor Suppressors

A tumor suppressor or anti-oncogene protects a cell from at least one step on the path to disregulated cell behavior, such as occurs in cancer. Mutations that result in a loss or decrease in the expression or function of a tumor suppressor protein can lead to cancer. Sometimes such a mutation is one of multiple genetic changes that ultimately lead to disregulated cell behavior. As used herein, a “tumor suppressor protein” or “tumor suppressor” is a protein, the loss of or decrease in expression and/or function of which, increases the likelihood of or ultimately leads to unregulated or disregulated cell proliferation, migration, or other changes indicative of hyperplastic or neoplastic transformation.

Unlike oncogenes, tumor suppressor genes often, although not exclusively, follow the “two-hit”, which implies that both alleles that code for a particular protein must be affected before a phenotype is discernable. This is because if only one allele for the gene is damaged, the second can sometimes still produce the correct protein in an amount sufficient to maintain proper function. There are exceptions to the “two-hit” model for tumor suppressors. For example, certain mutations in some tumor suppressors can function as a “dominant negative”, thus preventing the normal functioning of the protein produced from the wild type allele. Other examples include tumor suppressors that exhibit haploinsufficiency, such as patched (PTCH). Tumor suppressors that exhibit haploinsufficiency are sensitive to decreased levels or activity, such that even reduction in function following mutation in one allele is sufficient to result in a discernable phenotype.

Functional tumor suppressor proteins either have a dampening or repressive effect on the regulation of the cell cycle or promote apoptosis, and sometimes do both. Exemplary endogenous functions for tumor suppressor proteins generally fall into categories, such as the following:
Some tumor suppressor proteins repress the activity or expression of proteins or genes essential for continuing the cell cycle. In the absence of control by the tumor suppressor, the cell cycle may continue unchecked—leading to inappropriate cell division.
Some tumor suppressor proteins function to couple the cell cycle to DNA damage, such that the cell cycle will arrest if there is DNA damage and will only continue if that damage can be repaired. In the absence of control by the tumor suppressor, cells can divide in the presence of damaged DNA.
Some tumor suppressors are also referred to as metastasis suppressors because of their role in cell adhesion, which functions to prevent tumor cells from dispersing and losing contact inhibition properties. In the absence of this control, the risk and extent of metastasis increases.
Some tumor suppressors function as DNA repair proteins.
   There are numerous examples of tumor suppressor proteins belonging to any one or more of the foregoing classes, as well as tumor suppressors that can be separately characterized. One of skill in the art can readily envision numerous proteins characterized as tumor suppressor proteins. Exemplary tumor suppressor proteins include, but are not limited to, p16, patched (PTCH), and ST5. The disclosure contemplates that any tumor suppressor protein, including any of these specific tumor suppressor proteins and/or any of the foregoing category(ies) of tumor suppressor proteins are suitable for use as the cargo portion in the complexes of the disclosure.

In certain embodiments, the cargo portion (the tumor suppressor portion) does not include a transcription factor. In other words, in certain embodiments, the tumor suppressor protein is not also a transcription factor. In certain embodiments, the tumor suppressor portion does not include p53.

Complexes of the disclosure are useful for delivering a tumor suppressor protein to cells and tissues in vitro or in vivo. In certain embodiments, delivery is for augmenting or replacing missing or decreased function or expression of the endogenous tumor suppressor protein. Thus, although the function or expression of the tumor suppressor protein may not be decreased in all cells and tissue in culture or in an organism, the disclosure contemplates that the complexes deliver tumor suppressor protein to cells and tissue—at least a portion of which are characterized by decreased or missing function or expression of that tumor suppressor protein. In certain embodiments, the decreased or missing function and/or expression is due, at least in part, to a mutation in the gene encoding the tumor suppressor protein. In certain embodiments, the decreased or missing function and/or expression is not due to a mutation in the gene encoding the tumor suppressor protein.

To further describe the tumor suppressor portion of the complexes of the disclosure, exemplary tumor suppressor proteins are described below.

Patched (PTCH)

Protein patched homolog 1 (patched or PTCH) is encoded by the ptch gene and is a tumor suppressor protein. Mutations of this gene have been associated with nevoid basal cell carcinoma syndrome, basal cell carcinoma, medulloblastoma, esophageal squamous cell carcinoma, transitional cell carcinomas of the bladder, and rhabdomyosarcoma. Moreover, hereditary mutations in PTCH cause Gorlin syndrome, an autosomal dominant disorder. In addition, misregulation of this tumor suppressor protein can lead to other defects of growth regulation, such as holoprosencephaly and cleft lip and palate.

Given the role of PTCH as a tumor suppressor protein, in certain embodiments, complexes of the disclosure comprise PTCH or a functional fragment thereof. In other words, the tumor suppressor portion of the complex comprises, in certain embodiments, PTCH (such as human PTCH) or a functional fragment thereof.

ST5

Suppression of tumorigenicity 5 is a protein that in humans is encoded by the ST5 gene. This gene was identified by its ability to suppress the tumorigenicity of Hela cells in nude mice. The protein encoded by this gene contains a C-terminal region that shares similarity with the Rab 3 family of small GTP binding proteins. ST5 protein preferentially binds to the SH3 domain of c-Abl kinase, and acts as a regulator of MAPK1/ERK2 kinase, which may contribute to its ability to reduce the tumorigenic phenotype in cells.

Three alternatively spliced transcript variants of this gene encoding distinct isoforms exist. In certain embodiments, the cargo portion comprises ST5 or a functional fragment thereof. Isoform 3 (p70) of ST5 (see www.uniprot.org/uniprot/P78524) has been shown to restore contact inhibition in mouse fibroblast cell lines. Accordingly, in certain embodiments, the cargo portion of a complex of the disclosure comprises isoform 3 of ST5, preferably isoform 3 of human ST5.

ST5 was found downregulated following LH and FSH stimulation of human granulosa cells which comprise the main bulk of the ovarian follicular somatic cells. Rimon et al., Int J Oncol. 2004 May; 24(5):1325-38. Without being bound by theory, given that hypergonadotropin stimulation is believed to increase risk for ovarian cancer, administration of ST5 protein may help offset this down regulation. In such a context, ST5 administration may be useful not only as a therapeutic, but also as a prophylactic measure. However, therapeutic use in ovarian cancer is just one example. Given the tumor suppressor function of ST5, the disclosure contemplates providing ST5 in any context characterized to decreased expression and/or function of or mutation in ST5.

p16

p16 is a tumor suppressor protein and, in certain embodiments, complexes of the disclosure are useful for delivering a tumor suppressor protein, specifically p16 or a functional fragment thereof, to cells and tissues in vitro or in vivo. In other words, in certain embodiments, the cargo portion comprises p16 or a functional fragment thereof. In certain embodiments, delivery is for augmenting or replacing missing or decreased function or expression of endogenous p16 protein. Thus, although the function or expression of the tumor suppressor protein may not be decreased in all cells and tissue in culture or in an organism, the disclosure contemplates that the complexes deliver tumor suppressor protein to cells and tissue—at least a portion of which are characterized by decreased or missing function or expression of that p16 tumor suppressor protein. In certain embodiments, the decreased or missing function and/or expression is due, at least in part, to a mutation in the gene encoding p16 tumor suppressor protein. In certain embodiments, the decreased or missing function and/or expression is not due to a mutation in the gene encoding p16 tumor suppressor protein.

Tumor suppressors for use in the complexes of the disclosure comprise, in certain embodiments, p16, or a functional fragment thereof. The full length amino acid sequence of human p16 is set forth in SEQ ID NO: 5. Cyclin-dependent kinase inhibitor 2A, (CDKN2A, p16^{Ink4A}) is a tumor suppressor protein that, in humans, is encoded by the CDKN2A gene. This tumor suppressor protein is commonly referred to in the art and will be referred to herein as “p16” or “p16Ink4”. p16 plays an important role in regulating the cell cycle, and mutations in p16 increase the risk of developing a variety of cancers.

p16 has 5 isoforms (www.uniprot.org/uniprot/P42771), however, isoform 4 is a completely different protein arising from an alternate reading frame and expression of isoform 5 is generally undetectable in non-tumor cells. Isoforms 1, 2, 3, and 5 bind to CDK4/6 and are of interest and may be useful as the p16 portion of the complexes of the disclosure. A full length amino acid sequence of isoform 1 of human p16 (often referred to as the canonical p16 amino acid sequence) is of particular interest and is set forth in SEQ ID NO: 5. Isoform 2 is essentially a functional fragment of this canonical sequence—missing amino acids 1-51 relative to isoform 1. Isoform 3 is expressed specifically in the pancreas and, in certain embodiments, may be used to replace p16 function in subjects with a pancreatic tumor. The term “p16 tumor suppressor protein” or p16 refers to isoform 1, 2, 3, or 5 of p16, unless a specific isoform or sequence is specified. In certain embodiments, isoform 1 of human p16 (a protein having an amino acid sequence set forth in SEQ ID NO: 5) is used in a complex of the disclosure. In certain embodiments, the p16 portion comprises or consists of an amino acid sequence at least 80%, 85%, 90%, 91%, 92%, 93%, 94%, 95%, 96%, 97%, 98%, 99%, or 100% identical to SEQ ID NO: 5. Regardless of the particular p16 protein used in the complex, the protein must retain p16 bioactivity, such as the functions of p16 described herein and known in the art (e.g., binding to CDK6; ability to inhibit cyclin D-CDK4 kinase activity, etc.).

The CDKN2A gene generates several transcript variants that differ in their first exons. At least three alternatively spliced variants encoding distinct proteins have been reported, two of which encode structurally related isoforms known to function as inhibitors of CDK4. The remaining transcript includes an alternate exon 1 located 20 kilobases upstream of the remainder of the gene. This transcript contains an alternative open reading frame (ARF) that specifies a protein that is structurally unrelated to the products of the other variants. The ARF product functions as a stabilizer of the tumor suppressor protein p53. In spite of their structural and functional differences, the CDK inhibitor isoforms and the ARF product encoded by this gene, through the regulatory roles of CDK4 and p53 in cell cycle progression, share a common functionality in control of the G1 phase of the cell cycle. This gene is frequently mutated or deleted in a wide variety of tumors and is known to be an important tumor suppressor gene.

The present disclosure provides complexes comprising a p16 tumor suppressor protein, or a functional fragment or functional variant thereof, associated with a Surf+ Penetrating Polypeptide portion. In certain embodiments, the Surf+ Penetrating Polypeptide portion and/or the complex does not include a protein that is an endogenous substrate or binding partner for p16. In certain embodiments, the complex comprising a Surf+ Penetrating Polypeptide portion and a p16 portion does not include a transcription factor. In certain embodiments, the complex does not include p53.

Complexes of the disclosure are useful for delivering a tumor suppressor protein, specifically p16 or a functional fragment thereof, to cells and tissues in vitro or in vivo. In certain embodiments, delivery is for augmenting or replacing missing or decreased function or expression of endogenous p16 protein. Thus, although the function or expression of the tumor suppressor protein may not be decreased in all cells and tissue in culture or in an organism, the disclosure contemplates that the complexes deliver tumor suppressor protein to cells and tissue—at least a portion of which are characterized by decreased or missing function or expression of that p16 tumor suppressor protein. In certain embodiments, the decreased or missing function and/or expression is due, at least in part, to a mutation in the gene encoding p16 tumor suppressor protein. In certain embodiments, the decreased or missing function and/or expression is not due to a mutation in the gene encoding p16 tumor suppressor protein.

Tumor suppressors for use in the complexes of the disclosure comprise p16, or a functional fragment or functional variant thereof. Cyclin-dependent kinase inhibitor 2A, (CDKN2A, p16^{Ink4A}) is a tumor suppressor protein that, in humans, is encoded by the CDKN2A gene. This tumor suppressor protein is commonly referred to in the art and will be referred to herein as “p16” or “p16Ink4”. p16 plays an important role in regulating the cell cycle, and mutations in p16 increase the risk of developing a variety of cancers. The full length amino acid sequence of human p16, isoform 1 is set forth in SEQ ID NO: 5.

The disclosure contemplates the use of p16, such as human p16. In certain embodiments, the p16 portion comprises a full length, native p16 protein, such as a protein comprising the amino acid sequence of SEQ ID NO: 5. However, variants of native p16 that retain function (e.g., functional variants) can also be used. Exemplary variants retain the activity of p16 (e.g., retain greater than 50%, preferably greater than 70% of the native activity) and include 1, 2, 3, 4, 5, 6, 7, 8, 9, or 10 amino acid substitutions, deletions, or additions relative to the native p16 sequence. Each such change is independently selected (e.g., each substitution is independently selected). Further exemplary variants retain the activity of p16 and comprise an amino acid sequence at least 80%, 85%, 90%, 91%, 92%, 93%, 94%, 95%, 96%, 97%, 98%, 99%, or greater than 99% identical to SEQ ID NO: 5. Functional variants may also be a functional variant of a functional fragment of p16. Functional variants or the full length or fragment of native p16 also include variants, such as amino acid additions, deletions, substitutions, or truncations intended to increase protein stability improve biochemical or biophysical characteristics, or improve binding to CDK4 and/or CDK 6.

Contemplated functional fragments include fragments comprising: a fragment of p16 lacking the first ankyrin repeat, native isoform 2 (residues 52 to 156 of SEQ ID NO: 5), residues 10 to 134 of SEQ ID NO: 5, and residues 10 to 101 of SEQ ID NO: 5.

The p16 portion may be phosphorylated either during complex formation or in a post-production step. In certain embodiments, the p16 portion is not phosphorylated or is under phosphorylated (e.g., less phosphorylated then native p16). In certain embodiments, the p16 portion is hyper-phosphorylated (e.g., more phosphorylated then native p16).

Since its discovery as a CDKI (cyclin-dependent kinase inhibitor) in 1993, the importance in cancer of the tumor suppressor p16 (INK4A/MTS-1/CDKN2A) has gained widespread appreciation. The frequent mutations and deletions of p16 in human cancer cell lines first suggested an important role for p16 in carcinogenesis. This genetic evidence for a causal role was significantly strengthened by the observation that p16 was frequently inactivated in familial melanoma kindreds. Since then, a high frequency of p16 gene alterations were observed in many primary tumors.

In human neoplasms, p16 is silenced in at least three ways: homozygous deletion, methylation of the promoter, and point mutation. The first two mechanisms comprise the majority of inactivation events in most primary tumors. Additionally, the loss of p16 may be an early event in cancer progression, because deletion of at least one copy is quite high in some premalignant lesions. p16 is a major target in carcinogenesis, rivaled in frequency only by the p53 tumor-suppressor gene. Its mechanism of action as a CDKI has been elegantly elucidated and involves binding to and inactivating the cyclin D-cyclin-dependent kinase 4 (or 6) complex, and thus renders the retinoblastoma protein inactive. This effect blocks the transcription of important cell-cycle regulatory proteins and results in cell-cycle arrest.

Mutations in the CDKN2A gene and other factors that decrease the expression and/or function of a p16 protein isoform correlate with increased risk of a wide range of cancers. Exemplary cancers often associated with mutations or alterations in p16 include, but are not limited to, melanoma, pancreatic ductal adenocarcinoma, gastric mucinous cancer, primary glioblastoma, mantle cell lymphoma, hepatocellular carcinoma and ovarian cancer. Additionally, mutations or deletions in p16 are frequently found in, for example, esophageal and gastric cancer cell lines.

p16 misregulation is implicated in numerous cancers. Once such cancer is ovarian cancer, where the cancers of greater than half the patients have p16 misregulation. Accordingly, in certain embodiments, p16 portion complexes of the disclosure are particularly suitable for treating and studying ovarian cancer, as well as metastases from primary ovarian cancer. Additional discussion on ovarian cancer and p16 is provided below by way of a specific example of a cancer that could be treated and studied using the complexes of the disclosure. This is not meant to limit the claims, but merely to provide an example of a p16 deficient cancer that could be studied and/or treated.

Ovarian cancer is the most lethal of the gynecological malignancies. Novel-targeted therapies are needed to improve outcomes in ovarian cancer patients, where 75% of patients present with advanced (stage III or IV) disease. Although more than 80% of women treated benefit from first-line therapy, tumor recurrence occurs in almost all these patients at a median of 15 months from diagnosis (Hennessy B T, Coleman R L, Markman M. Ovarian cancer. Lancet 2009; 374:1371-8).

Cell cycle dysregulation is a common molecular finding in ovarian cancer. Under normal control, the cell cycle functions as a tightly regulated process consisting of several distinct phases. Progression through the G1-S phase requires phosphorylation of the retinoblastoma (Rb) protein by CDK4 or CDK6 (Harbour et al. Cdk phosphorylation triggers sequential intramolecular interactions that progressively block Rb functions as cells move through G1. Cell 1999; 98: 859-69; Lundberg A S, Weinberg R A. Functional inactivation of the retinoblastonma protein requires sequential modification by at least two distinct cyclin-cdk complexes. Mol Cell Biol 1998; 18:753-61; Chen et al. Overexpression of Cdk6-cyclin D3 highly sensitizes cells to physical and chemical transformation. Oncogene 2003; 22:992-1001) in complex with their activating subunits, the D type cyclins, D1, D2, or D3 (Meyerson M, Harlow E. Identification of G1 kinase activity for cdk6, a novel cyclin D partner. Mol Cell Biol 1994; 14:2077-86). Hyperphosphorylation of Rb diminishes its ability to repress gene transcription and consequently allows synthesis of several genes that encode proteins, which are necessary for DNA replication (Harbour J W, Dean D C. The Rb/E2F pathway: expanding roles and emerging paradigms. Genes Dev 2000; 14:2393-409).

Deregulation of the CDK4/6-cyclin D/p16-Rb signaling pathway is among the most common aberrations found in human cancer (Hanahan D. Weinberg R A. The hallmarks of cancer. Cell 2000; 100: 57-70). Mutations in p16 have been found in >70 different types of tumor cells (as reviewed in Cordon-Cardo, 1995). In the case of ovarian cancer, p16 (also called MTS1 or CDKN2) expression is most commonly altered due to promoter methylation, and less commonly by homozygous deletion or mutation. A recent report indicates that of 249 ovarian cancer patients, 100 (40%) tested positive for p16 promoter methylation (Katsaros D, Cho W, Singal R, Fracchioli S, Rigault De La Longrais I A, Arisio R, et al. Methylation of tumor suppressor gene p16 and prognosis of epithelial ovarian cancer. Gynecol Oncol 2004; 94:685-92). Homozygous deletions of the p16 gene (CDKN2A) were detected in 16/115 (14%) or 8/45 (18%) (Schultz D C, Vanderveer L. Buetow K H, Boente M P, Ozols R F, Hamilton T C, et al. Characterization of chromosome 9 in human ovarian neoplasia identifies frequent genetic imbalance on 9q and rare alterations involving 9p, including CDKN2. Cancer Res 1995; 55:2150-7; Kudoh K, Ichikawa Y, Yoshida S, Hirai M, Kikuchi Y, Nagata I, et al. Inactivation of p16/CDKN2 and p15/MTS2 is associated with prognosis and response to chemotherapy in ovarian cancer. Int J Cancer 2002; 99:579-82), and mutations in 53/673 (8%) of ovarian cancers (www.sanger.ac.uk/genetics/CGP/cosmic). Thus, by these estimates, greater than 60% of ovarian cancers have misregulation of p16.

A novel opportunity to intervene in ovarian and other cancers, including pancreatic where DNA replication is affected due to a decrease in expression of p16 or mutations that affect its activity, is to replace functional p16 protein. In certain embodiments, functional p16 protein is replaced in cells or tissues that are Rb⁺ tumor cells. Functional replacement would thereby inhibit assembly of active cyclin D-CDK4/6 complexes, and thus inhibit the phosphorylation of the Rb protein. The present disclosure provides an approach for p16 replacement therapy using cell penetration proteins that facilitate delivery of therapeutics into cells. Moreover, the present disclosure provides evidence that, depending on the particular cell penetration protein (e.g., Surf+ Penetrating Polypeptide) chosen, delivery is not ubiquitous. Rather, there is a level of specificity and preferential localization to some tissues over others. Without wishing to be bound by theory, this not only facilitates delivery, but may also decrease side effects and decrease the required effective dosage.

Thus, we describe a novel approach for replacement of p16 function through direct delivery of a functional p16 protein, or functional fragment thereof) to tumor cells that are, optionally, Rb⁺ tumor cells by fusion to a Surf+ Penetrating Polypeptide portion. For example, a cell penetrating domain of FGF-10 can be used to delivery p16 and therefore replace deficient levels of this tumor suppressor due to, for example, promoter methylation or homozygous deletion or mutation.

Importantly, in knock out mouse studies, p16 has been demonstrated to be a haplo-insufficient locus, meaning that cells are sensitive to the levels of p16. This suggests that altering levels through direct delivery of the protein will have meaningful effect on apoptosis induction.

Additionally, as detailed above, functional variants and functional fragments of p16 that, for example, display less conformational flexibility and/or less tendency to aggregate may be delivered as the p16 portion of the fusion protein instead of a native human sequence.

Evaluation of anti-tumor efficacy of a complex of the disclosure comprising a p16 tumor suppressor protein, or a functional fragment or variant thereof, as a novel cancer therapeutic can be performed in preclinical cancer models or in in vitro biochemical or cell biological assays of p16 function. Demonstration of the effects of p16 replacement therapy through a fusion with a Surf+ Penetrating Polypeptide portion can be through evaluation of apoptosis induction, evaluation of the effects on Rb phosphorylation, and effects on the cell cycle. Initially, these effects can be evaluated on human cancer cell lines in vitro, with follow up studies in human tumor xenografts, including explants from human derived tissues, following either systemic or intraperitoneal delivery. Assays may be carried our using, for example, ovarian, pancreatic, or ovarian cancer cell lines and/or xenograft models.

For a human therapeutic intervention, a complex of the disclosure would be expected to provide a maximized therapeutic effect while allowing patients to minimize chemotherapy side effects by avoiding drugs that cause excessive toxicity.

Furthermore, intraperitoneal delivery would be expected to maximize the delivery of drug to tumor cells, particularly when treating ovarian cancer, or a primary or metastatic lesion in the abdominal cavity (e.g., liver mets). The ability to administer complexes of the disclosure, such as fusion proteins, directly to the intraperitoneal cavity will provide for the highest concentrations to be achieved at the tumor site, including the ovaries and fallopian tubes, and sites of typical metastases. As ovarian cancer tends to recur and progress within the abdominal cavity, regional intraperitoneal therapy for ovarian cancer is attractive. Furthermore the opportunity for repeated regional IP delivery by placement of an IP catheter for multiple courses of treatment provides further advantage. In certain embodiments, a complex of the disclosure is administered intraperitoneally. In other embodiments, a complex of the disclosure is administered intratumorally. Intratumoral administration provides many of the benefits of IP administration in terms of maximizing dose to the tumor and minimizing exposure to healthy tissues. However, systemic administration is also contemplated.

Subpopulations of patients most likely to respond to treatment may be identified for specific intervention. Selection of such patients can be through immunohistochemistry studies for alterations in p16 expression. Thus, a p16 fusion as a therapeutic can taking advantage of personalized therapy. Furthermore, patients can be selected through immunohistochemistry studies for alternations in Rb expression where patients who are Rb competent as more likely to respond to a p16 replacement protein.

As mentioned, recurrence following treatment of ovarian cancer is frequent, and is complicated by the emergence of drug resistance. As Surf+ Penetrating Polypeptides deliver their cargo by entering cells through an endocytic process involving heparan sulphate proteoglycans, typical emergence of drug resistance is unlikely to affect this class of drugs.

Additionally, in early or advanced stages of disease, a p16 therapeutic of the disclosure can be used in novel combination regimens with existing approved therapeutics or new agents, for example combining with CDK4/6 inhibitors or other therapeutics specifically affecting the cell cycle, or tumor cell growth in general.

Given the role of p16 as a tumor suppressor protein, in certain embodiments, complexes of the disclosure comprise p16 or a functional fragment or functional variant thereof. In other words, the tumor suppressor portion of the complex comprises, in certain embodiments, p16 (such as human p16) or a functional fragment or functional variant thereof. Such complexes may be particularly suitable for in vitro studies of cells deficient in p16 expression and/or function as models of tumorogenesis. Additionally or alternatively, such complexes may be administered to a subject comprising cells and tissues in which p16 expression and/or function is deficient. Such studies could be used to deliver p16 protein to cells, including cells deficient for or having low expression of p16 and cell that are Rb+. Moreover, such studies could be used to increase p16 expression and/or function in patients in need thereof (e.g., patients having a p16 deficiency—particularly a deficiency associated with a hyperplastic or neoplastic state—including a hyperplastic or neoplastic state where cells have a deficiency in p16 but are Rb+). In certain embodiments, the patient in need thereof has p16 deficiency associated with melanoma, ovarian cancer, pancreatic cancer, cervical cancer, or hepatocellular carcinoma. In certain embodiments, the patient has a p16 deficient cancer that has metastasized to the liver.

The foregoing are merely exemplary of tumor suppressor proteins that can be the cargo portion of a complex of the disclosure.

Transcription Factors

In certain embodiments, the cargo portion comprises a transcription factor. Without being bound by theory, complexes in which the cargo portion is a transcription factor are suitable for replacement strategies in which subjects have a deficiency in the quantity or function of a transcription factor, such as due to mutation, and this deficiency causes (directly or indirectly) some undesirable symptoms or condition.

When provided as a complex with the FGF-10 portion, the transcription factor portion (e.g., the cargo portion comprises a transcription factor) is delivered into cells where it can provide needed activity. Generally, transcription factors function in the nucleus of a cell, and thus, preferably the transcription factor is delivered into the nucleus of a cell. Such deliver may be facilitated by inclusion of an NLS on some portion of the complex, or by retaining an endogenous NLS from the selected transcription factor. In certain embodiments, the transcription factor being delivered is needed in the liver, kidney, or pancreas. In certain embodiments, the transcription factor being delivered is one that is endogenously expressed in the liver, kidney, and/or pancreas of healthy subjects. Of course, it will be understood that the transcription factor may but need not be endogenously expressed only in those tissues.

A transcription factor is a protein that binds to specific nucleic acid sequences, directly or via one or more additional proteins, to modulate transcription. Transcription factors perform this function alone or with other proteins in a complex. Transcription factors sometimes function to promote or activate transcription and sometimes to block or repress transcription. Some transcription factors are either activators or repressors, and others can perform either function depending on the context (e.g., promote expression of some targets but repress expression of other targets). The effect of a transcription factor may be binary (e.g., transcription is turned on or off) or a transcription factor may modulate the level, timing, or spatio-temporal regulation of transcription.

A defining feature of transcription factors is that they contain one or more DNA-binding domains (DBDs). DBDs recognize and bind to specific sequences of DNA adjacent to the gene(s) being regulated by the transcription factor. Transcription factors are often classified based on their DBDs which help define the sequences bound, and thus, help define possible target genes.

Generally, transcription factors bind to either enhancer or promoter regions of DNA adjacent to the genes that they regulate. As noted above, depending on the transcription factor, the transcription of the adjacent gene is either up- or down-regulated. Transcription factors use a variety of mechanisms for the regulation of gene expression.

Transcription factors play a key role in many important cellular processes. As such, their misregulation can be deleterious to the subject. Some of the important functions and biological roles transcription factors are involved in include, but are not limited to, mediating differential enhancement of transcription, development, mediating responses to intercellular signals, facilitating the response to the environment, cell cycle control, and pathogenesis. These functions for transcription factors are briefly summarized below.

Some transcription factors differentially regulate the expression of various genes by binding to enhancer regions of DNA adjacent to regulated genes. These transcription factors are critical to making sure that genes are expressed in the right cell at the right time and in the right amount, depending on the changing requirements of the organism.

Many transcription factors are involved in development. In response to various internal or external stimuli, these transcription factors turn on/off the transcription of the appropriate genes, and help mediate processes such as changes in cell morphology, cell fate determination, proliferation, and differentiation.

Some transcription factors also help cells communicate with each other. This is often mediated via signaling cascaded initiated by cell-cell interactions and/or ligand-receptor interactions. Transcription factors are often downstream components of signaling cascades and, help up or down-regulate transcription in response to the signaling cascade.

Not only do transcription factors act downstream of signaling cascades related to biological stimuli but they can also be downstream of signaling cascades involved in environmental stimuli. Examples include heat shock factor (HSF), which upregulates genes necessary for survival at higher temperatures, hypoxia inducible factor (HIF), which upregulates genes necessary for cell survival in low-oxygen environments, and sterol regulatory element binding protein (SREBP), which helps maintain proper lipid levels in the cell.

Transcription factors can also be used to alter gene expression in a host cell to promote pathogenesis. A well studied example of this are the transcription-activator like effectors (TAL effectors) secreted by Xanthomonas bacteria.

The foregoing are exemplary of categories of transcription factors and, in certain embodiments, a member of any one or more of such categories of transcription factors may be used as a cargo portion.

Transcription factors are modular in structure and contain the following domains:
DNA-binding domain (DBD)
Trans-activating or Trans-activation domain (TAD)
(optional) Signal sensing domain (SSD).

In certain embodiments, the cargo portion is a transcription factor, and the transcription factor is a human protein. In certain embodiments, the cargo portion does not include a transcription factor. In certain embodiments, the complex does not include a transcription factor.

Target Binding Moiety

In certain embodiments, the cargo portion comprises a target-binding moiety. A target-binding moiety is polypeptide or peptide that binds to a target. Typically, the target-binding moiety binds to and inhibits an activity of the target. Exemplary target-binding moieties include antibodies, antibody mimics, ligand binding domains of a receptor, and receptor binding domains of a ligand. In certain embodiments, the target is expressed in or present intracellularly. In certain embodiments, the target is expressed or present in the liver, kidney, pancreas, or ovaries.

In certain embodiments, the target-binding moiety is an antibody or an antibody mimic molecule that specifically binds to a target. An antibody-mimic molecule is also referred to as an antibody-like molecule. An antibody-mimic binds to a target, but binding is mediated by binding units other than antigen binding portions comprising at least a variable heavy or variable light chain of an antibody. Thus, in an antibody mimic, binding to target is mediated by a different antigen-binding unit, such as a protein scaffold or other engineered binding unit. Numerous categories of antibody-mimics are well known in the art and are described in further detail below.

In certain embodiments, the target-binding moiety is an adhesin molecule. In certain embodiments, the term “adhesin” refers to a chimeric molecule which combines the “binding domain” (e.g., the extracellular domain) of a heterologous “adhesion” protein (e.g., a receptor, ligand, or enzyme) with an immunoglobulin sequence. In certain embodiments, the immunoglobulin sequence is an immunoglobulin effector or constant domain (e.g., Fc domain). Structurally, the immunoadhesins comprise a fusion of the adhesion amino acid sequence with the desired binding specificity which is other than the antigen recognition and binding site of an antibody (i.e., is “heterologous”) and an immunoglobulin effector or constant domain sequence. The immunoglobulin constant domain sequence in the adhesin molecule may be obtained from any immunoglobulin, such as IgG1, IgG2, IgG3, or IgG4 subtypes. IgA, IgE, IgD or IgM. Such adhesin molecule has the ability of specifically binding to the target. Numerous categories of such polypeptides (e.g., adhesin molecules) are well known in the art and are described in further detail below.

In certain embodiments, a complex of the disclosure comprises a target binding moiety, wherein the target binding moiety is an antibody or an antibody mimic molecule that binds to a target molecule. In certain embodiments, a complex of the disclosure comprises a target binding moiety, wherein the target binding moiety is an antibody-mimic (e.g., a protein comprising a protein scaffold or other binding unit that binds to a target). In certain embodiments, a complex of the disclosure comprises a target binding moiety, wherein the target binding moiety comprises a ligand or a receptor-binding domain of the ligand. In certain embodiments, a complex of the disclosure comprises a target binding moiety, wherein the target binding moiety comprises a receptor, or a ligand-binding domain of the receptor, or an extracellular domain of the receptor.

In certain embodiments, a target binding moiety is an antibody-mimic comprising a protein scaffold. Scaffold-based target binding moieties have positioning or structural components and target-contacting components in which the target contacting residues are largely concentrated. Thus, in an embodiment, a scaffold-based target binding moiety comprises a scaffold comprising two types of regions, structural and target contacting. The target contacting region shows more variability than does the structural region when a scaffold-based target binding moiety to a first target is compared with a scaffold-based target binding moiety of a second target. The structural region tends to be more conserved across target binding moieties that bind different targets. This is analogous to the CDRs and framework regions of antibodies. In the case of an Anticalin®, the first class corresponds to the loops, and the second class corresponds to the anti-parallel strands.

In certain embodiments the target binding moiety is a subunit-based target binding moiety. These target binding moieties are based on an assembly of subunits which provide distributed points of contact with the target that form a domain that binds with high affinity to the target (e.g. as seen with DARPins).

In certain embodiments a target binding moiety for use as part of a complex of the disclosure has a molecular weight of 5-250, 10-200, 5-15, 10-30, 15-30, 20-25 kD. Target binding moieties can comprise one or more polypeptide chains.

Target binding moieties can be antibody-based or non-antibody-based.

Target binding moieties suitable for use in the compositions and methods featured in the disclosure include antibody molecules, such as full-length antibodies and antigen-binding fragments thereof, and single domain antibodies, such as camelids. For example, an antibody molecule is the cargo portion of a complex of the disclosure, and complexed with an FGF-10 portion for delivery of the antibody molecule into a cell. The antibody molecule binds a target, such as to inhibit the target, e.g., for treatment of a disease or a condition.

Other suitable target binding moieties include polypeptides engineered to contain a scaffold protein, such as a DARPin or an Anticalin®. These are exemplary of antibody-mimic moieties that, in the context of the disclosure, may be complexed with an FGF-10 portion to promote delivery of a target, to which the target binding moiety binds, into a cell. The scaffold protein (e.g., the target binding moiety portion of the complex) binds a target, such as to inhibit the target, e.g., for treatment of a disease or condition. Inhibition can be, e.g., by steric inhibition, e.g., by blocking protein interaction with a substrate (e.g., interaction between the target and its corresponding receptor molecule), or inhibition can be, e.g., by causing target protein degradation.

Antibody Molecules

As used herein, the term “antibody” or “antibody molecule” refers to a protein that includes sufficient sequence (e.g., antibody variable region sequence) to mediate binding to a target, and in embodiments, includes at least one immunoglobulin variable region or an antigen binding fragment thereof.

An antibody molecule can be, for example, a full-length, mature antibody, or an antigen binding fragment thereof. An antibody molecule, also known as an antibody or an immunoglobulin, encompass monoclonal antibodies (including full-length monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain Fvs (scFv), Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the disclosure), intrabodies, and epitope-binding fragments of any of the above. In particular, antibodies include immunoglobulin molecules and immunologically active fragments of immunoglobulin molecules, i.e., molecules that contain at least one antigen-binding site. Immunoglobulin molecules can be of any isotype (e.g., IgG, IgE, IgM, IgD, IgA and IgY), subisotype (e.g., IgG1, IgG2, IgG3, IgG4, IgA1 and IgA2) or allotype (e.g., Gm, e.g., G1m(f, z, a or x), G2m(n), G3m(g, b, or c), Am, Em, and Km(1, 2 or 3)). Antibodies may be derived from any mammal, including, but not limited to, humans, monkeys, pigs, horses, rabbits, dogs, cats, mice, etc., or other animals such as birds (e.g. chickens). The antibody molecule can be a single domain antibody, e.g., a nanobody, such as a camelid, or a llama- or alpaca-derived single domain antibody, or a shark antibody (IgNAR). The single domain antibody comprises, e.g., only a variable heavy domain (VHH). An antibody molecule can also be a genetically engineered single domain antibody. Typically, the antibody molecule is a human, humanized, chimeric, camelid, shark or in vitro generated antibody.

Examples of fragments include (i) an Fab fragment having a VL, VH, constant light chain domain (CL) and constant heavy chain domain 1 (CH1) domains; (ii) an Fd fragment having VH and CH1 domains; (iii) an Fv fragment having VL and VH domains of a single antibody; (iv) a dAb fragment (Ward, E. S. et al., Nature 341, 544-546 (1989); McCafferty et al (1990) Nature, 348, 552-55; and Holt et al (2003) Trends in Biotechnology 21, 484-490), having a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988 and Huston et al, PNAS USA, 85, 5879-5883, 1988) (viii) bispecific single chain Fv dimers (for example as disclosed in WO 1993/011161) and (ix) “diabodies”, multivalent or multispecific fragments constructed by gene fusion (for example as disclosed in WO94/13804 and Holliger. P. et al, Proc. Natl. Acad. Sci. USA 90 6444-6448, 1993). Fv, scFv or diabody molecules may be stabilized by the incorporation of disulphide bridges linking the VH and VL domains (Reiter, Y. et al. Nature Biotech, 14, 1239-1245, 1996). Minibodies comprising a scFv joined to a CH3 domain may also be made (Hu, S. et al, Cancer Res., 56, 3055-3061, 1996). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group. These antibody fragments are obtained using conventional techniques known to those with skill in the art, and the fragments are screened for utility in the same manner as are intact antibodies. Suitable fragments may, in certain embodiments, be obtained from human or rodent antibodies.

The term “antibody molecule” includes intact molecules as well as functional fragments thereof. Constant regions of the antibody molecules can be altered, e.g., mutated, to modify the properties of the antibody (e.g., to increase or decrease one or more of: Fc receptor binding, antibody glycosylation, the number of cysteine residues, effector cell function, or complement function). In certain embodiments, antibodies for use in the present disclosure are labeled, modified to increase half-life, and the like. For example, in certain embodiments, the antibody is chemically modified, such as by PEGylation, or by incorporation in a liposome.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, light chains devoid of heavy chains, single domain antibodies derived from conventional 4-chain antibodies, and engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In one aspect of the disclosure, a single domain antibody can be derived from a variable region of the immunoglobulin found in fish, such as, for example, that which is derived from the immunoglobulin isotype known as Novel Antigen Receptor (NAR) found in the serum of shark. Methods of producing single domain antibodies derived from a variable region of NAR (“IgNARs”) are described in WO 03/014161 and Streltsov (2005) Protein Sci. 14:2901-2909. According to another aspect, a single domain antibody is a naturally occurring single domain antibody known as a heavy chain antibody devoid of light chains. Such single domain antibodies are disclosed in WO 9404678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; and such VHHs are within the scope of the disclosure.

The VH and VL regions can be subdivided into regions of hypervariability, termed “complementarity determining regions” (CDR), interspersed with regions that are more conserved, termed “framework regions” (FR). The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modelling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). Each VH and VL typically includes three CDRs and four FRs, arranged from amino-terminus to carboxy-terminus in the following order: FR1, CDR1, FR2, CDR2, FR3, CDR3, FR4.

The VH or VL chain of the antibody molecule can further include all or part of a heavy or light chain constant region, to thereby form a heavy or light immunoglobulin chain, respectively. In one embodiment, the antibody molecule is a tetramer of two heavy immunoglobulin chains and two light immunoglobulin chains. The heavy and light immunoglobulin chains can be connected by disulfide bonds. The heavy chain constant region typically includes three constant domains. CH1, CH2 and CH3. The light chain constant region typically includes a CL domain. The variable region of the heavy and light chains contains a binding domain that interacts with an antigen. The constant regions of the antibody molecules typically mediate the binding of the antibody to host tissues or factors, including various cells of the immune system (e.g., effector cells) and the first component (C1q) of the classical complement system.

The term “immunoglobulin” comprises various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgD, or IgE, respectively. The immunoglobulin subclasses (isotypes) e.g., IgG1, IgG2, IgG3, IgG4, IgA1, etc. are well characterized and are known to confer functional specialization. Modified versions of each of these classes and isotypes are readily discernable to the skilled artisan in view of the instant disclosure and, accordingly, are within the scope of the present disclosure. All immunoglobulin classes are also within the scope of the present disclosure. Light chains are classified as either kappa or lambda (κ, λ). Each heavy chain class may be bound with either a kappa or lambda light chain.

The term “antigen-binding fragment” refers to one or more fragments of a full-length antibody that retain the ability to specifically bind to a target of interest. Examples of binding fragments encompassed within the term “antigen-binding fragment” of a full length antibody include (i) a Fab fragment, a monovalent fragment having VL, VH, CL and CH1 domains; (ii) a F(ab′)₂ fragment, a bivalent fragment including two Fab fragments linked by a disulfide bridge at the hinge region; (iii) an Fd fragment having VH and CH1 domains; (iv) an Fv fragment having VL and VH domains of a single arm of an antibody, (v) a dAb fragment (Ward et al., (1989) Nature 341:544-546), which has a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The term “antigen-binding site” refers to the part of an antibody molecule that comprises determinants that form an interface that binds to a target antigen, or an epitope thereof. With respect to proteins (or protein mimetics), the antigen-binding site typically includes one or more loops (of at least four amino acids or amino acid mimics) that form an interface that binds to the target antigen or epitope thereof. Typically, the antigen-binding site of an antibody molecule includes at least one or two CDRs, or more typically at least three, four, five, or six CDRs.

Regardless of the type of antibody used, in certain embodiments, the antibody may comprise replacing one or more amino acid residue(s) with a non-naturally occurring or non-standard amino acid, modifying one or more amino acid residue into a non-naturally occurring or non-standard form, or inserting one or more non-naturally occurring or non-standard amino acid into the sequence. Examples of numbers and locations of alterations in sequences are described elsewhere herein. Naturally occurring amino acids include the 20 “standard” L-amino acids identified as G, A, V, L, I, M, P, F, W, S, T, N, Q, Y, C, K, R, H, D, E by their standard single-letter codes. Non-standard amino acids include any other residue that may be incorporated into a polypeptide backbone or result from modification of an existing amino acid residue. Non-standard amino acids may be naturally occurring or non-naturally occurring. Several naturally occurring non-standard amino acids are known in the art, such as 4-hydroxyproline, 5-hydroxylysine, 3-methylhistidine, N-acetylserine, etc. (Voet & Voet, Biochemistry, 2nd Edition, (Wiley) 1995). Those amino acid residues that are derivatised at their N-alpha position will only be located at the N-terminus of an amino-acid sequence. Normally, an amino acid is an L-amino acid, but it may be a D-amino acid. Alteration may therefore comprise modifying an L-amino acid into, or replacing it with, a D-amino acid. Methylated, acetylated and/or phosphorylated forms of amino acids are also known, and amino acids in the present disclosure may be subject to such modification. Additionally, the derivative can contain one or more non-natural or unusual amino acids by using the Ambrx ReCODE™ technology (see, e.g., Wolfson, 2006, Chem. Biol. 13(10):1011-2).

In certain embodiments, the antibodies used in the claimed methods are generated using random mutagenesis of one or more selected VH and/or VL genes to generate mutations within the entire variable domain. Such a technique is described by Gram et al., 1992, Proc. Natl. Acad. Sci., USA, 89:3576-3580 who used error-prone PCR. In some embodiments one or two amino acid substitutions are made within an entire variable domain or set of CDRs.

Another method that may be used is to direct mutagenesis to CDR regions of VH or VL genes. Such techniques are disclosed by Barbas et al., 1994, Proc. Natl. Acad. Sci., USA, 91:3809-3813 and Schier et al., 1996, J. Mol. Biol. 263:551-567.

Preparation of Antibodies

Suitable antibodies for use as a target binding moiety can be prepared using methods well known in the art. For example, antibodies can be generated recombinantly, made using phage display, produced using hybridoma technology, etc. Non-limiting examples of techniques are described briefly below.

In general, for the preparation of monoclonal antibodies or their functional fragments, especially of murine origin, it is possible to refer to techniques which are described in particular in the manual “Antibodies” (Harlow and Lane, Antibodies: A Laboratory Manual, Cold Spring Harbor Laboratory, Cold Spring Harbor N.Y., pp. 726, 1988) or to the technique of preparation from hybridomas described by Köhler and Milstein, Nature, 256:495-497, 1975.

Monoclonal antibodies can be obtained, for example, from a cell obtained from an animal immunized against the target antigen, or one of its fragments. Suitable fragments and peptides or polypeptides comprising them may be used to immunise animals to generate antibodies against the target antigen.

The monoclonal antibodies can, for example, be purified on an affinity column on which the target antigen or one of its fragments containing the epitope recognized by said monoclonal antibodies, has previously been immobilized. More particularly, the monoclonal antibodies can be purified by chromatography on protein A and/or G, followed or not followed by ion-exchange chromatography aimed at eliminating the residual protein contaminants as well as the DNA and the lipopolysaccharide (LPS), in itself, followed or not followed by exclusion chromatography on Sepharose™ gel in order to eliminate the potential aggregates due to the presence of dimers or of other multimers. In one embodiment, the whole of these techniques can be used simultaneously or successively.

It is possible to take monoclonal and other antibodies and use techniques of recombinant DNA technology to produce other antibodies or chimeric molecules that bind the target antigen. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400, and a large body of subsequent literature. A hybridoma or other cell producing an antibody may be subject to genetic mutation or other changes, which may or may not alter the binding specificity of antibodies produced.

Further techniques available in the art of antibody engineering have made it possible to isolate human and humanised antibodies. For example, human hybridomas can be made as described by Kontermann, R & Dubel, S, Antibody Engineering, Springer-Verlag New York, LLC; 2001, ISBN: 3540413545. Phage display, another established technique for generating antagonists has been described in detail in many publications, such as Kontermann & Dubel, supra and WO92/01047 (discussed further below), and U.S. Pat. No. 5,969,108, U.S. Pat. No. 5,565,332, U.S. Pat. No. 5,733,743, U.S. Pat. No. 5,858,657, U.S. Pat. No. 5,871,907. U.S. Pat. No. 5,872,215, U.S. Pat. No. 5,885,793, U.S. Pat. No. 5,962,255, U.S. Pat. No. 6,140,471, U.S. Pat. No. 6,172,197, U.S. Pat. No. 6,225,447, U.S. Pat. No. 6,291,650, U.S. Pat. No. 6,492,160 and U.S. Pat. No. 6,521,404.

Transgenic mice in which the mouse antibody genes are inactivated and functionally replaced with human antibody genes while leaving intact other components of the mouse immune system, can be used for isolating human antibodies Mendez, M. et al. (1997) Nature Genet, 15(2): 146-156. Humanised antibodies can be produced using techniques known in the art such as those disclosed in, for example, WO91/09967, U.S. Pat. No. 5,585,089, EP592106, U.S. Pat. No. 5,565,332 and WO93/17105. Further, WO2004/006955 describes methods for humanising antibodies, based on selecting variable region framework sequences from human antibody genes by comparing canonical CDR structure types for CDR sequences of the variable region of a non-human antibody to canonical CDR structure types for corresponding CDRs from a library of human antibody sequences, e.g. germline antibody gene segments. Human antibody variable regions having similar canonical CDR structure types to the non-human CDRs form a subset of member human antibody sequences from which to select human framework sequences. The subset members may be further ranked by amino acid similarity between the human and the non-human CDR sequences. In the method of WO2004/006955, top ranking human sequences are selected to provide the framework sequences for constructing a chimeric antibody that functionally replaces human CDR sequences with the non-human CDR counterparts using the selected subset member human frameworks, thereby providing a humanized antibody of high affinity and low immunogenicity without need for comparing framework sequences between the non-human and human antibodies. Chimeric antibodies made according to the method are also disclosed.

Synthetic antibody molecules may be created by expression from genes generated by means of oligonucleotides synthesized and assembled within suitable expression vectors, for example as described by Knappik et al. J. Mol. Biol. (2000) 296, 57-86 or Krebs et al. Journal of Immunological Methods 254 2001 67-84.

Note that regardless of how an antibody of interest is initially identified or made, any such antibody can be subsequently produced using recombinant techniques. For example, a nucleic acid sequence encoding the antibody may be expressed in a host cell. Such methods include expressing nucleic acid sequence encoding the heavy chain and light chain from separate vectors, as well as expressing the nucleic acid sequences from the same vector. These and other techniques using a variety of cell types are well known in the art.

Using these and other techniques known in the art, antibodies that specifically bind to any target can be made. Once made, antibodies can be tested to confirm that they bind to the desired target antigen and to select antibodies having desired properties. Such desired properties include, but are not limited to, selecting antibodies having the desired affinity and cross-reactivity profile. Given that large numbers of candidate antibodies can be made, one of skill in the art can readily screen a large number of candidate antibodies to select those antibodies suitable for the intended use. Moreover, the antibodies can be screened using functional assays to identify antibodies that bind the target and have a particular function, such as the ability to inhibit an activity of the target or the ability to bind to the target without inhibiting its activity. Thus, one can readily make antibodies that bind to a target and are suitable for an intended purpose.

The nucleic acid (e.g., the gene) encoding an antibody can be cloned into a vector that expresses all or part of the nucleic acid. For example, the nucleic acid can include a fragment of the gene encoding the antibody, such as a single chain antibody (scFv), a F(ab′)₂ fragment, a Fab fragment, or an Fd fragment.

Antibodies may also include modifications, e.g., modifications that alter Fc function, e.g., to decrease or remove interaction with an Fc receptor or with C1q, or both. For example, the human IgG4 constant region can have a Ser to Pro mutation at residue 228 to fix the hinge region.

In another example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some antibodies that include an Fc domain, the antibody production system may be designed to synthesize antibodies in which the Fc region is glycosylated. In another example, the Fc domain of IgG molecules is glycosylated at asparagine 297 in the CH2 domain. This asparagine is the site for modification with biantennary-type oligosaccharides. This glycosylation participates in effector functions mediated by Fcγ receptors and complement C1q (Burton and Woof (1992) Adv. Immunol. 51:1-84; Jefferis et al. (1998) Immunol. Rev. 163:59-76). The Fc domain can be produced in a mammalian expression system that appropriately glycosylates the residue corresponding to asparagine 297. The Fc domain can also include other eukaryotic post-translational modifications.

Antibodies can be modified, e.g., with a moiety that improves its stabilization and/or retention in circulation, e.g., in blood, serum, lymph, bronchoalveolar lavage, or other tissues, e.g., by at least 1.5, 2, 5, 10, or 50 fold.

For example, an antibody generated by a method described herein can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 daltons (or about 1,000 to about 15,000, and 2,000 to about 12,500) can be used.

For example, an antibody generated by a method described herein can be conjugated to a water soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon.

Antibody-Mimic Molecules

Antibody-mimic molecules are antibody-like molecules comprising a protein scaffold or other non-antibody target binding region with a structure that facilitates binding with target molecules, e.g., polypeptides. When an antibody mimic comprises a scaffold, the scaffold structure of an antibody-mimic is reminiscent of antibodies, but antibody-mimics do not include the CDR and framework structure of immunoglobulins. Like antibodies, however, a pool of scaffold proteins having different amino acid sequence (but having the same basic scaffold structure) can be made and screened to identify the antibody-mimic molecule having the desired features (e.g., ability to bind a particular target; ability to bind a particular target with a certain affinity; ability to bind a particular target to produce a certain result, such as to inhibit activity of the target). In this way, antibody-mimics molecules that bind a target and that have a desired function can be readily made and tested in much the same way that antibodies can be. There are numerous examples of classes of antibody-mimic molecules; each of which is characterized by a unique scaffold structure. Any of these classes of antibody-mimic molecules may be used as the target binding moiety portion of a complex of the disclosure. Exemplary classes are described below and include, but are not limited to, DARPin polypeptides and Anticalins® polypeptides.

In certain embodiments, an antibody-mimic moiety molecule can comprise binding site portions that are derived from a member of the immunoglobulin superfamily that is not an immunoglobulin (e.g., a T-cell receptor or a cell-adhesion protein such as CTLA-4, N-CAM, and telokin). Such molecules comprise a binding site portion which retains the conformation of an immunoglobulin fold and is capable of specifically binding to the target antigen or epitope. In some embodiments, antibody-mimic moiety molecules of the disclosure also comprise a binding site with a protein topology that is not based on the immunoglobulin fold (e.g., such as ankyrin repeat proteins) but which nonetheless are capable of specifically binding to a target antigen or epitope.

Antibody-mimic moiety molecules may be identified by selection or isolation of a target-binding variant from a library of binding molecules having artificially diversified binding sites. Diversified libraries can be generated using completely random approaches (e.g., error-prone PCR, exon shuffling, or directed evolution) or aided by art-recognized design strategies. For example, amino acid positions that are usually involved when the binding site interacts with its cognate target molecule can be randomized by insertion of degenerate codons, trinucleotides, random peptides, or entire loops at corresponding positions within the nucleic acid which encodes the binding site (see e.g., U.S. Pub. No. 20040132028). The location of the amino acid positions can be identified by investigation of the crystal structure of the binding site in complex with the target molecule. Candidate positions for randomization include loops, flat surfaces, helices, and binding cavities of the binding site. In certain embodiments, amino acids within the binding site that are likely candidates for diversification can be identified by their homology with the immunoglobulin fold. For example, residues within the CDR-like loops of fibronectin may be randomized to generate a library of fibronectin binding molecules (see, e.g., Koide et al., J. Mol. Biol., 284: 1141-1151 (1998)). Other portions of the binding site which may be randomized include flat surfaces. Following randomization, the diversified library may then be subjected to a selection or screening procedure to obtain binding molecules with the desired binding characteristics. For example, selection can be achieved by art-recognized methods such as phage display, yeast display, or ribosome display.

In one embodiment, an antibody-mimic molecule of the disclosure comprises a binding site from a fibronectin binding molecule. Fibronectin binding molecules (e.g., molecules comprising the Fibronectin type I, II, or III domains) display CDR-like loops which, in contrast to immunoglobulins, do not rely on intra-chain disulfide bonds. The FnIII loops comprise regions that may be subjected to random mutation and directed evolutionary schemes of iterative rounds of target binding, selection, and further mutation in order to develop useful therapeutic tools. Fibronectin-based “addressable” therapeutic binding molecules (“FATBIM”) may be developed to specifically or preferentially bind the target antigen or epitope. Methods for making fibronectin binding polypeptides are described, for example, in WO 01/64942 and in U.S. Pat. Nos. 6,673,901, 6,703,199, 7,078,490, and 7,119,171, which are incorporated herein by reference.

In another embodiment, an antibody-mimic molecule of the disclosure comprises a binding site from an affibody. As used herein “Affibody®” molecules are derived from the immunoglobulin binding domains of staphylococcal Protein A (SPA) (see e.g., Nord et al., Nat. Biotechnol., 15: 772-777 (1997)). An Affibody® is an antibody mimic that has unique binding sites that bind specific targets. Affibody® molecules can be small (e.g., consisting of three alpha helices with 58 amino acids and having a molar mass of about 6 kDa), have an inert format (no Fc function), and have been successfully tested in humans as targeting moieties. Affibody® molecules have been shown to withstand high temperatures (90° C.) or acidic and alkaline conditions (pH 2.5 or pH 11, respectively). Affibody® binding sites employed in the disclosure may be synthesized by mutagenizing an SPA-related protein (e.g., Protein Z) derived from a domain of SPA (e.g., domain B) and selecting for mutant SPA-related polypeptides having binding affinity for a target antigen or epitope. Other methods for making affibody binding sites are described in U.S. Pat. Nos. 6,740,734 and 6,602,977 and in WO 00/63243, each of which is incorporated herein by reference. In certain embodiments, the disclosure provides a complex comprising a Surf+ Penetrating Polypeptide associated with an Affibody, wherein the Affibody binds to an intracellularly expressed target.

In another embodiment, an antibody-mimic molecule of the disclosure comprises a binding site from an anticalin. As used herein, “Anticalins®” are antibody functional mimetics derived from human lipocalins. Lipocalins are a family of naturally-occurring binding proteins that bind and transport small hydrophobic molecules such as steroids, bilins, retinoids, and lipids. The main structure of Anticalins® is similar to wild type lipocalins. The central element of this protein architecture is a beta-barrel structure of eight antiparallel strands, which supports four loops at its open end. These loops form the natural binding site of the lipocalins and can be reshaped in vitro by extensive amino acid replacement, thus creating novel binding specificities.

Anticalins® possess high affinity and specificity for their prescribed ligands as well as fast binding kinetics, so that their functional properties are similar to those of antibodies. Anticalins® however, have several advantages over antibodies, including smaller size, composition of a single polypeptide chain, and a simple set of four hypervariable loops that can be easily manipulated at the genetic level. Anticalins®, for example, are about eight times smaller than antibodies. Anticalins® have better tissue penetration than antibodies and are stable at temperatures up to 70° C. and also unlike antibodies, Anticalins® can be produced in bacterial cells (e.g., E. coli cells) in large amounts. Further, while antibodies and most other antibody mimetics can only be directed at macromolecules like proteins, Anticalins® are able to selectively bind to small molecules as well. Anticalins® are described in, e.g., U.S. Pat. No. 7,723,476. In certain embodiments, the disclosure provides a complex comprising a Surf+ Penetrating Polypeptide associated with an Affibody, wherein the Affibody binds to an intracellularly expressed target.

In another embodiment, an antibody-mimic molecule of the disclosure comprises a binding site from a cysteine-rich polypeptide. Cysteine-rich domains employed in the practice of the present disclosure typically do not form an alpha-helix, a beta-sheet, or a beta-barrel structure. Typically, the disulfide bonds promote folding of the domain into a three-dimensional structure. Usually, cysteine-rich domains have at least two disulfide bonds, more typically at least three disulfide bonds. An exemplary cysteine-rich polypeptide is an A domain protein. A-domains (sometimes called “complement-type repeats”) contain about 30-50 or 30-65 amino acids. In some embodiments, the domains comprise about 35-45 amino acids and in some cases about 40 amino acids. Within the 30-50 amino acids, there are about 6 cysteine residues. Of the six cysteines, disulfide bonds typically are found between the following cysteines: C1 and C3, C2 and C5, C4 and C6. The A domain constitutes a ligand binding moiety. The cysteine residues of the domain are disulfide linked to form a compact, stable, functionally independent moiety. Clusters of these repeats make up a ligand binding domain, and differential clustering can impart specificity with respect to the ligand binding. Exemplary proteins containing A-domains include, e.g., complement components (e.g., C6, C7, C8, C9, and Factor I), serine proteases (e.g., enteropeptidase, matriptase, and corin), transmembrane proteins (e.g., ST7, LRP3, LRP5 and LRP6) and endocytic receptors (e.g. Sortilin-related receptor, LDL-receptor, VLDLR, LRP1, LRP2, and ApoER2). Methods for making A-domain proteins of a desired binding specificity are disclosed, for example, in WO 02/088171 and WO 04/044011, each of which is incorporated herein by reference.

In another embodiment, an antibody-mimic molecule of the disclosure comprises a binding site from a repeat protein. Repeat proteins are proteins that contain consecutive copies of small (e.g., about 20 to about 40 amino acid residues) structural units or repeats that stack together to form contiguous domains. Repeat proteins can be modified to suit a particular target binding site by adjusting the number of repeats in the protein. Exemplary repeat proteins include designed ankyrin repeat proteins (i.e., a DARPins) (see e.g., Binz et al., Nat. Biotechnol., 22: 575-582 (2004)) or leucine-rich repeat proteins (i.e., LRRPs) (see e.g., Pancer et al., Nature, 430: 174-180 (2004)).

As used here, “DARPins” are genetically engineered antibody mimetic proteins that typically exhibit highly specific and high-affinity target protein binding. DARPins were first derived from natural ankyrin proteins. In certain embodiments, DARPins comprise three, four or five repeat motifs of an ankyrin protein. In certain embodiments, a unit of an ankyrin repeat consists of 30-34 amino acid residues and functions to mediate protein-protein interactions. In certain embodiments, each ankyrin repeat exhibits a helix-turn-helix conformation, and strings of such tandem repeats are packed in a nearly linear array to form helix-turn-helix bundles connected by relatively flexible loops. In certain embodiments, the global structure of an ankyrin repeat protein is stabilized by intra- and inter-repeat hydrophobic and hydrogen bonding interactions. The repetitive and elongated nature of the ankyrin repeats provides the molecular bases for the unique characteristics of ankyrin repeat proteins in protein stability, folding and unfolding, and binding specificity. While not wishing to be bound by theory, it is believed that the ankyrin repeat proteins do not recognize specific sequences, and interacting residues are discontinuously dispersed into the whole molecules of both the ankyrin repeat protein and its target protein. In addition, the availability of thousands of ankyrin repeat sequences has made it feasible to use rational design to modify the specificity and stability of an ankyrin repeat domain for use as a DARPin to target any number of proteins. The molecular mass of a DARPin domain is typically about 14 or 18 kDa for four- or five-repeat DARPins, respectively. DARPins are described in, e.g., U.S. Pat. No. 7,417,130. All so far determined tertiary structures of ankyrin repeat units share a characteristic composed of a beta-hairpin followed by two antiparallel alpha-helices and ending with a loop connecting the repeat unit with the next one. Domains built of ankyrin repeat units are formed by stacking the repeat units to an extended and curved structure. LRRP binding sites from part of the adaptive immune system of sea lampreys and other jawless fishes and resemble antibodies in that they are formed by recombination of a suite of leucine-rich repeat genes during lymphocyte maturation. Methods for making DARpin or LRRP binding sites are described in WO 02/20565 and WO 06/083275, each of which is incorporated herein by reference.

Another example of a target binding moiety suitable for use in the present disclosure is based on technology in which binding regions are engineered into the Fc domain of an antibody molecule. These antibody-like molecules are another example of target binding moieties for use in the present disclosure. In certain embodiments, antibody mimics include all or a portion of an antibody like molecule, comprising the CH2 and CH3 domains of an immunoglulin, engineered with non-CDR loops of constant and/or variable domains, thereby mediating binding to an epitope via the non-CDR loops. Exemplary technology includes technology from F-Star, such as antigen binding Fc molecules (termed Fcab™) or full length antibody like molecules with dual functionality (mAb²™). Fcab™ (antigen binding Fc) are a “compressed” version of these antibody like molecules. These molecules include the CH2 and CH3 domains of the Fc portion of an antibody, naturally folded as a homodimer (50 kDa). Antigen binding sites are engineered into the CH3 domains, but the molecules lack traditional antibody variable regions.

Similar antibody like molecules are referred to as mAb²™ molecules. Full length IgG antibodies with additional binding domains (such as two) engineered into the CH3 domains. Depending on the type of additional binding sites engineered into the CH3 domains, these molecules may be bispecific or multispecific or otherwise facilitate tissue targeting.

This technology is described in, for example, WO08/003103, WO12/007167, and US application 20090298195, the disclosures of which are hereby incorporated by reference.

In other embodiments, an antibody-mimic molecule of the disclosure comprises binding sites derived from Src homology domains (e.g. SH2 or SH3 domains), PDZ domains, beta-lactamase, high affinity protease inhibitors, or small disulfide binding protein scaffolds such as scorpion toxins. Methods for making binding sites derived from these molecules have been disclosed in the art, see e.g., Panni et al., J. Biol. Chem., 277: 21666-21674 (2002), Schneider et al., Nat. Biotechnol., 17: 170-175 (1999); Legendre et al., Protein Sci., 11:1506-1518 (2002); Stoop et al., Nat. Biotechnol., 21: 1063-1068 (2003); and Vita et al., PNAS, 92: 6404-6408 (1995). Yet other binding sites may be derived from a binding domain selected from the group consisting of an EGF-like domain, a Kringle-domain, a PAN domain, a Gla domain, a SRCR domain, a Kunitz/Bovine pancreatic trypsin Inhibitor domain, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an Immunoglobulin-like domain, a C-type lectin domain, a MAM domain, a von Willebrand factor type A domain, a Somatomedin B domain, a WAP-type four disulfide core domain, a F5/8 type C domain, a Hemopexin domain, a Laminin-type EGF-like domain, a C2 domain, a binding domain derived from tetranectin in its monomeric or trimeric form, and other such domains known to those of ordinary skill in the art, as well as derivatives and/or variants thereof. Exemplary antibody-mimic moiety molecules, and methods of making the same, can also be found in Stemmer et al., “Protein scaffolds and uses thereof”, U.S. Patent Publication No. 20060234299 (Oct. 19, 2006) and Hey, et al., Artificial, Non-Antibody Binding Proteins for Pharmaceutical and Industrial Applications, TRENDS in Biotechnology, vol. 23, No. 10, Table 2 and pp. 514-522 (October 2005).

In one embodiment, an antibody-mimic molecule comprises a Kunitz domain. “Kunitz domains” as used herein, are conserved protein domains that inhibit certain proteases, e.g., serine proteases. Kunitz domains are relatively small, typically being about 50 to 60 amino acids long and having a molecular weight of about 6 kDa. Kunitz domains typically carry a basic charge and are characterized by the placement of two, four, six or eight or more that form disulfide linkages that contribute to the compact and stable nature of the folded peptide. For example, many Kunitz domains have six conserved cysteine residues that form three disulfide linkages. The disulfide-rich α/β fold of a Kunitz domain can include two, three (typically), or four or more disulfide bonds.

Kunitz domains have a pear-shaped structure that is stabilized the, e.g., three disulfide bonds, and that contains a reactive site region featuring the principal determinant P1 residue in a rigid confirmation. These inhibitors competitively prevent access of a target protein (e.g., a serine protease) for its physiologically relevant macromolecular substrate through insertion of the P1 residue into the active site cleft. The P1 residue in the proteinase-inhibitory loop provides the primary specificity determinant and dictates much of the inhibitory activity that particular Kunitz protein has toward a targeted proteinase. Typically, the N-terminal side of the reactive site (P) is energetically more important that the P′ C-terminal side. In most cases, lysine or arginine occupy the P1 position to inhibit proteinases that cleave adjacent to those residues in the protein substrate. Other residues, particularly in the inhibitor loop region, contribute to the strength of binding. Generally, about 10-12 amino acid residues in the target protein and 20-25 residues in the proteinase are in direct contact in the formation of a stable proteinase-inhibitor complex and provide a buried area of about 600 to 900 A. By modifying the residues in the P site and surrounding residues Kunitz domains can be designed to target and inhibit a protein of choice. Kunitz domains are described in, e.g., U.S. Pat. No. 6,057,287.

In another embodiment, an antibody-mimic molecule of the disclosure is an Affilin®. As used herein “Affilin®” molecules are small antibody-mimic proteins which are designed for specific affinities towards proteins and small compounds. New Affilin® molecules can be very quickly selected from two libraries, each of which is based on a different human derived scaffold protein. Affilin® molecules do not show any structural homology to immunoglobulin proteins. There are two commonly-used Affilin® scaffolds, one of which is gamma crystalline, a human structural eye lens protein and the other is “ubiquitin” superfamily proteins. Both human scaffolds are very small, show high temperature stability and are almost resistant to pH changes and denaturing agents. This high stability is mainly due to the expanded beta sheet structure of the proteins. Examples of gamma crystalline derived proteins are described in WO200104144 and examples of “ubiquitin-like” proteins are described in WO2004106368.

In another embodiment, an antibody-mimic moiety molecule of the disclosure is an Avimer. Avimers are evolved from a large family of human extracellular receptor domains by in vitro exon shuffling and phage display, generating multidomain proteins with binding and inhibitory properties. Linking multiple independent binding domains has been shown to create avidity and results in improved affinity and specificity compared with conventional single-epitope binding proteins. In certain embodiments, Avimers consist of two or more peptide sequences of 30 to 35 amino acids each, connected by linker peptides. The individual sequences are derived from A domains of various membrane receptors and have a rigid structure, stabilised by disulfide bonds and calcium. Each A domain can bind to a certain epitope of the target protein. The combination of domains binding to different epitopes of the same protein increases affinity to this protein, an effect known as avidity (hence the name). Other potential advantages include simple and efficient production of multitarget-specific molecules in Escherichia coli, improved thermostability and resistance to proteases. Avimers with sub-nanomolar affinities have been obtained against a variety of targets. Alternatively, the domains can be directed against epitopes on different target proteins. This approach is similar to the one taken in the development of bispecific monoclonal antibodies. In a study, the plasma half-life of an anti-interleukin 6 avimer could be increased by extending it with an anti-immunoglobulin G domain. Additional information regarding Avimers can be found in U.S. patent application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

The foregoing provides numerous examples of classes of antibody-mimics. In certain embodiments, the disclosure provides complexes in which the target binding moiety portion is an antibody-mimic that binds to a target, such as any of the foregoing classes antibody-mimics. Any of these antibody-mimics may be complexed with a Surf+ Penetrating Polypeptide or a portion comprising a Surf+ Penetrating Polypeptide (or a cell penetrating peptide), including any of the sub-categories or specific examples of Surf+ Penetrating Polypeptides (or cell penetrating peptides).

Adhesin Molecules

Adhesin molecules comprise a ligand, a receptor, or portions thereof (an “adhesin”). In certain embodiments, the disclosure provides complexes in which the target binding moiety is an adhesin molecule.

In certain embodiments, adhesins are chimeric molecules which combine the binding domain of a protein such as a cell-surface receptor or a ligand with a portion of an immunoglobulin molecule, e.g., the effector domain or constant domain. Adhesins can possess many of the valuable chemical and biological properties of antibodies.

A binding domain of a ligand refers to any native cell-surface receptor or any region or derivative thereof retaining at least a qualitative ligand binding ability, and preferably the biological activity of a corresponding native receptor. In a specific embodiment, the receptor is from a cell-surface polypeptide having an extracellular domain which is homologous to a member of the immunoglobulin supergenefamily. Other typical receptors, are not members of the immunoglobulin supergenefamily but are nonetheless specifically covered by this definition, are receptors for cytokines, and in particular receptors with tyrosine kinase activity (receptor tyrosine kinases), members of the hematopoietin and nerve growth factor receptor superfamilies, and cell adhesion molecules, e.g. (E-, L- and P-) selectins.

A binding domain of a receptor is used to designate any native ligand for a receptor, including cell adhesion molecules, or any region or derivative of such native ligand retaining at least a qualitative receptor binding ability, and preferably the biological activity of a corresponding native ligand.

Adhesins can be constructed from a human protein sequence with a desired specificity linked to an appropriate human immunoglobulin hinge and constant domain (Fc) sequence and thus, the binding specificity of interest can be achieved using entirely human components. Such adhesins are minimally immunogenic to the patient, and are safe for chronic or repeated use.

Adhesins reported in the literature include fusions of the T cell receptor (Gascoigne et al., Proc. Natl. Acad. Sci. USA 84:2936-2940 (1987)); CD4 (Capon et al., Nature 337:525-531 (1989); Traunecker et al., Nature 339:68-70 (1989); Zettmeissl et al., DNA Cell Biol. USA 9:347-353 (1990); and Byrn et al., Nature 344:667-670 (1990)); L-selectin or homing receptor (Watson et al., J. Cell. Biol. 110:2221-2229 (1990); and Watson et al., Nature 349:164-167 (1991)); CD44 (Aruffo et al., Cell 61:1303-1313 (1990)); CD28 and B7 (Linsley et al., J. Exp. Med. 173:721-730 (1991)); CTLA-4 (Lisley et al., J. Exp. Med. 174:561-569 (1991)); CD22 (Stamenkovic et al., Cell 66:1133-1144 (1991)); TNF receptor (Ashkenazi et al., Proc. Natl. Acad. Sci. USA 88:10535-10539 (1991); Lesslauer et al., Eur. J. Immunol. 27:2883-2886 (1991); and Peppel et al., J. Exp. Med. 174:1483-1489 (1991)); NP receptors (Bennett et al., J. Biol. Chem. 266:23060-23067 (1991)); inteferon .gamma. receptor (Kurschner et al., J. Biol. Chem. 267:9354-9360 (1992)); 4-1BB (Chalupny et al., PNAS (USA) 89:10360-10364 (1992)) and IgE receptor .alpha. (Ridgway and Gorman, J. Cell. Biol. Vol. 115, Abstract No. 1448 (1991)).

Preparation of Adhesin Molecules

Chimeras constructed from an adhesin binding domain sequence linked to an appropriate immunoglobulin constant domain sequence (adhesins) are known in the art.

The simplest and most straightforward adhesin design combines the binding domain(s) of the adhesin (e.g., the extracellular domain (ECD) of a receptor) with the hinge and Fc regions of an immunoglobulin heavy chain. Ordinarily, when preparing the adhesins of the present invention, nucleic acid encoding the binding domain of the adhesin will be fused C-terminally to nucleic acid encoding the N-terminus of an immunoglobulin constant domain sequence, however N-terminal fusions are also possible.

Typically, in such fusions the encoded chimeric polypeptide will retain at least functionally active hinge, CH2 and CH3 domains of the constant region of an immunoglobulin heavy chain. Fusions are also made to the C-terminus of the Fc portion of a constant domain, or immediately N-terminal to the CH1 of the heavy chain or the corresponding region of the light chain. The precise site at which the fusion is made is not critical; particular sites are well known and may be selected in order to optimize the biological activity, secretion, or binding characteristics of the Ia.

In a specific embodiment, the adhesin sequence is fused to the N-terminus of the Fc domain of immunoglobulin G1 (IgG1). It is possible to fuse the entire heavy chain constant region to the adhesin sequence. In another embodiment, a sequence beginning in the hinge region just upstream of the papain cleavage site which defines IgG Fc chemically (i.e. residue 216, taking the first residue of heavy chain constant region to be 114), or analogous sites of other immunoglobulins is used in the fusion. In another specific embodiment, the adhesin amino acid sequence is fused to (a) the hinge region and CH2 and CH3 or (b) the CH1, hinge, CH2 and CH3 domains, of an IgG1, IgG2, or IgG3 heavy chain. The precise site at which the fusion is made is not critical, and the optimal site can be determined by routine experimentation.

Small Organic Molecules

Virtually any small molecule, such as a small organic or inorganic molecule, can be conjugated to the FGF10 portion to generate a complex of the disclosure. In certain embodiments, the small molecule is a small organic molecule. In certain embodiments, the small molecule is less than 1000, less than 750, less than 650, or less than 550 amu. In other embodiments, the small molecule is less than 500 amu.

In certain embodiments, it is advantageous to prevent the small molecule from crossing the blood-brain barrier. Conjugation to a protein would be useful to prevent the small molecule from crossing the blood-brain barrier. However, the molecule would still be available to other tissues. Additionally, given that FGF10 portions are not ubiquitously taken up by all cells, conjugation of the small molecule to an FGF10 portion can be used to help decrease side effects and help target the small molecule to particular tissues.

Exemplary small molecules include, but are not limited to methotrexate (for treating autoimmune diseases), small molecules for delivery to liver, such as therapies for hepatitis (e.g., telaprevir and boceprevir for HCV and entecavir or lamivudine for HBV).

Further exemplary small molecules include chemotherapeutics or other small molecules for treating cancer, particularly liver and kidney cancers (given the preferential uptake of FGF10 to those tissues). A particular example of a small molecule useful for liver and kidney cancers is sorafenib.

A particular example of small molecules where it would be advantageous to limit crossing of the blood-brain barrier are platelet inhibitors, such as integrilin or aggrastat. Limiting access to the blood brain barrier is useful for preventing intracerebral bleeding.

The foregoing are merely exemplary of the small molecules (including organic and inorganic molecules that can be used as cargo).

The forgoing provides examples of polypeptides, peptides, and small molecules, as well as classes of such molecules, suitable for use as a cargo portion in the complexes of the disclosure. The disclosure contemplates complexes comprising any of the foregoing cargo portions or categories of such cargo portions. As detailed above, complexes of the disclosure comprise such cargo portion and an FGF-10 portion. The cargo portion may be located N- or C-terminal to the FGF-10 portion.

(iv) Complexes

The present disclosure provides complexes comprising (i) an FGF10 portion and (ii) a cargo portion. The FGF10 portion comprises or consists of a domain or variant of an FGF10-related, such as an FGF-10 related Surf+ Penetrating Polypeptide. The complexes are useful, for example, for delivery into a cell, and thus to facilitate delivery of the cargo into a cell. Given that cell penetrating domains of FGF10 preferentially localize to particular organs and tissues, in a manner inconsistent with the expression of the cognate receptor used for FGF10's mitogenic function, the complexes are particularly useful for delivering cargo into cells of tissues and organs to which FGF10 preferentially localizes and penetrates. Suitable complexes are complexes in which the FGF10 portion comprises a domain of FGF10 of at least 4 kDa that has net positive charge and having a charge per molecular weight ratio larger than that of the corresponding full length, unprocessed, naturally occurring FGF10 polypeptide. Suitable complexes are also those in which the cargo portion comprises a polypeptide, peptide, or small organic molecule.

Below are provided examples of complexes of the disclosure and how the portions of the complexes are associated and/or made. The present disclosure provides complexes comprising (i) an FGF-10 portion and (ii) a cargo portion associated with the FGF-10 portion. The cargo portion comprises a heterologous polypeptide, peptide or small molecule suitable for delivery into cells and the FGF-10 portion comprises or consists of a Surf+ Penetrating Polypeptide that facilitates entry of the complex, and thus entry of the cargo portion, into cells. Once inside the cell, the cargo portion can function. For example, the cargo portion may comprise a polypeptide or peptide that is endogenously expressed in a cell, and delivery of that cargo can supplement the function of the endogenously produced polypeptide (e.g., particularly in cases where that endogenous polypeptide is mutated and/or expressed at low levels and/or misexpressed). In certain embodiments, the association between the cargo portion and the FGF-10 portion is disruptable. Thus, in certain embodiments, once the complex enters the cell and/or a subcellular compartment of the cell, the association can be disrupted. However, the association need not be disrupted or disruptable, and the complex may remain intact after entry into the cell and/or entry into a subcellular compartment.

Complexes of the disclosure may, in certain embodiments, include portions in addition to the FGF-10 portion and the cargo portion. For example, the complexes may include one or more linkers, such as a linker interconnecting the FGF-10 portion and the cargo portion. Additionally or alternatively, the complexes may include sequence that helps target the complex to a subcellular compartment, such as the mitochondria or nucleus (e.g., a mitochondrial localization signal or a nuclear localization signal. Additionally or alternatively, the complex may include one or more tags to facilitate detection and/or purification of the complex or a portion of the complex (e.g., polyHis tag, HA tag, FLAG tag, myc tag, etc.). In certain embodiments, the complex includes 1, 2, or 3 tags. When present, additional sequences may be located at the N-terminus, at the C-terminus, internally, or some combination thereof. For example, a complex may include a nuclear localization sequence on the N-terminus and a myc tag on the C-terminus. Further, complexes may include portions that increase the in vivo half life of the complex or of the cargo portion.

Three exemplary complexes of the disclosure are provided in the sequence listing. SEQ ID NO: 4 comprises an FGF-10 portion and an HSV TK portion. The disclosure contemplates a complex that comprises or consists of the amino acid sequence set forth in SEQ ID NO: 4, in the presence or absence of a polyHis tag. Moreover, depending on the expression system used, the N-terminal methionine is optional and may not be present in the final protein product.

SEQ ID NOs 8 and 9 comprises an FGF-10 portion and a p16 tumor suppressor portion.

The disclosure contemplates a complex comprising or consisting of the amino acid sequence set forth in SEQ ID NO: 8 or 9, in the presence or absence of a polyHis tag.

In certain embodiments, the FGF-10 portion and the cargo portion of the complex are associated covalently. For example, these two portions may be fused (e.g., the complex comprises a fusion protein). Covalent interactions may be direct or indirect (via a linker). Thus, in some embodiments, such covalent interactions are mediated by one or more linkers. In some embodiments, the linker is a cleavable linker. In certain embodiments, the cleavable linker comprises an amide, an ester, or a disulfide bond. For example, the linker may be an amino acid sequence that is cleavable by a cellular enzyme. In certain embodiments, the enzyme is a protease. In other embodiments, the enzyme is an esterase. In some embodiments, the enzyme is one that is more highly expressed in certain cell types than in other cell types. In certain embodiments, the enzyme that cleaves the linker is expressed in a particular subcellular compartment so that cleavage occurs after the complex has entered that sub cellular compartment. In certain embodiments, the linker is cleavable and cleavage is the result of a change in pH (e.g., a change in pH from the outside of the cell to the inside of the cell or from the inside of the cell to a subcellular compartment causes the cleavage). Exemplary sequences that can be used in linkers and enzymes that cleave those linkers are presented in the Table below.

TABLE
Exemplary cleavable linker sequences.

| Cleavable | SEQ ID | |
|---|---|---|
| sequencer | NO: | Enzymes that Target the Linker |
| X-AGVF-X | 24 | Lysosomal thiol proteinases (see, e.g., Duncan et al., |
| | | Biosci. Rep., 2: 1041-46, 1982; incorporated herein by |
| | | reference) |
| X-GFLG-X | 25 | Lysosomal cysteine proteinases (see, e.g., Vasey et al., |
| | | Clin. Canc. Res., 5: 83-94, 1999; incorporated herein by |
| | | reference) |
| X-FK-X | | Cathepsin B-ubiquitous, overexpressed in many solid |
| | | tumors, such as breast cancer (see, e.g., Dubowchik et al., |
| | | Bioconjugate Chem., 13: 855-69, 2002; incorporated |
| | | herein by reference) |
| X-A*L-X | | Lysosomal hydrolases (see, e.g., Trouet et al., Proc. Natl, |
| | | Acad. Sci., USA, 79: 626-29, 1982; incorporated herein by |
| | | reference) |
| X-A*LA*L-X | 26 | Cathepsin B-ubiquitous, overexpressed in many solid |
| | | tumors, such as breast cancer (see, e.g., Schmid et al., |
| | | Bioconjugate Chemistry, 18: 702-16, 2007; incorporated |
| | | herein by reference) |
| X-AL*AL*A-X | 27 | Cathepsin D-ubiquitous (see, e.g., Czerwinski et al., Proc. |
| | | Natl. Acad. Sci. USA, 95: 11520-25, 1998; incorporated |
| | | herein by reference) |

“X” denotes the FGF-10 portion or cargo portion.
“*” refers to observed cleavage site.

Other exemplary linkers include flexible linkers, such as one or more repeats of glycine and serine (Gly/Ser linkers).

In certain embodiments, the FGF-10 portion and the cargo portion are fused by using a construct that comprises an intein, which is self-spliced out to join the FGF-10 portion and the cargo portion via a peptide bond.

In another embodiment, e.g., where expression of a fusion construct is not practical (e.g., is inefficient) or not possible, the FGF-10 portion and the cargo portion may be synthesized by using a viral 2A peptide construct that comprises the FGF-10 portion and the cargo portion for bicistronic expression. In this embodiment, the FGF-10 portion and the cargo portion genes may be expressed on the bicistronic construct, and the 2A peptide results in cotranslational “cleavage” of the two proteins (Trichas et al., BMC Biology 6:40, 2008).

The disclosure contemplates complexes in which the FGF-10 portion and the cargo portion are associated by a covalent or non-covalent linkage. In either case, the association may be direct or via one or more additional intervening linkers or moieties.

In some embodiments, an FGF-10 portion and a cargo portion are associated through chemical or proteinaceous linkers or spacers. Exemplary linkers and spacers include, but are not restricted to, substituted or unsubstituted alkyl chains, polyethylene glycol derivatives, amino acid spacers, sugars, or aliphatic or aromatic spacers common in the art.

Suitable linkers include, for example, homobifunctional and heterobifunctional cross-linking molecules. The homobifunctional molecules have at least two reactive functional groups, which are the same. The reactive functional groups on a homobifunctional molecule include, for example, aldehyde groups and active ester groups. Homobifunctional molecules having aldehyde groups include, for example, glutaraldehyde and subaraldehyde.

Homobifunctional linker molecules having at least two active ester units include esters of dicarboxylic acids and N-hydroxysuccinimide. Some examples of such N-succinimidyl esters include disuccinimidyl suberate and dithio-bis-(succinimidyl propionate), and their soluble bis-sulfonic acid and bis-sulfonate salts such as their sodium and potassium salts.

Heterobifunctional linker molecules have at least two different reactive groups. Examples of heterobifunctional reagents containing reactive disulfide bonds include N-succinimidyl 3-(2-pyridyl-dithio)propionate (Carlsson et al., 1978. Biochem. J., 173:723-737), sodium S-4-succinimidyloxycarbonyl-alpha-methylbenzylthiosulfate, and 4-succinimidyloxycarbonyl-alpha-methyl-(2-pyridyldithio)toluene. Examples of heterobifunctional reagents comprising reactive groups having a double bond that reacts with a thiol group include succinimidyl 4-(N-maleimidomethyl)cyclohexahe-1-carboxylate and succinimidyl m-maleimidobenzoate. Other heterobifunctional molecules include succinimidyl 3-(maleimido)propionate, sulfosuccinimidyl 4-(p-maleimido-phenyl)butyrate, sulfosuccinimidyl 4-(N-maleimidomethyl-cyclohexane)-1-carboxylate, maleimidobenzoyl-5N-hydroxy-succinimide ester.

Other means of cross-linking proteins utilize affinity molecule binding pairs, which selectively interact with acceptor groups. One entity of the binding pair can be fused or otherwise linked to the FGF-10 portion and the other entity of the binding pair can be fused or otherwise linked to the cargo portion. Exemplary affinity molecule binding pairs include biotin and streptavidin, and derivatives thereof; metal binding molecules; and fragments and combinations of these molecules. Exemplary affinity binding pairs include StreptTag (WSHPQFEK)/SBP (streptavidin binding protein), cellulose binding domain/cellulose, chitin binding domain/chitin, S-peptide/S-fragment of RNAseA, calmodulin binding peptide/calmodulin, and maltose binding protein/amylose.

In one embodiment, the FGF-10 portion and the cargo portion are linked by ubiquitin (and ubiquitin-like) conjugation. In other embodiments, the FGF-10 portion and the cargo portion may be fused through an enzymatic reaction, through a disulfide bond, or through an artificial amino acid.

The disclosure also provides nucleic acids encoding an FGF-10 portion and a cargo portion. The complex of a FGF-10 portion and a cargo portion can be expressed as a fusion protein, optionally separated by a peptide linker. The peptide linker can be cleavable or not cleavable. A nucleic acid encoding a fusion protein can express the fusion in any orientation. For example, the nucleic acid can express an N-terminal FGF-10 portion fused to a C-terminal cargo portion, or can express an N-terminal cargo portion fused to a C-terminal FGF-10 portion.

A nucleic acid encoding an FGF-10 portion can be on a vector that is separate from a vector that carries a nucleic acid encoding a cargo portion. The FGF-10 portion and the cargo portion can be expressed separately, and complexed (including chemically linked) prior to introduction to a cell for intracellular delivery. The isolated complex can be formulated for administration to a subject, as a pharmaceutical composition.

The disclosure also provides host cells comprising a nucleic acid encoding the FGF-10 portion or the cargo portion, or comprising the complex as a fusion protein. The host cells can be, for example, prokaryotic cells (e.g., E. coli) or eukaryotic cells. The two portion can be made in the same or in different host cells.

In certain embodiments, the recombinant nucleic acids encoding a complex, or the portions thereof, may be operably linked to one or more regulatory nucleotide sequences in an expression construct. Regulatory nucleotide sequences will generally be appropriate for a host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used. In certain aspects, this disclosure relates to an expression vector comprising a nucleotide sequence encoding a complex of the disclosure (e.g., a complex comprising an FGF-10 portion and a cargo portion) polypeptide and operably linked to at least one regulatory sequence. Regulatory sequences are art-recognized and are selected to direct expression of the encoded polypeptide. Accordingly, the term regulatory sequence includes promoters, enhancers, and other expression control elements. Exemplary regulatory sequences are described in Goeddel; Gene Expression Technology: Methods in Enzymology, Academic Press, San Diego, Calif. (1990). It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

The disclosure also provides host cells comprising or transfected with a nucleic acid encoding the complex as a fusion protein. The host cells can be, for example, prokaryotic cells (e.g., E. coli) or eukaryotic cells. Other suitable host cells are known to those skilled in the art.

In addition to the nucleic acid sequence encoding the complex or portions of the complex, a recombinant expression vector may carry additional nucleic acid sequences, such as sequences that regulate replication of the vector in a host cells (e.g., origins of replication) and selectable marker genes. The selectable marker gene facilitates selection of host cells into which the vector has been introduced. Exemplary selectable marker genes include the ampicillin and the kanamycin resistance genes for use in E. coli.

The present disclosure further pertains to methods of producing fusion proteins of the disclosure. For example, a host cell transfected with an expression vector can be cultured under appropriate conditions to allow expression of the polypeptide to occur. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art. The polypeptides can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptides. In a preferred embodiment, the polypeptide is a fusion protein containing a domain which facilitates its purification.

A nucleic acid encoding an FGF-10 portion can be on a vector that is separate from a vector that carries a nucleic acid encoding a cargo portion. The portions of the complex can be expressed separately, and complexed prior to introduction to a cell for intracellular delivery. The isolated complex can be formulated for administration to a subject, as a pharmaceutical composition. As noted above, when expressed separately, the FGF-10 portion and the cargo portion may be expressed using the same or differing vectors and/or using the same or differing host cells.

Recombinant nucleic acids of the disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli. The preferred mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pH EBo, pREP-derived and p205) can be used for transient expression of proteins in eukaryotic cells. The various methods employed in the preparation of the plasmids and transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

It should be understood that fusion polypeptides or protein of the present disclosure can be made in numerous ways. For example, an FGF-10 portion and a cargo portion can be made separately, such as recombinantly produced in two separate cell cultures from nucleic acid constructs encoding their respective proteins. Once made, the proteins can be chemically conjugated directly or via a linker. By way of another example, the fusion polypeptide can be made as an inframe fusion in which the entire fusion polypeptide, optionally including one or more linker, tag or other moiety, is made from a nucleic acid construct that includes nucleotide sequence encoding both an FGF-10 portion and a cargo portion of the complex.

In certain embodiments, a complex of the disclosure is formed under conditions where the linkage (e.g., by a covalent or non-covalent linkage) is formed, while the activity of the cargo portion is maintained. In certain embodiments, the complex maintains at least 50% of a native activity (e.g., of at least one native activity) of the cargo portion alone. For example, where the cargo portion is an enzyme, the complex retains at least 50% of the native activity of that enzyme.

Further, in certain embodiments, where the complex comprises a cleavable linker, the enzyme that cleaves that linker does not have a significant affect on the cargo portion.

In other embodiments, the FGF-10 portion and the cargo portion of the complex are separated, e.g., within the cell, under conditions where the linkage (e.g., a covalent or non-covalent linkage) is dissociated, while the activity of the cargo portion is maintained. For example, the FGF-10 portion and the cargo portion can be joined by a cleavable peptide linker that is subject to a protease that does not interfere with activity of the cargo portion.

In some embodiments the FGF-10 portion and the cargo portion are separated in the endosome due to the lower pH of the endosome. Thus in these embodiments, the linker is cleaved or broken in response to the lower pH, but the activity of the cargo portion is not significantly affected.

It should be noted that the disclosure contemplates that the foregoing description of complexes is applicable to any of the embodiments and combinations of embodiments described herein.

Modifications

As detailed above, the disclosure contemplates that FGF10-related Surf+ Penetrating Polypeptides may be modified chemically or biologically. For example one or more amino acids may be added, deleted, or changed from the primary sequence. This includes changes intended to supercharge a polypeptide (e.g., to increase surface positive charge, net charge or charge/molecular weight). However, modifications to the FGF10-related Surf+ Penetrating Polypeptides also include variation that is not intended to supercharge the protein.

In this section, additional modifications are described. The modifications may be modifications to a complex of the disclosure, and the modification may be appended directly or indirectly to either or both of the FGF-10 portion or the cargo portion. For example, a polyhistidine tag or other tag may be added to the complex or to either portion of the complex to aid in the purification of the complex or of either portion of the complex. Other peptides, protein or small molecules may be added onto the complex to alter the biological, biochemical, and/or biophysical properties of the complex. For example, a targeting peptide may be added to the primary sequence of the complex, such as to further promote delivery to the nucleus, mitochondria, or other subcellular compartment.

Other modifications of the Surf+ Penetrating Polypeptides or complex include, but are not limited to, post-translational or post-production modifications (e.g., glycosylation, phosphorylation, acylation, lipidation, farnesylation, acetylation, proteolysis, etc.). In certain embodiments, the FGF-10 portion or complex may be modified to reduce its immunogenicity. In certain embodiments, the FGF-10 portion or complex may be modified to improve half-life or bioavailability.

In certain embodiments, the complex or either portion of the complex may be conjugated to a soluble polymer or carbohydrate, e.g., to increase serum half life of the either portion and/or the complex. For example, the FGF-10 portion, cargo portion, or complex may be conjugated to a polyethylene glycol (PEG) polymer, e.g., a monomethoxy PEG. Other polymers useful as stabilizing materials may be of natural, semi-synthetic (modified natural) or synthetic origin. Exemplary natural polymers include naturally occurring polysaccharides, such as, for example, arabinans, fructans, fucans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrin, dextrose, glucose, polyglucose, polydextrose, pustulan, chitin, agarose, keratin, chondroitin, dermatan, hyaluronic acid, alginic acid, xanthin gum, starch and various other natural homopolymer or heteropolymers, such as those containing one or more of the following aldoses, ketoses, acids or amines: erythose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Accordingly, suitable polymers include, for example, proteins, such as albumin, polyalginates, and polylactide-coglycolide polymers. Exemplary semi-synthetic polymers include carboxymethylcellulose, hydroxymethylcellulose, hydroxypropylmethylcellulose, methylcellulose, and methoxycellulose. Exemplary synthetic polymers include polyphosphazenes, hydroxyapatites, fluoroapatite polymers, polyethylenes (such as, for example, polyethylene glycol (including for example, the class of compounds referred to as PLURONIC™, commercially available from BASF, Parsippany, N.J.), polyoxyethylene, and polyethylene terephthalate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof.

One of skill in the art can envision a multitude of ways of modifying the FGF-10 portion, the cargo portion, or the complexes of the disclosure without departing from the scope of the present disclosure. In certain embodiments, the primary purpose of the modification is a purpose other than to further supercharge the complex versus that of the unmodified complex. The disclosure contemplates that any of the foregoing modifications may be to the FGF-10 portion of a complex or to the cargo portion of a complex. Moreover, the modification may be made prior to complex formation, concurrently with complex formation, such as fusion protein formation, or as a post-production step following complex formation (such as fusion protein formation).

Additional examples of modifications include targeting domains to facilitate targeting of the complex to the intended location. Once again, the targeting domain may be appended directly or indirectly to the FGF-10 portion or to the cargo portion. Exemplary targeting domains include, a mitochondrial matrix localization signal or a nuclear localization signal. In certain embodiments, it may be preferable to append the targeting domain to the cargo portion so that, in the event that the association between the FGF-10 portion and the cargo portion is disrupted (such as by cleavage of a cleavable linker) after entry into the cell, the cargo portion will still include the targeting domain.

In certain the FGF10 portion comprises a variant (e.g., an FGF10-related Surf+ Penetrating Polypeptide) which includes 1, 2, 3, 4, 5, 6, 7, 8, 9, 10 (or even more than 10) amino acid substitutions, deletions, and/or additions (each of which is independently selected) relative to all or a corresponding portion 2, 8, or 9. In certain embodiments, the variant comprises an amino acid sequence at least 80%, 85%, 90%, 91%, 92%, 93%, 94%, 95%, 96%, 97%, 98%, or 99% identical to all or a corresponding portion of SEQ ID NO: 2, 8, or 9. Suitable variants for use in the context of the present disclosure retain cell penetrating activity.

In certain embodiments, the in addition to being a FGF10-related Surf+ Penetrating Polypeptide, an endogenous activity of FGF-10 is decreased or substantially eliminated in the variant polypeptide. For example, the variant may have decreased mitogenic activity and/or decreased affinity for its cognate receptor FGFR2b. Exemplary variants in which an endogenous activity of FGF-10 is decreased are set forth in SEQ ID NO: 8 and SEQ ID NO: 9. For example, an FGF-10 variant or fragment having two substitutions (E158K/K195A; where the numbering of the amino acid residues is relative to the full length, unprocessed, naturally occurring protein) displays decreased binding to the FGFR2b receptor by approximately a factor of 4 without affecting the binding of FGF10 to heparin. By way of further example, an FGF-10 variant or fragment having one substitution (R78A; where the numbering of the amino acid residues is relative to the full length, unprocessed, naturally occurring protein) displays an approximately 4-fold decrease in binding to the FGFR2b receptor along with a significant decrease in mitogenic activity. By way of further example, an FGF-10 variant or fragment having one substitution (T114 modified to either arginine or alanine; where the numbering of the amino acid residues is relative to the full length, unprocessed, naturally occurring protein) displays reduced binding to FGFR2b relative to the wild-type protein as well as reduced mitogenic activity.

The foregoing are merely exemplary of modification of the complexes of the disclosure whose primary purpose is other than to further supercharge the complex, relative to the unmodified complex.

Detectable Moieties

It is further contemplated that complexes of the disclosure can be modified to comprise a detectable moiety. Detectable moieties include fluorescent or otherwise detectable polypeptides, peptide, radioactive or other moieties which allow for detection of the complex or the portions of the complex. Such detectable moieties can be included in the polypeptide sequence of the complex, or operably linked thereto, such as in a fusion protein, or by covalent or non-covalent linkages. The disclosure contemplates that the detectable moiety may be appended directly or indirectly to the FGF-10 portion of the complex and/or the cargo portion of the complex and/or to any linker portion.

Exemplary fluorescent proteins include green fluorescent protein, blue fluorescent protein, cyan fluorescent protein or yellow fluorescent protein. Other exemplary fluorescent proteins include, but are not limited to, enhanced green fluorescent protein (EGFP), split GFP, AcGFP, TurboGFP, Emerald, Azami Green, ZsGreen, EBFP, Sapphire, T-Sapphire, ECFP, mCFP, Cerulean, CyPet, AmCyan1, Midori-Ishi Cyan, mTFP1 (Teal), enhanced yellow fluorescent protein (EYFP), Topaz, Venus, mCitrine, YPet, PhiYFP, ZsYellow1, mBanana, Kusabira Orange, mOrange, dTomato, dTomato-Tandem, DsRed, DsRed2, DsRed-Express (T1), DsRed-Monomer, mTangerine, mStrawberry, AsRed2, mRFP 1, JRed, mCherry, HcRed1, mRaspberry, HcRed1, HcRed-Tandem, mPlum, and AQ143.

Additional suitable labels that can be used in accordance with the disclosure include, but are not limited to, fluorescent, chemiluminescent, chromogenic, phosphorescent, and/or radioactive labels. In addition, when an epitope tag is included in a complex, the complex is detectable using an antibody that is immunoreactive with the epitope tag.

Any complex of the disclosure can be readily tested to confirm that, following complex formation, the complex retains the ability to penetrate cells and retains at least 50% of an activity of the cargo portion. This testing can be done regardless of whether the complex is a fusion protein (directly or via a linker) or a chemical fusion or otherwise associated. By way of example, the FGF-10 portion may be tested for cell penetration activity alone and the cargo portion may be tested for one or more activities. After confirming that the selected FGF-10 portion does penetrate cells and the cargo portion retains its function, a complex is generated using any suitable method. Following complex formation, cell penetration activity is again assessed to confirm that complex formation did not interfere with cell penetration activity, and that the FGF-10 portion penetrates cells in association with this cargo. Additionally, following complex formation, activity of the cargo portion (present in the complex) is tested to confirm complex formation does not interfere with activity (e.g., that the cargo portion provided in the complex retains at least 50%, 55%, 60%, 70%, 75%, 80%, 85%, 90%, 92%, 93%, 94%, 95%, 96%, 97%, 98%, 99%, 100%, or greater than 100% of an activity of the native cargo portion alone).

Regardless of whether a complex of the disclosure is being used in vitro or in vivo, or for research or therapeutic use, the disclosure contemplates that an endosomal escape agent (e.g., a compound that promotes escape from the endosome) may be administered at the same time, before, or after administration of the complex. Exemplary agents that promote escape from the endosome include, without limitation, chloroquine, cytolysins, and PFO or PFO-related agents, such as any of the agents disclosed in the following PCT application: WO2012/094653. These are merely exemplary of endosome escape agents, and numerous other are available to one of skill in the art. One of skill in the art can readily determine (i) whether an endosomal escape agent is beneficial in a particular research or therapeutic context and, if so, (ii) the amount of endosomal escape agent that facilitate availability of the complex to cells while minimizing unwanted toxicity.

(v) Applications

The present disclosure provides complexes comprising (i) an FGF-10 portion and (ii) a cargo portion. Complexes of the disclosure have numerous uses, including for delivering cargo into cells in vitro or in vivo. Complexes of the disclosure are suitable for preferentially delivering cargo to cell and tissues efficiently penetrated by FGF-10 polypeptides (See Tables 1 and 2). Depending on the particular cargo portion, complexes are useful for therapeutic, diagnostic, research, and other purposes. For example, complexes can be used in vitro for studying the biology of the cargo portion, protein interactions involving the cargo, and the like.

Moreover, the complexes of the disclosure have numerous applications, including research uses, therapeutic uses, diagnostic uses, imaging uses, and the like, and such uses are applicable over a wide range of targets and disease indications.

Particular diagnostic, therapeutic, imaging, and research uses for complexes of the disclosure depend on the cargo portion selected. For example, in certain embodiments, the cargo is a target binding moiety. Exemplary target binding moieties bind to and inhibit activity of a target. Exemplary targets are present or expressed in a particular cell or tissue, such as a cell or tissue to which FGF-10 portions preferentially localize. In certain embodiments, the target bound by the target binding moiety is expressed or otherwise present in a tissue that the FGF10 portion preferentially penetrate, such as liver or kidney (See tables 1 and 2 providing localization data).

By way of example, in certain embodiments, the cargo portion is an enzyme and the complex is administered to supplement endogenous enzyme expressions. Complexes comprising an enzyme are also useful for identifying, in a cell-based system, substrates, cofactors, or binding partners of that enzyme.

By way of further example, in certain embodiments, the cargo portion is small organic molecule, such as a chemotherapeutic agent. Complexes comprising such a small molecule as a cargo portion are suitable for preferential, non-ubiquitous delivery of a cancer therapeutic. This helps reduce off-target toxicity.

By way of further example, in certain embodiments, the cargo portion is a tumor suppressor protein. Such complexes are useful for studying the function of the tumor suppressor protein, as well as methods of treating cancers comprising a change of expression and/or activity of the tumor suppressor protein. One such tumor suppressor protein is p16.

In certain embodiments, the cargo portion is target binding moiety. For example, a target binding moiety that binds to and inhibits a target or a target binding moiety that binds to and inhibits a target present or expressed in a cell.

Any target binding moiety may be provided as a complex with an FGF10 portion, such as an FGF-related Surf+ Penetrating Polypeptide, and delivered to a cell using the inventive system. Given the ability to readily make and test antibodies and antibody-mimics, and thus, to generate target binding moieties capable of binding to a target and having a desired activity (e.g., inhibiting the function of the target, promoting the function of the target, binding without interfering or altering the function of the target), the present system may be used in combination with virtually any target, such as a polypeptide or peptide, expressed in a cell. Accordingly, the complexes of the disclosure have numerous applications, including research uses, therapeutic uses, diagnostic uses, imaging uses, and the like, and such uses are applicable over a wide range of targets and disease indications.

The following provides specific examples, including examples of specific targets. However, the potential uses of complexes of the disclosure are not limited to specific target polypeptides or peptides. Rather, the general uses include, at least, the following. Complexes of the disclosure are useful for delivering target binding moieties into cells where they are useful for labeling a target protein, such as for imaging cells, tissues and whole organisms. Labeling may be useful when performing research studies of protein expression, disease progression, cell fate, protein localization and the like. Labeling may be useful diagnostically or prognostically, such as in cases where target expression correlates with a particular condition. In certain embodiments, an target binding moiety intended for labeling may be selected such that it does not interfere with the function of the target (e.g., a moiety that binds to a target but does not alter the activity of the target).

In addition, complexes of the disclosure may be used in research setting to study target expression, presence/absence of target in a disease state, impact of inhibiting or promoting target activity, etc. Complexes of the disclosure are suitable for these studies in vitro or in vivo.

Further, complexes of the disclosure have therapeutic uses by promoting delivery of target binding moieties into cells in humans or animals (including animal models of a disease or condition). Once again, the use of complexes of the disclosure decrease failure of an target binding moiety due to inability to effectively penetrate cells or due to the inability to effectively penetrate cells at concentrations that are not otherwise toxic to the organism.

Regardless of whether a complex of the disclosure is used in a research, diagnostic, prognostic or therapeutic context, the result is that the cargo portion (e.g., target binding moiety or other cargo portion) is delivered into a cell following contacting the cell with the complex (e.g., either contacting a cell in culture or administrated to a subject).

In certain embodiments, the target binding moiety binds a target expressed in the nucleus or in the cytosol of a cell. In some embodiments, target binding moiety binds a membrane associated target, e.g., a target localized on the cytosolic side of the cell membrane, the cytosolic side of the nuclear membrane, or the cytosolic side of the mitochondrial membrane.

In certain embodiment, an FGF-10 portion, such as an FGF10-related Surf+ Penetrating Polypeptide, is complexed with target binding moiety that binds a target in the nucleus of a cell, such as an NFAT (Nuclear Factor of Activated T cells) (e.g., NFAT-2), a STAT (Signal Transducer and Activator of Transcription) (e.g., STAT-3, STAT-5, or STAT-6) or RORgammaT (retinoic acid-related orphan receptor).

In certain embodiments, a FGF-10 portion, such as an FGF10-related Surf+ Penetrating Polypeptide, is complexed with a target binding moiety that binds a target in the cytosol of the cell, such as FK506, calcineurin, or a Janus Kinase (e.g., JAK-1 or JAK-2.

In another embodiment, a FGF-10 portion, such as an FGF10-related Surf+ Penetrating Polypeptide, is complexed with a target binding moiety that binds a target localized on the cytosolic side of the cell membrane, such as ras, a PI3K (phosphoinositide-3-kinase), or fins-related tyrosine kinase 1 (vascular endothelial growth factor/vascular permeability factor receptor).

In yet other embodiments, a FGF-10 portion, such as an FGF10-related Surf+ Penetrating Polypeptide, is complexed with a target binding moiety that binds a target localized on the cytosoloic side of the mitochondrial membrane, such as Bcl-2.

In some embodiments, the target binding moiety binds a kinase, a transcription factor or an oncoprotein. For example, the target binding moiety can bind a kinase, such as a JAK kinase (e.g., JAK-1 or JAK-2) or b-raf (v-raf murine sarcoma viral oncogene homolog B1) or Erk (mitogen-activated protein kinase 1). By way of further example, the target binding moiety can bind a transcription factor, such as Hif1-alpha, a STAT (e.g., STAT-3, STAT-5 or STAT-6), or IRF-1 (Interferon Regulatory Factor 1). In some embodiments, the target binding moiety binds an oncogene, such as ras, b-raf or Akt (v-akt murine thymoma viral oncogene homolog 1).

In some embodiments, a complex comprising (i) an FGF10 portion and (ii) a cargo portion in accordance with the present disclosure may be used for therapeutic purposes, or may be used for diagnostic purposes. The disease or condition that may be treated depends on the target (e.g., the target is one for which binding by a target binding moiety has a therapeutic benefit).

For example, a complex in accordance with the present disclosure may be used for treatment of any of a variety of diseases, disorders, and/or conditions, including but not limited to one or more of the following: autoimmune disorders; inflammatory disorders; and proliferative disorders, including cancers. In one embodiment, the disease treated by the complex is a cardiovascular disorder, or an angiogenic disorder such as macular degeneration. In another embodiment, the disease treated by the complex is an eye disease, such as age-related macular degeneration (AMD), diabetic macular edema (DME), retinitis pigmentosa, or uveitis.

In some embodiments, a complex is useful for treating one or more of the following: an infectious disease; a neurological disorder; a respiratory disorder; a digestive disorder, a musculoskeletal disorder; an endocrine, metabolic, or nutritional disorders; a urological disorder; psychological disorder, a skin disorder; a blood and lymphatic disorder; etc.

In certain embodiments, the complex of the disclosure targets, via the target binding moiety, a protein set forth in Table C. In other words, the target binding moiety portion of the complex binds (e.g., specifically binds) to the target expressed or otherwise located inside the cell (the intracellular target). In certain embodiments, targeting the protein may be useful in the research, diagnosis, prognosis, monitoring or treatment of the listed disease.

TABLE C
Exemplary target proteins.

| | | | Intracellular |
|---|---|---|---|
| Diseases | Target | Protein class | Location of Target |
| cancer, age-related | Hif1-alpha | Txn factor | nuclear |
| macular degeneration, | | | |
| ischemia, rheumtoid | | | |
| arthritis | | | |
| dry eye, psoriasis | Calcineurin | Phosphatase | cytosol |
| psoriasis | peptidylprolyl isomerase A | peptidylprolyl | cytosol |
| | (cyclophilin A) | isomerase | |
| psoriasis | peptidylprolyl isomerase A | peptidylprolyl | cytosol |
| | (FK506 binding | isomerase | |
| | protein/immunophilin) | | |
| dry eye, psoriasis | NFATs (NFAT-2) | Txn factor | nuclear |
| cancer, Transplant | mechanistic/mammalian | serine/threonine | cytosol |
| Rejection, Restenosis, | target of rapamycin mTOR, | kinase | |
| glycogen storage | FRAP1; (serine/threonine | | |
| disease | kinase) | | |
| myelofibrosis, cancer, | Janus Kinases (such as | non-receptor tyrosine | cytosol |
| inflammation | JAK-1 and JAK-2) | kinase | |
| inflammatory diseases | SOCS1, SOCS3 (suppressor | STAT binding | cytosol |
| (rheumatoid arthritis, | of cytokine signaling) | protein | |
| gout, crohn's disease), | | | |
| epilespy, Huntington | | | |
| Disease | | | |
| autoimmune diseases | STAT-3 (signal transducer | Txn factor | nuclear |
| such as multiple | and activator of | | |
| sclerosis and cancer, | transcription) | | |
| age-related macular | | | |
| degeneration, uveitis | | | |
| cancer (Sezary | STAT-5 | Txn factor | nuclear |
| disease) | | | |
| autoimmune diseases | STAT-6 | Txn factor | nuclear |
| such as atopic | | | |
| dermatitis and | | | |
| emphysema, COPD, | | | |
| lung fibrosis, acute | | | |
| asthma | | | |
| cancer | Ras | GTPase, signal | cytosolic-side of cell |
| | | transducing protein | membrane |
| cancer such as | b-raf | serine/threonine | cytosol |
| melanoma | | kinase | |
| cancer, prion diseases | Erk | Txn factor | multiple locations |
| such as Creutzfeldt- | | | depending on cell- |
| Jakob Disease | | | type and disease |
| cancer | MAP Kinases (mitogen | serine/threonine | cytosol |
| | activated kinases) | kinase | |
| cancer | Jnk (C-Jun N-terminal | serine/threonine | cytosol |
| | kinase) | kinase | |
| cancer | MEK (MAP/Erk kinase) | serine/threonine | cytosol |
| | | kinase | |
| cancer | PI3K (phosphatidyl inositol | lipid kinase | cytosolic-side of cell |
| | 3 kinase) | | membrane |
| cancer | AKT | serine/threonine | cytosol |
| | | kinase | |
| inflammatory diseases | Caspase-1 (cysteine-aspartic | protease | cytosol |
| (arthritis, gout, | proteases) | | |
| inflammatory bowel | | | |
| disease), | | | |
| neurodiseases | | | |
| (Huntington Disease, | | | |
| epilepsy) and | | | |
| metabolic diseases | | | |
| such as diabetes type | | | |
| 2 and obesity, | | | |
| cryopyrin-associated | | | |
| periodic syndromes, | | | |
| chronic obstructive | | | |
| pulmonary disease | | | |
| inflammatory diseases | NEMO also known as IKK [symbol] | regulatory binding | cytosol |
| such as psoriasis, | (IKK gamma) | protein/adaptor | |
| rheumatoid arthritis, | | scaffold protein | |
| age-related macular | | | |
| degeneration, cancer, | | | |
| duchene muscular | | | |
| dystrophy, ALS, and | | | |
| cachexia-induced | | | |
| cardiac atrophy | | | |
| inflammatory diseases | MyD88 (Myeloid | regulatory binding | cytosol |
| (rhuematoid arthritis, | differentiation primary | protein/adaptor | |
| gout, crohn's disease), | response) | scaffold protein | |
| epilespy, Huntington | | | |
| Disease; pyogenic | | | |
| bacterial infections | | | |
| inflammatory diseases | ASC | regulatory binding | cytosol |
| (arthritis, gout, | | protein/adaptor | |
| inflammatory bowel | | scaffold protein | |
| disease), | | | |
| neurodiseases | | | |
| (Huntington Disease, | | | |
| epilepsy) and | | | |
| metabolic diseases | | | |
| such as diabetes type | | | |
| 2 and obesity, | | | |
| cryopyrin-associated | | | |
| periodic syndromes, | | | |
| chronic obstructive | | | |
| pulmonary disease | | | |
| inflammatory diseases | NLRP3 (inflammasome | regulatory binding | cytosol |
| (arthritis, gout, | component) | protein/adaptor | |
| inflammatory bowel | | scaffold protein | |
| disease), | | | |
| neurodiseases | | | |
| (Huntington Disease, | | | |
| epilepsy) and | | | |
| metabolic diseases | | | |
| such as diabetes type | | | |
| 2 and obesity, | | | |
| cryopyrin-associated | | | |
| periodic syndromes, | | | |
| chronic obstructive | | | |
| pulmonary disease | | | |
| inflammatory and | retinoic acid-related orphan | Txn factor | nuclear |
| autoimmune diseases | receptor (ROR [symbol] T) | | |
| such as inflammatory | (RORgammaT) | | |
| bowel disease, | | | |
| multiple sclerosis, | | | |
| Gout, Arthritis, | | | |
| psoriasis | | | |
| cancer | Thymidylate synthase | metabolic enzyme | cytosol & nucleus |
| cancer | abl tyrosine kinase; bcr-abl | tyrosine kinase | cytosol |
| | (product of chromosomal | | |
| | translocation) | | |
| | Interferon Regulatory Factor | Txn factor | nucleus |
| | 1 (IRF-1) - transcription | | |
| | factor | | |
| cancer | fms-related tyrosine kinase | tyrosine kinase | cytosolic-side of cell |
| | 1 (vascular endothelial | | membrane |
| | growth factor/vascular | | |
| | permeability factor receptor) | | |
| cancer | fms-related tyrosine kinase 3 | tyrosine kinase | cytosolic-side of cell |
| | | | membrane |
| cancer | kinase insert domain | tyrosine kinase | cytosolic-side of cell |
| | receptor (a type III receptor | | membrane |
| | tyrosine kinase) | | |
| cancer | macrophage stimulating 1 | tyrosine kinase | cytosolic-side of cell |
| | receptor (c-met-related | | membrane |
| | tyrosine kinase) | | |
| cancer, diabetic | protein kinase C family | serine/threonine | multiple locations |
| retinopathy | (alpha, beta) | kinase | depending on cell- |
| | | | type and disease |
| | | | (cytosolic, associated |
| | | | with cell membrane |
| Cancer | beta tubulin/microtubule | cytoskeletal | cytosol |
| | | structural protein | |
| Cancer, Charcot- | kinesins and chromosome- | microtubule | cytosol |
| Marie-Tooth, | associated KIF | associated motor | |
| neurogenerative | | protein | |
| diseases, eye disorder | | | |
| Cancer, kidney | Dynein | microtubule | cytosol |
| diseases, respiratory | | associated motor | |
| diseases, hearing loss | | protein | |
| inflammation, pain | prostaglandin-endoperoxide | cyclooxygenase | cytosolic face of |
| | synthase 2 (prostaglandin | | membranes |
| | G/H synthase and | | |
| | cyclooxygenase) COX-2 | | |
| cancer | Rho associated protein | serine/threonine | cytosol |
| | kinases | kinase | |
| cancer | Aurora protein kinases | serine/threonine | nucleus-cytosol |
| | | kinase | (functions before and |
| | | | during nuclear |
| | | | envelope breakdown) |
| | Insulin receptor substrates | regulatory binding | cytosolic face of |
| | (IRS) | protein/adaptor | plasma membrane |
| | | scaffold protein | |
| cancer | focal adhesion kinases | tyrosine kinase | cytosol |
| | (PTK2) | | |
| cancer | cyclin dependent kinases | serine/threonine | nucleus |
| | | kinase | |
| Cancer | Bcl-2 | regulatory binding | outer mitochondrial |
| | | protein/adaptor | membrane |
| | | scaffold protein | |
| cancer | Telomerase | reverse transcriptase | nuclear |
| cancer | cytochrome c | electron transport | cytosol (when |
| | | pathway component, | released from |
| | | regulatory binding | mitochondria) |
| | | protein/adaptor | |
| | | scaffold protein (only | |
| | | in context of | |
| | | stimulating | |
| | | apoptosis) | |

The foregoing are merely exemplary of targets that can be bound and inhibited by a cargo portion. The present disclosure is applicable to any target

Regardless of the target or the particular use, in certain embodiments, a complex is administered to a cell or organism in an effective amount. The term “effective amount” means an amount of an agent to be delivered that is sufficient, when administered to a cell or a subject to have the desired effect. In the context of the present disclosure, an effective amount may be the amount sufficient to promote delivery of the complex into a cell and to promote binding of the target binding moiety to its target. In a therapeutic setting, an effective amount is the amount sufficient to treat (e.g., alleviate, improve or delay onset of one or more symptoms of) a disease, disorder, and/or condition.

In one embodiment, the target binding moiety is bispecific, e.g., is a bispecific antibody, or bispecific fragment thereof. A complex comprising a bispecific antibody can bind two different target polypeptides at the same time, or at different times. For example, a bispecific target binding moiety can bind an extracellular target prior to internalization of the complex into the cell, and a second target after internalization into the cell. In another embodiment, the bispecific agent binds two targets, e.g., two intracellular targets simultaneously or consecutively.

A complex of the disclosure may be used in a clinical setting, such as for therapeutic purposes. Therapeutic complexes may include an target binding moiety that binds to and reduces the activity of one or more targets (e.g., polypeptide targets). Such target binding moieties are particularly useful for treating a disease, disorder, and/or condition associated with high levels of one or more particular targets, or high activity levels of one or more particular targets.

In some embodiments, the complex is detectable (e.g., one or both of the FGF-10 portion and the cargo portion are modified with a detectable label). For example, one or both portions of the complex may include at least one fluorescent moiety. In some embodiments, the FGF-10 portion has inherent fluorescent qualities. In some embodiments, one or both portions of the complex may be associated with at least one fluorescent moiety (e.g., conjugated to a fluorophore, fluorescent dye, etc.). Alternatively or additionally, one or both portions of the complex may include at least one radioactive moiety (e.g., protein may comprise iodine-131 or Yttrium-90; etc.). Such detectable moieties may be useful for detecting and/or monitoring delivery of the complex to a target site.

A complex associated with a detectable label can be used in detection, imaging, disease staging, diagnosis, or patient selection. Suitable labels include fluorescent, chemiluminescent, enzymatic labels, colorimetric, phosphorescent, density-based labels, e.g., labels based on electron density, and in general contrast agents, and/or radioactive labels.

In some embodiments, the complexes featured in the disclosure may be used for research purposes, e.g., to efficiently deliver a cargo portion to cells in a research context. In some embodiments, the complexes may be used as research tools to efficiently transduce cells with antibody molecules or with other target binding moieties or other polypeptides or peptides. In some embodiments, complexes may be used as research tools to efficiently introduce a cargo portion, such as a target binding moiety, into cells for purposes of studying the effect of the cargo portion on cellular activity. In certain embodiments, a complex can be used to deliver a target binding moiety into a cell for the purpose of studying the biological activity of the target peptide or protein (e.g., what happens if the target is inhibited or agonized, etc.). In certain embodiments, a complex may be introduced into a cell for the purpose of studying the biological activity of the target binding moiety (e.g., does it inhibit target activity, does it promote target activity, etc.).

Below is described further applications of the complexes of the disclosure. To illustrate, below are examples for uses when the cargo portion of the complex comprises a p16 tumor suppressor protein. However, similar concepts in terms of treating, efficacy, and the like are applicable when other cargo is used to treat or study other indications.

(a) Research Methods of Use

In certain embodiments, the complexes of the disclosure comprise a p16 tumor suppressor, or a functional fragment or functional variant thereof, and such complexes are used for research purposes. For example, such proteins can penetrate cells, and thus, provide a more accurate assessment of protein-protein and protein-nucleic acid interactions involving p16 than can be achieved in cell-free systems or with uncomplexed p16. In this context, complexes of the disclosure are particularly useful for identifying and purifying proteins and nucleic acids that bind p16 directly or that form a complex, endogenously, with p16.

Such complexes of the disclosure are also useful in vitro and in animal models to evaluate the role of p16 in changes to cell behavior and tumorigenesis in various genetic backgrounds (e.g., Rb+ versus Rb−), as well as the ability to replace the function of p16 via a protein replace technology.

(b) Therapeutic Methods of Uses

In certain embodiments, the disclosure contemplates that complexes of the disclosure (as well as formulations thereof) described herein may be used therapeutically, for example, in the treatment of human or non-human subjects. In certain embodiments, the complexes of the disclosure comprise a p16 portion as the cargo portion. Such complexes of the disclosure may be administered to a patient in need thereof. Specifically, complexes of the disclosure may be used therapeutically (alone or in combination with one or more other agents) in the treatment of cancer. In certain embodiments, the cancer is associated with decreased expression and/or activity of p16. In certain embodiments, the cancer comprises a mutation in p16 that decreases expression and/or activity of p16. In certain embodiments, the cancer is primary or metastatic cancer in the abdominal cavity. For example, the primary or metastatic cancer may be of or associated with liver, pancreas, or ovaries. In other embodiments, the primary or metastatic cancer is or is associated with head or neck.

In certain aspects, the complexes and formulations of the disclosure comprising a p16 portion may be administered for treatment of a subject in need thereof, such as a human subject.

Since its discovery as a CDKI (cyclin-dependent kinase inhibitor) in 1993, the importance in cancer of the tumor suppressor p16 (INK4A/MTS-1/CDKN2A) has gained widespread appreciation. The frequent mutations and deletions of p16 in human cancer cell lines first suggested an important role for p16 in carcinogenesis. This genetic evidence for a causal role was significantly strengthened by the observation that p16 was frequently inactivated in familial melanoma kindreds. Since then, a high frequency of p16 gene alterations were observed in many primary tumors.

Mutations in the CDKN2A gene and other factors that decrease the expression and/or function of a p16 protein isoform correlate with increased risk of a wide range of cancers. Exemplary cancers often associated with mutations or alterations in p16 include, but are not limited to, melanoma, pancreatic ductal adenocarcinoma, gastric mucinous cancer, primary glioblastoma, mantle cell lymphoma, hepatocellular carcinoma and ovarian cancer. Additionally, mutations or deletions in p16 are frequently found in, for example, esophageal and gastric cancer cell lines. Any such cancers, primary or metastatic, can be treated using the complexes and methods of the disclosure.

Complexes of the disclosure are suitable for delivering p16 into cells in a subject. When the complex s administered in a therapeutic context, the subject in need thereof has or is suspected of having cancer (either primary or metastatic). Complex is administered to such subjects. The amount of complex administered at each dosage will be determined by the health care provider, consistent with, for example, the size of the patient, the status of and patient's disease. Moreover, the optimal dosage regimen may be evaluated. It is readily appreciated by one of skill in the art, that when the application or claims refers to an effective dose, that does not require that each dose of a multi-dose therapeutic regimen much itself be sufficient to generate an objective effect on the subject.

“Treating” a condition or disease refers to curing as well as ameliorating at least one symptom of the condition or disease, and includes administration of a composition which reduces the frequency of, or delays the onset of, symptoms of a medical condition in a subject in need relative to a subject which does not receive the composition. Thus, treating cancer includes, for example, reducing the number of detectable cancerous growths in a population of patients receiving a treatment relative to an untreated control population, and/or delaying the appearance of detectable cancerous growths in a treated population versus an untreated control population, e.g., by a statistically and/or clinically significant amount. By way of further example, treating cancer includes, for example, delaying disease progression, delaying or preventing metastases, reducing the number of metastases, increase life span, reducing pain (e.g., such as by reducing the size of tumor(s) that are causing pain). As another example, treatment of pain includes reducing the magnitude of, or alternatively delaying, pain sensations experienced by subjects in a treated population versus an untreated control population.

The present disclosure provides, in part, complexes (and methods for using such complexes). For example, the complexes are used to deliver p16, such as to augment expression and/or activity. In a particular embodiment, the formulations of the disclosure are used to treat a neoplastic disease.

Additionally, in early or advanced stages of disease, a p16 therapeutic of the disclosure can be used in novel combination regimens with existing approved therapeutics or new agents, for example combining with CDK4/6 inhibitors or other therapeutics specifically affecting the cell cycle, or tumor cell growth in general.

Further, the disclosure contemplates that any such formulations can be used as part of a therapeutic regimen appropriate for the particular condition. By way of example, suitable regimens may include, in addition to a complex of the disclosure one or more of (i) one or more other agents, such as chemotherapeutic agents, other antibodies or other small molecule inhibitors; (ii) radiotherapy; (iii) surgery; (iv) a dietary regimen; (v) bone marrow transplant; (vi) stem cell transplant; (vii) dialysis; (viii) physical therapy; (ix) skin grafting; (x) acupuncture; (xi) oxygen therapy; (xii) insulin therapy; (xiii) smoking cessation; and the like. Therapeutic interventions that are not drugs or biological agents are also referred to herein as other therapeutic modalities or other therapies. Exemplary agents and combinations of agents are described below.

In certain embodiments, a complex of the disclosure is part of a combination therapy suitable for treating a condition. When a therapeutic regimen involves more than one agent, the agents may be administered at the same or differing times. The agents may even be administered together in a composition that comprises both active ingredients. Agents may be administered by the same route of administration or by differing routes of administration. In the context of a therapeutic regimen for treating cancer, such as a cancer in which p16 expression and/or activity is diminished, a complex of the disclosure may be used as part of a combination therapy with the then current standard of care for the particular cancer (and particular stage of cancer) being treated. In the context of ovarian cancer, the current standard of care includes administration of carboplatin and/or paclitaxel. However, the disclosure contemplates combinations with, for example, any suitable chemotherapeutic agents, including chemotherapeutic agents that work via the same or differing mechanism as carboplatin or paclitaxel (or variants, derivatives, or analogs thereof). Additional examples include CDK4/6 inhibitors, such as the small molecule inhibitor designated PD-0332991 in development by Pfizer.

By way of further example, in the context of liver cancer suitable additional agents include the then current standard of care. For example, the current standard of care is administration of sorafenib, and thus, in certain embodiments, the combination therapy includes administration of sorafenib.

In the context of a combination therapy, a physician will modulate the dosing schedule and dose of the two agents to maximize therapeutic benefit while minimizing untolerable or dangerous side effects.

Regardless of the particular formulation administered, an effective amount is administered to patients. As used herein, the term “effective amount” refers to the amount of a therapy which is sufficient to reduce and/or ameliorate the severity and/or duration of a disease or disorder; prevent or delay the advancement of said disease or disorder, cause regression of said disease or disorder; prevent or delay the recurrence, development, or onset of one or more symptoms associated with said disease or disorder, or enhance or improve the effect(s) of another therapy. It is understood that measurable signs of effectiveness may not be observable following a single dose.

For any methods of treating involving administering a combination of agents and/or therapies, such conjoint treatment may be achieved by way of the simultaneous, sequential or separate dosing of the individual components of the treatment.

Regardless of whether a complex of the disclosure is being used in vitro or in vivo, or for research or therapeutic use, the disclosure contemplates that an endosomal escape agent (e.g., a compound that promotes escape from the endosome) may be administered at the same time, before, or after administration of the complex. Exemplary agents that promote escape from the endosome include, without limitation, chloroquine, cytolysins, and PFO or PFO-related agents, such as any of the agents disclosed in the following PCT application: WO2012/094653. These are merely exemplary of endosome escape agents, and numerous other are available to one of skill in the art. One of skill in the art can readily determine (i) whether an endosomal escape agent is beneficial in a particular research or therapeutic context and, if so, (ii) the amount of endosomal escape agent that facilitate availability of the complex to cells while minimizing unwanted toxicity.

(vi) Pharmaceutical Compositions

The present disclosure provides complexes of the disclosure (e.g., FGF10 portion-associated with a cargo portion, where the FGF10 portion comprises or consists of an FGF10-related Surf+ Penetrating Polypeptide). This section describes exemplary compositions, such as compositions of a complex of the disclosure formulated in a pharmaceutically acceptable carrier. Any of the complexes described herein comprising FGF10 portion and a cargo portion may be formulated in accordance with this section of the disclosure.

Thus, in certain aspects, the present disclosure provides compositions, such as pharmaceutical compositions, comprising one or more such complexes, and one or more pharmaceutically acceptable excipients. Pharmaceutical compositions may optionally include one or more additional therapeutically active substances. In some embodiments, the compositions are suitable for administration to humans. In other embodiments, the compositions are suitable for administration to non-human animals, or are suitable for laboratory use but not for animal use. For the purposes of the present disclosure, the phrase “active ingredient” generally refers to a cargo portion complexed with an FGF-10 portion, as described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts, as well as suitable or adaptable for research use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects or patients to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition in accordance with the disclosure may be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100% (w/w) active ingredient.

Pharmaceutical formulations may additionally include a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, Md., 2006; incorporated herein by reference) discloses various excipients used in formulating pharmaceutical compositions and known techniques for the preparation thereof.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of active ingredient may be as high as the solubility limit of the active ingredient in the solvent.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (w/w) solution and/or suspension of the active ingredient in an aqueous or oily liquid excipient. Such drops may further comprise buffering agents, salts, and/or one or more other of any additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

In certain embodiments, complexes of the disclosure and compositions of the disclosure, including pharmaceutical preparations, are non-pyrogenic. In other words, in certain embodiments, the compositions are substantially pyrogen free. In one embodiment, the formulations of the disclosure are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in relatively large dosages and/or over an extended period of time (e.g., such as for the patient's entire life), even small amounts of harmful and dangerous endotoxin could be dangerous. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

General considerations in the formulation and/or manufacture of pharmaceutical agents may be found, for example, in Remington: The Science and Practice of Pharmacy 21 ed., Lippincott Williams & Wilkins, 2005 (incorporated herein by reference).

(vii) Administration

The present disclosure provides methods for delivering a cargo portion into a cell (or into cells or tissues). Cells or tissues are contacted with a complex comprising a cargo portion and an FGF-10 portion, thereby promoting delivery of the cargo portion into the cell.

The present disclosure provides methods comprising administering complexes of the disclosure to a subject in need thereof, as well as methods of contacting cells or cells in culture with such complexes. The disclosure contemplates that any of the complexes of the disclosure may be administrated, such as described herein. Complexes of the disclosure, including as pharmaceutical compositions, may be administered or otherwise used for research, diagnostic, imaging, prognostic, or therapeutic purposes, and may be used or administered using any amount and any route of administration effective for preventing, treating, diagnosing, researching or imaging a disease, disorder, and/or condition. The exact amount required will vary from subject to subject, depending on the species, age, and general condition of the subject, the severity of the disease, the particular composition, its mode of administration, its mode of activity, and the like. Compositions in accordance with the disclosure are typically formulated in dosage unit form for ease of administration and uniformity of dosage. It will be understood, however, that the total daily usage of the compositions of the present disclosure will be decided by the attending physician within the scope of sound medical judgment. The specific therapeutically effective, prophylactically effective, or appropriate imaging dose level for any particular patient will depend upon a variety of factors including the disorder being treated and the severity of the disorder, the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

FGF10 portion/cargo portion complexes (e.g., complexes of the disclosure) comprising at least one agent to be delivered and/or pharmaceutical, prophylactic, diagnostic, research or imaging compositions thereof may be administered to animals, such as mammals (e.g., humans, domesticated animals, cats, dogs, mice, rats, etc.). In some embodiments, complexes of the disclosure comprising at least one agent to be delivered, and/or pharmaceutical, prophylactic, diagnostic, research or imaging compositions thereof are administered to humans.

Complexes of the disclosure comprising at least one agent to be delivered and/or pharmaceutical, prophylactic, research diagnostic, or imaging compositions thereof in accordance with the present disclosure may be administered by any route and may be formulated in a manner suitable for the selected route of administration or in vitro application. In some embodiments, complexes of the disclosure, and/or pharmaceutical, prophylactic, diagnostic, research or imaging compositions thereof, are administered by one or more of a variety of routes, including oral, intravenous, intramuscular, intra-arterial, intramedullary, intrathecal, subcutaneous, intraventricular, transdermal, intradermal, rectal, intravaginal, intraperitoneal, topical (e.g. by powders, ointments, creams, gels, lotions, and/or drops), mucosal, nasal, buccal, enteral, vitreal, intratumoral, sublingual; by intratracheal instillation, bronchial instillation, and/or inhalation; as an oral spray, nasal spray, and/or aerosol, and/or through a portal vein catheter (e.g., via the portal vein), a transurethral or transureter catheter, or laproscopically. Other devices suitable for administration include, e.g., microneedles, intradermal specific needles, Foley's catheters (e.g., for bladder instillation), and pumps, e.g., for continuous release.

In certain embodiments, administration is intraperitoneal administration. Generally, intraperitoneal administration involves a much larger volume than is seen with, for example, intravenous administration. For example, it is not unusual for 1-2 liters of fluid to be administered when the route of delivery is intraperitoneal.

In some embodiments, complexes of the disclosure, and/or pharmaceutical, prophylactic, diagnostic, research or imaging compositions thereof, are administered by systemic intravenous injection. In specific embodiments, complexes of the disclosure and/or pharmaceutical, prophylactic, research diagnostic, or imaging compositions thereof may be administered intravenously and/or orally. In specific embodiments, complexes of the disclosure, and/or pharmaceutical, prophylactic, research diagnostic, or imaging compositions thereof, may be administered in a way which allows the complex to cross the blood-brain barrier, vascular barrier, or other epithelial barrier.

Complexes of the disclosure comprising at least one cargo portion to be delivered may be used in combination with one or more other therapeutic, prophylactic, diagnostic, research or imaging agents. By “in combination with,” it is not intended to imply that the agents must be administered at the same time and/or formulated for delivery together, although these methods of delivery are within the scope of the disclosure. Compositions of the disclosure can be administered concurrently with, prior to, or subsequent to, one or more other desired therapeutics, other reagents or medical procedures. In general, each agent will be administered at a dose and/or on a time schedule determined for that agent. In some embodiments, the disclosure encompasses the delivery of pharmaceutical, prophylactic, diagnostic, research or imaging compositions in combination with agents that improve their bioavailability, reduce and/or modify their metabolism, inhibit their excretion, and/or modify their distribution within the body.

It will further be appreciated that therapeutic, prophylactic, diagnostic, research or imaging active agents utilized in combination may be administered together in a single composition or administered separately in different compositions. In general, it is expected that agents utilized in combination with be utilized at levels that do not exceed the levels at which they are utilized individually. In some embodiments, the levels utilized in combination will be lower than those utilized individually.

When complexes of the disclosure are used in combination with one or more other agents, the other agent is selected based on the particular application (e.g., the disease or condition being treated or other application). In certain embodiments, when a complex of the disclosure is used in the treatment of cancer, the one or more other agents is the current standard of care for that cancer.

(viii) Assays and Models

Complexes of the disclosure are tested using assays and models to confirm that the complexes retain the cell penetration activity of the FGF-10 portion and the functional activity (at least 50% of the functional activity) of the cargo portion. Exemplary cell penetration assays are provided in the examples. Cell penetration of the FGF-10 portion alone and/or the complex is assessed to confirm that the domain of the FGF-10 polypeptide selected functions as a Surf+ Penetrating Polypeptide and retains that function when provided as a complex.

For confirming the activity of the cargo portion, an appropriate assay is selected based on the desired functional activity of the specific cargo and complex being evaluated. For example, if the cargo portion comprises a target binding moiety, suitable assays include cell free or cell based binding assays to confirm the target binding moiety binds the appropriate target. Further assays are conducted to confirm that the target binding moiety inhibits an activity of the target, if desired. If the cargo moiety is an enzyme, then suitable assays include in vitro assays of enzymatic activity. If the cargo moiety is a transcription factor, a suitable assay may include a DNA binding assay.

Moreover, depending on the particular complex and its intended use, complexes can be assayed in cell-based or animal models. Such models include wild type cells and animals to confirm cell penetration and proper cell/tissue localization, as well as functional activity of the cargo portion. Cell or tissue specific cell types may also be used. Additionally or alternatively, cancer cell lines or cells/tissue derived from an animal model of disease can be used to evaluate cell penetration and localization in the diseased context, as well as to assess functional activity of the cargo portion. These models may also be suitable to evaluate whether the cargo improves a deleterious process or phenotype in the cells or tissue.

Finally, animals models of a disease that the complex is intended to treat can be used.

Suitable assays and models are selected based on the cargo portion used in the particular complex and the intended use of the complex (e.g., diagnostic, therapeutic, research reagent, etc.).

(ix) Packages and Kits

The disclosure provides a variety of kits (or pharmaceutical packages) for conveniently and/or effectively carrying out methods of the present disclosure. Typically kits will comprise sufficient amounts and/or numbers of components to allow a user to perform multiple treatments of a subject(s) and/or to perform multiple experiments for desired uses (e.g., laboratory or diagnostic uses). Alternatively, a kit may be designed and intended for a single use. Components of a kit may be disposable or reusable.

In some embodiments, kits include one or more of (i) an FGF10-related Surf+ Penetrating Polypeptide as described herein and a cargo portion to be delivered; and (ii) instructions (or labels) for forming complexes comprising the FGF10 portion and the cargo portion. Optionally, such kits may further include instructions for using the complex in a research, diagnostic or therapeutic setting.

In some embodiments, a kit includes one or more of (i) an FGF10-related Surf+ Penetrating Polypeptide as described herein and a cargo portion to be delivered or a complex of such FGF10-related Surf+ Penetrating Polypeptide and such cargo portion; (ii) at least one pharmaceutically acceptable excipient; (iii) a syringe, needle, applicator, etc. for administration of a pharmaceutical, prophylactic, diagnostic, or imaging composition to a subject; and (iv) instructions and/or a label for preparing the pharmaceutical composition and/or for administration of the composition to the subject.

In some embodiments, a kit includes one or more of (i) a pharmaceutical composition comprising a complex of the disclosure; (ii) a syringe, needle, applicator, etc. for administration of the composition to a subject; and (iii) instructions and/or a label for administration of the composition to the subject. Optionally, the kit need not include the syringe, needle, or applicator, but instead provides the composition in a vial, tube or other container suitable for long or short term storage until use.

In some embodiments, a kit comprises two or more containers.

In some embodiments, a kit includes a number of unit dosages of a composition comprising a complex of the disclosure. In some embodiments, the unit dosage form is suitable for intravenous, intramuscular, intranasal, intraperitoneal, intratumoral, oral, topical or subcutaneous delivery. Thus, the disclosure herein encompasses solutions, preferably sterile solutions, suitable for each delivery route. A memory aid may be provided, for example in the form of numbers, letters, and/or other markings and/or with a calendar insert, designating the days/times in the treatment schedule in which dosages can be administered. Placebo dosages, and/or calcium dietary supplements, either in a form similar to or distinct from the dosages of the pharmaceutical compositions, may be included to provide a kit in which a dosage is taken every day.

In some embodiments, the kit may further include a device suitable for administering the composition according to a specific route of administration or for practicing a screening assay.

Kits may include one or more vessels or containers so that certain of the individual components or reagents may be separately housed. Exemplary containers include, but are not limited to, vials, bottles, pre-filled syringes, IV bags, blister packs (comprising one or more pills). A kit may include a means for enclosing individual containers in relatively close confinement for commercial sale (e.g., a plastic box in which instructions, packaging materials such as styrofoam, etc., may be enclosed). Kit contents can be packaged for convenient use in a laboratory.

In the case of kits sold for laboratory and/or diagnostic use, the kit may optionally contain a notice indicating appropriate use, safety considerations, and any limitations on use. Moreover, in the case of kits sold for laboratory and/or diagnostic use, the kit may optionally comprise one or more other reagents, such as positive or negative control reagents, useful for the particular diagnostic or laboratory use.

In the case of kits sold for therapeutic and/or diagnostic use, a kit may also contain a notice in the form prescribed by a governmental agency regulating the manufacture, use or sale of pharmaceuticals or biological products, which notice reflects (a) approval by the agency of manufacture, use or sale for human administration, (b) directions for use, or both.

In certain embodiments, the kits are designed for patient use at home. Such kits may optionally include more detailed instructions regarding use and/or storage that can be understood by lay-people using the product at home. In other embodiments, the kits are designed for use in an in- or out-patient medical setting, such as a doctor's office, clinic, or hospital.

In certain embodiments of any of the foregoing, a kit may include, in a separate container, an agent to promote endosomal escape (e.g., chloroquine, PFO, etc.). In other words, any of the foregoing kits for therapeutic, diagnostic, or research purposes may optionally include, in a separate container, an agent to promote endosomal escape. Thus, for example, the kit comprises separate containers for each of a complex of the disclosure and an agent to promote endosomal escape.

These and other aspects of the present disclosure will be further appreciated upon consideration of the following Examples, which are intended to illustrate certain particular embodiments of the disclosure but are not intended to limit its scope, as defined by the claims.

### EXAMPLES

### Example 1

### Production and Purification of a Tagged FGF10 Domain

A Hisx6-FGF10-myc protein was expressed and purified (where “FGF10” indicates the FGF10 portion). In this particular example, the FGF10 portion was a domain of human FGF10 with a net positive charge, surface positive charge, and charge/molecular weight ratio greater than that of full length, unprocessed, naturally occurring human FGF10. For this specific construct, the domain of FGF10 has an amino acid sequence corresponding to the amino acid sequence set forth in SEQ ID NO: 2.

Production and purification of this protein produced a yield of 66 mg/L (total yield of 350 mg) with greater than 95% purity.

The JExpress416 expression vector containing the coding sequence for Hisx6-FGF10-Myc was transformed into the BL21(DE3) strain of E. Coli cells. The cells were grown to a density of 1.7 (as measured by A₆₀₀) in ProGro media (Expression Technologies) containing 100 μg/mL ampicillin, induced with 0.5 mM IPTG and incubated at 22° C. with shaking at 350 rpm for 19 hours. Cells were harvested by centrifugation at 6,000×g for ten minutes.

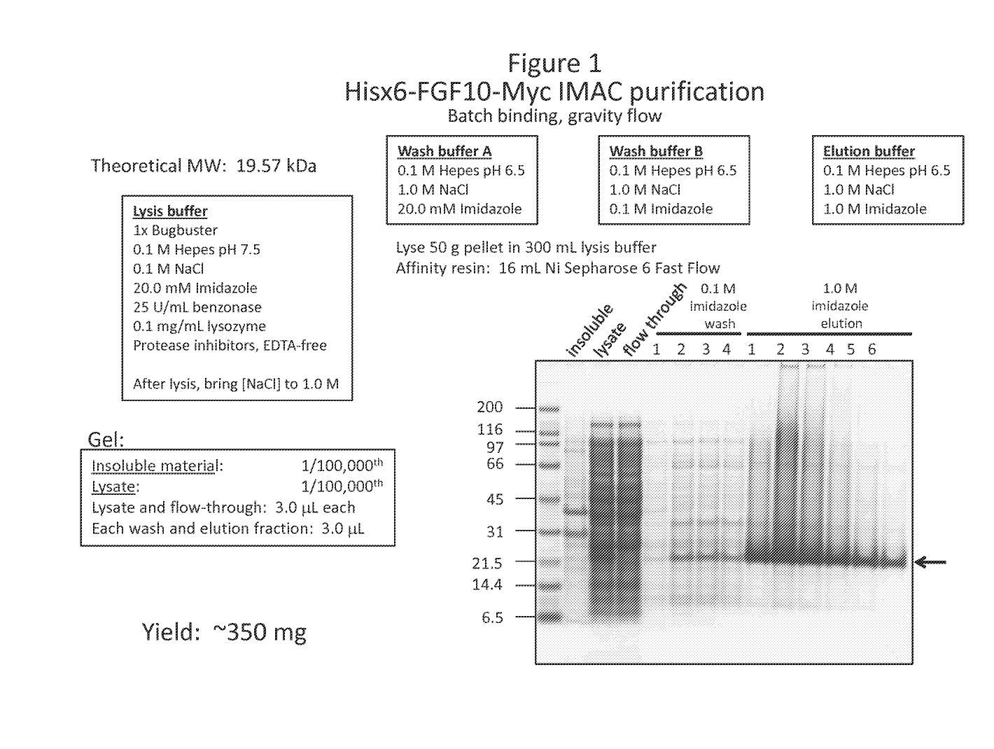

The resulting cell pellet was lysed in lysis buffer, the NaCl concentration was subsequently brought to 1.0 M (see FIG. 1), the lysate was clarified by centrifugation at 20,000×g for ten minutes, and the supernatant was applied to Ni sepharose 6 fast flow (GE Healthcare). The bound resin was washed with wash buffer A (see FIG. 1: Wash buffer A=0.1 M Hepes pH 6.5; 1.0 M NaCl; 20.0 mM imidazole), followed by wash buffer B (0.1 M Hepes pH 6.5; 1.0 M NaCl; 0.1 M imidazole), and eluted with elution buffer (0.1 M Hepes pH 6.5; 1.0 M NaCl; 1.0 M imidazole). Indicated aliquots of representative fractions were applied to 4-12% polyacrylamide gel and visualized with Instant Blue coomassie stain (Expedeon; FIG. 1).

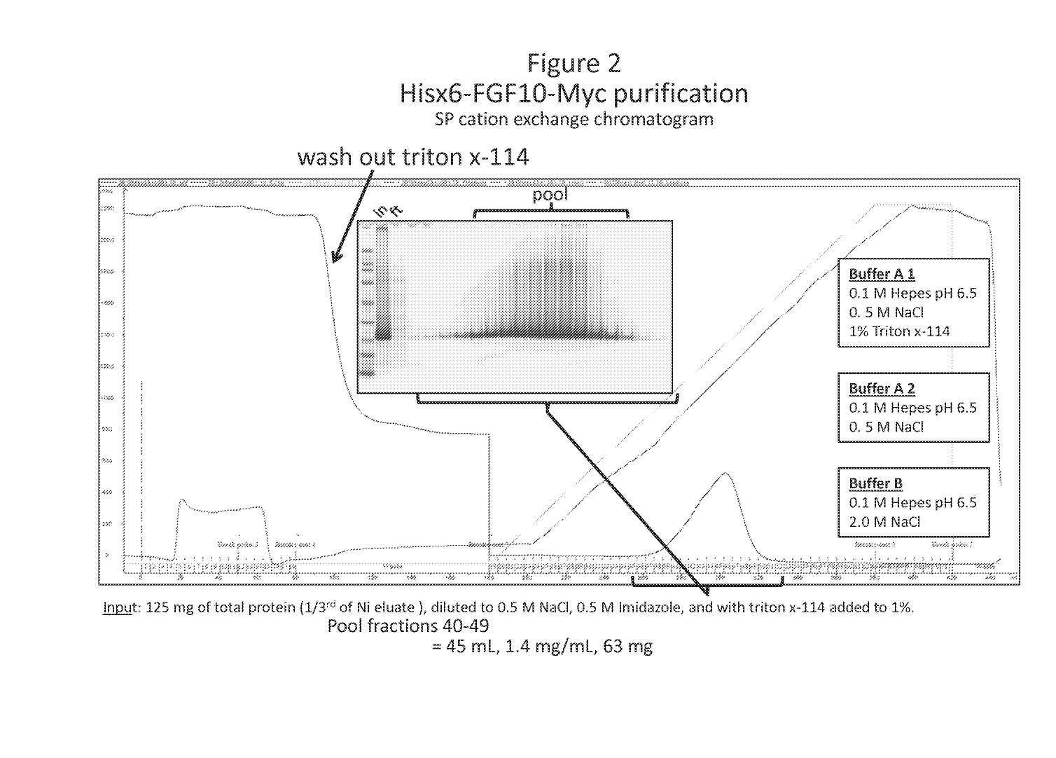

IMAC-purified material was then subjected to cation exchange chromatography. The concentration of NaCl was brought to 0.5M by dilution and triton x-114 was added to 1%. The protein was then applied to a HiPrep SP FF 16/10 column (GE Healthcare). The triton x-114 was washed away, and the protein was eluted with a gradient of NaCl from 0.5M to 2.0M over ten column volumes (FIG. 2).

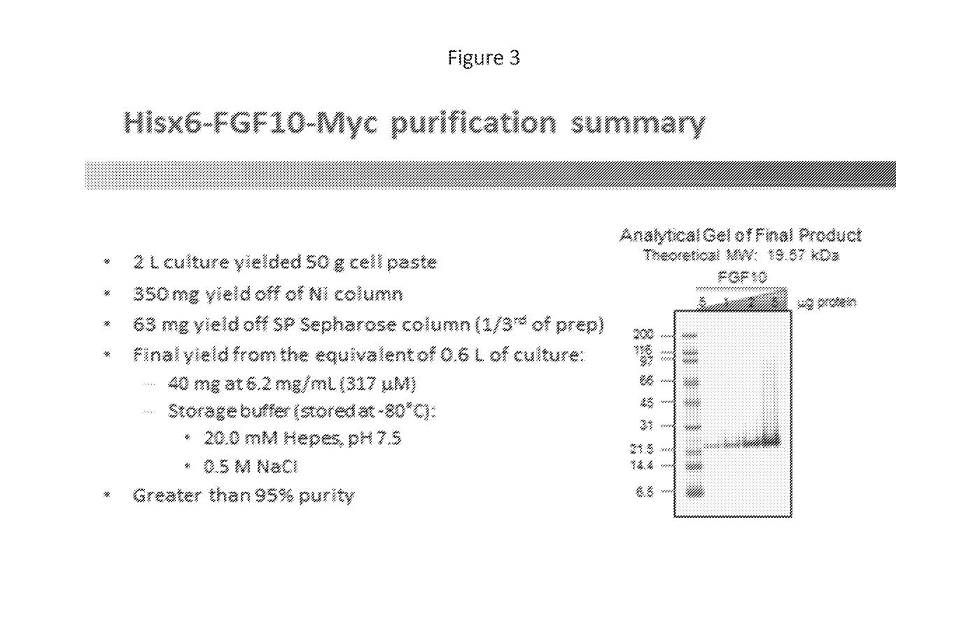

Indicated fractions from the cation exchange chromatography (FIG. 2) were pooled and dialyzed against the final storage buffer (20.0 mM Hepes, pH 7.5, 0.5 M NaCl). The protein was divided into small aliquots, snap-frozen in liquid nitrogen, and stored at −80° C. A summary of the purification is presented in FIG. 3.

### Example 2

### Production and Purification of a Complex—an FGF10 Portion Fused to a Cargo Portion

A complex comprising an FGF10 portion fused via a glycine-serine linker to a cargo portion was expressed and purified. The conjugate is also tagged on the N-terminus with a Hisx6 tag (SEQ ID NO: 28). The conjugate can be represented as: Hisx6-FGF10-GS10-TK (where “FGF10” represents the FGF10 portion and TK denotes the particular cargo portion used in this particular example). In this particular example, the FGF10 portion was a domain of human FGF10 with a net positive charge, surface positive charge, and a charge/molecular weight ratio greater than that of full length, unprocessed, naturally occurring human FGF10. The domain has an amino acid sequence corresponding to the amino acid sequence set forth in SEQ ID NO: 2. The cargo portion in this example is the enzyme thymidine kinase (TK), in this case HSV TK, specifically the TK SR39 mutant. The SR39 mutant of HSV TK has enhanced catalysis of the prodrug ganciclovir relative to the wild type HSV TK. See, Kokoris and Black, Characterization of Herpes Simples Virus type 1 thymidine kinase mutants engineered for improved ganciclovir or acyclovir activity. Protein Science (2002) 11:2267-2272. This property is advantageous for the HSV-TK assays used to examine these fusion proteins, described below.

In this example, the complex is a fusion protein and the FGF10 portion and the cargo portion are interconnected via a peptide linker. Here, the peptide linker connecting the FGF10 portion and the cargo portion was a ten amino acid linker, specifically (G₄S)₂ (SEQ ID NO: 29). In this particular example, the FGF10 portion is N-terminal to the cargo portion. However, in other embodiments, the FGF10 portion may be C-terminal to the cargo portion. Moreover, the linker sequence and/or length can be varied, and the fusion protein may or may not have a tag. The amino acid sequence of the cargo portion (the HSV TK protein described above) used in this particular example is set forth in SEQ ID NO: 3. The amino acid sequence for this particular FGF10-TK fusion protein (Hisx6-FGF10-GS10-TK) is set forth in SEQ ID NO: 4.

Production and purification of this conjugate yielded 24 mg/L of protein having greater than 95% purity.

The JExpress416 expression vector containing the coding sequence for Hisx6-FGF10-GS10-TK was transformed into the BL21(DE3) strain of E. Coli cells. The cells were grown to a density of 1.7 (as measured by A₆₀₀) in ProGro media (Expression Technologies) containing 50 g/mL kanamycin, induced with 0.5 mM IPTG and incubated at 22° C. with shaking at 350 rpm for 16 hours. Cells were harvested by centrifugation at 6,000×g for ten minutes.

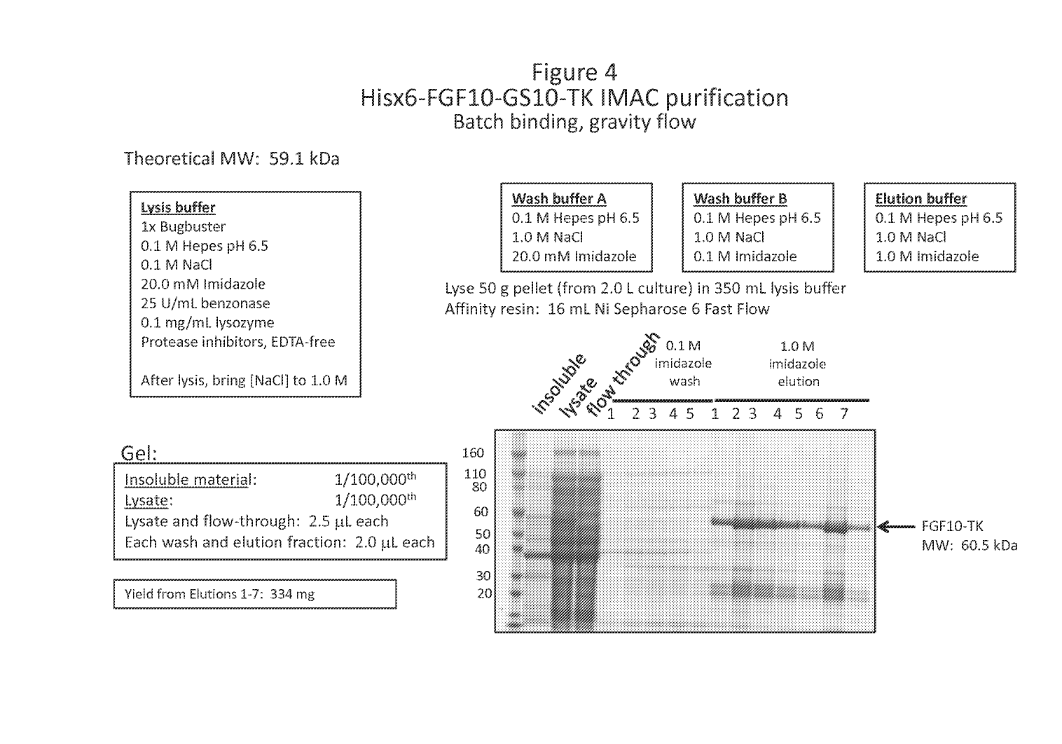

The resulting cell pellet was lysed in lysis buffer, the NaCl concentration was subsequently brought to 1.0 M (see FIG. 4), the lysate was clarified by centrifugation at 20,000×g for ten minutes, and the supernatant was applied to Ni sepharose 6 fast flow (GE Healthcare). The bound resin was washed with wash buffer A (see FIG. 4; buffer A=0.1M Hepes pH 6.5; 1.0 M NaCl; 20.0 mM imidazole), followed by wash buffer B (0.1 M Hepes pH 6.5; 1.0 M NaCl; 0.1 M imidazole), and eluted with elution buffer (0.1 M Hepes pH 6.5; 1.0 M NaCl; 1.0 M imidazole). Indicated aliquots of representative fractions were applied to 4-12% polyacrylamide gel and visualized with Instant Blue coomassie stain (Expedeon; FIG. 4).

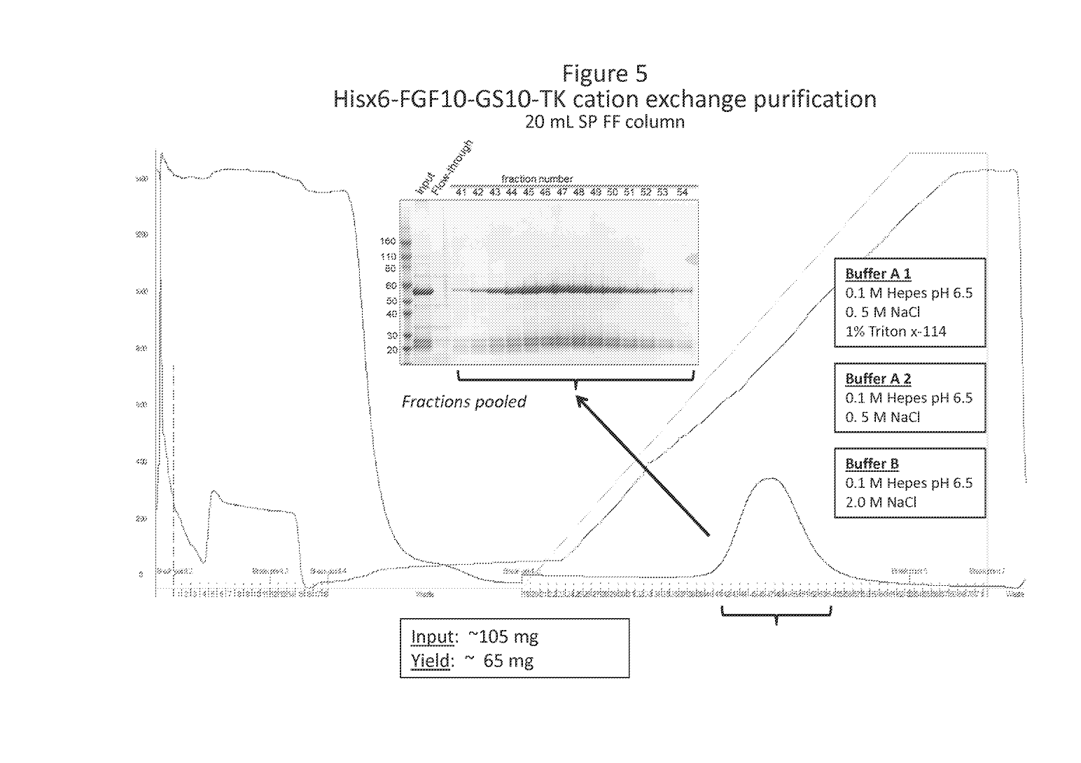

IMAC-purified material was then subjected to cation exchange chromatography. The concentration of NaCl was brought to 0.5M by dilution and triton x-114 was added to 1%. The protein was then applied to a HiPrep SP FF 16/10 column (GE Healthcare). The triton x-114 was washed away, and the protein was eluted with a gradient of NaCl from 0.5M to 2.0M over ten column volumes (FIG. 5).

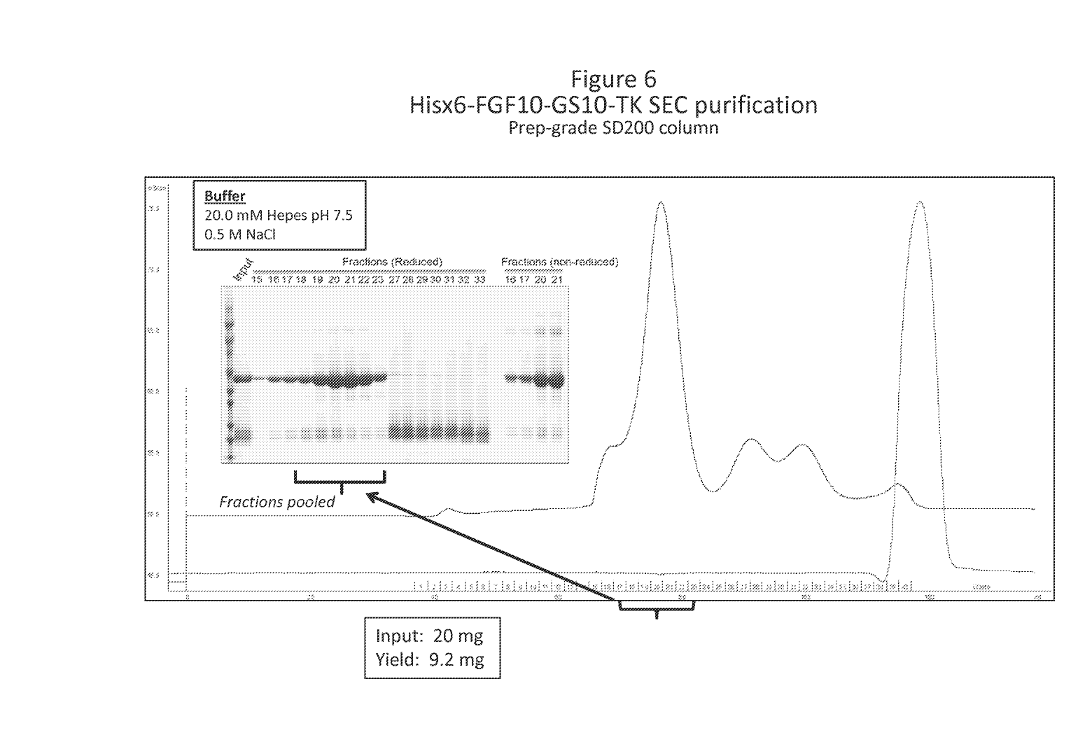

Indicated fractions from the cation exchange chromatography (FIG. 5) were pooled and subjected to SEC chromatography. The protein was concentrated using Amicon ultracentrifugation concentrators (Millipore) and applied to a sephadex 200 16/60 SEC column (GE Healthcare) in 20 mM Hepes, pH 7.5 and 0.5 M NaCl (FIG. 6). The indicated fractions were pooled.

The pooled fractions were concentrated as above, divided into small aliquots, snap-frozen in liquid nitrogen, and stored at −80° C. A summary of the purification is as follows:
25 g cell paste was produced per liter of culture
The Ni column yielded 167 mg protein from 1 L culture
Subsequently, the SP cation exchange column yielded 95 mg protein from the equivalent of 1 L culture
Finally, the SEC column yielded 38 mg protein from the equivalent of 1 L culture
The protein was divided into small aliquots, snap-frozen in liquid nitrogen, and stored at −80° C. in 20 mM Hepes, pH 7.5, 0.5 M NaCl.
The final protein was greater than 95% pure

### Example 3

### Stability of Hisx6-FGF10-Myc Following Multiple Freeze/Thaw Cycles

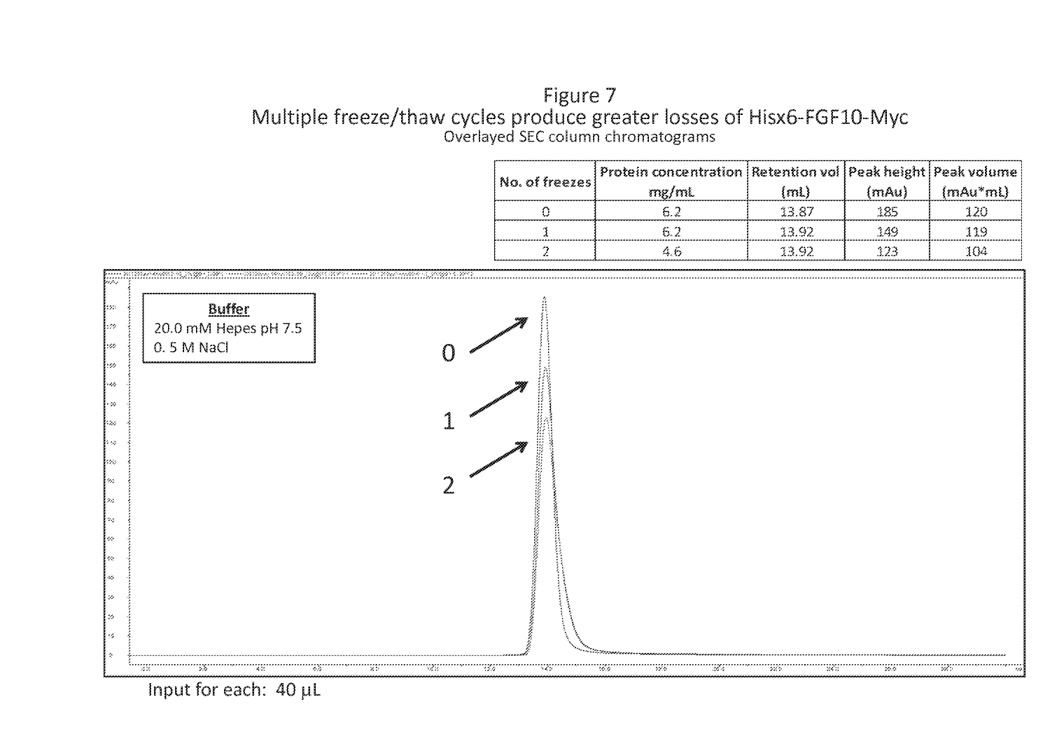

The stability of this cell penetrating domain of FGF10 was evaluated following multiple freeze/thaw cycles. To do this, protein concentration measurements and analytical size exclusion chromatography were carried out comparing protein which had undergone zero, one, or two freeze/thaw cycles. The results depicted in FIG. 7 demonstrate that this particular protein construct, described in detail in Example 1, has sufficient stability for further experimental evaluation. The study indicated about 75% stability of this protein after two freeze/thaw cycles.

Purified protein was subjected to the indicated number of freeze/thaw cycles (each freeze/thaw cycle consisted of snap-freezing the protein in liquid nitrogen, placing it at −80° C. for a minimum of two hours, and then thawing it on ice) and analyzed by size exclusion chromatography. After each thaw, the protein was subjected to 20,000×g centrifugation to remove any precipitation. The protein concentration of each sample was measured by A₂₈₀, considering the extinction coefficient (24540) and molecular weight (19.57 kDa). In each case 40 μL of the protein was applied to a Superdex 75 10/300 GL column (GE Healthcare). The column running buffer was the same as the protein storage buffer (20 mM Hepes, pH 7.5, 0.5 M NaCl). The resulting chromatograms are shown in FIG. 7. The measured protein concentrations, SEC retention volume, peak height, and peak volume are also indicated in FIG. 7.

### Example 4

### Cell Penetrating Activity

A domain of FGF10 having surface positive charge, net positive charge, and a charge/molecular weight ratio greater than that of the full length, unprocessed, naturally occurring FGF10 protein functions as a Surf+ Penetrating Polypeptide and effectively penetrates cells. The protein used in these experiments was the same as that detailed in Example 1.

On the day prior to the assay, 10⁶ Hela cells were plated in each well of a 6-well plate and incubated in the 37° C. CO₂ incubator overnight. The cells were washed once with PBS and then were replenished with 1 mL of serum-free DEMEM (A) or media containing 1 uM of the FGF10 domain construct FGF10-myc (B, C, and D) and were incubated for 20 minutes in the 37° C. CO₂ incubator. The cells were then washed three times with ice-cold PBS. For (C) and (D), cells were treated with 0.25% trypsin/EDTA and washed three times with PBS. The cells in (D) were fixed for 10 minutes with 1 mL 4% formaldehyde and permealized for 5 minutes with 0.2% saponin. Then 10 uL of 0.1 mg/mL FITC labeled chicken polyclonal anti-myc tag antibody (Abcam #1394) was added to all the cells and incubated at 4° C. in the dark for 2 hours. The cells were washed three times with ice cold PBS. Cells in (A) and (B) were detached with 1 mL 10 mM EDTA. Cells were analyzed using a BD Accuri™ C6 flow cytometer.

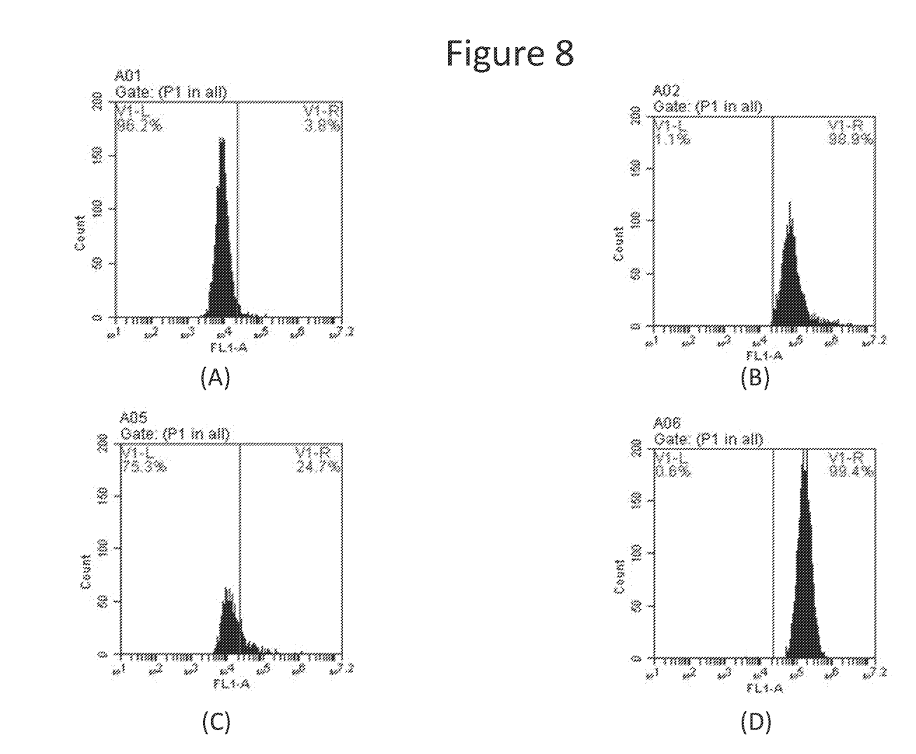

The results of these experiments are depicted in FIG. 8. 98.8% of cells treated with this FGF10 domain had protein present on the cell membrane (B) compared to untreated cells (A). The majority of cell membrane-bound FGF10 can be removed by trypsin (C). However, almost all the cells stained positive for FGF10 after typsin treatment, indicating that protein was internalized into the cells via endocytosis (D). These results confirm that this domain of FGF10 has both the structural and functional characteristics of a Surf+ Penetrating Polypeptide, and is an example of Intraphilin™ Technology. In other words, the domain of FGF10 penetrates cells.

### Example 5

### The Activity of the Cargo Portion was Maintained when Complexed with FGF10

Experiments were conducted to confirm that the activity of the cargo portion was maintained following fusion to the FGF10 portion. In this experiment, the enzymatic activity of TK was evaluated in the context of the complex Hisx6-FGF10-GS10-TK (the same complex described above in Example 2). As summarized in the table below, these studies demonstrated that the enzymatic activity of TK was maintained and is similar to that observed in other TK fusion proteins (e.g., fusions with moieties that are not cell penetrating; fusions with TAT).

The ADP Quest kinase assay kit (Discover Rx) was used to determine Km and relative Vmax values of the TK fusion proteins of interest. In general, the manufacturer's instructions were carried out, using the following parameters:
0.5 mM ATP
20.0 nM of each indicated TK enzyme
Thymidine titration starting at 50 μM, followed by a series of 1:3 dilutions for a total of five points
The reaction was carried out in black, clear-bottom 96-well plates and read in a Synergy 2 plate reader (Biotek)
Kinetic values were calculated from Eadie-Hofstee plots.
   The results indicated that the FGF10-linker-TK fusion protein exhibited activity similar to TK alone or other TK fusion proteins (see table below). As described above, HSV-TK SR39 mutant (TK-SR39) is the TK protein used in these constructs. The reported Km value for TK-SR39 is 2.64 uM as reported in the literature (Kokoris and Black, Characterization of Herpes Simples Virus type 1 thymidine kinase mutants engineered for improved ganciclovir or acyclovir activity. Protein Science (2002) 11:2267-2272), and a value that is comparable to the values reported in Table 1 below. The differences in Km values and Vmax values of Table 1 are not significant relative to the expected assay variability. Accordingly, the enzymatic activity of TK is not compromised as a result of fusion an FGF10 portion.

TABLE
Kinetic values of indicated TK-fusion proteins. FGF-TK exhibits
similar activity as other fusion proteins tested.

| | Km | |
|---|---|---|
| Protein | (μM) | Vmax |
| Hisx6-FGF10- | 2.1 | 240 |
| linker-TK | | |
| Hisx6-TK (expt 1) | 6.0 | 300 |
| Hisx6-TK (expt 2) | 6.0 | 194 |
| Hisx6-TK (expt 3) | 2.5 | 294 |
| Hisx6-TAT-TK | 3.8 | 269 |
| Hisx6-⁺36GFP-TK | 6.2 | 257 |

The gly/ser linkers used for the TAT-TK and +36GFP-TK fusion proteins (G₂S(G₄S)₂) (SEQ ID NO: 30) were nearly identical to that used for the FGF10-TK fusion protein—differing by only three amino acid residues.

### Example 6

### FGF10-Cargo Complexes Internalize into Cells and Retain the Functional Activity of the Cargo

In these experiments, the ability of a complex comprising an FGF10 portion and a cargo portion were evaluated to determine whether the conjugate retained the cell penetrating activity of the FGF10 domain and the functional activity of the cargo. The Hisx6-FGF10-GS10-TK complex (the same fusion protein described above in Example 2) was used in these experiments. Importantly, a cell-based, functional assay was used in these studies to evaluate both the cell penetrating activity of the FGF10 portion and the enzymatic, functional activity of the cargo portion (TK).

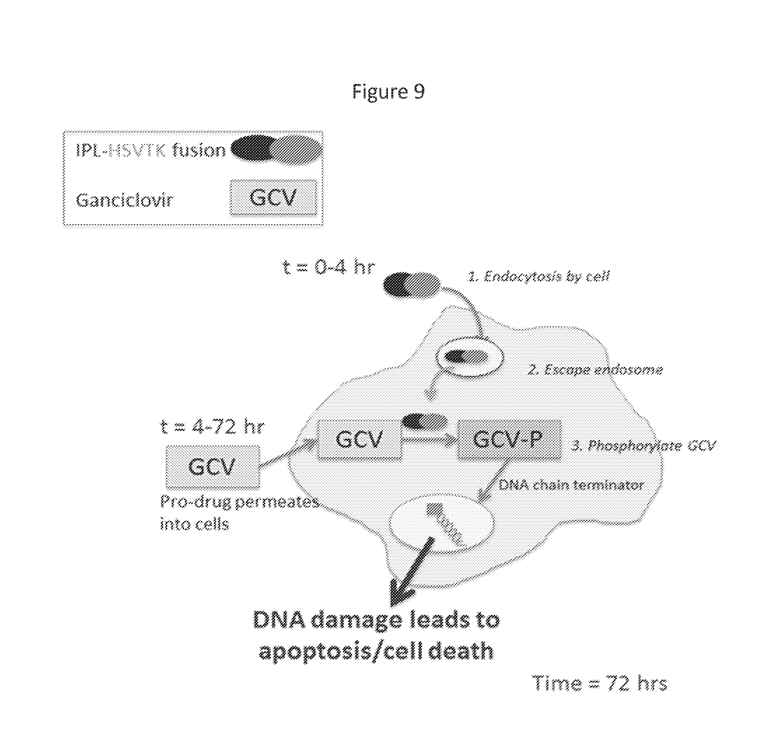

Assay Overview: To measure functional activity upon internalization, the herpes simplex virus “thymidine kinase” enzyme (HSV-TK)-ganciclovir system was applied. TK is a phosphotransferase that normally catalyzes the formation of 2′-deoxythymidine that is required for vital cellular DNA replication. This can be exploited by the addition of a prodrug specific for viral TK (ganciclovir), which after undergoing phosphorylation by TK, competitively inhibits guanosine incorporation into DNA and leads to cellular apoptosis (See FIG. 9). In our cell-based assay, a Surf+ Penetrating Polypeptide (in this case a domain of human FGF10 having a charge/molecular weight ratio greater than that of the naturally occurring, unprocessed, full length human FGF10) genetically fused to TK enzyme was added to live cells where it underwent endocytic uptake by various mechanisms over the course of 4 hours. TK that escapes from the endosome, assisted by chloroquine, then enters the cytosol. Upon addition of ganciclovir, TK catalyzes the phosphorylation of the prodrug and the death of the cells over the course of 72 hours. The degree of cell death is assessed by a cell proliferation dye-based assay—MTT (3-(4,5-Dimethylthiazol-2-yl)-2,5-diphenyltetrazolium bromide). The absorbance of metabolized MTT dye of live cells is measured by visible absorbance. Accordingly, in this assay, cell death following administration of the fusion protein and addition of ganciclovir indicates both than the fusion protein successfully penetrate the cells and was present in the cytosol, and indicates that the enzyme was functionally active.

The purpose of this experiment was to determine whether the cargo enzyme TK is functionally active following intracellular delivery mediated by the FGF10 portion. In addition, the experiment indicated enhancement of cytosolic access by chloroquine co-administration.

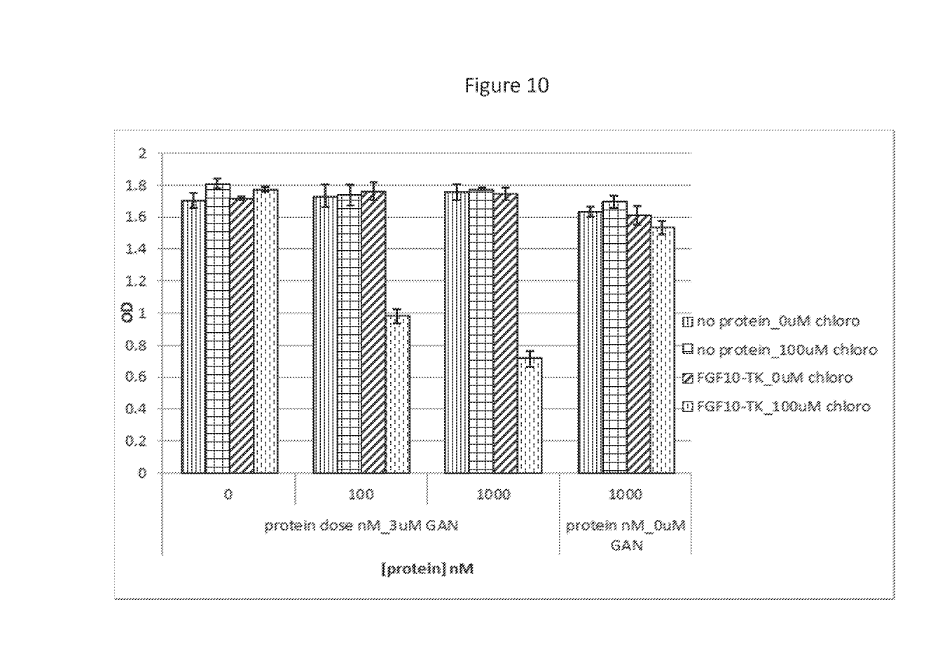

These experiments indicated that FGF10-TK is functionally active following internalization. At 1 μM, TK induced 60% cell death of 4T1 cells in the presence of chloroquine. At 100 nM, FGF10-TK induced 45% cell death in the presence of chloroquine. These data demonstrate that an FGF10 portion can be used to deliver a cargo portion, even a non-native functional enzyme, into cells, and that the enzyme is functionally active in the cytosol. FIG. 10 summarizes the data for FGF10-TK-induced cell death in the HSV-TK MTT assay. OD values measure the amount of MTT dye metabolized by live cells, and thus is a measure of the degree of cell death in the well. Results from experiments in which additional conjugates were evaluated are provided in the table below.

% cell death

| | protein dose | | | protein |
|---|---|---|---|---|
| | (nM) 3 μM | | | (nM) No |
| | GAN | | | GAN |
| | 0 | 100 | 1000 | 1000 |
| no protein 0 μM chloro | 0 | −1.80 | −3.05 | 4.01 |
| no protein 100 μM | 0 | 3.83 | 2.03 | 6.07 |
| chloro | | | | |
| FGF10-TK 0 μM chloro | 0 | −2.99 | −1.64 | 6.21 |
| FGF10-TK 100 μM | 0 | 44.54 | 59.82 | 13.53 |
| chloro | | | | |
| no protein 0 μM chloro | 0 | 17.14 | 0.12 | 1.63 |
| no protein 100 μM | 0 | 4.77 | 2.30 | 16.06 |
| chloro | | | | |
| +36GFP-TK 0 μM | 0 | −1.40 | 59.51 | 10.11 |
| chloro | | | | |
| +36GFP-TK 100 μM | 0 | 61.61 | 83.92 | 17.14 |
| chloro | | | | |
| no protein 0 μM chloro | 0 | −0.30 | 2.67 | 2.86 |
| no protein 100 μM | 0 | −6.33 | −6.39 | −1.11 |
| chloro | | | | |
| TAT-TK 0 μM chloro | 0 | 2.20 | 15.12 | 12.35 |
| TAT-TK 100 μM chloro | 0 | 5.18 | 36.85 | 4.74 |

### Materials and Methods:

Reagents: DPBS (Gibco Cat#14190-144), RPMI 1640 media (Gibco Cat#11875-093), Fetal Bovine Serum (FBS Gibco Cat#1600-044), Pen/strep (Gibco cat#15140-122), Promega Non-radioactive Cell Proliferation Assay (Cat#G4001), Chloroquine diphosphate (Sigma #C6628), Heparin (Sigma-#H3149 200 U/mg), ganciclovir (Invivogen-#sud-gcv), purified intraphilin proteins
Other Materials: mouse mammary 4T1 Cells (ATCC #CRL-2539), BD-Falcon 96-well
Black/Clear bottom tissue culture-treated plates (#353219)

### Protocol:

1) 4T1 cells were plated on 96-well plates at a density of 3,500 cells per (in 100 μL) well 1 day prior to addition of test fusion proteins (FGF10-TK). The cells were incubated overnight in a 37° C. incubator at 95% humidity and 5% CO₂.
2) The following day, media was removed from the wells and 90 μL of serum-free media (RPMI) containing (or not) 100 μM chloroquine was added to the wells. The cells were allowed to pre-incubate with chloroquine for 30 minutes at 37° C.
3) After the 30 minutes, 10 μL of a solution containing 10× concentrated FGF10-TK was added to the relevant wells (to give final concentrations of 0, 0.1 and 1 μM FGF10-TK). The cells were allowed to internalize the proteins for 4 hours at 37° C.
4) Cells were then washed 3 times with 0.1 mg/mL heparin in cold D-PBS on ice.
5) Following the washing step, 150 μL of full RPMI media (containing 10% FBS, pen/strep) with or without 3 μM ganciclovir was added to the wells and incubated at 37° C. for 72 hours.
6) 15 μL of MTT dye solution (Promega) was added to each well and the dye allowed to internalize/metabolize for 4 hours at 37° C.
7) 100 μL of Stop Solution (Promega) was added to each well and the cells allowed to lyse for 16 hours.
8) Plates were then read on a BioTek Synergy plate reader using absorbances of 570 nm and 650 nm. The corrected OD values was then normalized and converted to percent cell death.

### Example 7

### Evaluating Pharmacokinetics in Mice

A number of proteins with cell penetrating activity were evaluated in mice to assess pharmacokinetics. The primary objective was to confirm that a cell penetrating FGF10 portion, alone and complexed with a cargo portion, circulated beyond the injection site in vivo. The secondary objective was to compare the in vivo behavior of these cell penetrating FGF10 constructs to other proteins, including the Surf+ Penetrating Polypeptide +36GFP, and Tat. Additional studies will evaluate how rapidly these proteins are cleared from the blood compartment relative to each other.

FGF10 and FGF10-TK, as described in Examples 1 and 2, respectively, where the FGF10 portion is an example of a Surf+ Penetrating Polypeptide) along with control test articles Tat-TK, +36GFP-TK and +36GFP (where the +36GFP portion is also an example of a Surf+ Penetrating Polypeptide) were produced as previously described with sufficiently low endotoxin levels and at sufficiently high concentrations to enable dosing mice. Low endotoxin levels in the final protein were achieved by performing the cation exchange purification at 4° C., with protein loaded to the column in the presence of 1% Triton X-114. The column was washed with a buffer containing no Triton X-114 until the UV absorbance baseline stabilized and the protein was eluted in the absence of detergent. The proteins were radiolabeled by the Iodogen method that attaches iodine to tyrosine amino acid residues. All of these proteins are known to have at least 9 tyrosine residues. Radioactive isotope ¹²⁵I was used for this study such that protein detection could occur using the radioactive emissions of the isotope. Radiolabeling was performed by Perkin Elmer.

The test articles were prepared for dosing animals by thawing [125I]-Labeled proteins on ice and centrifuging at 20,000×g for 10 minutes at 4° C. The target dose level for each protein was 10 mg/kg. The animals dosed were CD-1 male mice with body weights ranging from 20-30 grams at dosing. Test articles were administered to each mouse by an IV bolus dose via tail vein injection. At a set time after dosing, animals were deeply anesthetized via isoflurane inhalation, a blood sample (≈1 mL) was obtained via cardiac puncture from all animals. The blood tubes contain K3EDTA as an anticoagulant. Blood was maintained for <1 h of the blood collection time, on wet ice, until centrifuged at approximately 4° C. to obtain plasma. The plasma was quick frozen on dry ice and stored at approximately −20° C. until gamma counting and Trichloroacetic Acid (TCA) precipitation analysis. Plasma analysis was performed by gamma counting to determine the concentration of radioactivity.

Duplicate aliquots of plasma (≈50 μL) were incubated in TCA, centrifuged and the resulting supernatants and pellets analyzed by gamma counting to determine the % of precipitable radioactivity in each sample. The TCA provides an estimate of the stability of the radiolabel for each test article at each time point.

Data for plasma concentration is converted into ug equivalent of protein per ml of blood plasma. The TCA precipitable radioactivity is reported as a percentage. The multiplication of the TCA precipitable percentage by the plasma concentration gives the ug protein per ml of blood expected to be attributable to radioactivity associated with radiolabeled proteins.

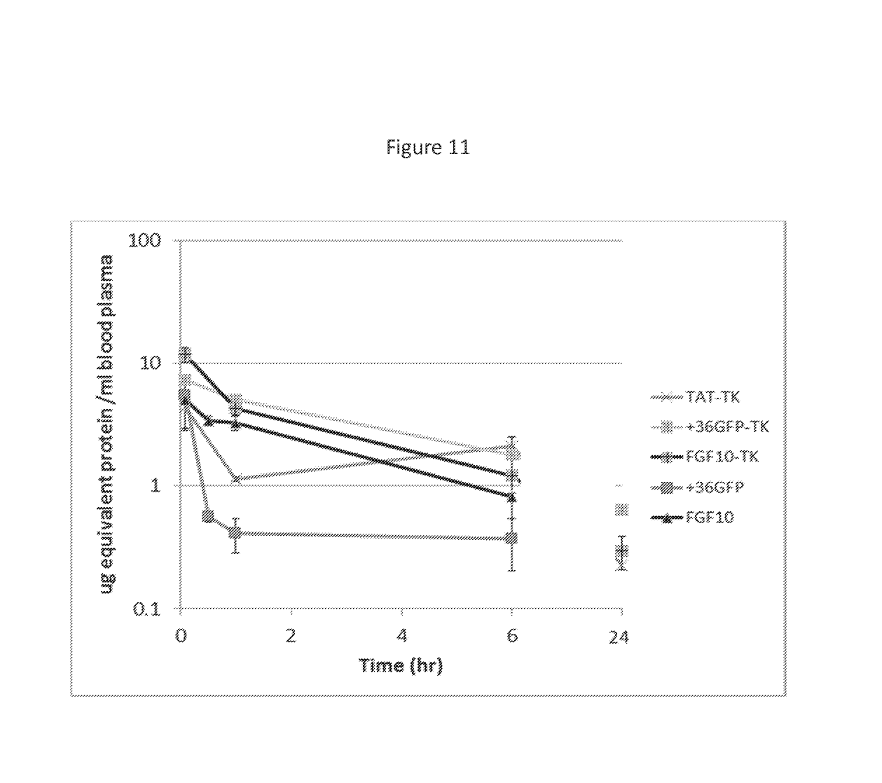

The results of these experiments are summarized in FIGS. 11-13. FIG. 11 provides a graph depicting the ug of 125I-protein per ml of blood plasma where blood samples were collected from mice at 5 minutes, 30 minutes, 1 hour, and 6 hours for +36GFP and FGF10 and then at 5 minutes, 1 hour, 6 hours and 24 hours for Tat-TK, +36GFP-TK, and FGF10-TK. Concentration was determined by a measurement of TCA precipitable radioactivity. Error bars represent standard deviation of data from 2 mice where data was available.

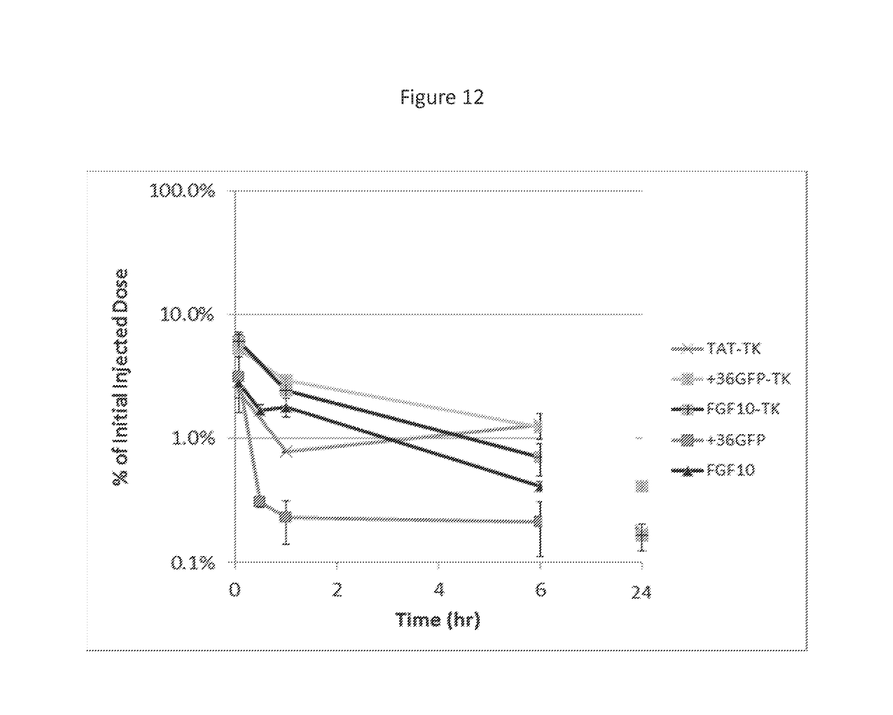

FIG. 12 provides a graph depicting percent of initial dose present in the blood plasma where blood samples were collected from mice at 5 minutes, 30 minutes, 1 hour, and 6 hours for +36GFP and FGF10 and then at 5 minutes, 1 hour, 6 hours and 24 hours for Tat-TK, +36GFP-TK, and FGF10-TK. This protein concentration data was adjusted by TCA precipitable counts. The initial dose concentration was determined by taking the initial dose given to the animal as determined by counting radioactivity in an aliquot of the formulated dose and then assuming this dose is distributed uniformly in the blood compartment of a mouse, estimated at 1.7 ml. Error bars represent the standard deviation of data from 2 mice where data was available.

These experiments demonstrated that Surf+ Penetrating Polypeptides, including FGF10 domains and +36GFP, alone or as a complex with cargo, can be administered systemically without being trapped at the injection site.

Following IV administration (via tail vein), which allows the total dose to be available in the circulation, the maximum concentration in the serum (Cmax) was rapidly observed at 5 minutes. However, in contrast to typical biologics including monoclonal antibodies administered IV, whose Cmax approximates the injected dose, both FGF10 and FGF10-TK in the study achieved blood plasma protein concentration levels approximating 3.5% or 12% of injected dose, respectively at 5 minutes post-injection. These data suggest a larger volume of distribution for Surf+ Penetrating Polypeptides compared to typical biologics, such as monoclonal antibodies, which normally show poor overall distribution outside serum.

6 hours post-injection, FGF10 and FGF10-TK appear to drop to roughly 2.2% and 1.2%, respectively, of injected dose. These results indicate that the FGF10 portion containing proteins (FGF10 and FGF10-TK) may be present at higher concentrations in plasma 1 hour post-injection than Tat-TK or +36GFP. However, factors affecting plasma concentrations at these later time points include not only volume of distribution, but also rate of cell internalization, size of the test article, renal clearance and other mechanisms of elimination.

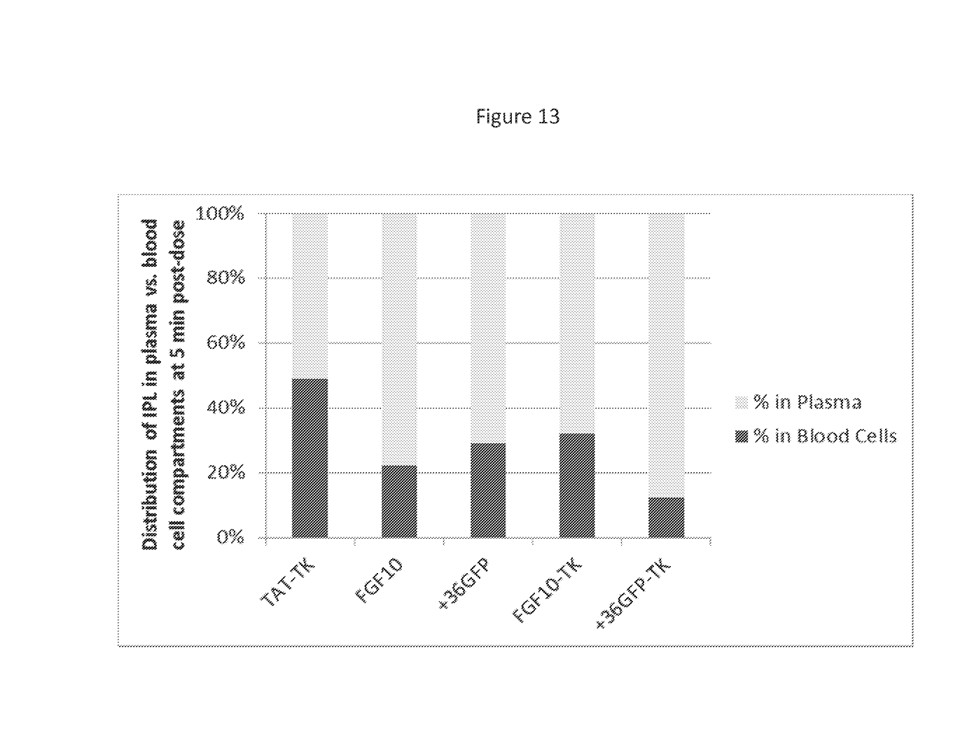

The partitioning of FGF10 and FGF10TK in the blood to either the blood cell compartment or the plasma compartment, in part, indicates if decrease in plasma concentration is due to blood cell uptake or due to other reasons such as tissue uptake. FGF10, FGF10TK, +36GFP and +36GFP-TK favor the plasma compartment over the blood cell compartment at 5 minutes post-injection as shown in FIG. 13. Tat-TK, on the other hand, shows equal partitioning to the blood cell and plasma compartments at 5 minutes post-injection. The partitioning calculation is based on the protein concentrations in plasma from the PK data described above with no adjustment for TCA precipitation and then the whole blood protein concentrations determined from Quantitative Whole Body Autoradiography studies described in the next example where N=1 animal. It is recognized that this is not the preferred method in determining partitioning, but with available data, this provides an indication of partitioning. The whole blood composition is assumed to be 55% plasma and 45% blood cells. With this partitioning, more FGF10 and FGF10TK is available to bind in tissues than Tat-TK since less FGF10 or FGF10TK is being taken up by blood cells on a percent injected dose basis.

### Example 8

### Biodistribution Study

A number of proteins with cell penetrating activity were evaluated in mice to assess ability to significantly travel beyond the injection site, and to evaluate tissue distribution. In addition, rate of uptake into tissues was evaluated and compared to other proteins, include a Tat-TK control.

FGF10 and FGF10-TK along with control test articles Tat-TK, +36GFP-TK and +36GFP were produced as previously described with sufficiently low endotoxin levels and at sufficiently high concentrations to enable dosing mice. The proteins were radiolabeled by the Iodogen method that attaches iodine to tyrosine amino acid residues. All proteins are known to have at least 9 tyrosine residues. Radioactive isotope ¹²⁵I was used for this study such that protein detection could occur using the radioactive emissions of the isotope. Radiolabeling was performed by Perkin Elmer.

The test articles were prepared for dosing animals by thawing [125I]-Labeled proteins on ice and centrifuging at 20,000×g for 10 min at 4° C. The target dose level for each protein was 10 mg/kg. The animals dosed were CD-1 male mice with body weights ranging from 20-30 grams at dosing. Test articles were administered to each mouse by an IV bolus dose via tail vein injection. At a set time after dosing, animals were deeply anesthetized via isoflurane inhalation. After a blood sample was obtained via cardiac puncture from all animals, the deeply anesthetized animals were euthanized by freezing in a hexane/solid carbon dioxide bath for at least 15 minutes for quantitative whole body autoradiography (QWBA) analysis. Each carcass was drained, blotted dry, and placed into a labeled bag along with the animal's cage card and stored at approximately 20° C. at least overnight prior to embedding. The pinna, distal limbs, and hair of each frozen carcass were removed and the remaining carcass along with the dosing site (tail) were embedded in refrigerated approximately 2% (w/v) aqueous carboxymethylcellulose and frozen into a block. The blocks were stored at approximately −20° C. prior to sectioning. In addition, blood smears were applied to slides for use in determining protein associated with peripheral blood mononuclear cells (PBMCs). These smears were analyzed using phosphoimaging as described below.

A number of sections (approximately 40 μm thick) were taken in the sagittal plane using a Leica CM3600 cryomicrotome or Vibratome 9800 cryostat set at −20° C. All of the major tissues, including the tail with injection site, organs, and biological fluids were represented. The sections were collected on adhesive tape and dehydrated prior to removal for mounting and exposure.

A set of representative sections for each mouse were mounted on thin cardboard supports. The mounted sections were then wrapped with plastic wrap and exposed to phosphorimaging screens. [125I]-spiked blood calibration standards at 3-4 different concentrations were co-exposed with all sections and were used to calibrate the image analysis software.

The exposed screens were scanned using a Molecular Dynamics Typhoon 9410 Phosphor Imager and data was acquired as Molecular Dynamics Counts/area² (MDC/mm²). The autoradiographic standard image data (calibration standards) were sampled using MCID software (Interfocus Imaging, Inc.) to create a calibrated standard curve. Specified tissues, organs, and fluids were analyzed and the tissue concentrations were interpolated from each standard curve as microcuries per gram (μCi/g). Tissue concentration data has been determined for the following tissues and/or contents whenever possible: adipose (brown and white), adrenal gland, bile (in gall bladder), blood, bone, bone marrow, brain (cerebrum, cerebellum, medulla), cecum (and contents), colon (and contents), dorsal root ganglion, epididymis, esophagus, eye (uvea and lens), Harderian gland, heart, injection site (tail), kidney cortex and medulla, liver, lung, lymph node, pancreas, pituitary gland, prostate gland, salivary gland, sciatic nerve, seminal vesicles, skeletal muscle, skin (pigmented and non-pigmented), small intestine (and contents), stomach (gastric mucosa and contents), spleen, spinal cord, testis, thymus, thyroid, and urinary bladder (and contents).

Tissue values that fall below the lowest standard on the calibration curve or that cannot be visualized on the autoradioluminograph were identified as below the quantification limit (BQL). If a tissue cannot be identified on a given section then these were identified as not identified (NI). The concentrations were converted to microgram equivalents of the test article per gram of tissue based on the specific activity of the test article in the dosing formulation; a quantitation limit was employed for these data. Data above the upper limit of quantitation (ULOQ) are marked (*). To bring data above the ULOQ into the range of calibration curve, the sections were exposed for a shorter time period and to confirm absolute numbers from the autoradioluminograph a subset of tissues from certain animal were removed from frozen blocks and directly counted in a gamma counter. In addition, a percent of injected dose of equivalent protein per gram of tissue was calculated by dividing microgram equivalent concentrations by the amount of protein in micrograms that was dosed on a by animal basis.

Based on these results, the constructs containing the cell penetrating FGF10 portion (FGF10 and FGF10-TK) were able to travel significantly beyond the area of the injection site. Moreover, the distribution of these proteins to tissue was not ubiquitous. Rather, these FGF10 portion containing proteins preferentially, although not exclusively, localized to certain tissues. Shortly after dosing with either FGF10 or FGF10-TK, high levels of the injected protein were detected in the following tissues, and these levels persisted for at least one or more hours: liver, spleen, kidney (cortex and medulla), urinary bladder, adrenal gland, and thyroid gland.

In addition, the uptake of the FGF10 portion constructs was higher in certain tissues than the protein uptake observed following injection of Tat-TK. Although Tat has cell penetrating activity, it is not a Surf+ Penetrating Polypeptide, and is also not a human protein. Uptake of FGF10 portion constructs was higher, relative to uptake of Tat-TK, in numerous tissues including: spleen, adrenal gland, pituitary gland, thyroid, harderian gland, pancreas, heart, lung, large intestine, small intestine, stomach mucosa, uveal tract, and cartilage. Although uptake in some of these tissues was not as high as that observed for liver, spleen, kidney, urinary bladder, adrenal gland, and thyroid gland, uptake was still considered moderate.

Similarly, even in certain tissues where uptake of FGF10 constructs, such as FGF10-TK, was not higher than that of Tat-TK, the FGF10-TK complex had a longer duration within liver, kidney (renal cortex and medulla), and urinary bladder. In spleen, FGF10-TK outperformed Tat-TK based on both uptake and duration.

Finally, we compared the FGF10 portion containing constructs to another Surf+ Penetrating Polypeptide (+36GFP). FGF10 and +36GFP had similar overall tissue distribution patterns. For both Surf+ Penetrating Polypeptides, uptake to liver, kidney, spleen, adrenal gland, and stomach mucosa is high. However, the tissue distribution patterns are not identical. For example, uptake to pancreas is higher for the FGF10 portion constructs than for +36 GFP.

Tables 1 and 2 summarize the results of these studies.

Tissue Concentrations (ug equivalent protein/gram of tissue)

| | | TAT-TK | | | | FGF-10 | | | | FGF10-TK | | | | +36GFP | | | | +36GFP-TK | | | |
|---|---|---|---|---|---|---|---|---|---|---|---|---|---|---|---|---|---|---|---|---|---|
| Organ | Animal | # 1 | # 3 | # 5 | # 7 | # 49 | # 51 | # 53 | # 55 | # 33 | # 35 | # 37 | # 39 | # 41 | # 43 | # 45 | # 47 | # 9 | # 11 | # 13 | # 15 |
| System | Tissue | 5 min | 1 h | 6 h | 24 h | 5 min | 30 min | 1 h | 6 h | 5 min | 1 h | 6 h | 24 h | 5 min | 30 min | 1 h | 6 h | 5 min | 1 h | 6 h | 24 h |
| Vascular/ | Blood | 9.250 | 5.083 | 3.667 | 0.167 | 6.129 | 6.387 | 8.000 | 3.548 | 15.104 | 8.333 | 3.646 | 0.417 | 4.780 | 3.956 | 2.527 | 1.484 | 6.538 | 5.154 | 4.385 | 1.538 |
| Lymphatic | | | | | | | | | | | | | | | | | | | | | |
| Vascular/ | Bone | 5.000 | 2.167 | 1.833 | 0.083 | 7.935 | 7.097 | 7.226 | 3.290 | 7.396 | 3.021 | 1.771 | 0.208 | 11.154 | 7.088 | 5.330 | 3.791 | 6.000 | 5.538 | 3.308 | 1.462 |
| Lymphatic | Marrow | | | | | | | | | | | | | | | | | | | | |
| Vascular/ | Lymph Node | 29.333 | 5.917 | 1.667 | 0.083 | 4.194 | 4.323 | 4.968 | 2.581 | 4.271 | 3.958 | 1.458 | BQL | 3.407 | 2.527 | 3.242 | 1.758 | 2.538 | 1.385 | 2.077 | 0.462 |
| Lymphatic | | | | | | | | | | | | | | | | | | | | | |
| Vascular/ | Spleen | 2.833 | 2.667 | 4.417 | 0.333 | 38.258 | 29.871 | 23.161 | 7.871 | 52.604 | 31.667 | 6.771 | 0.729 | 32.747 | 37.857 | 39.615 | 23.571 | 70.846 | 33.231 | 27.692 | 14.538 |
| Lymphatic | | | | | | | | | | | | | | | | | | | | | |
| Vascular/ | Thymus | 2.083 | 1.833 | 1.167 | 0.000 | 1.677 | 2.452 | 3.032 | 1.677 | 4.063 | 4.792 | 1.354 | 0.104 | 2.253 | 1.429 | 1.593 | 0.934 | 1.615 | 1.077 | 2.154 | 0.231 |
| Lymphatic | | | | | | | | | | | | | | | | | | | | | |
| Excretory/ | Bile (in gail | 6.667 | NI | 15.500 | NI | 10.452 | 15.355 | 7.677 | 4.581 | 8.021 | 7.188 | 8.854 | 0.417 | NI | 11.099 | 11.923 | 9.670 | 0.000 | 7.846 | 6.923 | 3.538 |
| Metabolic | bladder) | | | | | | | | | | | | | | | | | | | | |
| Excretory/ | Renal Cortex | 32.083 | 8.250 | 5.667 | 0.750 | 200.774 | 109.742 | 112.387 | 63.742 | 147.604 | 84.688 | 36.146 | 2.292 | 143.736 | 96.648 | 89.286 | 46.703 | 66.769 | 43.615 | 22.231 | 4.077 |
| Metabolic | | | | | | | | | | | | | | | | | | | | | |
| Excretory/ | Renal | 19.667 | 4.833 | 4.083 | 0.417 | 141.742 | 66.452 | 74.452 | 46.065 | 101.354 | 49.792 | 22.708 | 1.667 | 159.835 | 81.209 | 57.582 | 31.593 | 47.846 | 29.769 | 13.769 | 3.231 |
| Metabolic | Medulla | | | | | | | | | | | | | | | | | | | | |
| Excretory/ | Liver | 55.667 | 2.500 | 3.583 | 0.583 | 82.774 | 69.806 | 59.226 | 10.774 | 93.646 | 53.333 | 8.854 | 0.625 | 107.527 | 99.011 | 96.429 | 73.187 | 67.692 | 53.154 | 33.692 | 25.154 |
| Metabolic | | | | | | | | | | | | | | | | | | | | | |
| Excretory/ | Urinary | 1.333 | 15.167 | 0.083 | NI | 8.194 | 41.613 | 8.000 | 178.903 | 6.771 | 40.313 | 22.604 | 0.000 | 2.473 | 9.725 | 32.308 | 28.956 | 3.769 | 17.692 | 30.615 | 0.846 |
| Metabolic | Bladder | | | | | | | | | | | | | | | | | | | | |
| Excretory/ | Bladder | NI | 32.167 | 119.500 | 0.333 | 9.032 | 49.161 | 76.194 | 454.323 | 1.354 | 72.500 | 75.729 | 0.000 | 1.813 | 19.341 | 33.626 | 153.462 | 1.154 | 80.231 | 34.923 | 9.154 |
| Metabolic | (contents) | | | | | | | | | | | | | | | | | | | | |
| Central | Brain | 0.417 | 0.417 | 0.333 | 0.000 | 0.387 | 0.968 | 0.903 | 0.387 | 0.938 | 1.146 | 0.313 | 0.000 | 0.440 | 0.385 | 0.440 | 0.330 | 0.308 | 0.231 | 0.308 | 0.077 |
| Nervous | (cerebellum) | | | | | | | | | | | | | | | | | | | | |
| System | | | | | | | | | | | | | | | | | | | | | |
| Central | Brain | 0.250 | 0.500 | 0.250 | BQL | 0.452 | 0.710 | 0.839 | 0.323 | 0.625 | 1.146 | 0.208 | 0.000 | 0.220 | 0.275 | 0.330 | 0.165 | 0.154 | 0.231 | 0.231 | 0.077 |
| Nervous | (cerebrum) | | | | | | | | | | | | | | | | | | | | |
| System | | | | | | | | | | | | | | | | | | | | | |
| Central | Brain | 0.500 | 0.500 | 0.250 | BQL | 0.903 | 1.484 | 0.968 | 0.387 | 0.938 | 1.250 | 0.313 | 0.000 | 0.495 | 0.440 | 0.714 | 0.549 | 0.231 | 0.231 | 0.308 | 0.077 |
| Nervous | (medulla) | | | | | | | | | | | | | | | | | | | | |
| System | | | | | | | | | | | | | | | | | | | | | |
| Central | Spinal Cord | 1.167 | 0.750 | 0.500 | 0.000 | 1.097 | 1.161 | 2.452 | 1.032 | 1.667 | 3.229 | 0.521 | 0.000 | 0.824 | 1.044 | 1.044 | 0.495 | 0.769 | 0.385 | 0.846 | 0.077 |
| Nervous | | | | | | | | | | | | | | | | | | | | | |
| System | | | | | | | | | | | | | | | | | | | | | |
| Endocrine | Adrenal | 75.750 | 2.917 | 5.167 | 0.583 | 142.387 | 113.806 | 74.581 | 20.194 | 149.896 | 97.917 | 48.438 | 2.604 | 200.989 | 89.780 | 105.385 | 76.209 | 71.154 | 38.000 | 51.846 | 30.538 |
| | Gland | | | | | | | | | | | | | | | | | | | | |
| Endocrine | Pituitary | 3.250 | 1.917 | 1.167 | BQL | 20.581 | 30.452 | 7.935 | 4.258 | 10.417 | 12.396 | 4.063 | 0.417 | 1.538 | 12.747 | 19.835 | 6.813 | 4.769 | 3.000 | 4.077 | 1.231 |
| | Gland | | | | | | | | | | | | | | | | | | | | |
| Endocrine | Thyroid | 4.500 | 2.750 | 10.667 | 0.667 | 18.774 | 20.774 | 17.548 | 20.645 | 16.146 | 11.042 | 16.146 | 13.125 | 8.242 | 2.637 | NI | 9.121 | 5.154 | 3.154 | 3.462 | 4.154 |
| Secretory | Harderian | 2.583 | 2.417 | 1.167 | 0.083 | 9.484 | 10.129 | 9.032 | 4.194 | 4.479 | 4.479 | 1.458 | 0.104 | 4.670 | 3.626 | 2.418 | 1.429 | 1.923 | 1.385 | 1.615 | 0.154 |
| | Gland | | | | | | | | | | | | | | | | | | | | |
| Secretory | Pancreas | 3.750 | 2.583 | 2.833 | 0.000 | 15.742 | 12.323 | 11.161 | 6.903 | 7.708 | 15.938 | 3.438 | 0.313 | 4.341 | 5.220 | 4.670 | 6.319 | 3.462 | 3.000 | 3.692 | 0.538 |
| Secretory | Salivary | 3.417 | 3.833 | 8.500 | 0.083 | 5.097 | 7.871 | 8.194 | 8.903 | 7.292 | 9.792 | 8.542 | 0.313 | 4.615 | 4.725 | 3.187 | 4.560 | 2.077 | 3.615 | 3.769 | 0.462 |
| | Gland | | | | | | | | | | | | | | | | | | | | |
| Fatty | Adipose | 2.417 | 1.667 | 1.333 | BQL | 2.387 | 3.871 | 3.097 | 1.742 | 2.292 | 5.625 | 1.458 | 0.104 | 1.209 | 5.440 | 2.143 | 2.802 | 5.615 | 2.308 | 5.231 | 1.154 |
| | (brown) | | | | | | | | | | | | | | | | | | | | |
| Fatty | Adipose | 0.917 | 0.750 | 0.667 | BQL | 0.258 | 1.419 | 1.484 | 1.677 | 1.458 | 2.396 | 0.625 | 0.000 | 1.429 | 1.538 | 0.549 | 0.385 | 0.385 | 0.231 | 0.538 | 0.154 |
| | (white) | | | | | | | | | | | | | | | | | | | | |
| Dermal | Skin (non- | 1.667 | 3.250 | 1.833 | 0.083 | 0.839 | 3.161 | 5.742 | 1.677 | 1.563 | 7.292 | 1.250 | 0.104 | 1.099 | 0.934 | 2.308 | 0.989 | 1.385 | 1.385 | 2.308 | 0.231 |
| | pigmented) | | | | | | | | | | | | | | | | | | | | |
| Reproductive | Epididymis | 4.167 | 2.667 | 2.917 | 0.083 | 1.484 | 7.871 | 4.000 | NI | 2.813 | 15.938 | 2.292 | 0.521 | 9.451 | 11.209 | 15.604 | 0.769 | 5.000 | 3.462 | 2.385 | 0.846 |
| Reproductive | Prostate | 0.583 | 3.250 | 2.250 | BQL | BQL | 1.419 | 3.548 | 4.129 | 2.604 | 5.833 | 3.958 | 0.000 | 1.264 | 4.286 | 1.978 | BQL | 2.538 | 2.923 | 3.077 | 0.538 |
| | Gland | | | | | | | | | | | | | | | | | | | | |
| Reproductive | Seminal | 0.833 | 1.250 | 2.500 | 0.000 | 4.645 | 3.935 | 2.968 | 5.806 | 2.708 | 4.271 | 3.958 | 0.104 | 2.033 | 2.857 | 3.462 | 1.923 | 1.615 | 2.462 | 2.615 | 0.231 |
| | Vesicles | | | | | | | | | | | | | | | | | | | | |
| Reproductive | Testis | 1.333 | 1.417 | 1.667 | 0.000 | 0.645 | 1.419 | 2.065 | 1.613 | 1.354 | 2.813 | 1.458 | 0.104 | 0.769 | 0.659 | 0.385 | 0.604 | 0.538 | 0.923 | 1.538 | 0.077 |
| Skeleto- | Bone | 2.917 | 2.000 | 1.417 | BQL | 1.806 | 2.323 | 4.645 | 2.065 | 3.125 | 5.729 | 1.354 | 0.000 | 1.868 | 1.758 | 1.154 | BQL | 1.692 | 2.154 | 1.769 | 0.231 |
| Muscular | | | | | | | | | | | | | | | | | | | | | |
| Skeleto- | Heart | 7.333 | 3.333 | 2.250 | 0.083 | 8.258 | 6.323 | 5.161 | 3.677 | 12.813 | 8.542 | 2.292 | 0.208 | 12.253 | 4.066 | 4.231 | 2.143 | 5.846 | 3.385 | 4.462 | 1.385 |
| Muscular | | | | | | | | | | | | | | | | | | | | | |
| Skeleto- | Skeletal | 1.333 | 1.000 | 0.833 | 0.000 | 1.032 | 2.323 | 2.710 | 1.226 | 1.563 | 2.604 | 1.042 | 0.104 | 0.934 | 0.604 | 0.879 | 0.659 | 1.000 | 1.000 | 1.000 | 0.077 |
| Muscular | Muscle | | | | | | | | | | | | | | | | | | | | |
| Skeleto- | Lung | 11.167 | 3.667 | 2.917 | 0.167 | 10.323 | 8.516 | 7.548 | 3.290 | 22.500 | 13.438 | 3.438 | 0.417 | 16.429 | 8.462 | 5.989 | 4.175 | 14.846 | 9.462 | 6.615 | 1.846 |
| Muscular | | | | | | | | | | | | | | | | | | | | | |
| Alimentary | Cecum | 2.583 | 1.917 | 2.500 | BQL | 7.097 | 8.581 | 8.710 | 5.290 | 5.667 | 5.938 | 2.708 | 0.000 | 4.396 | 4.560 | 4.176 | 2.967 | 3.000 | 4.077 | 2.462 | 0.385 |
| Canal | | | | | | | | | | | | | | | | | | | | | |
| Alimentary | Cecum | 0.667 | 0.750 | 3.083 | BQL | 0.839 | 1.290 | 1.613 | 3.097 | 1.146 | 2.083 | 3.021 | 0.208 | 0.879 | 0.769 | 0.549 | 1.374 | 0.615 | 1.154 | 2.308 | 0.231 |
| Canal | (contents) | | | | | | | | | | | | | | | | | | | | |
| Alimentary | Large | 1.750 | 2.500 | 3.417 | BQL | 17.032 | 9.032 | 8.129 | 5.032 | 5.729 | 6.458 | 4.688 | 0.000 | 4.121 | 10.879 | 3.462 | 2.967 | 2.769 | 3.000 | 2.615 | 0.462 |
| Canal | Intestine | | | | | | | | | | | | | | | | | | | | |
| Alimentary | Intestine | 0.750 | 0.500 | 5.167 | BQL | 4.323 | 4.774 | 2.581 | 4.258 | 2.813 | 2.813 | 6.563 | 0.521 | 1.209 | 4.341 | 1.209 | 2.418 | 0.538 | 1.615 | 2.769 | 0.923 |
| Canal | (contents) | | | | | | | | | | | | | | | | | | | | |
| Alimentary | Small | 5.750 | 4.250 | 1.833 | BQL | 13.419 | 26.000 | 15.226 | 7.677 | 13.229 | 6.250 | 6.875 | 0.000 | 7.308 | 15.385 | 9.560 | 5.220 | 3.615 | 5.923 | 2.692 | 1.231 |
| Canal | Intestine | | | | | | | | | | | | | | | | | | | | |
| Alimentary | Intestine | 2.917 | 4.250 | 4.333 | 0.000 | 7.226 | 4.387 | 6.387 | 5.161 | 1.979 | 4.583 | 3.229 | 0.104 | 2.637 | 4.560 | 2.143 | 0.934 | 2.308 | 3.231 | 2.000 | 0.308 |
| Canal | (contents) | | | | | | | | | | | | | | | | | | | | |
| Alimentary | (gastric | 9.417 | 5.750 | 36.500 | 0.083 | 31.226 | 28.323 | 45.419 | 22.129 | 34.375 | 25.313 | 14.063 | 0.833 | 16.593 | 22.912 | 12.418 | 26.758 | 2.077 | 21.769 | 11.077 | 1.308 |
| Canal | mucosa) | | | | | | | | | | | | | | | | | | | | |
| Alimentary | Stomach | 2.083 | 2.583 | 31.667 | 0.083 | 5.032 | 3.097 | 5.806 | 11.871 | 6.667 | 13.229 | 24.896 | 0.625 | 2.857 | 3.901 | 1.923 | 14.725 | 1.308 | 12.308 | 6.385 | 0.769 |
| Canal | (contents) | | | | | | | | | | | | | | | | | | | | |
| Ocular | Eye (lens) | 0.750 | 1.333 | 1.000 | 0.083 | 0.968 | 1.484 | 2.581 | 1.419 | 1.458 | 2.083 | 0.938 | 0.104 | 0.769 | 0.549 | 1.209 | 0.549 | 0.385 | 0.385 | 1.000 | 0.154 |
| Ocular | Eye (uveal | 2.833 | 2.417 | 1.333 | 0.000 | 5.806 | 8.000 | 8.516 | 2.194 | 7.083 | 5.208 | 2.292 | 0.000 | 4.670 | 5.330 | 3.462 | 2.582 | 1.231 | 1.692 | 2.077 | 0.308 |
| | tract) | | | | | | | | | | | | | | | | | | | | |
| Misc | Injection Site | 6.833 | 6.333 | 2.000 | 0.083 | 2.645 | 4.710 | 6.581 | 2.323 | 4.271 | 5.104 | 2.188 | 0.208 | 2.088 | 2.363 | 2.088 | 1.538 | 4.000 | 2.385 | 66.000 | 34.615 |
| | (tail) | | | | | | | | | | | | | | | | | | | | |
| Misc | Sciatic | 2.167 | 0.000 | 1.333 | BQL | NI | NI | 2.839 | NI | NI | NI | NI | NI | NI | NI | NI | NI | NI | NI | NI | NI |
| | Nerve | | | | | | | | | | | | | | | | | | | | |
| Misc | Dorsal Root | 2.833 | 1.750 | 0.083 | BQL | NI | NI | 3.484 | NI | NI | NI | NI | NI | NI | NI | NI | NI | NI | NI | NI | NI |
| | Ganglion | | | | | | | | | | | | | | | | | | | | |
| Misc | Cartilage | 3.333 | 3.000 | 2.500 | 0.083 | 6.452 | 7.548 | 6.903 | 3.226 | 6.146 | 6.979 | 2.917 | 0.313 | 2.802 | 4.725 | 6.209 | 2.473 | 3.538 | 3.385 | 2.462 | 0.615 |

NI = tissue not identified on sections;
NS = Not sampled due to values that were BQI
BQL = Value is below the LLOQ or tissue could not be visualized on autoradioluminograph due to BQL radioactivit
lower limit of quantitation (LLC 0.0006 μCi/g/0.018 μCi/μg =
Upper limit of quantitation (UL 1.9300 μCi/g/0.018 μCi/μg =
* = value is above the ULOQ

Tissue Concentrations (% Injected Dose/gram of tissue)

| | | TAT-TK | | | | FGF-10 | | | | FGF10-TK | | | | +36GFP | | | | +36GFP-TK | | | |
|---|---|---|---|---|---|---|---|---|---|---|---|---|---|---|---|---|---|---|---|---|---|
| | | MW | | | | MW | | | | MW | | | | MW | | | | MW | | | |
| | | (kDa | 44.0 | | | (kDa | 19.3 | | | (kDa | 59.4 | | | (kDa | 29.9 | | | (kDa) | 70.2 | | |
| Organ | Animal | # 1 | # 3 | # 5 | # 7 | # 49 | # 51 | # 53 | # 55 | # 33 | # 35 | # 37 | # 39 | # 41 | # 43 | # 45 | # 47 | # 9 | # 11 | # 13 | # 15 |
| System | Tissue | 5 min | 1 h | 6 h | 24 h | 5 min | 30 min | 1 h | 6 h | 5 min | 1 h | 6 h | 24 h | 5 min | 30 min | 1 h | 6 h | 5 min | 1 h | 6 h | 24 h |
| Vascular/ | Blood | 3.70% | 2.14% | 1.33% | 0.08% | 2.05% | 1.87% | 2.66% | 1.02% | 4.60% | 3.11% | 1.08% | 0.13% | 1.57% | 1.26% | 0.87% | 0.48% | 3.26% | 1.82% | 1.96% | 0.58% |
| Lymphatic | | | | | | | | | | | | | | | | | | | | | |
| Vascular/ | Bone | 2.00% | 0.91% | 0.67% | 0.04% | 2.65% | 2.07% | 2.41% | 0.95% | 2.25% | 2.99% | 0.52% | 0.06% | 3.66% | 2.25% | 1.83% | 1.23% | 2.99% | 1.95% | 1.48% | 0.55% |
| Lymphatic | Marrow | | | | | | | | | | | | | | | | | | | | |
| Vascular/ | Lymph | 11.73% | 2.49% | 0.61% | 0.04% | 1.40% | 1.26% | 1.65% | 0.75% | 1.30% | 1.48% | 0.43% | BQL | 1.12% | 0.80% | 1.11% | 0.57% | 1.26% | 0.49% | 0.93% | 0.17% |
| Lymphatic | Node | | | | | | | | | | | | | | | | | | | | |
| Vascular/ | Spleen | 1.13% | 1.12% | 1.61% | 0.15% | 12.77% | 8.72% | 7.71% | 2.27% | 16.02% | 11.81% | 2.00% | 0.23% | 10.75% | 12.01% | 13.57% | 7.65% | 35.31% | 11.73% | 12.36% | 5.50% |
| Lymphatic | | | | | | | | | | | | | | | | | | | | | |
| Vascular/ | Thymus | 0.83% | 0.77% | 0.42% | 0.00% | 0.56% | 0.72% | 1.01% | 0.48% | 1.24% | 1.79% | 0.40% | 0.03% | 0.74% | 0.45% | 0.55% | 0.30% | 0.80% | 0.38% | 0.56% | 0.09% |
| Lymphatic | | | | | | | | | | | | | | | | | | | | | |
| Excretory/ | Bile | 2.67% | NI | 5.64% | NI | 3.49% | 4.48% | 2.56% | 1.32% | 2.44% | 2.68% | 2.62% | 0.13% | NI | 3.52% | 4.08% | 3.14% | 0.00% | 2.77% | 3.09% | 1.34% |
| Metabolic | (in gall | | | | | | | | | | | | | | | | | | | | |
| | bladder) | | | | | | | | | | | | | | | | | | | | |
| Excretory/ | Renal | 12.83% | 3.47% | 2.06% | 0.34% | 66.99% | 32.05% | 37.41% | 18.41% | 44.96% | 31.59% | 10.70% | 0.71% | 47.20% | 30.67% | 30.59% | 15.17% | 33.28% | 15.39% | 9.92% | 1.54% |
| Metabolic | Cortex | | | | | | | | | | | | | | | | | | | | |
| Excretory/ | Renal | 7.86% | 2.03% | 1.48% | 0.19% | 47.30% | 19.41% | 24.79% | 13.30% | 30.87% | 18.57% | 6.72% | 0.52% | 52.49% | 25.77% | 19.73% | 10.26% | 23.85% | 10.51% | 6.15% | 1.22% |
| Metabolic | Medulla | | | | | | | | | | | | | | | | | | | | |
| Excretory/ | Liver | 22.26% | 1.05% | 1.30% | 0.27% | 27.62% | 20.39% | 19.72% | 3.11% | 28.53% | 19.89% | 2.62% | 0.19% | 35.31% | 31.42% | 33.04% | 23.77% | 33.74% | 18.76% | 15.04% | 9.52% |
| Metabolic | | | | | | | | | | | | | | | | | | | | | |
| Excretory/ | Urinary | 0.53% | 6.37% | 0.03% | NI | 2.73% | 12.15% | 2.66% | 51.66% | 2.06% | 15.04% | 6.69% | 0.00% | 0.81% | 3.09% | 11.07% | 9.40% | 1.88% | 6.24% | 13.67% | 0.32% |
| Metabolic | Bladder | | | | | | | | | | | | | | | | | | | | |
| Excretory/ | Bladder | NI | 13.52% | 43.45% | 0.15% | 3.01% | 14.36% | 25.37% | 131.19% | 0.41% | 27.04% | 22.42% | 0.00% | 0.60% | 6.14% | 11.52% | 49.84% | 0.58% | 28.32% | 15.59% | 3.46% |
| Metabolic | (contents) | | | | | | | | | | | | | | | | | | | | |
| Nervous | Brain | 0.17% | 0.18% | 0.12% | 0.00% | 0.13% | 0.28% | 0.30% | 0.11% | 0.29% | 0.43% | 0.09% | 0.00% | 0.14% | 0.12% | 0.15% | 0.11% | 0.15% | 0.08% | 0.14% | 0.03% |
| System | (cere- | | | | | | | | | | | | | | | | | | | | |
| | bellum) | | | | | | | | | | | | | | | | | | | | |
| Nervous | Brain | 0.10% | 0.21% | 0.09% | BQL | 0.15% | 0.21% | 0.28% | 0.09% | 0.19% | 0.43% | 0.06% | 0.00% | 0.07% | 0.09% | 0.11% | 0.05% | 0.08% | 0.08% | 0.10% | 0.03% |
| System | (cere- | | | | | | | | | | | | | | | | | | | | |
| | brum) | | | | | | | | | | | | | | | | | | | | |
| Central | Brain | 0.20% | 0.21% | 0.09% | BQL | 0.30% | 0.43% | 0.32% | 0.11% | 0.29% | 0.47% | 0.09% | 0.00% | 0.16% | 0.14% | 0.24% | 0.18% | 0.12% | 0.08% | 0.14% | 0.03% |
| Nervous | (medulla) | | | | | | | | | | | | | | | | | | | | |
| System | | | | | | | | | | | | | | | | | | | | | |
| Central | Spinal | 0.47% | 0.32% | 0.18% | 0.00% | 0.37% | 0.34% | 0.82% | 0.30% | 0.51% | 1.20% | 0.15% | 0.00% | 0.27% | 0.33% | 0.36% | 0.16% | 0.38% | 0.14% | 0.38% | 0.03% |
| Nervous | Cord | | | | | | | | | | | | | | | | | | | | |
| System | | | | | | | | | | | | | | | | | | | | | |
| Endocrine | Adrenal | 30.29% | 1.23% | 1.88% | 0.27% | 47.51% | 33.24% | 24.83% | 5.83% | 45.66% | 36.52% | 14.34% | 0.81% | 66.00% | 28.49% | 36.11% | 24.75% | 35.46% | 13.41% | 23.14% | 11.55% |
| | Gland | | | | | | | | | | | | | | | | | | | | |
| Endocrine | Pituitary | 1.30% | 0.81% | 0.42% | BQL | 6.87% | 8.89% | 2.64% | 1.23% | 3.17% | 4.62% | 1.20% | 0.13% | 0.51% | 4.05% | 6.80% | 2.21% | 2.38% | 1.06% | 1.82% | 0.47% |
| | Gland | | | | | | | | | | | | | | | | | | | | |
| Endocrine | Thyroid | 1.80% | 1.16% | 3.88% | 0.31% | 6.26% | 6.07% | 5.84% | 5.96% | 4.92% | 4.12% | 4.78% | 4.09% | 2.71% | 0.84% | NI | 2.96% | 2.57% | 1.11% | 1.55% | 1.57% |
| Secretory | Harderian | 1.03% | 1.02% | 0.42% | 0.04% | 3.16% | 2.96% | 3.01% | 1.21% | 1.36% | 1.67% | 0.43% | 0.03% | 1.53% | 1.15% | 0.83% | 0.46% | 0.96% | 0.49% | 0.72% | 0.06% |
| | Gland | | | | | | | | | | | | | | | | | | | | |
| Secretory | Pancreas | 1.50% | 1.09% | 1.03% | 0.00% | 5.25% | 3.60% | 3.72% | 1.99% | 2.35% | 5.94% | 1.02% | 0.10% | 1.43% | 1.66% | 1.60% | 2.05% | 1.73% | 1.06% | 1.65% | 0.20% |
| Secretory | Salivary | 1.37% | 1.61% | 3.09% | 0.04% | 1.70% | 2.30% | 2.73% | 2.57% | 2.22% | 3.65% | 2.53% | 0.10% | 1.52% | 1.50% | 1.09% | 1.48% | 1.04% | 1.28% | 1.68% | 0.17% |
| | Gland | | | | | | | | | | | | | | | | | | | | |
| Fatty | Adipose | 0.97% | 0.70% | 0.48% | BQL | 0.80% | 1.13% | 1.03% | 0.50% | 0.70% | 2.10% | 0.43% | 0.03% | 0.40% | 1.73% | 0.73% | 0.91% | 2.80% | 0.81% | 2.34% | 0.44% |
| | (brown) | | | | | | | | | | | | | | | | | | | | |
| Fatty | Adipose | 0.37% | 0.32% | 0.24% | BQL | 0.09% | 0.41% | 0.49% | 0.48% | 0.44% | 0.89% | 0.19% | 0.00% | 0.47% | 0.49% | 0.19% | 0.13% | 0.19% | 0.08% | 0.24% | 0.06% |
| | (white) | | | | | | | | | | | | | | | | | | | | |
| Dermal | Skin (non- | 0.67% | 1.37% | 0.67% | 0.04% | 0.28% | 0.92% | 1.91% | 0.48% | 0.48% | 2.72% | 0.37% | 0.03% | 0.36% | 0.30% | 0.79% | 0.32% | 0.69% | 0.49% | 1.03% | 0.09% |
| | pig- | | | | | | | | | | | | | | | | | | | | |
| | mented) | | | | | | | | | | | | | | | | | | | | |
| Repro- | Epi- | 1.67% | 1.12% | 1.06% | 0.04% | 0.50% | 2.30% | 1.33% | NI | 0.86% | 5.94% | 0.68% | 0.16% | 3.10% | 3.56% | 5.35% | 0.25% | 2.49% | 1.22% | 1.06% | 0.32% |
| ductive | didymis | | | | | | | | | | | | | | | | | | | | |
| Repro- | Prostate | 0.23% | 1.37% | 0.82% | BQL | BQL | 0.41% | 1.18% | 1.19% | 0.79% | 2.18% | 1.17% | 0.00% | 0.42% | 1.36% | 0.68% | BQL | 1.26% | 1.03% | 1.37% | 0.20% |
| ductive | Gland | | | | | | | | | | | | | | | | | | | | |
| Repro- | Seminal | 0.33% | 0.53% | 0.91% | 0.00% | 1.55% | 1.15% | 0.99% | 1.68% | 0.82% | 1.59% | 1.17% | 0.03% | 0.67% | 0.91% | 1.19% | 0.62% | 0.80% | 0.87% | 1.17% | 0.09% |
| ductive | Vesicles | | | | | | | | | | | | | | | | | | | | |
| Repro- | Testis | 0.53% | 0.60% | 0.61% | 0.00% | 0.22% | 0.41% | 0.69% | 0.47% | 0.41% | 1.05% | 0.43% | 0.03% | 0.25% | 0.21% | 0.13% | 0.20% | 0.27% | 0.33% | 0.69% | 0.03% |
| ductive | | | | | | | | | | | | | | | | | | | | | |
| Skeleto- | Bone | 1.17% | 0.84% | 0.52% | BQL | 0.60% | 0.68% | 1.55% | 0.60% | 0.95% | 2.14% | 0.40% | 0.00% | 0.61% | 0.56% | 0.40% | BQL | 0.84% | 0.76% | 0.79% | 0.09% |
| Muscular | | | | | | | | | | | | | | | | | | | | | |
| Skeleto- | Heart | 2.93% | 1.40% | 0.82% | 0.04% | 2.76% | 1.85% | 1.72% | 1.06% | 3.90% | 3.19% | 0.68% | 0.06% | 4.02% | 1.29% | 1.45% | 0.70% | 2.91% | 1.19% | 1.99% | 0.52% |
| Muscular | | | | | | | | | | | | | | | | | | | | | |
| Skeleto- | Skeletal | 0.53% | 0.42% | 0.30% | 0.00% | 0.34% | 0.68% | 0.90% | 0.35% | 0.48% | 0.97% | 0.31% | 0.03% | 0.31% | 0.19% | 0.30% | 0.21% | 0.50% | 0.35% | 0.45% | 0.03% |
| Muscular | Muscle | | | | | | | | | | | | | | | | | | | | |
| Skeleto- | Lung | 4.47% | 1.54% | 1.06% | 0.08% | 3.44% | 2.49% | 2.51% | 0.95% | 6.85% | 5.01% | 1.02% | 0.13% | 5.40% | 2.69% | 2.05% | 1.36% | 7.40% | 3.34% | 2.95% | 0.70% |
| Muscular | | | | | | | | | | | | | | | | | | | | | |
| Alimen- | Cecum | 1.03% | 0.81% | 0.91% | BQL | 2.37% | 2.51% | 2.90% | 1.53% | 2.03% | 2.21% | 0.80% | 0.00% | 1.44% | 1.45% | 1.43% | 0.96% | 1.50% | 1.44% | 1.10% | 0.15% |
| tary | | | | | | | | | | | | | | | | | | | | | |
| Canal | | | | | | | | | | | | | | | | | | | | | |
| Alimen- | Cecum | 0.27% | 0.32% | 1.12% | BQL | 0.28% | 0.38% | 0.54% | 0.89% | 0.35% | 0.78% | 0.89% | 0.06% | 0.29% | 0.24% | 0.19% | 0.45% | 0.31% | 0.41% | 1.03% | 0.09% |
| tary | (contents) | | | | | | | | | | | | | | | | | | | | |
| Canal | | | | | | | | | | | | | | | | | | | | | |
| Alimen- | Large | 0.70% | 1.05% | 1.24% | BQL | 5.68% | 2.64% | 2.71% | 1.45% | 1.75% | 2.41% | 1.39% | 0.00% | 1.35% | 3.45% | 1.19% | 0.96% | 1.38% | 1.06% | 1.17% | 0.17% |
| tary | Intestine | | | | | | | | | | | | | | | | | | | | |
| Canal | | | | | | | | | | | | | | | | | | | | | |
| Alimen- | Large | 0.30% | 0.21% | 1.88% | BQL | 1.44% | 1.39% | 0.86% | 1.23% | 0.86% | 1.05% | 1.94% | 0.16% | 0.40% | 1.38% | 0.41% | 0.79% | 0.27% | 0.57% | 1.24% | 0.35% |
| tary | Intestine | | | | | | | | | | | | | | | | | | | | |
| Canal | (contents) | | | | | | | | | | | | | | | | | | | | |
| Alimen- | Small | 2.30% | 1.79% | 0.67% | BQL | 4.48% | 7.59% | 5.07% | 2.22% | 4.03% | 2.33% | 2.04% | 0.00% | 2.40% | 4.88% | 3.28% | 1.70% | 1.80% | 2.09% | 1.20% | 0.47% |
| tary | Intestine | | | | | | | | | | | | | | | | | | | | |
| Canal | | | | | | | | | | | | | | | | | | | | | |
| Alimen- | Small | 1.17% | 1.79% | 1.58% | 0.00% | 2.41% | 1.28% | 2.13% | 1.49% | 0.60% | 1.71% | 0.96% | 0.03% | 0.87% | 1.45% | 0.73% | 0.30% | 1.15% | 1.14% | 0.89% | 0.12% |
| tary | Intestine | | | | | | | | | | | | | | | | | | | | |
| Canal | (contents) | | | | | | | | | | | | | | | | | | | | |
| Alimen- | Stomach | 3.77% | 2.42% | 13.27% | 0.04% | 10.42% | 8.27% | 15.12% | 6.39% | 10.47% | 9.44% | 4.16% | 0.26% | 5.45% | 7.27% | 4.25% | 8.69% | 1.04% | 7.68% | 4.94% | 0.49% |
| tary | (gastric | | | | | | | | | | | | | | | | | | | | |
| Canal | mucosa) | | | | | | | | | | | | | | | | | | | | |
| Alimen- | Stomach | 0.83% | 1.09% | 11.51% | 0.04% | 1.68% | 0.90% | 1.93% | 3.43% | 2.03% | 4.93% | 7.37% | 0.19% | 0.94% | 1.24% | 0.66% | 4.78% | 0.65% | 4.34% | 2.85% | 0.29% |
| tary | (contents) | | | | | | | | | | | | | | | | | | | | |
| Canal | | | | | | | | | | | | | | | | | | | | | |
| Ocular | Eye (lens) | 0.30% | 0.56% | 0.36% | 0.04% | 0.32% | 0.43% | 0.86% | 0.41% | 0.44% | 0.78% | 0.28% | 0.03% | 0.25% | 0.17% | 0.41% | 0.18% | 0.19% | 0.14% | 0.45% | 0.06% |
| Ocular | Eye (uveal | 1.13% | 1.02% | 0.48% | 0.00% | 1.94% | 2.34% | 2.84% | 0.63% | 2.16% | 1.94% | 0.68% | 0.00% | 1.53% | 1.69% | 1.19% | 0.84% | 0.61% | 0.60% | 0.93% | 0.12% |
| | tract) | | | | | | | | | | | | | | | | | | | | |
| Misc | Injection | 2.73% | 2.66% | 0.73% | 0.04% | 0.88% | 1.38% | 2.19% | 0.67% | 1.30% | 1.90% | 0.65% | 0.06% | 0.69% | 0.75% | 0.72% | 0.50% | 1.99% | 0.84% | 29.46% | 13.10% |
| | Site | | | | | | | | | | | | | | | | | | | | |
| | (tail) | | | | | | | | | | | | | | | | | | | | |
| Misc | Sciatic | 0.87% | 0.00% | 0.48% | BQL | NI | NI | 0.95% | NI | NI | NI | NI | NI | NI | NI | NI | NI | #VAL- | NI | NI | NI |
| | Nerve | | | | | | | | | | | | | | | | | UE! | | | |
| Misc | Dorsal | 1.13% | 0.74% | 0.03% | BQL | NI | NI | 1.16% | NI | NI | NI | NI | NI | NI | NI | NI | NI | #VAL- | NI | NI | NI |
| | Root | | | | | | | | | | | | | | | | | UE! | | | |
| | Ganglion | | | | | | | | | | | | | | | | | | | | |
| Misc | Cartilage | 1.33% | 1.26% | 0.91% | 0.04% | 2.15% | 2.20% | 2.30% | 0.93% | 1.87% | 2.60% | 0.86% | 0.10% | 0.92% | 1.50% | 2.13% | 0.80% | 1.76% | 1.19% | 1.10% | 0.23% |

NI = tisue not identified on sections;
NS = Not sampled due to values that were BQL
BQL = Value is below the LLOQ or tissue could not be visualized on autoradioluminograph due to BQL radioactivit
* = value is above the ULOQ

### Example 9

### Cellular Distribution

The study detailed above evaluated tissue distribution of various constructs, including two FGF10 portion-containing constructs. In addition to tissue distribution, cell-type specific uptake information will be evaluated to ascertain which cells in a particular tissue are being penetrated.

To obtain specific cell-type uptake information in select organs, microautoradiography analysis was performed on selected organs collected at various time points. For microautoradiography analysis (MARG), animals were anesthetized and blood collected, as described above. Tissue samples are trimmed to be approximately 0.5 cm², placed into cryosectioning embedding media on a cryosectioning sample holder, and frozen in isopentane that is cooled with liquid nitrogen. Frozen samples were stored in liquid nitrogen until analysis by MARG. For analysis, tissues were cryosectioned at 5 μm under darkroom conditions, and collected onto glass slides that are pre-coated with photographic emulsion (Kodak NTB photographic emulsion). Slides were placed in sealed light-safe slide boxes and allowed to expose the photographic emulsion for 7 days before being developed, fixed and stained with hematoxylin and eosin. All slides were examined for cellular localization using light microscopy.

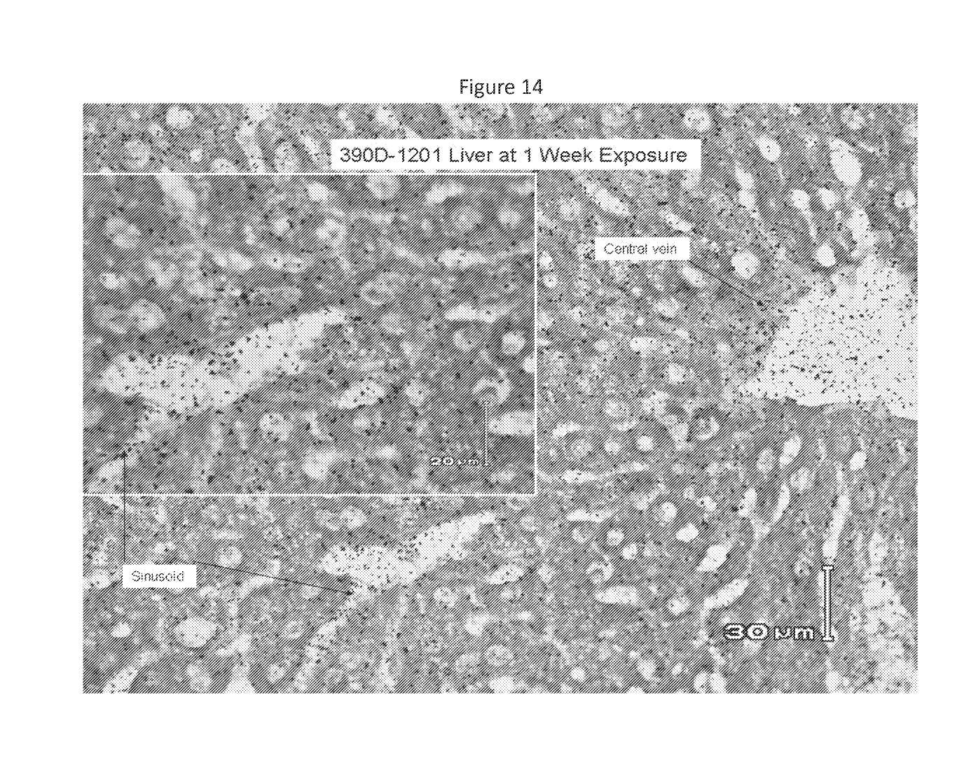

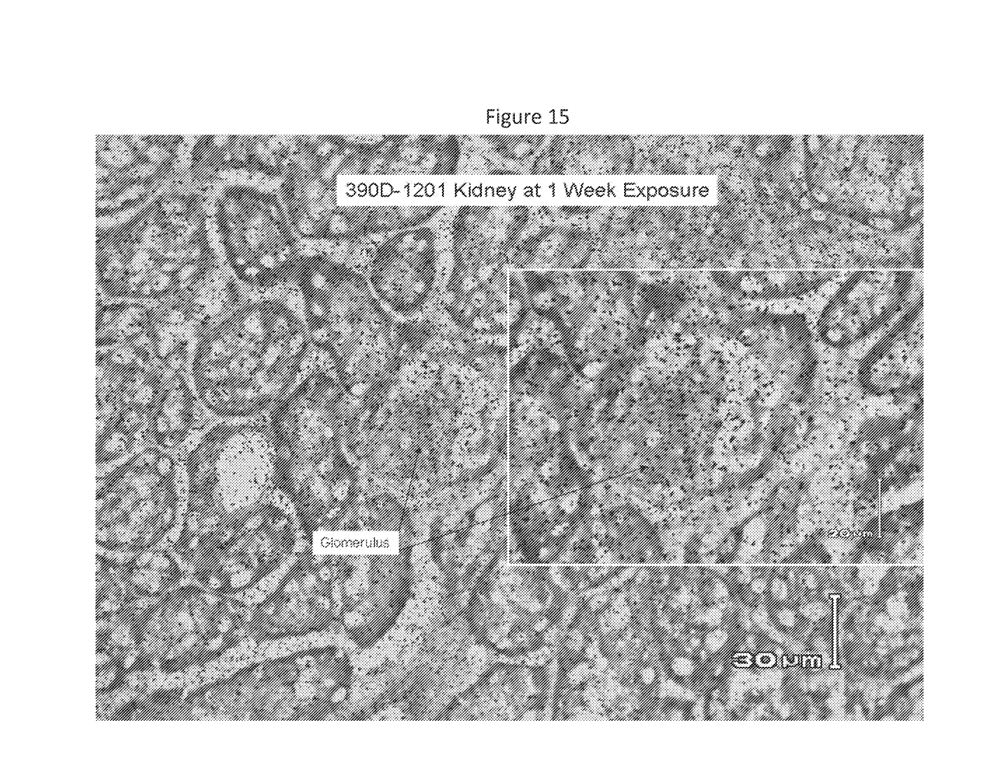

Images from liver MARG in FIG. 14 suggest that FGF10 is uniformly distributed throughout the liver tissue including in hepatocytes. In addition, the images suggest that FGF10 is both inside cells and in the extracellular space between cells or on cell surfaces. Similarly, images from kidney MARG (FIG. 15) suggest that FGF10 is uniformly distributed throughout the kidney, both within cells and in the extracellular space between cells or on cell surfaces.

### Example 10

### Tissue Distribution of FGF10 Portion does not Correlate with Native Expression of FGF10 Receptor

The PK and tissue distribution studies described above using ¹²⁵I labeled, cell penetrating FGF10 domain constructs, alone or complexed with a cargo portion, demonstrated that the proteins were highly and preferentially (although not exclusively) distributed to particular tissues, including liver, kidney, spleen, and pancreas following an i.v. administration through tail vein of mice. Interestingly, these data do not tightly correlate with the expression profile of FGFR2, the main receptor for FGF10, in various organs/tissues of human or mice (http://biogps.org). For example, FGFR2 is not highly expressed in liver and kidney, but these are major tissues of uptake for systemically administered FGF10 with or without fused protein such as thymidine kinase. It should be noted that human FGFR2 is 92% identical to mouse FGFR2, and that human FGF10 was shown by others to be functional in mice (Greenwood-Van Meerveld B, et al. J Pharm Pharmacol. 2003 January).

These data suggest that the tissue distribution of FGF10-cargo fusion proteins administered intravenously or by other routes of administration mainly depend on the pharmacokinetics of the Surf+ Penetrating Polypeptide portion (here, a domain of full length, unprocessed, naturally occurring FGF10) and its function as a Surf+ Penetrating Polypeptide. In other words, tissue distribution is largely a function of the binding and internalization of the FGF10 portion based on cell surface proteoglycan interactions; rather than being solely or primarily a function of expression of FGFR2 on the surface of cells and subsequent binding of the FGF10 portion to those FGFR2-expressing cells via FGFR2.

### Example 11

### Liver Enzyme as Cargo Portion

Enzyme replacement strategies for treating conditions have been hampered by difficulties delivering sufficient enzyme into the appropriate cells and tissue. Delivery into liver is considered particularly challenging, and yet, replacing defective enzymes that are typically active (solely or to a significant degree) in liver represents a significant approach for addressing diseases caused by enzyme deficiency. Amongst the enzymes that endogenously function in the liver of healthy individuals are enzymes involved in metabolism.

A complex, such as a fusion protein, is made by fusing an FGF10 portion and a cargo portion directly or via a linker. The cargo portion is a liver enzyme, or functional fragment thereof. In other words, the cargo portion is an enzyme that, in healthy subjects, endogenously functions in the liver. The FGF10 portion comprises a domain of FGF10 having surface positive charge, an overall net positive charge, and a charge/molecular weight ratio greater than that of full length, naturally occurring FGF10. The FGF10 portion may be N- or C-terminal to the cargo portion. Both fusion proteins are made and tested.

The fusion protein is tested to confirm that it retains cell penetration activity, particularly ability to internalize into liver cells. Suitable cell penetration testing is done in, for example, primary hepatocytes and/or hepatoma cell lines. Cell penetration of the fusion protein is compared to controls, such as the FGF10 portion alone and the cargo portion alone. In addition, enzymatic activity of the cargo portion is tested to confirm that activity is retained in the context of the fusion protein. Enzymatic activity can be evaluated in an in vitro assay and/or a cell-free assay where activity is compared to the cargo portion alone. Preferably, at least 50% of the activity of the enzyme is maintained in the context of the fusion protein.

Following confirmation that this fusion protein retains the enzymatic activity of the cargo portion and successfully penetrates liver cells, the fusion proteins are tested in (i) healthy animals to confirm enhanced localization to liver and internalization in vivo and (ii) an animal model of the enzyme deficiency to evaluate ability of delivered enzyme to improve one or more symptoms. Note that enhanced localization to one or more target tissues does not mean or imply that the delivered fusion protein exclusively localizes to a tissue. However, enhanced localization reflects localization that is not ubiquitous across all tissues.

As detailed above, given the observed enhanced localization of FGF10 portion-containing constructs to liver, other constructs (e.g., having different cargo; comprising a different domain of FGF10; a variant) can be tested in cell-based assays, such as primary hepatocytes or cell lines.

For example, fresh or cryopreserved human and mouse hepatocytes are obtained and plated into 6-well plates. FGF10 containing a C-Myc terminal tag is added to the cells to allow for binding and internalization over the course of 4 hours. Cells will be detached off the plate either with trypsin, or PBS-EDTA solution, resulting in cells in suspension. Trypsin-treated cells in suspension would also have any surface-bound FGF10 removed and retain only internalized FGF10, whereas EDTA-detached cells will have both surface-associated FGF10 and internalized protein. Both sets of cells are fixed/permeabilized and labeled with fluorescently-labeled anti-C-Myc antibodies to label FGF10 protein. All cells will be analyzed by flow cytometry. Cells in which FGF10 is present and the FGF10 is labeled with the anti-Myc antibody will have higher fluorescence intensity than those cells in which FGF10 is not present or in cells not incubated with FGF10 protein. This experiment is useful for demonstrating and/or confirming that a particular FGF10 portion-containing construct or fusion protein binds to and penetrates hepatocytes.

### Example 12

### p16 Tumor Suppressor as Cargo Portion

p16 is a tumor suppressor protein of therapeutic interest. Of particular interest is regional administration to the abdominal cavity, such as by intraperitoneal injection for treating p16 deficient primary and metastatic tumors of the abdominal cavity. Additionally or alternatively, local delivery, such as intratumoral delivery is also of particular interest. Note, however, that deliver may also be systemic.

A complex, such as a fusion protein, is made by fusing an FGF10 portion and a cargo portion directly or via a linker. The cargo portion is a tumor suppressor, or functional fragment thereof. In this example, the tumor suppressor is p16. The FGF10 portion comprises a domain of FGF10 having surface positive charge, an overall net positive charge, and a charge/molecular weight ratio greater than that of full length, unprocessed, naturally occurring FGF10. The FGF10 portion may be N- or C-terminal to the cargo portion. Both fusion proteins are made and tested.

As noted above, the fusion proteins optionally include a linker that interconnects the FGF10 portion to the cargo portion. Suitable linkers include a glycine/serine rich linker. When present, the linker may also include a serum-stable proteolytic cleavage site, such as a site cleavable by cathepsin class proteases. Cleavable linkers permit the separation of the cargo portion from the FGF10 portion following internalization.

The following exemplary fusion proteins have been generated:
Myc-FGF10 portion-(G₄S)₂-p16-Hiss
His₆-p16-(G₄S)₂-FGF10 portion-Myc

Where, for example:
FGF10 portion is the domain of full length, naturally occurring human FGF10 set forth in SEQ ID NO: 2 (residues 64 to 208 of the full-length sequence);
p16 is the p16^{Ink4A} coding sequence from residues 1 to 156 of NCBI ref NP_{—}000068.1 (SEQ ID NO: 5);
(G₄S) is the linker amino acid sequence “GGGGS” (SEQ ID NO: 31);
His6 is the tag “HHHHHH” (SEQ ID NO: 28);
Myc is the tag “EQKLISEEDL” (SEQ ID NO: 32).
   In this example, the p16 refers to the full-length, human p16^{INK4A} sequence (NCBI refseq ID NP_{—}000068.1). However, in certain embodiments, p16 truncations may be similarly used. Exemplary truncations omit ankyrin repeat 1 of p16. The amino acid sequence of the full length, human p16 is set forth in SEQ ID NO: 5. The amino acid sequence of the Myc-FGF10 portion-(G₄S)₂-p16-His₆ construct is set forth in SEQ ID NO: 6. The amino acid sequence of the His₆-p16-(G₄S)₂-FGF10 portion-Myc construct is set forth in SEQ ID NO: 7.

The pJexpress-416 expression vector containing the coding sequence for Myc-FGF10-(G4S)2-p16^{Ink4}-His6 was transformed into the BL21(DE3) strain of E. coli cells. The cells were grown to a density of 1.5 (as measured by A600) in ProGro media (Expression Technologies) containing 50 micrograms/mL kanamycin, induced with 0.5 mM IPTG and incubated at 22° C. with shaking at 350 rpm for 17 hours. Cells were harvested by centrifugation at 6,000×g for ten minutes.

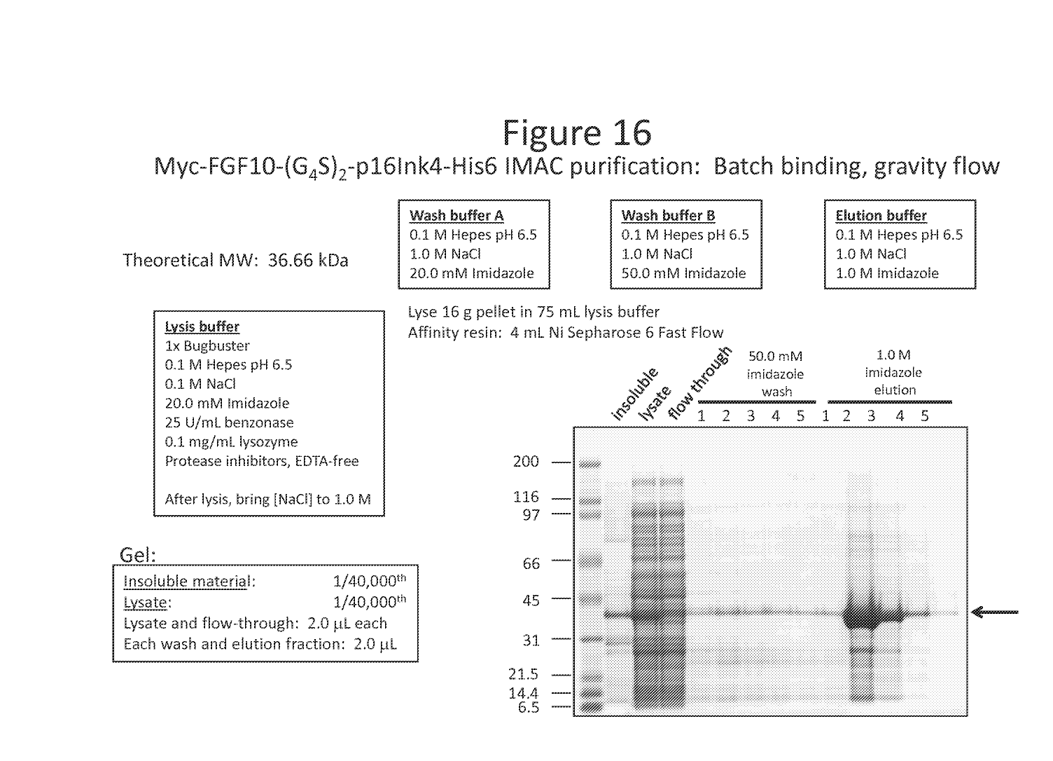

The resulting cell pellet was lysed in lysis buffer, the NaCl concentration was subsequently brought to 1.0 M (see FIG. 16), the lysate was clarified by centrifugation at 20,000×g for ten minutes, and the supernatant was applied to Ni sepharose 6 fast flow (GE Healthcare). The bound resin was washed with wash buffer A (see FIG. 16), followed by wash buffer B, and eluted with elution buffer. Indicated aliquots of representative fractions were applied to 4-12% polyacrylamide gel and visualized with Instant Blue coomassie stain (Expedeon; FIG. 16).

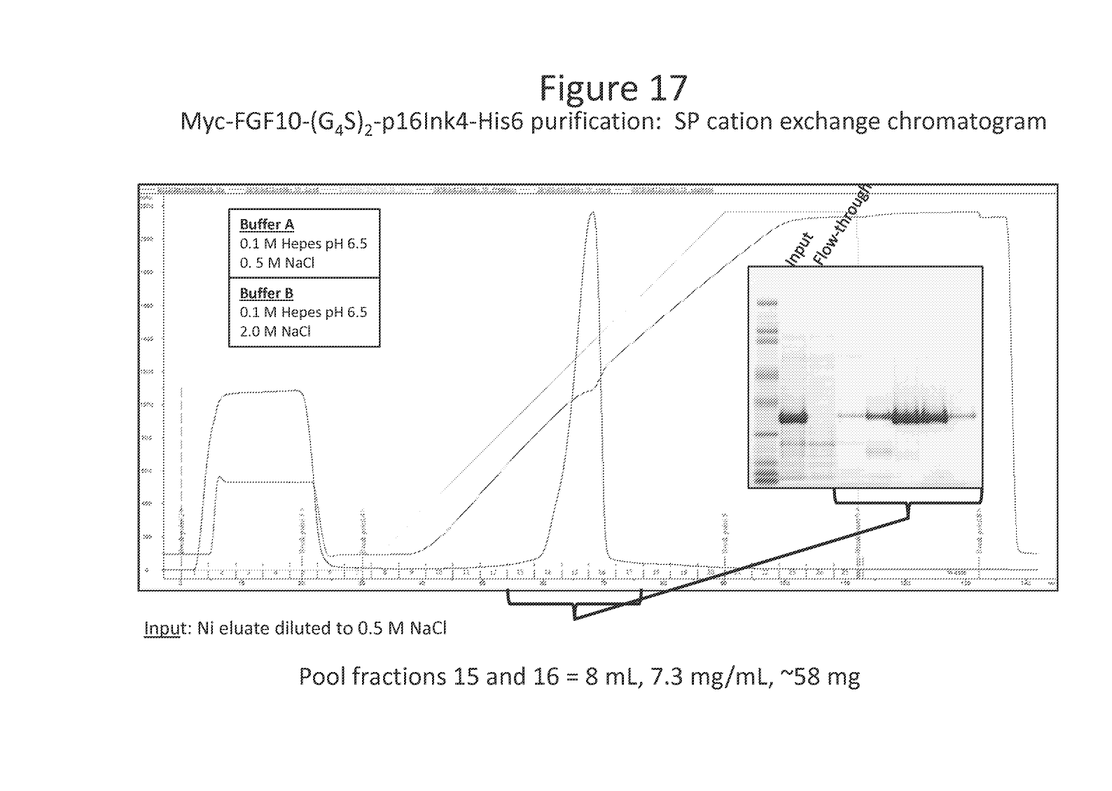

IMAC-purified material was then subjected to cation exchange chromatography. The concentration of NaCl was brought to 0.5M by dilution and the protein was then applied to a 5.0 mL HiPrep SP HP column (GE Healthcare). The protein was eluted with a gradient of NaCl from 0.5M to 2.0M over 12 column volumes (FIG. 17). The peak-containing fractions were pooled, dialyzed against buffer (20.0 mM Hepes, pH 7.5, 0.5 M NaCl, and 5.0% glycerol), divided into small aliquots, snap-frozen in liquid nitrogen, and stored at −80° C.

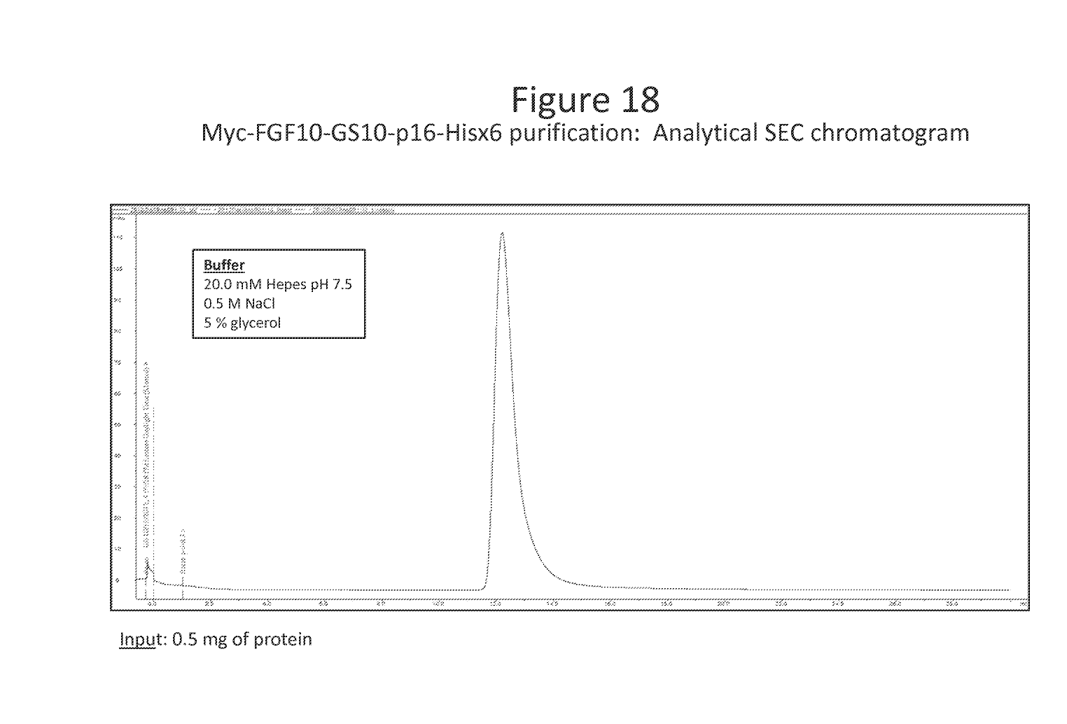

A 0.5 mg aliquot of the cation-exchange purified and dialyzed product was applied to a Superdex 75 10/300 GL size exclusion chromatography column (GE Healthcare) for analysis. The column was run in 20.0 mM Hepes, pH 7.5, 0.5 M NaCl, and 5.0% glycerol. The protein eluted in a single, sharp peak corresponding to about 28 kDa, a size similar to the expected molecular weight of about 37 kDa (FIG. 18)

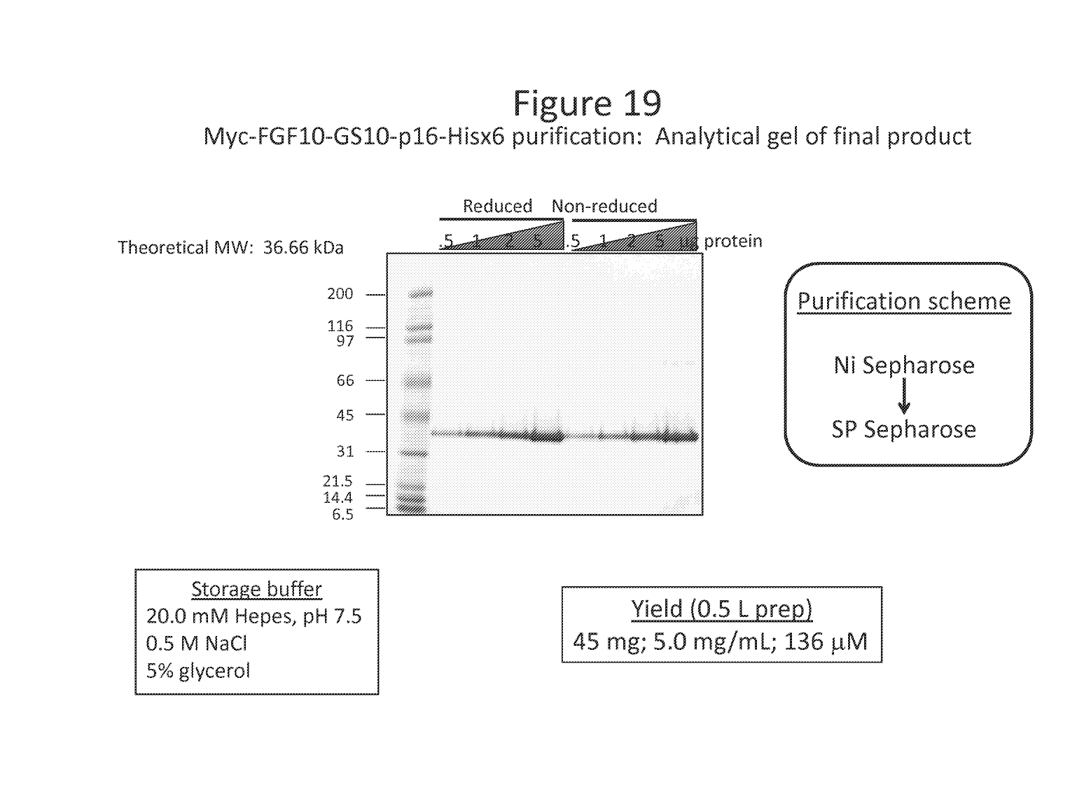

A summary of the purification is as follows, and gel analysis of the final product is shown in FIG. 19:
32 g cell paste was produced per liter of culture
The yield after the SP cation exchange purification was about 100 mg protein from the equivalent of 1 L culture
The protein was divided into small aliquots, snap-frozen in liquid nitrogen, and stored at −80° C. in 20 mM HEPES, pH 7.5, 0.5 M NaCl, and 5% glycerol.
The final protein was greater than 95% pure

### Example 13

### FGF10-p16 Complexes Internalize into Cells

The FGF10-p16 fusion protein previously described was demonstrated to be readily taken up and internalized by the cells.

On the day prior to the assay, HepG2 (hepatoma), Hela cells, and 10⁶ SW626 cells (ovarian) were plated in each well of a 6-well plate and incubated in the 37° C. CO₂ incubator. The cells were washed once with PBS and then were replenished with 2 mL of growth media containing 2 uM of p16, +36GFP-p16 or FGF10-p16 proteins and were incubated overnight in the 37° C. CO₂ incubator. The cells were then washed once with PBS and detached with 0.25% trypsin/EDTA. The cells were fixed for 10 minutes with 1 mL 4% formaldehyde and permeabilized for 5 minutes with 0.5 mL 0.4% saponin. Then 2.5 uL of 1 mg/mL FITC labeled chicken polyclonal anti-myc tag antibody (Abcam #1394) was added to all the cells and incubated at 4° C. in the dark for 2 hours, except for cells treated with +36GFP-p16 (GFP itself was used for detection). The cells were washed twice with ice cold PBS and were analyzed using a BD Accuri™ C6 flow cytometer.

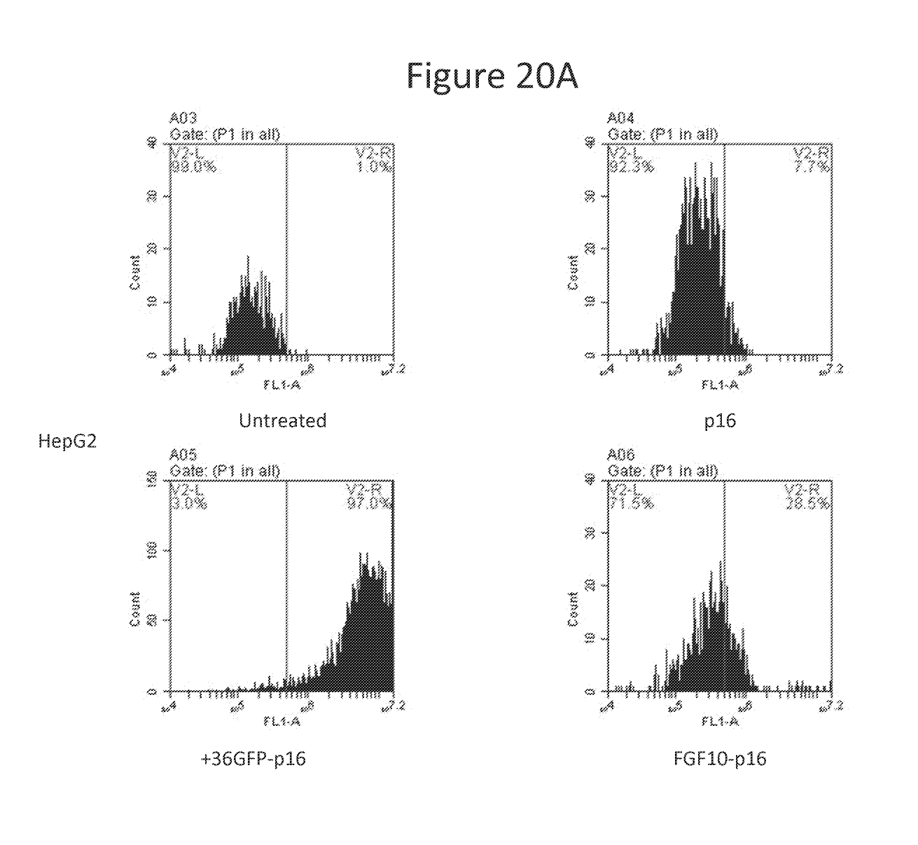

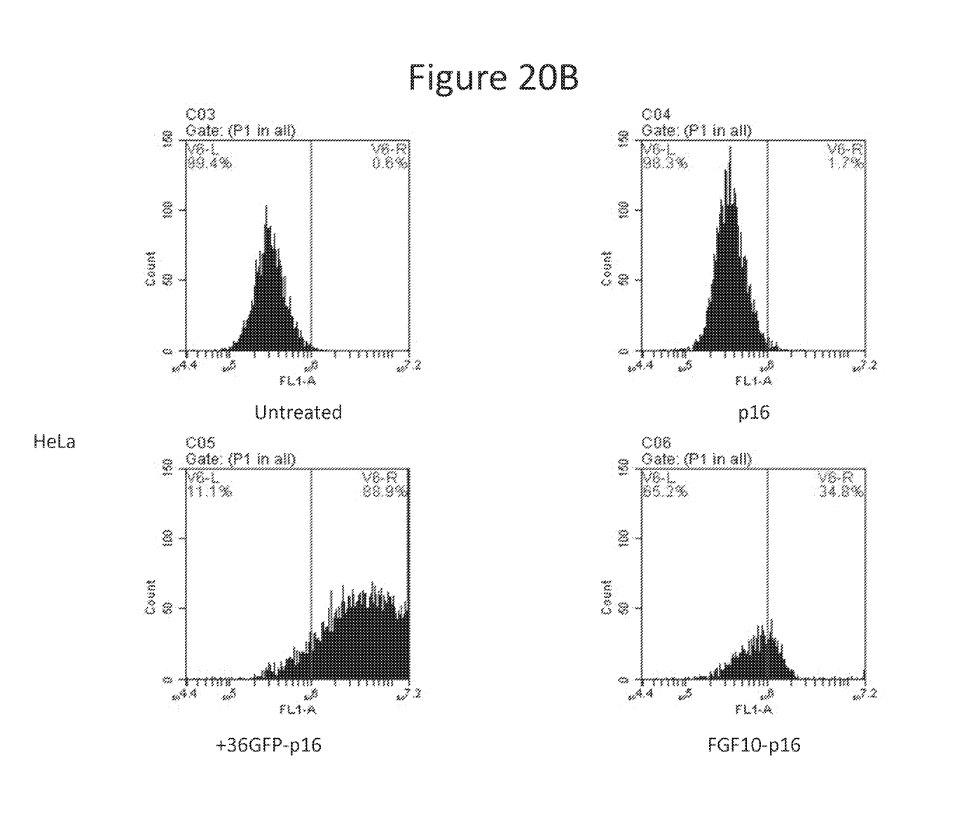

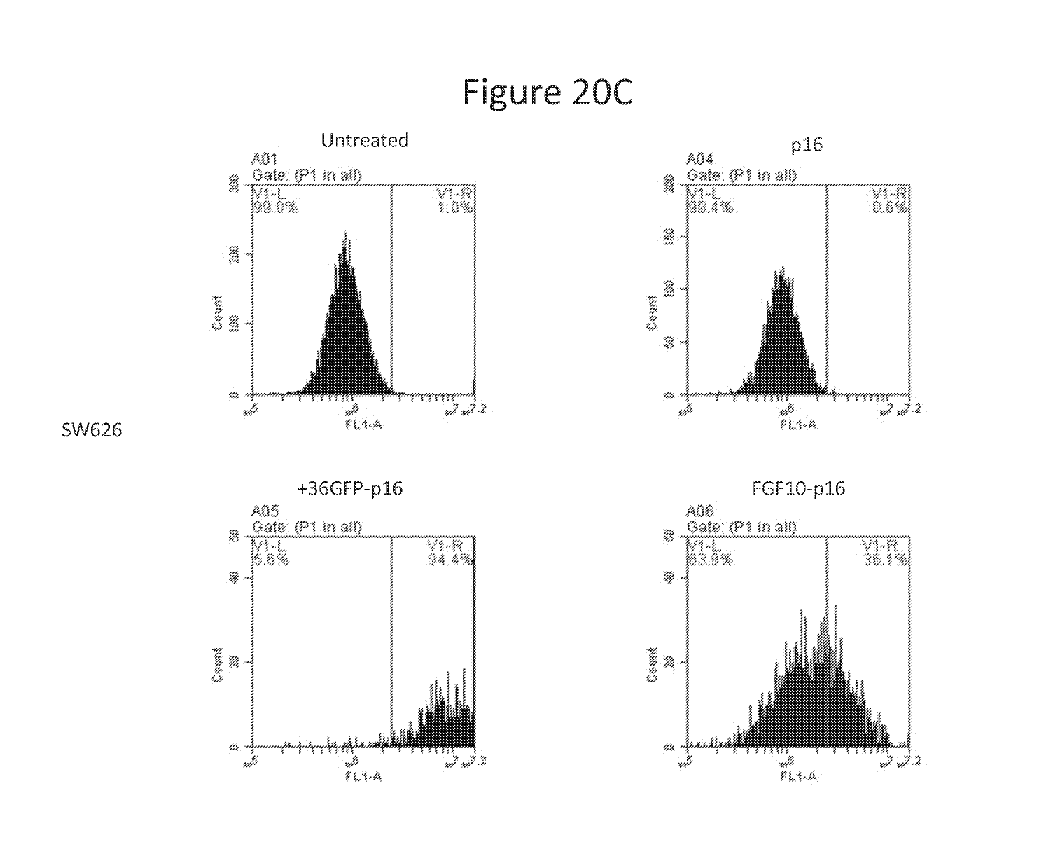

The results of these experiments are depicted in FIGS. 20A-20C. >90% of +36GFP-p16 treated cells or ˜30% of FGF10-p16 treated cells were stained positive compared to untreated or p16 alone treated cells. The mean intensity of fluorescence of internalized proteins is also shown to be significantly increased (excluding comparison to +36GFP-p16 samples). In other words, the p16 fusion proteins with +36GFP and FGF10 penetrate cells, while p16 alone does not.

### Example 14

### FGF10-p16 Complexes as Anti-Cancer Agents In Vitro

The fusion proteins outlined in Example 13 were evaluated for anti-tumor efficacy. Initial evaluation is performed in preclinical cancer models. Demonstration of the effects of the fusion proteins can be through evaluation of apoptosis induction, evaluation of the effects on Rb phosphorylation, and effects on the cell cycle. Initially, these effects are evaluated on human ovarian cancer cell lines in vitro, with follow up studies in human tumor xenografts, including explants from human derived tissues, following either systemic or intraperitoneal delivery.

In these experiments, functional activity of p16 fusion proteins was evaluated by demonstrating that p16 fusion proteins exhibit anti-proliferative effect, i.e. complementation of p16 deficiency, in p16 deficient, but Rb competent, cell lines.

Materials & Methods

SKOV-3 ovarian cancer cells were plated in a 96-well format and grown overnight. Cells were incubated with test articles: a) 1 uM of p16, b) 1 uM of +36GFP-p16, c) 1 uM of FGF10-p16 or d) no treatment in the presence of various concentrations of an endosome escape agent such as PFO (five 3× dilution starting from 40 nM; exemplary endosomal escape agents, such as PFO or PFO-like agents, are disclosed in WO2012/094653) for 4 hours in serum-free medium. One group of cells, called “untreated cells” received no test article treatment and no PFO treatment. After incubation, the media from all cell wells was aspirated and fresh complete medium was added for 3 hours. Cell viability was assessed using an MTS assay. Results are reported as number of viable cells as % relative to the untreated cells.

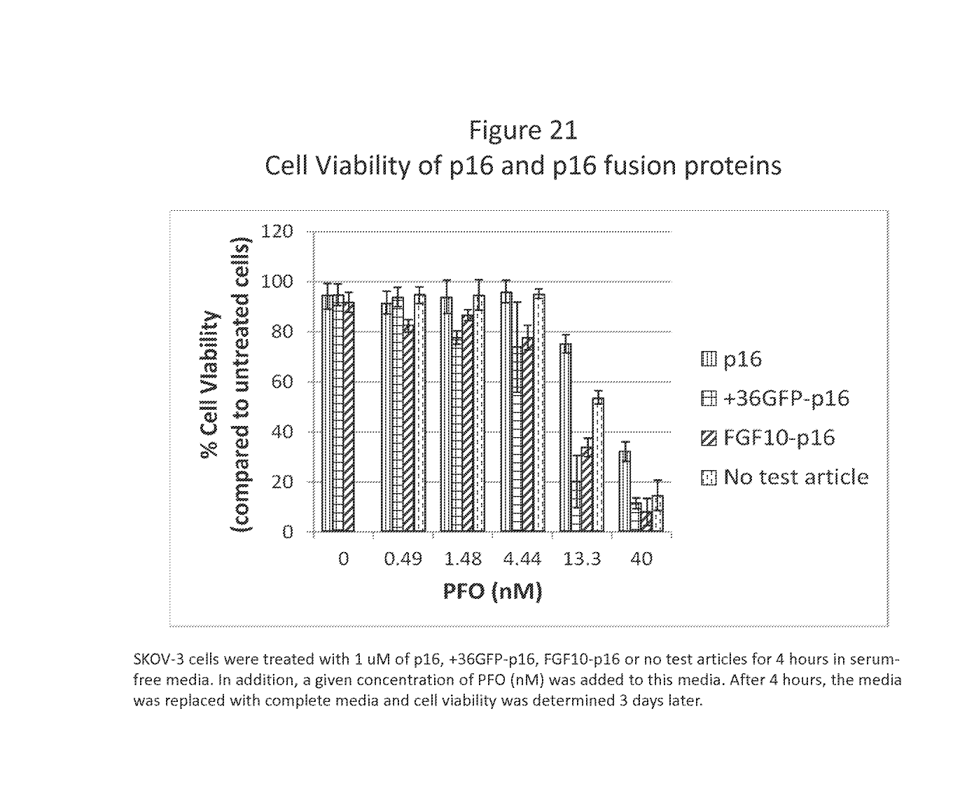

Results: Treatment of SKOV-3 cells with p16 and p16 fusion proteins demonstrated the ability of the p16 fusions (fusions with a Surf+ Penetrating Polypeptide) to inhibit cell proliferation, by complementing p16 deficiency (FIG. 21). The complementation required the presence of an endosome escape agent (such as, PFO) as evidenced by the lack of effect in the presence of p16 fusions alone. p16 alone failed to block cell proliferation at levels higher than the background toxicity of an endosomal escape agent, suggesting that the presence of a Surf+ Penetrating Polypeptide is essential for the p16 complementation to be successful. FGF10-p16 and +36GFP-p16 both led to significant reduction in the number of viable cells not accounted for by background toxicity due to the endosomal escape agent with the effect being the strongest at 13.3 nM of endosomal escape agent (FIG. 21).

In FIG. 21, a set of bars represents cell viability following administration of p16, a p16 fusion protein, or a no protein control at a given concentration of endosomal escape agent. Within each set of bars (for a given concentration of endosomal escape agent), the bars depict from left to right: left—the results for p16 alone, +36GFP-p16, FGF10-p16, and no protein control.

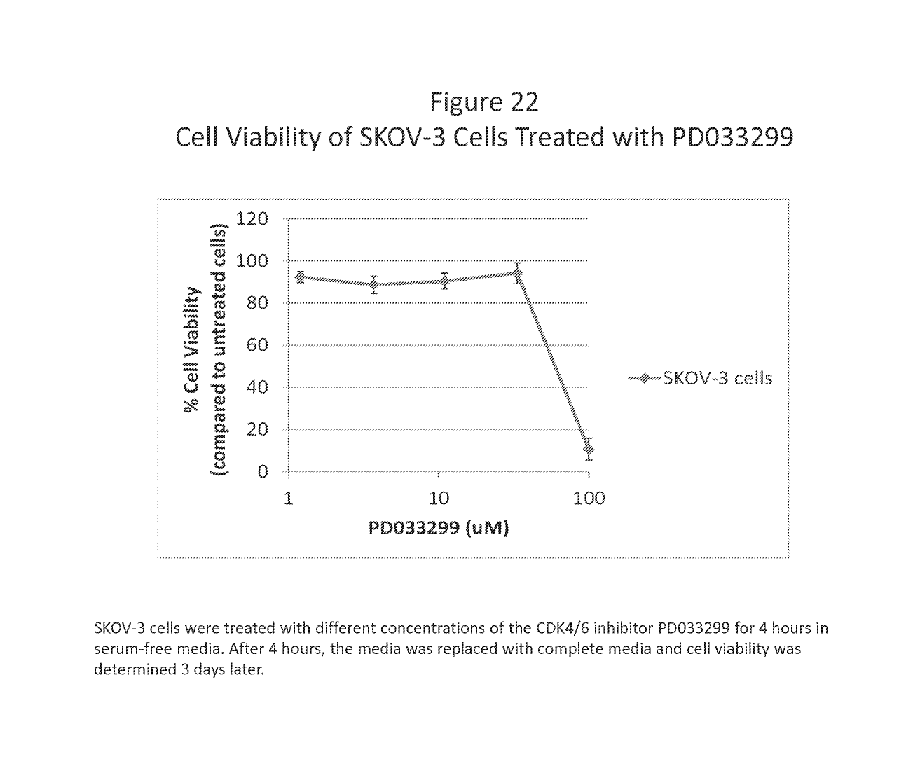

The CDK4/6 inhibitor PD-0332991 was used as positive control for inhibition of cell proliferation in the p16 deficient SKOV-3 cell line (FIG. 22). 4-hour treatment of SKOV-3 cells with 100 uM PD-0332991 in serum-free media, followed by 3 days of growth in complete media led to complete inhibition of cell proliferation.

In vitro studies may also be performed in other cell lines, such as panels of ovarian cancer cell lines having differing genotypes. Suitable cell lines, any one or more of which can be used, include: p16−/Rb+ cells (SKOV-3, RMG-1, OVTOKO, HEY, OVCAR5, DOV13, TYK-nu, PEO6, and OVCA429); p16−/Rb− cells (e.g., PEO-6, EFO21, and CAOV3); p16+/Rb+ cells (e.g., ES-2, PA-1, and NIH OVCAR-3).

### Example 15

### FGF10-p16 Complexes as Anti-Cancer Agents In Vivo

Following in vitro studies indicative of therapeutic efficacy, in vivo experiments, such as in a xenograft model, are performed. To demonstrate the effect of the p16-containing fusion protein(s) in vivo, 2-10×10⁶ cells of the above cell lines are injected intraperitoneal (i.p.) into female nude or SCID mice. After 4-8 days, the mice are treated with 1-300 mg/kg fusion protein, p16 alone or vehicle control through weekly i.p. injection for up to 20 weeks. Mice are monitored daily for morbidity and mortality. Injection of fusion protein, but not p16 alone, is expected to prolong the survival of the nude mice implanted with p16-sensitive ovarian cancer cell lines. Biopsy can be taken from the tumors to assess necrosis and apoptosis induced by the fusion protein.

In another study, mice are injected i.p. with 1-5×10⁶ ovarian cancer cells stably expressing a reporter gene such as the a luciferase reporter gene (e.g. SKOV3-luc-D3 cell line by Caliper, a Perkin Elmer company, is an ovarian cancer cell line stably transfected with the luciferase gene). After eight days, mice are imaged for bioluminescence using the reporter and then randomized for treatment with fusion protein, p16 alone or vehicle control through i.p. injection. Thereafter, all mice are imaged weekly for bioluminescence and monitored for morbidity and mortality. Injection of fusion protein, but not p16 alone, is expected to significantly reduce tumor growth as measured by bioluminescence signal from the reporter gene, and is expected to prolong the survival of treated mice.

The foregoing are merely exemplary of the xenograft studies that can be carried out.

Following successful in vitro and animal studies, the clinical efficacy of the p16-containing fusion protein(s) is evaluated. A cohort of 10-30 patients with recurrent ovarian cancer demonstrating Rb-proficiency and low p16 expression are enrolled and given 10-1000 mg FGF10-p16 (fusion protein) by i.p. (or i.v.) once weekly or every four weeks for six cycles. The level of CA125, i.e. biochemical response, can be monitored as the primary end point, while PET-CT scan of tumor growth, and progression free survival can be considered as secondary end points.

In another study, the efficacy of the fusion protein can be evaluated in combination with the current standard of care. For example, 10-1000 mg fusion protein is given to patients in combination with cisplatin/carboplatin, taxol/taxene, or doxorubicin. The response rate of the combination treatment will be compared to that of standard of care alone.

### Example 16

### Cell Penetration Mediated by a Domain of FGF10 does not Require the FGF10 Receptor

As described above, the preferential uptake of FGF10 domain constructs in a manner that does not correlate with FGFR2 expression suggests that uptake of FGF10 is not wholly dependent on FGFR expression. To further evaluate this model, uptake in the absence of FGFR2 is assessed.

A cell type that expresses FGFR2-IIIb (also referred to as FGFR2b), the highest affinity and main cell-surface receptor for FGF10, is treated with a cell penetrating domain of FGF10 containing a Myc tag in the presence or absence of an antibody to FGFR2-IIIb (specifically GP369—an antibody that is known to block binding of FGF10; Bai et al. 2010 J. Cancer. Res. 70: 7630). GP369 is also known not to activate the receptor and thus is an appropriate tool for uptake studies. By comparing the amount of FGF10 internalization in the presence or absence of blocking antibody, the relative contribution of FGFR2-IIIb-mediated cell uptake will be assessed.

Internalization of FGF10 will be measured by flow cytometry through use of a fluorescently-labeled Myc tag as described earlier. If FGF10 is significantly endocytosed through proteoglycan-interactions at the cell surface, as expected, then cell uptake will be minimally attenuated by blocking the receptor.

To measure whether FGF10 is inducing downstream signaling by binding to the FGF receptor, the amount of Erk1/2 phosphorylation is measured by Western blotting using phospo-Erk or Erk antibodies. Cell types that do not express FGFR2-IIIb will also be included to assess the contribution of FGF10 interactions with FGFR2-IIIb versus proteoglycan-interactions with the presumption that Erk1/2 phosphorylation is not a downstream signal of FGF10 interactions with cell surface proteoglycans.

### Example 17

### Minimizing the Native Function of FGF10

In the context of the present disclosure, FGF10 is being harnessed to enhance cell penetration, thereby facilitating delivery of therapeutic cargo into cells. Since the FGF10 portion of complexes of the disclosure is not being used for the mitogenic activity of FGF10, it may be useful to minimize that endogenous activity in the context of these fusion proteins. The structure of FGF10 bound to the FGFR2b receptor has been described in detail and, based on this structure and modeling studies, several mutants with reduced biological activity (e.g., reduced mitogenic and/or FGFR2b binding activity) have been generated or proposed. These variants include the mutant protein FGF10 E158K/K195A, which decreases binding to the FGFR2b receptor by approximately a factor of 4 without affecting the binding of FGF10 to heparin. An additional mutant is FGF10 R78A which shows an approximately 4-fold decrease in binding to the FGFR2b receptor along with a significant decrease in mitogenic activity. Mutant FGF10 proteins with T114 modified to either arginine or alanine demonstrated reduced binding to FGFR2b relative to the wild-type protein as well as reduced mitogenic activity.

An additional variant was generated. This tetramutant (4 amino acid substitutions relative to the native sequence) includes amino acid substitutions at each of the foregoing positions (78, 114, 158, and 195). This tetramutant is a variant of human FGF10 comprising four amino acid substitutions in the human FGF10 domain set forth in SEQ ID NO: 2 (R78A, T114A, E158A, K195A). The amino acid sequence of the tetramutant is set forth in SEQ ID NO: 10. We note that the numbering of the variant residues is the numbering, relatively to the full length, unprocessed FGF10. This tetramutant has reduced mitogenic activity (e.g., helps eliminate the intrinsic mitogenic effects of FGF10).

A fusion protein of this variant FGF10 having four amino acid substitutions and mCherry was expressed and purified similarly to Hisx6-FGF10-Myc as described in Example 1. This fusion protein is set forth in SEQ ID NO: 11.

This fusion protein comprising this variant FGF10 sequence was demonstrated to be readily taken up and internalized by cells. On the day prior to the assay, 10⁶ HepG2 or Hela cells were plated in each well of a 6-well plate and incubated in the 37° C. CO₂ incubator. The cells were washed once with PBS and then were replenished with 2 mL of growth media containing 2 uM of wild-type (wt) or mutant (mut) FGF10-mCherry fusion proteins and were incubated for 2 h in the 37° C. CO₂ incubator. The cells were then washed twice with PBS and detached with 0.25% trypsin/EDTA. The cells were washed one more time with PBS and then analyzed using a BD Accuri™ C6 flow cytometer. The level of internalization was quantified as the mean intensity of mCherry fluorescence in the cells. As shown in the table below, the mCherry fusion proteins that included the tetramutant FGF10 internalized equally well as the mCherry fusion proteins that included wild-type FGF10 in both HepG2 and Hela cells. For both tested fusion proteins, the FGF10 portion is the domain of FGF10 set forth in SEQ ID NO: 2. The mut FGF10 has the following four substitutions: R78A, T114A, E158A, K195A).

| Cells | HepG2 | Hela |
|---|---|---|
| Untreated | 2,234 | 3,920 |
| wt FGF10-mCherry | 57,016 | 58,699 |
| mut FGF10-mCherry | 42,566 | 62,950 |

SEQUENCE LISTING
SEQ ID NO: 1 - fibroblast growth factor 10 (FGF-10)
precursor (full length, unprocessed, naturally
occurring human FGF-10)
MWKWILTHCASAFPHLPGCCCCCFLLLFLVSSVPVTCQALGQDMVSPEATNSSSSSFSSP
SSAGRHVRSYNHLQGDVRWRKLFSFTKYFLKIEKNGKVSGTKKENCPYSILEITSVEIGV
VAVKAINSNYYLAMNKKGKLYGSKEFNNDCKLKERIEENGYNTYASFNWQHNGRQMYVA
LNGKGAPRRGQKTRRKNTSAHFLPMVVHS
SEQ ID NO: 2 - domain of FGF-10 (residues 64-208
of full length, unprocessed, naturally occurring
human FGF-10)
GRHVRSYNHLQGDVRWRKLFSFTKYFLKIEKNGKVSGTKKENCPYSILEITSVEIGVVAV
KAINSNYYLAMNKKGKLNGSKEFNNDCKLKERIEENGYNTYASFNWQHNGRQMYVALNG
KGAPRRGQKTRRKNTSAHFLPMVVHS
SEQ ID NO: 3 - SR39 mutant of HSV TK
MASYPCHQHASAFDQAARSRGHNNRRTALRPRRQQKATEVRLEQKMPTLLRVYIDGPHGM
GKTTTTQLLVALGSRDDIVYVPEPMTYWRVLGASETIANIYTTQHRLDQGEISAGDAAVVMT
SAQITMGMPYAVTDAVLAPHIGGEAGSSHAPPPALTIFLDRHPIAFMLCYPAARYLMGSMTP
QAVLAFVALIPPTLPGTNIVLGALPEDRHIDRLAKRQRPGERLDLAMLAAIRRVYGLLANTVR
YLQGGGSWREDWGQLSGAAVPPQGAEPQSNAGPRPHIGDTLFTLFRAPELLAPNGDLYNVF
AWALDVLAKELRPMHVFILDYDQSPAGCRDALLQLTSGMVQTHVTTPGSIPTICDLARTFAR
EMGEAN
SEQ ID NO: 4 - His6-FGF10-(G4S)2-TK (the gly/ser linker
is underlined; the His tag is double underlined)
MHHHHHHMGRHVRSYNHLQGDVRWRKLFSFTKYFLKIEKNGKVSGTKKENCPYSILEITSV
EIGVVAVKAINSNYYLAMNKKGKLYGSKEFNNDCKLKERIEENGYNTYASFNWQHNGRQM
YVALNGKGAPRRGQKTRRKNTSAHFLPMVVHSGGGGSGGGGSASYPCHQHASAFDQAARS
RGHNNRRTALRPRRQQKATEVRLEQKMPTLLRVYIDGPHGMGKTTTTQLLVALGSRDDIVY
VPEPMTYWRVLGASETIANIYTTQHRLDQGEISAGDAAVVMTSAQITMGMPYAVTDAVLAP
HIGGEAGSSHAPPPALTIFLDREPIAFMLCYPAARYLMGSMTPQAVLAFVALIPPTLPGTNIVL
GALPEDRHIDRLAKRQRPGERLDLAMLAAIRRVYGLLANTVRYLQGGGSWREDWGQLSGA
AVPPQGAEPQSNAGPRPHIGDTLFTLFRAPELLAPNGDLYNVFAWALDVLAKRLRPMHVFIL
DYDQSPAGCRDALLQLTSGMVQTHVTTPGSIPTICDLARTFAREMGEAN
SEQ ID NO: 5 - Human p16
MEPAAGSSMEPSADWLATAAARGRVEEVRALLEAGALPNAPNSYGRRPIQVMMMGSARVA
ELLLLHGAEPNCADPATLTRPVHDAAREGF LDTLVVLHRAGARLDVRDAWGRLPVDLAEE
LGHRDVARYLRAAAGGTRGSNHARIDAAEGPSDIPD
SEQ ID NO: 6 - Myc-FGF10 portion-(G₄S)₂-p16-His₆
MEQKLISEEDLGSGRHVRSYNHLQGDVRWRKLFSFTKYFLKIEKNGKVSGTKKENCPYSI
LEITSVEIGVVAVKAINSNYYLAMNKKGKLYGSKEFNNDCKLKERIEENGYNTYASFNWQ
HNGRQMYVALNGKGAPRRGQKTRRKNTSAHFLPMVVHSGHGGGGSGGGGSMEPAAGSSM
E
PSADWLATAAARGRVEEVRALLEAGALPNAPNSYGRRPIQVMMMGSARVAELLLLHGAEP
NCADPATLTRPVHDAAREGFLDTLVVLHRAGARLDVRDAWGRLPVDLAEELGHRDVARYL
RAAAGGTRGSNHARIDAAEGPSDIPDGHGHHHHHH
SEQ ID NO: 7 - His₆-p16-(G₄S)₂-FGF10 portion-Myc
MHHHHHHGSMERAAGSSMEPSADWLATAAARGRVEEVRALLEAGALPNAPNSYGRRPIQV
MMMGSARVAELLLLHGAEPNCADPATLTRPVHDAAREGFLDTLVVLHRAGARLDVRDAW
G
RLPVDLAEELGHRDVARYLRAAAGGTRGSNHARIDAAEGPSDIPDGHGGGGSGGGGSGRH
VRSYNHLQGDVRWRKLFSFTKYFLKIEKNGKVSGTKKENCPYSILEITSVEIGVVAVKAI
NSNYYLAMNKKGKLYGSKEFNNDCKLKERIEENGYNTYASFNWQHNGRQMYVALNGKGA
P
RRGQKTRRKNTSAHFLPMVVHSGHGEQKLISEEDL
SEQ ID NO: 8 - variant domain of FGF-10 (residues 64-208
of full length, unprocessed, naturally occurring human
FGF-10) having E158K/K195A [where number of the variant
residue is relative to full length, unprocessed, naturally
occurring human FGF-10]
GRHVRSYNHLQGDVRWRKLFSFTKYFLKIEKNGKVSGTKKENCPYSILEITSVEIGVVAV
KAINSNYYLAMNKKGKLYGSKEFNNDCKLKERIEKNGYNTYASFNWQHNGRQMYVALNG
KGAPRRGQKTRRANTSAHFLPMVVHS
SEQ ID NO: 9 - variant domain of FGF-10 (residues 64-208
of full length, unprocessed, naturally occurring human
FGF-10) having R78A [where number of the variant residue
is relative to full length, unprocessed, naturally
occurring human FGF-10]
GRHVRSYNHLQGDVAWRKLFSFTKYFLKIEKNGKVSGTKKENCPYSILEITSVEIGVVAV
KAINSNYYLAMNKKGKLYGSKEFNNDCKLKERIEENGYNTYASFNWQHNGRQMYVALNG
KGAPRRGQKTRRKNTSAHFLPMVVHS
SEQ ID NO: 10 - variant domain of FGF-10 (residues 64-208
of full length, unprocessed, naturally occurring human
FGF10) having four amino acid substitutions (shown bolded
and underlined)
GRHVRSYNHLQGDVAWRKLFSFTKYFLKIEKNGKVSGTKKENCPYSILEIRSVEIGV
VAVKAINSNYYLAMNKKGKLYGSKEFNNDCKLKERIEANGYNTYASFNWQHNGR
QMYVALNGKGAPRRGQKTRRANTSAHFLPMVVHS
Note: this tetramutant has the following substitutions -
where the residue number for the variant positions in
this variant is relative to residue numbering of full
length, unprocessed, naturally occurring human FGF10.
This tetramutant does not change the net theoretical
charge of the FGF10 portion but decreases intrinsic
mitogenic activity of FGF10.
- R to A (position 78; R78A)
- T to R (position 114; T114R)
- E to A (position 158; E158A)
- K to A (position 195; K195A)
SEQ ID NO: 11 - myc-(mut)FGF10-GS10-mCherry-Hisx6;
The (mut)FGF10 region is shown in bold and is the
tetramutant presented in SEQ ID NO: 10. Variant
residues are underlined. (Mutated residues relative
to wild type sequence are highlighted in yellow.)
MEQKLISEEDLGSGRHVRSYNHLQGDVAWRKLESFTKYFLKIEKNGKVSGTKKE
NCPYSILEIRSVEIGVVAVKAINSNYYLAMNKKGKLYGSKEFNNDCKLKERIEAN
GYNTYASFNWQHNGRQMYVALNGKGAPRRGQKTRRANTSAHFLPMVVHSGH
GGGGSGGGGSVSKGEEDNMAIIKEFMRFKVHMEGSVNGHEFEIEGEGEGRPYEGTQ
TAKLKVTKGGPLPFAWDILSPQFMYGSKAYVKHPADIPDYIKLSFPEGFKWERVMN
FEDGGVVTVTQDSSLQDGEFIYKVKLRGTNFPSDGPVMQKKTMGWEASSERMYPE
DGALKGEIKQRLKLKDGGHYDAEVKTTYKAKKPVQLPGAYNVNIKLDITSHNEDYT
IVEQYERAEGRHSTGGMDELYKLEGHGHHHHHH

We claim:

1. A complex comprising
an FGF-10 portion comprising a domain of a full length, unprocessed, naturally occurring fibroblast growth factor receptor 10 (FGF-10) polypeptide having a net positive charge, surface positive charge, a molecular weight of at least 4 kDa, and a charge per molecular weight ratio greater than that of a corresponding full length, unprocessed, naturally occurring FGF-10 polypeptide, which domain is a variant having one, two, three, four, or five amino acid substitutions, deletions, or additions relative to the corresponding domain of the naturally occurring FGF-10 polypeptide and that retains cell penetrating activity and
a cargo portion comprising a heterologous protein or peptide or a small organic molecule;
wherein the complex does not include a full length, unprocessed, naturally occurring FGF-10 polypeptide.

2. A complex comprising
an FGF-10 portion comprising the amino acid sequence set forth in SEQ ID NO: 2, in the presence of one, two, three, four, or five amino acid substitutions relative to the amino acid set forth in SEQ ID NO: 2, wherein the FGF-10 portion (i) has decreased binding affinity for FGFR2b relative to a naturally occurring, mature FGF-10 polypeptide and/or (ii) has decreased mitogenic activity relative to a naturally occurring, mature FGF-10 polypeptide and
a cargo portion comprising a heterologous protein or peptide or a small organic molecule.

3. The complex of claim 1 or 2, wherein the FGF-10 portion has decreased binding affinity for FGFR2b relative to a naturally occurring, mature FGF-10 polypeptide.

4. The complex of any of claims 1-3, wherein the FGF-10 portion has decreased mitogenic activity relative to a naturally occurring, mature FGF-10 polypeptide.

5. The complex of any of claims 1-4, wherein the FGF-10 portion comprises the amino acid sequence set forth in SEQ ID NO: 2, in the presence of one, two, three, or four amino acid substitutions relative to the amino acid sequence set forth in SEQ ID NO: 2.

6. The complex of any of claims 1-5, wherein the FGF-10 portion comprises the amino acid sequence set forth in SEQ ID NO: 8 or 9.

7. The complex of any of claims 1-6, wherein the FGF-10 portion comprises one, two, three, or four amino acid substitutions relative to the amino acid sequence set forth in SEQ ID NO: 2 selected from R78A, T114A, E158K, E158A, and K195A.

8. The complex of any of claims 1-7, wherein the complex does not include the amino acid sequence set forth in SEQ ID NO: 1.

9. The complex of any of claims 1-8, wherein the cargo portion or complex does not include an FGF receptor or a ligand binding domain of an FGF receptor or a polypeptide or peptide that endogenously binds FGF-10 in vivo under physiological conditions.

10. The complex of any of claims 1-9, wherein the cargo portion does not comprise an antibody or antibody fragment.

11. A complex comprising
an FGF-10 portion comprising SEQ ID NO: 10 and
a cargo portion comprising a heterologous protein or peptide or a small organic molecule, wherein the heterologous protein or peptide is a therapeutic protein, and wherein the cargo portion does not comprise an antibody or antibody fragment.

12. A complex comprising
an FGF-10 portion comprising a domain of a full length, unprocessed, naturally occurring fibroblast growth factor receptor 10 (FGF-10) polypeptide having a net positive charge, surface positive charge, a molecular weight of at least 4 kDa and a charge per molecular weight ratio greater than that of a corresponding full length, unprocessed, naturally occurring FGF-10 polypeptide and
a cargo portion comprising a heterologous protein or peptide or a small organic molecule;
wherein the complex does not include a full length, unprocessed, naturally occurring FGF-10 polypeptide.

13. A complex comprising
an FGF-10 portion comprising a domain of a full length, unprocessed, naturally occurring fibroblast growth factor receptor 10 (FGF-10) polypeptide having a net positive charge, surface positive charge, and a charge per molecular weight ratio greater than that of a corresponding full length, unprocessed, naturally occurring FGF-10 polypeptide, which domain is a variant that retains cell penetrating activity and
a cargo portion comprising a heterologous protein or peptide or a small organic molecule;
wherein the complex does not include a full length, unprocessed, naturally occurring FGF-10 polypeptide.

14. A complex comprising
an FGF-10 portion comprising a cell penetrating variant of a full length, unprocessed, naturally occurring fibroblast growth factor receptor 10 (FGF-10) polypeptide and
a cargo portion comprising a heterologous protein or peptide or a small organic molecule.

15. A complex suitable for cell penetration comprising
an FGF-10 portion comprising (i) a full length, unprocessed, naturally occurring fibroblast growth factor receptor 10 (FGF-10) polypeptide or (ii) a mature, naturally occurring fibroblast growth factor 10 (FGF-10) polypeptide and
a cargo portion comprising a heterologous protein or peptide or a small organic molecule for delivery into a cell.

16. The complex of any of claims 1-15, wherein the complex further comprises a linker that interconnects the FGF-10 portion and the cargo portion.

17. The complex of any of claims 1-16, wherein the FGF-10 polypeptide is a human FGF-10 polypeptide.

18. The complex of any of claims 1-17, wherein the domain of a full length, unprocessed, naturally occurring FGF-10 polypeptide is a domain of a full length, unprocessed, naturally occurring human FGF-10 polypeptide.

19. The complex of any of claims 1-18, wherein the variant comprises one, two, three, four, or five amino acid substitutions, deletions, and/or additions relative to the corresponding domain of the naturally occurring FGF-10 polypeptide.

20. The complex of claim 19, wherein the variant has decreased binding affinity for FGFR2b relative to a naturally occurring, mature FGF-10 polypeptide.

21. The complex of claim 19 or 20, wherein the variant has decreased mitogenic activity relative to a naturally occurring, mature FGF-10 polypeptide.

22. The complex of any of claims 1-18, wherein the FGF-10 portion, and/or the domain and/or the complex has decreased binding affinity for FGFR2b relative to a naturally occurring, mature FGF-10 polypeptide.

23. The complex of any of claims 1-18 or 22, wherein the FGF-10 portion, and/or the domain and/or the complex has decreased mitogenic activity relative to a naturally occurring, mature FGF-10 polypeptide.

24. The complex of any of claims 17-23, wherein the domain has a charge/molecular weight ratio of at least 0.75, but the full length, unprocessed, naturally occurring human FGF-10 polypeptide has a charge/molecular weight ratio of less than 0.75.

25. The complex of any of claims 1-24, wherein the domain has a charge per molecular weight ratio greater than that of the naturally occurring, mature form of the corresponding FGF-10 polypeptide.

26. The complex of any of claims 17-25, wherein, other than the domain, the complex does not include sufficient additional amino acid sequence from said FGF-10 polypeptide contiguous with said domain such that the charge/molecular weight ratio of the FGF-10 portion would be less than 0.75.

27. The complex of any of claims 1-26, wherein the domain is less than 171 amino acid residues.

28. The complex of any of claims 1-26, wherein the domain is less than 150 amino acid residues.

29. The complex of any of claims 1-26, wherein the domain is less than or equal to 145 amino acid residues.

30. The complex of any of claims 1-29, wherein the domain is greater than or equal to 141 amino acid residues.

31. The complex of any of claims 1-30, wherein the domain comprises an amino acid sequence set forth in SEQ ID NO: 2.

32. The complex of any of claims 1-30, wherein the domain is a variant having one, two, three, four, or five amino acid substitutions, deletions, or additions relative to the amino acid sequence set forth in SEQ ID NO: 2.

33. The complex of claim 32, wherein the variant has decreased binding affinity for FGFR2b relative to a naturally occurring, mature FGF-10 polypeptide.

34. The complex of claim 32 or 33, wherein the variant has decreased mitogenic activity relative to a naturally occurring, mature FGF-10 polypeptide.

35. The complex of claim 31 or 32, wherein the FGF-10 portion and/or the domain and/or the complex has decreased binding affinity for FGFR2b relative to a naturally occurring, mature FGF-10 polypeptide.

36. The complex of claim 31, 32, or 35, wherein the FGF-10 portion and/or the domain and/or the complex has decreased mitogenic activity relative to a naturally occurring, mature FGF-10 polypeptide.

37. The complex of any of claims 1-36, wherein the domain has a charge/molecular weight ratio of at least 1.0.

38. The complex of any of claims 1-36, wherein the domain has a charge/molecular weight ratio of at least 0.9.

39. The complex of any of claims 1-36, wherein the domain has a molecular weight of at least about 14 kDa.

40. The complex of any of claims 1-36, wherein the domain has a molecular weight of at least about 15 kDa.

41. The complex of any of claims 1-36, wherein the domain has a molecular weight of at least about 16 kDa.

42. The complex of any of claims 1-41, wherein the domain has a theoretical net charge of about +12.

43. The complex of any of claims 1-41, wherein the domain has a theoretical net charge of about +14.

44. The complex of any of claims 1-41, wherein the domain has a theoretical net charge of about +16.

45. The complex of any of claims 1-44, wherein the domain does not consist of residues 69-208 of SEQ ID NO: 1.

46. The complex of any of claims 1-44, wherein the domain does not consist of a mature, naturally occurring FGF-10 polypeptide.

47. The complex of any of claims 1-46, wherein the complex can penetrate a cell.

48. The complex of claim 47, wherein the complex can penetrate a liver cell.

49. The complex of any of claims 1-49, wherein the cargo portion comprises a heterologous polypeptide or peptide.

50. The complex of any of claims 1-49, wherein the cargo portion comprises a small organic molecule.

51. The complex of claim 49, wherein the cargo portion does not include a ligand binding domain of an FGF receptor.

52. The complex of claim 49 or 51, wherein the heterologous polypeptide or peptide is an enzyme.

53. The complex of claim 52, wherein the enzyme is selected from a kinase, a phosphatase, a ligase, a protease, an oxidoreductase, a transferase, a hydrolase, a hydroxylase, a lyase, an isomerase, a dehydrogenase, an aminotransferase, a hexosamidase, a glucosidase, or a glucosyltransferase.

54. The complex of claim 52 or 53, wherein the enzyme is selected from an enzyme that degrades glycosaminoglycans, glycolipids, or sphingolipids; an enzyme that degrades glycoproteins; an enzyme that degrades amino acids; or an enzyme that degrades fatty acids; or an enzyme involved in energy metabolism.

55. The complex of claim 52, 53, or 54, wherein the enzyme is an enzyme that is endogenously expressed in healthy subjects.

56. The complex of any of claims 52-55, wherein the enzyme is not a recombinase.

57. The complex of any of claims 52-56, wherein an endogenous activity of the enzyme in healthy subjects is in liver.

58. The complex of claim 53, wherein the enzyme is a thymidine kinase.

59. The complex of claim 49 or 51, wherein the heterologous polypeptide or peptide is a transcription factor.

60. The complex of claim 49 or 51, wherein the heterologous polypeptide or peptide is a tumor suppressor protein.

61. The complex of claim 60, wherein the tumor suppressor protein is p16, or a functional fragment thereof.

62. The complex of claim 49 or 51, wherein the heterologous polypeptide or peptide is a co-factor or member of a protein complex.

63. The complex of claim 49 or 51, wherein the heterologous polypeptide or peptide is a target binding moiety that binds to and inhibits a target.

64. The complex of claim 63, wherein the heterologous polypeptide or peptide comprises an antibody or antibody mimic.

65. The complex of claim 63, wherein the target binding moiety comprises a ligand binding domain of a receptor or a receptor binding domain of a ligand.

66. The complex of claim 63, wherein the target binding moiety comprises a full length antibody molecule.

67. The complex of claim 63, wherein the target binding moiety comprises an antibody fragment.

68. The complex of claim 67, wherein the antibody fragment is a single chain antibody (scFv), a F(ab′)2 fragment, a Fab fragment, or an Fd fragment.

69. The complex of claim 63, wherein the target binding moiety comprises a bispecific antibody.

70. The complex of claim 63, wherein the target binding moiety comprises an antibody-mimic comprising a protein scaffold.

71. The complex of claim 70, wherein the antibody mimic comprises a DARPin polypeptide or an Anticalin® polypeptide.

72. The complex of any of claims 63-71, wherein the target binding moiety binds to and inhibits a target expressed or present in liver.

73. The complex of claim 62, wherein an endogenous activity of the heterologous polypeptide or peptide is as a member of a complex with a polypeptide expressed or present in liver.

74. The complex of any of claims 1-73, wherein the FGF-10 portion and the cargo portion are associated non-covalently.

75. The complex of any of claims 1-73, wherein the FGF-10 portion and the cargo portion are associated via a covalent interconnection.

76. The complex of claim 75, wherein the FGF-10 portion and the cargo portion are interconnected by a linker.

77. The complex of claim 75, wherein the FGF-10 portion and the cargo portion are directly interconnected without a linker.

78. The complex of claim 76, wherein the linker is a peptide linker and the FGF-10 portion and the cargo portion form a fusion protein.

79. The complex of claim 76 or 78, wherein the linker comprises an amide, an ester, or a disulfide bond.

80. The complex of any of claims 76-79, wherein the linker is a cleavable linker.

81. The complex of claim 80, wherein the cleavable linker can be cleaved by a naturally occurring cellular enzyme.

82. The complex of any of claims 1-81, wherein the complex further comprises one or more tags to facilitate production, purification, or detection of the complex.

83. The complex of claim 82, wherein the complex comprises 1, 2, or 3 tags.

84. The complex of claim 83, wherein the complex comprises 2 tags.

85. The complex of any of claims 1-84, wherein the FGF-10 portion is N-terminal to the cargo portion.

86. The complex of any of claims 1-84, wherein the FGF-10 portion is C-terminal to the cargo portion.

87. The complex of any of claims 1-84, comprising the following formula:
FGF10 portion-linker-cargo portion.

88. The complex of claim 87, comprising the formula:
tag1-FGF10 portion-linker-cargo portion-tag2;
wherein the complex may comprise one or both of tag1 or tag2.

89. The complex of any of claims 1-84, comprising the following formula:
cargo portion-linker-FGF10 portion.

90. The complex of claim 89, comprising the formula:
tag1-cargo portion-linker-FGF10 portion-tag2;
wherein the complex may comprise one or both of tag1 or tag2.

91. The complex of any of claims 1-90, wherein the complex is a fusion protein comprising the FGF-10 portion and the cargo portion.

92. A nucleic acid comprising a nucleotide sequence encoding the complex of any of claims 1-91.

93. A nucleic acid comprising a nucleotide sequence encoding the fusion protein of claim 91.

94. A vector comprising the nucleic acid of claim 92 or 93.

95. A host cell comprising the vector of claim 94.

96. A method of making a fusion protein, comprising
(i) providing the host cell of claim 95 in culture media and culturing the host cell under suitable condition for expression of protein therefrom; and
(ii) expressing the fusion protein.

97. The method of claim 96, further comprising isolating the fusion protein from the culture media.

98. The complex of any of claims 1-91, wherein the complex has greater than 50% of the native activity of the cargo portion.

99. The complex of claim 98, wherein the cargo portion is an enzyme, and the complex has greater than 50% of the native activity of the enzyme.

100. The complex of any of claims 1-91, wherein the variant is modified to increase surface positive charge, net charge, and/or charge per molecular weight ratio.

101. The complex of any of claims 1-91 or 98-100, wherein the complex comprises a modification selected from glycosylation, phosphorylation or pegylation.

102. The complex of any of claims 1-91 or 98-100, wherein the complex is not glycosylated.

103. A composition comprising the complex of any of claims 1-91 or 98-100 and a pharmaceutically acceptable carrier.

104. A method of delivering a cargo portion into a cell, comprising
providing the complex of any of claims 1-91 or 98-100 and
contacting cells with the complex.

105. A method of delivering a cargo portion into a cell of the liver, comprising
providing the complex of any of claims 1-91 or 98-100 and
contacting cells with the complex.

106. A method of delivering a therapeutic protein into cells or tissues of the abdominal cavity, comprising
providing the complex of any of claims 1-91 or 98-100 and
administering said complex to a subject in need thereof via intraperitoneal administration.

107. The method of claim 106, wherein the subject in need thereof is a subject with primary or metastatic cancer in the abdominal cavity.

108. The method of claim 107, wherein the primary or metastatic cancer is associated with liver, kidney, pancreas, or ovary.

109. The method of claim 106 or 107, wherein the primary or metastatic cancer comprises a mutation that decreases the expression and/or activity of p16.

110. A method of delivering a target binding moiety into cells, comprising
providing the complex of any of claims 1-49, 51, 63-91, or 98-102 and
administering said complex to a subject in need thereof.

111. The method of claim 110, wherein the target binding moiety binds to and inhibits a target expressed inside the cells.

112. The method of claim 111, wherein the cells are cells of the liver, kidney, pancreas, or ovary.

113. The complex of any of claims 1-91, wherein the FGF-10 portion comprises an E158K/K195A FGF-10 variant.

114. The complex of any of claims 1-91, wherein the FGF-10 portion comprises an R78A FGF-10 variant.

115. The complex of claim 113, wherein the FGF-10 portion comprises an amino acid sequence set forth in SEQ ID NO: 8.

116. The complex of claim 114, wherein the FGF-10 portion comprises an amino acid sequence set forth in SEQ ID NO: 9.

117. A complex comprising the amino acid sequence set forth in SEQ ID NO: 4, in the presence or absence of the N-terminal tag.
